(19) United States
(12) Patent Application Publication
(10) Pub. No.: US 2012/0264636 A1
(43) Pub. Date: Oct. 18, 2012

(54) GENETIC VARIANTS INDICATIVE OF VASCULAR CONDITIONS

(75) Inventors: Hilma Holm, Mosfellsbaer (IS); Daniel Gudbjartsson, Reykjavik (IS)

(73) Assignee: deCODE Genetics ehf., Reykjavik (IS)

(21) Appl. No.: 13/500,264

(22) PCT Filed: Oct. 7, 2010
(86) PCT No.: PCT/IS2010/050008

§ 371 Date: Jun. 25, 2012

(30) Foreign Application Priority Data
Oct. 7, 2009 (IS) 8852

(51) Int. Cl. C40B 30/04; G06F 19/00; G01N 33/566; C40B 40/06; C12Q 1/68; G01N 33/573

(52) U.S. Cl. 506/9; 435/6.11; 435/7.4; 436/501; 506/16; 702/20

(57) ABSTRACT

The invention relates to procedures and methods of determining a susceptibility to certain vascular conditions, including Atrial Fibrillation, Atrial Flutter and Stroke, by assessing the presence or absence of alleles at polymorphic markers found to be associated with these conditions. The invention further relates to kits encompassing reagents for assessing such markers, and diagnostic methods, uses and procedures for utilizing such markers.

### INTRODUCTION

Genetic risk is conferred by subtle differences in the genome among individuals in a population. Variations in the human genome are most frequently due to single nucleotide polymorphisms (SNPs), although other variations are also important. SNPs are located on average every 1000 base pairs in the human genome. Accordingly, a typical human gene containing 250,000 base pairs may contain 250 different SNPs. Only a minor number of SNPs are located in exons and alter the amino acid sequence of the protein encoded by the gene. Most SNPs may have little or no effect on gene function, while others may alter transcription, splicing, translation, or stability of the mRNA encoded by the gene. Additional genetic polymorphisms in the human genome are caused by insertions, deletions, translocations or inversion of either short or long stretches of DNA. Genetic polymorphisms conferring disease risk may directly alter the amino acid sequence of proteins, may increase the amount of protein produced from the gene, or may decrease the amount of protein produced by the gene.

As genetic polymorphisms conferring risk of common diseases are uncovered, genetic testing for such risk factors is becoming increasingly important for clinical medicine. Examples are apolipoprotein E testing to identify genetic carriers of the apoE4 polymorphism in dementia patients for the differential diagnosis of Alzheimer's disease, and of Factor V Leiden testing for predisposition to deep venous thrombosis. More importantly, in the treatment of cancer, diagnosis of genetic variants in tumor cells is used for the selection of the most appropriate treatment regime for the individual patient. In breast cancer, genetic variation in estrogen receptor expression or heregulin type 2 (Her2) receptor tyrosine kinase expression determine if anti-estrogenic drugs (tamoxifen) or anti-Her2 antibody (Herceptin) will be incorporated into the treatment plan. In chronic myeloid leukemia (CML) diagnosis of the Philadelphia chromosome genetic translocation fusing the genes encoding the Bcr and Abl receptor tyrosine kinases indicates that Gleevec (STI571), a specific inhibitor of the Bcr-Abl kinase should be used for treatment of the cancer. For CML patients with such a genetic alteration, inhibition of the Bcr-Abl kinase leads to rapid elimination of the tumor cells and remission from leukemia. Furthermore, genetic testing services are now available, providing individuals with information about their disease risk based on the discovery that certain SNPs have been associated with risk of many of the common diseases.

The electrocardiogram (ECG) is a valuable tool in the assessment of the cardiac conduction system. The measurements routinely obtained with ECG include heart rate (HR), PR interval, QRS duration and the QT interval. These variables are indicative of the function of the conduction system and provide important prognostic information.

A strong correlation between elevated HR and cardiovascular morbidity and mortality has been reported in numerous studies (Palatini, P. & Julius, S. Clin Exp Hypertens 26:637-44 (2004); Bjornsson, S. et al., Laeknabladid 21-7 (1993)). This relationship has not only been demonstrated in patients with cardiovascular diseases (CVD) including hypertension and left ventricular dysfunction but also in the general population (Palatini, P. & Julius, S. Clin Exp Hypertens 26:637-44 (2004)). As an example, a significantly greater risk of sudden cardiac death (SCD) has been associated with resting HR higher than 75 beats per minute in men without history of CVD (Jouven, X. et al. N Engl J Med 352:1951-8 (2005)).

The PR interval and QRS complex are measures of electrical activation. The PR interval reflects the time required for the electrical impulse to travel from the atrial myocardium adjacent to the sinus node (SN), through the atrioventricular node (AVN), and to the Purkinje fibres (Saksena, S. & Camm. J A. Electrophysiological Disorders of the Heart (Elsevier Churchill Livingstone, Philadelphia (2004)). The QRS complex represents depolarization of the ventricles through the Purkinje system and ventricular myocardium (Saksena, S. & Camm. J A. Electrophysiological Disorders of the Heart (Elsevier Churchill Livingstone, Philadelphia (2004)). Delayed conduction in the respective segments of the conduction system, of any cause, results in prolongation of these parameters. Isolated prolongation of the PR interval has generally been perceived as a benign condition but was recently associated with increased risk of atrial fibrillation (AF), pacemaker (PM) implantation and mortality (Cheng. S. et al., JAMA 301:2571-7 (2009). Increased QRS duration, both in the presence and absence of bundle branch block (BBB), has long been associated with less survival (Hesse, B et al., Am J Med 110:253-9 (2001); Desai, A D et al. Am J Med 119:600-6 (2006)).

The QT interval reflects myocardial repolarization. Extremes of the QT interval duration are established risk factors of ventricular arrhythmias and SCD and include well known Mendelian long- and short-QT syndromes commonly due to rare mutations in ion channel genes. There is evidence for a substantial genetic contribution to the cardiac conduction system with a reported heritability for HR in the range of 29-77%, 34% for the PR interval and 30-40% for the QT interval (Newton-Cheh, C. et al., BMC Med Genet. 8 Suppl 1:S7 (2007); Hanson, B. et al., Am J Cariol. 63:606-9 (1989); Havlik, R J et al., J Electrocardio/13:45-8 (1980); Russell, M W et al. J Electrocardiol 30 Suppl:64-8 (1998); Li, J. et al. Ann Nonivvasive Electrocardiol 14:147-52 (2009)). Studies on the QRS duration have reported inconsistent results, ranging from no significant heritable component (Havlik, R J et al. J Electrocardiol 13:45-8 (1980); Russell, M W et al., J Electrocardiol 30 Suppl:64-8 (1998)) to 36-43% heritability (Li, J. et al., Ann Noninvasive Electrocardiol 14:147-52 (2009); Mutikainen, S. et al. Ann Nonivasive Electrocardiol 14:57-64 (2009)).

Several recent genome wide association studies (GWAS) have yielded associations between common sequence variants and ECG variables of which the QT interval has particularly been intensely studied. Variants at NOS1AP have shown the strongest association with the QT interval, identifying a previously unrecognized relationship between the nitric oxide synthase pathway and cardiac repolarization (Arking, D E et al. Nat Genet. 38:644-51 (2006)). Subsequently, association was observed between the NOS1AP variants and risk of SCD in white adults, demonstrating how associations with intermediate traits may not only uncover previously unknown biological mechanisms but also translate to clinical relevance (Kao, W H et al. Circulation 119:940-51 (2009)).

Many additional associations were recently reported by two large QT interval meta-analyses on individuals of European ancestry, including both novel associations and with variants in genes known to be involved in myocardial repolarization and Mendelian QT syndromes (Newton-Cheh, C. et al. Nat Genet. 41:399-406 (2009); Pfeufer, A. et al. Nat Genet. 41:407-14 (2009)). Additionally, a recent GWAS in two population based series recruited in Korea revealed two loci with genome-wide significant (GWS) association with HR (rs12731740 and rs12110693) (Cho, Y S et al. Nat Genet. 41:527-34 (2009)) and a study in a South Pacific islander population (Kosrae) showed suggestive association between the PR interval and common variations in SCN5A (rs7638909 and rs2070488) (Smith, J G et al. Heart Rhythm 6:634-41 (2009)). To our knowledge, the two latter associations have not been assessed in individuals of European origin.

Atrial fibrillation (AF) is an abnormal heart rhythm (cardiac arrhythmia) which involves the two small, upper heart chambers (the atria). Heart beats in a normal heart begin after electricity generated in the atria by the sinoatrial node spreads through the heart and causes contraction of the heart muscle and pumping of blood. In AF, the regular electrical impulses of the sinoatrial node are replaced by disorganized, rapid electrical impulses which result in irregular heart beat.

Atrial fibrillation is the most common cardiac arrhythmia. The risk of developing atrial fibrillation increases with age—AF affects four percent of individuals in their 80s. An individual may spontaneously alternate between AF and a normal rhythm (paroxysmal atrial fibrillation) or may continue with AF as the dominant cardiac rhythm without reversion to the normal rhythm (chronic atrial fibrillation). Atrial fibrillation is often asymptomatic, but may result in symptoms of palpitations, fainting, chest pain, or even heart failure. These symptoms are especially common when atrial fibrillation results in a heart rate which is either too fast or too slow. In addition, the erratic motion of the atria leads to blood stagnation (stasis) which increases the risk of blood clots that may travel from the heart to the brain and other areas. Thus, AF is an important risk factor for stroke, the most feared complication of atrial fibrillation.

The symptoms of atrial fibrillation may be treated with medications which slow the heart rate. Several medications as well as electrical cardioversion may be used to convert AF to a normal heart rhythm. Surgical and catheter-based therapies may also be used to prevent atrial fibrillation in certain individuals. People with AF are often given blood thinners such as warfarin to protect them from strokes.

Any patient with 2 or more identified episodes of atrial fibrillation is said to have recurrent atrial fibrillation. This is further classified into paroxysmal and persistent based on when the episode terminates without therapy. Atrial fibrillation is said to be paroxysmal when it terminates spontaneously within 7 days, most commonly within 24 hours. Persistent or chronic atrial fibrillation is AF established for more than seven days. Differentiation of paroxysmal from chronic or established AF is based on the history of recurrent episodes and the duration of the current episode of AF (Levy S., J Cardiovasc Electrophysiol. 8 Suppl, S78-82 (1998)).

Lone atrial fibrillation (LAF) is defined as atrial fibrillation in the absence of clinical or echocardiographic findings of cardiopulmonary disease.

Atrial fibrillation is usually accompanied by symptoms related to either the rapid heart rate or embolization. Rapid and irregular heart rates may be perceived as palpitations, exercise intolerance, and occasionally produce angina and congestive symptoms of shortness of breath or edema. Sometimes the arrhythmia will be identified with the onset of a stroke or a transient ischemic attack (TIA). It is not uncommon to identify atrial fibrillation on a routine physical examination or electrocardiogram (ECG/EKG), as it may be asymptomatic in some cases. Paroxysmal atrial fibrillation is the episodic occurrence of the arrhythmia and may be difficult to diagnose. Episodes may occur with sleep or with exercise, and their episodic nature may require prolonged ECG monitoring (e.g. a Holter monitor) for diagnosis.

Atrial fibrillation is diagnosed on an electrocardiogram, an investigation performed routinely whenever irregular heart beat is suspected. Characteristic findings include absence of P waves, unorganized electrical activity in their place and irregularity of R-R interval due to irregular conduction of impulses to the ventricles. If paroxysmal AF is suspected, episodes may be documented with the use of Holter monitoring (continuous ECG recording for 24 hours or longer).

While many cases of AF have no definite cause, it may be the result of various other problems (see below). Hence, renal function and electrolytes are routinely determined, as well as thyroid-stimulating hormone and a blood count. A chest X-ray is generally performed. In acute-onset AF associated with chest pain, cardiac troponins or other markers of damage to the heart muscle may be ordered. Coagulation studies (INR/aPTT) are usually performed, as anticoagulant medication may be commenced. A transesophageal echocardiogram may be indicated to identify any intracardiac thrombus (Fuster V., et al., Circulation; 104, 2118-2150 (2001)).

Atrial Flutter (AFI) is characterized by an abnormal fast heart rhythm in the atria. Patients who present with atrial flutter commonly also experience Atrial Fibrillation and vice versa (Waldo, A., Progr Cardiovasc Disease, 48:41-56 (2005)). Mechanistically and biologically, AF and AFI are thus likely to be highly related.

AF (and AFI) is linked to several cardiac causes, but may occur in otherwise normal hearts. Known associations include: High blood pressure, Mitral stenosis (e.g. due to rheumatic heart disease or mitrel valve prolapse), Mitral regurgitation, Heart surgery, Coronary artery disease, Hypertrophic cardiomyopathy, Excessive alcohol consumption (“binge drinking” or “holiday heart”), Hyperthyroidism, Hyperstimulation of the vagus nerve, usually by having large meals (“binge eating”), Lung pathology (such as pneumonia, lung cancer, pulmonary embolism, Sarcoidosis), Pericarditis, Intense emotional turmoil, and Congenital heart disease.

The normal electrical conduction system of the heart allows the impulse that is generated by the sinoatrial node (SA node) of the heart to be propagated to and stimulate the myocardium (muscle of the heart). When the myocardium is stimulated, it contracts. It is the ordered stimulation of the myocardium that allows efficient contraction of the heart, thereby allowing blood to be pumped to the body. In atrial fibrillation, the regular impulses produced by the sinus node to provide rhythmic contraction of the heart are overwhelmed by the rapid randomly generated discharges produced by larger areas of atrial tissue. An organized electrical impulse in the atrium produces atrial contraction; the lack of such an impulse, as in atrial fibrillation, produces stagnant blood flow, especially in the atrial appendage and predisposes to clotting. The dislodgement of a clot from the atrium results in an embolus, and the damage produced is related to where the circulation takes it. An embolus to the brain produces the most feared complication of atrial fibrillation, stroke, while an embolus may also lodge in the mesenteric circulation (the circulation supplying the abdominal organs) or digit, producing organ-specific damage.

Treatment of atrial fibrillation is directed by two main objectives: (i) prevent temporary circulatory instability; (ii) prevent stroke. The most common methods for achieving the former includes rate and rhythm control, while anticoagulation is usually the desired method for the latter (Prystowsky E. N., Am J Cardiol.; 85, 3D-11D (2000); van Walraven C, et al., Jama. 288, 2441-2448 (2002)). Common methods for rate control, i.e. for reducing heart rate to normal, include beta blockers (e.g., metotprolol), cardiac glycosides (e.g., digoxin) and calcium channel blockers (e.g., verapamil). All these medications work by slowing down the generation of pulses from the atria, and the conduction from the atria to the ventricles. Other drugs commonly used include quinidine, flecamide, propafenone, disopyramide, sotalol and amiodarone. Rhythm control can be achieved by electrical cardioversion, i.e. by applying DC electrical shock, or by chemical cardioversion, using drugs such as amiodarione, propafenone and flecamide.

Preventive measures for stroke include anticoagulants. Representative examples of anticoagulant agents are Dalteparin (e.g., Fragmin), Danaparoid (e.g., Orgaran), Enoxaparin (e.g., Lovenox), Heparin (various), Tinzaparin (e.g., Innohep), Warfarin (e.g., Coumadin). Some patients with lone atrial fibrillation are sometimes treated with aspirin or clopidogrel. There is evidence that aspirin and clopidogrel are effective when used together, but the combination is still inferior to warfarin (Connolly S., et al. Lancet; 367, 1903-1912 (2006)). (2) The new anticoagulant ximelagatran has been shown to prevent stroke with equal efficacy as warfarin, without the difficult monitoring process associated with warfarin and with possibly fewer adverse haemorrhagic events. Unfortunately, ximegalatran and other similar anticoagulant drugs (commonly referred to as direct thrombin inhibitors), have yet to be widely licensed.

Determining who should and should not receive anti-coagulation with warfarin is not straightforward. The CHADS2 score is the best validated method of determining risk of stroke (and therefore who should be anticoagulated). The UK NICE guidelines have instead opted for an algorithm approach. The underlying problem is that if a patient has a yearly risk of stroke that is less than 2%, then the risks associated with taking warfarin outweigh the risk of getting a stroke (Gage B. F. et al. Stroke 29, 1083-1091 (1998))

Atrial fibrillation can sometimes be controlled with treatment. The natural tendency of atrial fibrillation, however, is to become a chronic condition. Chronic AF leads to an increased risk of death. Patients with atrial fibrillation are at significantly increased chance of stroke.

Atrial fibrillation is common among older adults. In developed countries, the number of patients with atrial fibrillation is likely to increase during the next 50 years, due to the growing proportion of elderly individuals (Go A. S. et al., Jama., 285, 2370-2375 (2001))(3). In the Framingham study the lifetime risk for development of AF is 1 in 4 for men and women 40 years of age and older. Lifetime risks for AF are high (1 in 6). According to data from the National Hospital Discharge Survey (1996-2001) on cases that included AF as a primary discharge diagnosis found that 45% of the patients are male, and that the mean age for men was 66.8 years and 74.6 for women. The racial breakdown for admissions was found to be 71.2% white, 5.6% black, 2% other races, and 20% not specified. Furthermore, African American patients were, on average, much younger than other races. The incidence in men ranged from 20.58/100,000 persons per year for patients ages 15-44 years to 1203/100,000 persons per years for those ages 85 and older. From 1996-2001, hospitalizations with AF as the first listed diagnosis, has increased by 34%.

Stroke is a common and serious disease. Each year in the United States more than 600,000 individuals suffer a stroke and more than 160,000 die from stroke-related causes (Sacco, R. L. et al., Stroke 28, 1507-17 (1997)). Furthermore, over 300,000 individuals present with Transient Ischemic Attack, a mild form of stroke, every year in the US. In western countries stroke is the leading cause of severe disability and the third leading cause of death (Bonita, R., Lancet 339, 342-4 (1992)). The lifetime risk of those who reach the age of 40 exceeds 10%.

The clinical phenotype of stroke is complex but is broadly divided into ischemic (accounting for 80-90%) and hemorrhagic stroke (10-20%) (Caplan, L. R. Caplan's Stroke: A Clinical Approach, 1-556 (Butterworth-Heinemann, 2000)). Ischemic stroke is further subdivided into large vessel occlusive disease (referred to here as carotid stroke), usually due to atherosclerotic involvement of the common and internal carotid arteries, small vessel occlusive disease, thought to be a non-atherosclerotic narrowing of small end-arteries within the brain, and cardiogenic stroke due to blood clots arising from the heart usually on the background of atrial fibrillation or ischemic (atherosclerotic) heart disease (Adams, H. P., Jr. et al., Stroke 24, 35-41 (1993)). Therefore, it appears that stroke is not one disease but a heterogeneous group of disorders reflecting differences in the pathogenic mechanisms (Alberts, M. J. Genetics of Cerebrovascular Disease, 386 (Futura Publishing Company, Inc., New York, 1999); Hassan, A. & Markus, H. S. Brain 123, 1784-812 (2000)). However, all forms of stroke share risk factors such as hypertension, diabetes, hyperlipidemia, and smoking (Sacco, R. L. et al., Stroke 28, 1507-17 (1997); Leys, D. et al., J. Neurol. 249, 507-17 (2002)). Family history of stroke is also an independent risk factor suggesting the existence of genetic factors that may interact with environmental factors (Hassan, A. & Markus, H. S. Brain 123, 1784-812 (2000); Brass, L. M. & Alberts, M. J. Baillieres Clin. Neurol. 4, 221-45 (1995)).

The genetic determinants of the common forms of stroke are still largely unknown. There are examples of mutations in specific genes that cause rare Mendelian forms of stroke such as the Notch3 gene in CADASIL (cerebral autosomal dominant arteriopathy with subcortical infarctions and leukoencephalopathy) (Tournier-Lasserve, E. et al., Nat. Genet. 3, 256-9 (1993); Joutel, A. et al., Nature 383, 707-10 (1996)), Cystatin C in the Icelandic type of hereditary cerebral hemorrhage with amyloidosis (Palsdottir, A. et al., Lancet 2, 603-4 (1988)), APP in the Dutch type of hereditary cerebral hemorrhage (Levy, E. et al., Science 248, 1124-6 (1990)) and the KRIT1 gene in patients with hereditary cavernous angioma (Gunel, M. et al., Proc. Natl. Acad. Sci. USA 92, 6620-4 (1995); Sahoo, T. et al., Hum. Mol. Genet. 8, 2325-33 (1999)). None of these rare forms of stroke occur on the background of atherosclerosis, and therefore, the corresponding genes are not likely to play roles in the common forms of stroke which most often occur with atherosclerosis.

It is very important for the health care system to develop strategies to prevent stroke. Once a stroke happens, irreversible cell death occurs in a significant portion of the brain supplied by the blood vessel affected by the stroke. Unfortunately, the neurons that die cannot be revived or replaced from a stem cell population. Therefore, there is a need to prevent strokes from happening in the first place. Although we already know of certain clinical risk factors that increase stroke risk (listed above), there is an unmet medical need to define the genetic factors involved in stroke to more precisely define stroke risk. Further, if predisposing alleles are common in the general population and the specificity of predicting a disease based on their presence is low, additional loci such as protective loci are needed for meaningful prediction of disposition of the disease state. There is also a great need for therapeutic agents for preventing the first stroke or further strokes in individuals who have suffered a previous stroke or transient ischemic attack.

AF is an independent risk factor for stroke, increasing risk about 5-fold. The risk for stroke attributable to AF increases with age. AF is responsible for about 15-20% of all strokes. AF is also an independent risk factor for stroke recurrence and stroke severity. A recent report showed people who had AF and were not treated with anticoagulants had a 2.1-fold increase in risk for recurrent stroke and a 2.4 fold increase in risk for recurrent severe stroke. People who have stroke caused by AF have been reported as 2.23 times more likely to be bedridden compared to those who have strokes from other causes.

There is a need for an understanding of the susceptibility factors leading to increased predisposition to abnormal ECG measures, and their effect on cardiac arrhythmias and stroke. Identification of such variants can, for example, be useful for assessing which individuals are at particularly high risk of these disorders. Furthermore, preventive treatment and appropriate monitoring can be performed for individuals carrying one or more at-risk variants. Finally, identification of at-risk variants can lead to the identification of new targets for drug therapy, as well as the development of novel therapeutic measures.

### SUMMARY OF THE INVENTION

It has been discovered that certain genetic variants are correlated with electrocardiogram (ECG) measures and risk of disease states that are related with abnormal ECG measures. Such genetic variants are useful in a range of applications, as described further herein, including various diagnostic applications for determining risk of abnormal ECG measures and diseases such as Atrial Fibrillation, Atrial Flutter and Stroke.

In a first aspect, the invention provides a method of determining a susceptibility to a vascular condition selected from the group consisting of: an abnormal electrocardiogram measure, Atrial Fibrillation, Atrial Flutter, and Stroke, in a human individual, the method comprising: obtaining sequence data about a human individual identifying at least one allele of at least one polymorphic marker, wherein different alleles of the at least one polymorphic marker are associated with different susceptibilities to the condition in humans, and determining a susceptibility to the condition from the sequence data, wherein the at least one polymorphic marker is selected from the group consisting of rs3825214, rs6795970, rs3807989, rs7660702, rs132311, rs1733724 and rs365990, and markers in linkage disequilibrium therewith.

Another aspect relates to a method of determining a susceptibility to a vascular condition selected from the group consisting of: an abnormal electrocardiogram measure, Atrial Fibrillation, Atrial Flutter, and Stroke, in a human individual, the method comprising analyzing sequence data about a human individual identifying at least one allele of at least one polymorphic marker, wherein different alleles of the at least one polymorphic marker are associated with different susceptibilities to the condition in humans, and determining a susceptibility to the condition from the sequence data,

wherein the at least one polymorphic marker is selected from the group consisting of rs3825214, rs6795970, rs3807989, rs7660702, rs132311, rs1733724 and rs365990, and markers in linkage disequilibrium therewith.

In another aspect, the method comprises determining the presence or absence of at least one allele of at least one polymorphic marker in a nucleic acid sample obtained from the individual, or in a genotype dataset from the subject, wherein the at least one polymorphic marker is selected from the group consisting of rs3825214, rs6795970, rs3807989, rs7660702, rs132311, rs1733724 and rs365990, and markers in linkage disequilibrium therewith, and wherein determination of the presence of the at least one allele is indicative of a susceptibility to the condition. In certain embodiments, the allele is an allele that confers an increased risk of the condition (an at-risk allele). In such embodiments, determination of the presence of the allele is indicative of increased susceptibility to the condition, whereas determination of the absence of the allele is indicative of the subject not increased susceptibility to the condition that is conferred by the allele.

The invention further relates to a method of assessing a susceptibility to a vascular condition selected from the group consisting of: an abnormal electrocardiogram measure, Atrial Fibrillation, Atrial Flutter, and Stroke, in a human subject, comprising (i) obtaining sequence information about the subject for at least one polymorphic marker selected from the group consisting of rs3825214, rs6795970, rs3807989, rs7660702, rs132311, rs1733724 and rs365990, and markers in linkage disequilibrium therewith, wherein different alleles of the at least one polymorphic marker are associated with different susceptibilities to the condition in humans; and (ii) identifying the presence or absence of at least one allele in the at least one polymorphic marker that correlates with increased occurrence of the condition in humans; wherein determination of the presence of the at least one allele identifies the subject as having elevated susceptibility to the condition, and wherein determination of the absence of the at least one allele identifies the subject as not having the elevated susceptibility.

The invention also provides a method of assessing a subject's risk of a vascular condition selected from the group consisting of: an abnormal electrocardiogram measure, Atrial Fibrillation, Atrial Flutter, and Stroke, the method comprising: obtaining sequence information about the individual identifying at least one allele of at least one polymorphic marker in the genome of the individual; representing the sequence information as digital genetic profile data; transforming the digital genetic profile data to generate a risk assessment report of the condition for the subject; and displaying the risk assessment report on an output device; wherein the at least one polymorphic marker comprises at least one marker selected from the group consisting of rs3825214, rs6795970, rs3807989, rs7660702, rs132311, rs1733724 and rs365990, and markers in linkage disequilibrium therewith.

The digital genetic profile data suitably comprises data about at least one polymorphic marker in the genome of the subject, such as allele counts for at least one allele of the marker, or data identifying both alleles of the marker in the genome of the individual.

The genetic profile data is suitably transformed into a risk measure using a computer processor. The risk assessment report may be in any suitable format for delivering risk assessment information about the subject. In certain embodiments, the report comprises at least one identifier for the subject and a numerical value for at least one risk measure for the individual for at least one polymorphic marker.

Further provided is a method of determining a susceptibility to a vascular condition selected from the group consisting of: an abnormal electrocardiogram measure, Atrial Fibrillation, Atrial Flutter, and Stroke, the method comprising: obtaining sequence data about a human individual identifying at least one allele of at least one polymorphic marker, wherein different alleles of the at least one polymorphic marker are associated with different susceptibilities to the condition in humans; and determining a susceptibility to the condition from the sequence data; wherein the at least one polymorphic marker is a marker associated with a gene selected from the group consisting of: the human TBX5 gene, the human SCN10A gene, the human CAV1 gene, the human ARHGAP24 gene, the human CDKN1A gene and the human MYH6 gene.

In certain embodiments, the at least one marker associated with the human TBX5 gene is selected from the group consisting of rs3825214, and markers in linkage disequilibrium therewith; the at least one marker associated with the human SCN10A gene is selected from the group consisting of rs6795970, and markers in linkage disequilibrium therewith; the at least one marker associated with the human CAVI gene is selected from the group consisting of rs3807989, and markers in linkage disequilibrium therewith; the at least one marker associated with the human ARHGAP24 gene is selected from the group consisting of rs7660702, and markers in linkage disequilibrium therewith; the at least one marker associated with the human CDKN1A gene is selected from the group consisting of rs132311, and markers in linkage disequilibrium therewith; and the at least one marker associated with the human MYH6 gene is selected from the group consisting of rs365990, and markers in linkage disequilibrium therewith.

The invention also provides a method of identification of a marker for use in assessing susceptibility to a vascular condition selected from the group consisting of: an abnormal electrocardiogram measure, Atrial Fibrillation, Atrial Flutter, and Stroke, in human individuals, the method comprising
a. identifying at least one polymorphic marker in linkage disequilibrium with at least one marker selected from the group consisting of rs3825214, rs6795970, rs3807989, rs7660702, rs132311, rs1733724 and rs365990;
b. obtaining sequence information about the at least one polymorphic marker in a group of individuals diagnosed with the condition; and
c. obtaining sequence information about the at least one polymorphic marker in a group of control individuals;
   wherein determination of a significant difference in frequency of at least one allele in the at least one polymorphism in individuals diagnosed with the condition as compared with the frequency of the at least one allele in the control group is indicative of the at least one polymorphism being useful for assessing susceptibility to the condition. In certain embodiments, an increase in frequency of the at least one allele in the at least one polymorphism in individuals diagnosed with the condition, as compared with the frequency of the at least one allele in the control group, is indicative of the at least one polymorphism being useful for assessing increased susceptibility to the condition; and a decrease in frequency of the at least one allele in the at least one polymorphism in individuals diagnosed with the condition, as compared with the frequency of the at least one allele in the control group, is indicative of the at least one polymorphism being useful for assessing decreased susceptibility to, or protection against, the condition.

Disease management methods that are made possible by the present invention also include a method of predicting prognosis of an individual diagnosed with a vascular condition selected from the group consisting of: an abnormal electrocardiogram measure, Atrial Fibrillation, Atrial Flutter, and Stroke, the method comprising: obtaining sequence data about a human individual identifying at least one allele of at least one polymorphic marker selected from the group consisting of rs3825214, rs6795970, rs3807989, rs7660702, rs132311, rs1733724 and rs365990, and markers in linkage disequilibrium therewith, wherein different alleles of the at least one polymorphic marker are associated with different susceptibilities to the conditions in humans, and predicting prognosis of the condition from the sequence data. For example, determination of the presence of an allele that correlates with an abnormal ECG measure or confers increased risk of Atrial Fibrillation, Atrial Flutter and/or Stroke, may be predictive of a more severe prognosis of disease. For such individuals, a more aggressive course of clinical treatment or preventive measure may be appropriate, as described further herein.

The invention also provides a method for selecting a clinical course of therapy to treat a subject who is at risk for developing a vascular condition selected from the group consisting of: an abnormal electrocardiogram measure, Atrial Fibrillation, Atrial Flutter, and Stroke, comprising the steps of: (a) obtaining sequence data about a human subject identifying at least one allele of at least one polymorphic marker, wherein different alleles of the at least one polymorphic marker are associated with different susceptibilities to the condition in humans; (b) transforming the sequence data into a risk measure of the condition for the subject; (c) selecting a clinical course of therapy for treatment of a subject who is determined to be at an increased risk for developing the condition; wherein the at least one polymorphic marker is selected from the group consisting of rs3825214, rs6795970, rs3807989, rs7660702, rs132311, rs1733724 and rs365990, and markers in linkage disequilibrium therewith.

The invention also provides methods for assessing response to therapeutic agents. In one such aspect, a method of assessing probability of response of a human individual to a therapeutic agent for preventing, treating and/or ameliorating symptoms associated with a vascular condition selected from the group consisting of: an abnormal electrocardiogram measure, Atrial Fibrillation, Atrial Flutter, and Stroke, is provided, the method comprising the steps of: obtaining sequence data about a human individual identifying at least one allele of at least one polymorphic marker selected from the group consisting of rs3825214, rs6795970, rs3807989, rs7660702, rs132311, rs1733724 and rs365990, and markers in linkage disequilibrium therewith, wherein different alleles of the at least one polymorphic marker are associated with different probabilities of response to the therapeutic agent in humans; and determining the probability of a positive response to the therapeutic agent from the sequence data.

The methods of the invention, as described herein, may suitably be performed in connection with determination of biomarkers. In other words, the methods as described herein may comprise a further step of assaying or determining at least one biomarker. The biomarker may in a general sense be any suitable biomarker for any electrocardiogram measure, or the biomarker may be predictive of, or associated with, Atrial Fibrillation, Atrial Flutter and/or stroke.

The invention also provides kits. In one such aspect, the invention relates to a kit for assessing susceptibility to a vascular condition selected from the group consisting of: an abnormal electrocardiogram measure, Atrial Fibrillation, Atrial Flutter, and Stroke, the kit comprising: reagents for selectively detecting at least one allele of at least one polymorphic marker in the genome of the individual, wherein the polymorphic marker is selected from the group consisting of rs3825214, rs6795970, rs3807989, rs7660702, rs132311, rs1733724 and rs365990, and markers in linkage disequilibrium therewith; and a collection of data comprising correlation data between the at least one polymorphism and susceptibility to the condition.

It may also be convenient to use particular nucleotide probes for manufacturing diagnostic reagents as described herein. A further aspect of the invention thus relates to the use of an oligonucleotide probe in the manufacture of a diagnostic reagent for diagnosing and/or assessing a susceptibility to a vascular condition selected from the group consisting of: an abnormal electrocardiogram measure, Atrial Fibrillation, Atrial Flutter, and Stroke, wherein the probe is capable of hybridizing to a segment of a nucleic acid whose nucleotide sequence is given by any one of SEQ ID NO:1-3623, and wherein the segment is 15-500 nucleotides in length. In one embodiment, the segment is 15-400 nucleotides in length. In another embodiment, the segment is 15-180 nucleotides in length.

Yet another aspect of the invention relates to the use of the diagnostic markers described hererin for the selection of individuals to be treated with at least one therapeutic agent. One such aspect relates to the use of an agent for treating a vascular condition selected from the group consisting of: an abnormal electrocardiogram measure, Atrial Fibrillation, Atrial Flutter, and Stroke in a human individual that has been tested for the presence of at least one allele of at least one polymorphic marker selected from the group consisting of rs3825214, rs6795970, rs3807989, rs7660702, rs132311, rs1733724 and rs365990, and markers in linkage disequilibrium therewith. In one embodiment, an individual who is determined to have at least one copy of the allele that correlates with increased risk of Atrial Fibrillation, Atrial Flutter and Stroke, or an allele that correlates with an abnormal electrocardiogram measure, is selected for treatment with the therapeutic agent.

The invention may implemented on computerized systems. One such aspect relates to a computer-readable medium having computer executable instructions for determining susceptibility to a vascular condition selected from the group consisting of: an abnormal electrocardiogram measure, Atrial Fibrillation, Atrial Flutter, and Stroke, the computer readable medium comprising (i) data indicative of at least one polymorphic marker; and (ii) a routine stored on the computer readable medium and adapted to be executed by a processor to determine risk of developing the condition for the at least one polymorphic marker; wherein the at least one polymorphic marker is selected from the group consisting of rs3825214, rs6795970, rs3807989, rs7660702, rs132311, rs1733724 and rs365990, and markers in linkage disequilibrium therewith.

Another computer-implemented aspect relates to a system for generating a risk assessment report for a vascular condition selected from the group consisting of: an abnormal electrocardiogram measure, Atrial Fibrillation, Atrial Flutter, and Stroke, the system comprising: (a) a memory configured to store sequence data for at least one human subject, the sequence data identifying at least one allele of at least one polymorphic marker, wherein different alleles of the marker are associated with different susceptibilities to the condition in humans; and (b) a processor configured to: (i) receive information identifying the at least one allele of the at least one polymorphic marker; (ii) transform said information into a risk measure of the condition for the human subject; (iii) generate a risk assessment report based on the received information, and (iv) provide the risk assessment report on an output device, wherein the at least one polymorphic marker is selected from the group consisting of rs3825214, rs6795970, rs3807989, rs7660702, rs132311, rs1733724 and rs365990, and markers in linkage disequilibrium therewith. The output device may be any suitable device for providing a risk assessment report. The device is in certain embodiments a printer capable of printing the report: The device may also be a server, optionally containing access control, such that the report may be accessed via a web interface.

Yet another computerized aspect relates to an apparatus for determining a genetic indicator for a vascular condition selected from the group consisting of: an abnormal electrocardiogram measure, Atrial Fibrillation, Atrial Flutter, and Stroke, in a human individual, comprising (a) a processor; and (b) a computer readable memory having computer executable instructions adapted to be executed on the processor to analyze marker and/or haplotype information for at least one human individual with respect to at least one polymorphic marker selected from the group consisting of rs3825214, rs6795970, rs3807989, rs7660702, rs132311, rs1733724 and rs365990, and markers in linkage disequilibrium therewith, and generate an output based on the marker or haplotype information, wherein the output comprises a measure of susceptibility of the at least one marker or haplotype as a genetic indicator of the condition for the human individual.

The abnormal electrocardiogram measures in the various embodiments of the invention may suitably be selected from the group consisting of: an increased and/or decreased QRS interval, an increased and/or decreased PR interval, an increased and/or decreased QT interval, sick sinus syndrome and an increased and/or decreased heart rate. In certain embodiments, the abnormal electrocardiogram measure is selected from the group consisting of: an increased QRS interval, an increased PR interval, an increased QT interval, sick sinus syndrome and an increased heart rate.

In certain embodiments of the invention, the vascular condition is Atrial Fibrillation.

It should be understood that all combinations of features described herein are contemplated, even if the combination of feature is not specifically found in the same sentence or paragraph herein. This includes for example particular the use of all markers disclosed herein, alone or in combination, for analysis individually or in haplotypes, in all aspects of the invention as described herein.

### BRIEF DESCRIPTION OF THE DRAWINGS

The foregoing and other objects, features and advantages of the invention will be apparent from the following more particular description of preferred embodiments of the invention.

FIG. 1 provides a diagram illustrating a computer-implemented system utilizing risk variants as described herein.

### DETAILED DESCRIPTION

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometime referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, mini- or microsatellites, translocations and copy number variations (insertions, deletions, duplications). Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency, in particular copy number variations (CNVs). The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles (e.g., allele-specific sequences) for any given polymorphic marker, representative of each copy of the marker on each chromosome. Sequence codes for nucleotides used herein are: A=1, C=2, G=3, T=4. For microsatellite alleles, the CEPH sample (Centre d'Etudes du Polymorphisme Humain, genomics repository, CEPH sample 1347-O₂) is used as a reference, the shorter allele of each microsatellite in this sample is set as 0 and all other alleles in other samples are numbered in relation to this reference. Thus, e.g., allele 1 is 1 bp longer than the shorter allele in the CEPH sample, allele 2 is 2 bp longer than the shorter allele in the CEPH sample, allele 3 is 3 bp longer than the lower allele in the CEPH sample, etc., and allele-1 is 1 bp shorter than the shorter allele in the CEPH sample, allele-2 is 2 bp shorter than the shorter allele in the CEPH sample, etc.

Sequence conucleotide ambiguity as described herein is as proposed by IUPAC-IUB. These codes are compatible with the codes used by the EMBL, GenBank, and PIR databases.

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C, G or T |
| D | A, G or T |
| H | A, C or T |
| V | A, C or G |
| N | A, C, G or T (Any base) |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population. An “indel” is a common form of polymorphism comprising a small insertion or deletion that is typically only a few nucleotides long.

A “haplotype,” as described herein, refers to a segment of genomic DNA that is characterized by a specific combination of alleles arranged along the segment. For diploid organisms such as humans, a haplotype comprises one member of the pair of alleles for each polymorphic marker or locus along the segment. In a certain embodiment, the haplotype can comprise two or more alleles, three or more alleles, four or more alleles, or five or more alleles. Haplotypes are described herein in the context of the marker name and the allele of the marker in that haplotype, e.g., “3 rs3825214” refers to the 3 allele of marker rs3825214 being in the haplotype, and is equivalent to “rs3825214 allele 3”. Furthermore, allelic codes in haplotypes are as for individual markers, i.e. 1=A, 2=C, 3=G and 4=T.

The term “susceptibility”, as described herein, refers to the proneness of an individual towards the development of a certain state (e.g., a certain trait, phenotype or disease), or towards being less able to resist a particular state than the average individual. The term encompasses both increased susceptibility and decreased susceptibility. Thus, particular alleles at polymorphic markers and/or haplotypes of the invention as described herein may be characteristic of increased susceptibility (i.e., increased risk) of disease, as characterized by a relative risk (RR) or odds ratio (OR) of greater than one for the particular allele or haplotype. Alternatively, the markers and/or haplotypes of the invention are characteristic of decreased susceptibility (i.e., decreased risk) of disease, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or the can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “nucleic acid sample” as described herein, refers to a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including a blood sample, sample of amniotic fluid, sample of cerebrospinal fluid, or tissue sample from skin, muscle, buccal or conjunctival mucosa, placenta, gastrointestinal tract or other organs.

The term “abnormal electrocardiogram measure”, as described herein, refers to an electrocardiogram measure that is outside the normal range of the measure, as determined by the skilled artisan (e.g., a clinician). For example, an elevated heart rate, an increased QT interval, an increased QRS interval and an increased PT interval are examples of abnormal electrocardiogram measure. The variants described herein are correlated with electrocardiogram measures, such that a particular allele at particular SNPs are correlated with an increase in the measure, e.g., an increased interval or heart rate.

The term “antisense agent” or “antisense oligonucleotide” refers, as described herein, to molecules, or compositions comprising molecules, which include a sequence of purine an pyrimidine heterocyclic bases, supported by a backbone, which are effective to hydrogen bond to a corresponding contiguous bases in a target nucleic acid sequence. The backbone is composed of subunit backbone moieties supporting the purine and pyrimidine heterocyclic bases at positions which allow such hydrogen bonding. These backbone moieties are cyclic moieties of 5 to 7 atoms in size, linked together by phosphorous-containing linkage units of one to three atoms in length. In certain preferred embodiments, the antisense agent comprises an oligonucleotide molecule.

The term “LD Block CO₃”, as described herein, refers to the Linkage Disequilibrium (LD) block on Chromosome 3 between markers rs6599240 and rs10212338, corresponding to position 38,713,721-38,787,654 of NCBI (National Center for Biotechnology Information) Build 36.

The term “LD Block C04”, as described herein, refers to the Linkage Disequilibrium (LD) block on Chromosome 4 between markers rs7698203 and rs7693640, corresponding to position 86,823,753-86,942,405 of NCBI (National Center for Biotechnology Information) Build 36.

The term “LD Block C06”, as described herein, refers to the Linkage Disequilibrium (LD) block on Chromosome 6 between markers rs6457931 and rs7762245, corresponding to position 36,721,790-36,824,207 of NCBI (National Center for Biotechnology Information) Build 36.

The term “LD Block C07”, as described herein, refers to the Linkage Disequilibrium (LD) block on Chromosome 7 between markers rs2157799 and rs6978354, corresponding to position 115,791,226-116,013,658 of NCBI (National Center for Biotechnology Information) Build 36.

The term “LD Block C10”, as described herein, refers to the Linkage Disequilibrium (LD) block on Chromosome 10 between markers rs1149782 and rs1733724, corresponding to position 53,808,502-53,893,983 of NCBI (National Center for Biotechnology Information) Build 36.

The term “LD Block C12”, as described herein, refers to the Linkage Disequilibrium (LD) block on Chromosome 12 between markers rs6489952 and rs17731569, corresponding to position 113,243,156-113,312,090 of NCBI (National Center for Biotechnology Information) Build 36.

The term “LD Block C14”, as described herein, refers to the Linkage Disequilibrium (LD) block on Chromosome 14 between markers rs3811178 and rs2754163, corresponding to position 22,915,084-22,967,347 of NCBI (National Center for Biotechnology Information) Build 36.

### Assessment for Markers and Haplotypes

The genomic sequence within populations is not identical when individuals are compared. Rather, the genome exhibits sequence variability between individuals at many locations in the genome. Such variations in sequence are commonly referred to as polymorphisms, and there are many such sites within each genome. For example, the human genome exhibits sequence variations which occur on average every 500 base pairs. The most common sequence variant consists of base variations at a single base position in the genome, and such sequence variants, or polymorphisms, are commonly called Single Nucleotide Polymorphisms (“SNPs”). These SNPs are believed to have occurred in a single mutational event, and therefore there are usually two possible alleles possible at each SNPsite; the original allele and the mutated allele. Due to natural genetic drift and possibly also selective pressure, the original mutation has resulted in a polymorphism characterized by a particular frequency of its alleles in any given population. Many other types of sequence variants are found in the human genome, including mini- and microsatellites, and insertions, deletions and inversions (also called copy number variations (CNVs)). A polymorphic microsatellite has multiple small repeats of bases (such as CA repeats, TG on the complimentary strand) at a particular site in which the number of repeat lengths varies in the general population. In general terms, each version of the sequence with respect to the polymorphic site represents a specific allele of the polymorphic site. These sequence variants can all be referred to as polymorphisms, occurring at specific polymorphic sites characteristic of the sequence variant in question. In general, polymorphisms can comprise any number of specific alleles within the population, although each human individual has two alleles at each polymorphic site—one maternal and one paternal allele. Thus in one embodiment of the invention, the polymorphism is characterized by the presence of two or more alleles in any given population. In another embodiment, the polymorphism is characterized by the presence of three or more alleles in a population. In other embodiments, the polymorphism is characterized by four or more alleles, five or more alleles, six or more alleles, seven or more alleles, nine or more alleles, or ten or more alleles. All such polymorphisms can be utilized in the methods and kits of the present invention, and are thus within the scope of the invention.

Due to their abundance, SNPs account for a majority of sequence variation in the human genome. Over 6 million human SNPs have been validated to date (http://www.ncbi.nlm.nih.gov/projects/SNP/snp_summary.cgi). However, CNVs are receiving increased attention. These large-scale polymorphisms (typically 1 kb or larger) account for polymorphic variation affecting a substantial proportion of the assembled human genome; known CNVs covery over 15% of the human genome sequence (Estivill, X Armengol; L., PloS Genetics 3:1787-99 (2007); http://projects.tcag.ca/variation/). Most of these polymorphisms are however very rare, and on average affect only a fraction of the genomic sequence of each individual. CNVs are known to affect gene expression, phenotypic variation and adaptation by disrupting gene dosage, and are also known to cause disease (microdeletion and microduplication disorders) and confer risk of common complex diseases, including HIV-1 infection and glomerulonephritis (Redon, R., et al. Nature 23:444-454 (2006)). It is thus possible that either previously described or unknown CNVs represent causative variants in linkage disequilibrium with the disease-associated markers described herein. Methods for detecting CNVs include comparative genomic hybridization (CGH) and genotyping, including use of genotyping arrays, as described by Carter (Nature Genetics 39:S16-S21 (2007)). The Database of Genomic Variants (http://projects.tcag.ca/variation/) contains updated information about the location, type and size of described CNVs. The database currently contains data for over 21,000 CNVs.

In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a trait or disease phenotype).

Alleles for SNP markers as referred to herein refer to the bases A, C, G or T as they occur at the polymorphic site. The allele codes for SNPs used herein are as follows: 1=A, 2=C, 3=G, 4=T. Since human DNA is double-stranded, the person skilled in the art will realise that by assaying or reading the opposite DNA strand, the complementary allele can in each case be measured. Thus, for a polymorphic site (polymorphic marker) characterized by an A/G polymorphism, the methodology employed to detect the marker may be designed to specifically detect the presence of one or both of the two bases possible, i.e. A and G. Alternatively, by designing an assay that is designed to detect the complimentary strand on the DNA template, the presence of the complementary bases T and C can be measured. Quantitatively (for example, in terms of risk estimates), identical results would be obtained from measurement of either DNA strand (+strand or −strand).

Typically, a reference sequence is referred to for a particular sequence. Alleles that differ from the reference are sometimes referred to as “variant” alleles. A variant sequence, as used herein, refers to a sequence that differs from the reference sequence but is otherwise substantially similar. Alleles at the polymorphic genetic markers described herein are variants. Variants can include changes that affect a polypeptide. Sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence.

Such sequence changes can alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level. The polypeptide encoded by the reference nucleotide sequence is the “reference” polypeptide with a particular reference amino acid sequence, and polypeptides encoded by variant alleles are referred to as “variant” polypeptides with variant amino acid sequences.

A haplotype refers to a single-stranded segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

Detecting specific polymorphic markers and/or haplotypes can be accomplished by methods known in the art for detecting sequences at polymorphic sites. For example, standard techniques for genotyping for the presence of SNPs and/or microsatellite markers can be used, such as fluorescence-based techniques (e.g., Chen, X. et al., Genome Res. 9(5): 492-98 (1999); Kutyavin et al., Nucleic Acid Res. 34:e128 (2006)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave). Some of the available array platforms, including Affymetrix SNP Array 6.0 and Illumina CNV370-Duo and 1M BeadChips, include SNPs that tag certain CNVs. This allows detection of CNVs via surrogate SNPs included in these platforms. Thus, by use of these or other methods available to the person skilled in the art, one or more alleles at polymorphic markers, including microsatellites, SNPs or other types of polymorphic markers, can be identified.

In certain embodiments, polymorphic markers are detected by sequencing technologies. Obtaining sequence information about an individual identifies particular nucleotides in the context of a sequence. For SNPs, sequence information about a single unique sequence site is sufficient to identify alleles at that particular SNP. For markers comprising more than one nucleotide, sequence information about the nucleotides of the individual that contain the polymorphic site identifies the alleles of the individual for the particular site. The sequence information can be obtained from a sample from the individual. In certain embodiments, the sample is a nucleic acid sample. In certain other embodiments, the sample is a protein sample.

Various methods for obtaining nucleic acid sequence are known to the skilled person, and all such methods are useful for practicing the invention. Sanger sequencing is a well-known method for generating nucleic acid sequence information. Recent methods for obtaining large amounts of sequence data have been developed, and such methods are also contemplated to be useful for obtaining sequence information. These include pyrosequencing technology (Ronaghi, M. et al. Anal Biochem 267:65-71 (1999); Ronaghi, et al. Biotechniques 25:876-878 (1998)), e.g. 454 pyrosequencing (Nyren, P., et al. Anal Biochem 208:171-175 (1993)), Illumina/Solexa sequencing technology (http://www.illumina.com; see also Strausberg, R L, et al Drug Disc Today 13:569-577 (2008)), and Supported Oligonucleotide Ligation and Detection Platform (SOLID) technology (Applied Biosystems, http://www.appliedbiosystems.com); Strausberg, R L, et al Drug Disc Today 13:569-577 (2008).

It is possible to impute or predict genotypes for un-genotyped relatives of genotyped individuals. For every un-genotyped case, it is possible to calculate the probability of the genotypes of its relatives given its four possible phased genotypes. In practice it may be preferable to include only the genotypes of the case's parents, children, siblings, half-siblings (and the half-sibling's parents), grand-parents, grand-children (and the grand-children's parents) and spouses. It will be assumed that the individuals in the small sub-pedigrees created around each case are not related through any path not included in the pedigree. It is also assumed that alleles that are not transmitted to the case have the same frequency—the population allele frequency. Let us consider a SNP marker with the alleles A and G. The probability of the genotypes of the case's relatives can then be computed by:

$$Pr ( genotypes of relatives ; θ ) = {\sum }_{h ∈ \{ AA , AG , GA , GG \}} Pr ( h ; θ ) Pr ( genotypes of relatives \vert h ) ,$$

where θ denotes the A allele's frequency in the cases. Assuming the genotypes of each set of relatives are independent, this allows us to write down a likelihood function for θ:

$$L ( θ ) = {\prod }_{i} Pr ( genotypes of relatives of case i ; θ ) . \qquad (* )$$

This assumption of independence is usually not correct. Accounting for the dependence between individuals is a difficult and potentially prohibitively expensive computational task. The likelihood function in (*) may be thought of as a pseudolikelihood approximation of the full likelihood function for θ which properly accounts for all dependencies. In general, the genotyped cases and controls in a case-control association study are not independent and applying the case-control method to related cases and controls is an analogous approximation. The method of genomic control (Devlin, B. et al., Nat Genet. 36, 1129-30; author reply 1131 (2004)) has proven to be successful at adjusting case-control test statistics for relatedness. We therefore apply the method of genomic control to account for the dependence between the terms in our pseudolikelihood and produce a valid test statistic.

Fisher's information can be used to estimate the effective sample size of the part of the pseudolikelihood due to un-genotyped cases. Breaking the total Fisher information, I, into the part due to genotyped cases, I_{g}, and the part due to ungenotyped cases, Iᵤ, I=I_{g}+Iᵤ, and denoting the number of genotyped cases with N, the effective sample size due to the un-genotyped cases is estimated by

$$\frac{{I}_{u}}{{I}_{g}} N .$$

In the present context, an individual who is at an increased susceptibility (i.e., increased risk) for a disease, is an individual in whom at least one specific allele at one or more polymorphic marker or haplotype conferring increased susceptibility (increased risk) for the disease is identified (i.e., at-risk marker alleles or haplotypes). The at-risk marker or haplotype is one that confers an increased risk (increased susceptibility) of the disease. In one embodiment, significance associated with a marker or haplotype is measured by a relative risk (RR). In another embodiment, significance associated with a marker or haplotye is measured by an odds ratio (OR). In a further embodiment, the significance is measured by a percentage. In one embodiment, a significant increased risk is measured as a risk (relative risk and/or odds ratio) of at least 1.2, including but not limited to: at least 1.2, at least 1.3, at least 1.4, at least 1.5, at least 1.6, at least 1.7, 1.8, at least 1.9, at least 2.0, at least 2.5, at least 3.0, at least 4.0, and at least 5.0. In a particular embodiment, a risk (relative risk and/or odds ratio) of at least 1.2 is significant. In another particular embodiment, a risk of at least 1.3 is significant. In yet another embodiment, a risk of at least 1.4 is significant. In a further embodiment, a risk of at least 1.5 is significant. In another further embodiment, a risk of at least 1.7 is significant. However, other cutoffs are also contemplated, e.g., at least 1.15, 1.25, 1.35, and so on, and such cutoffs are also within scope of the present invention. In other embodiments, a significant increase in risk is at least about 20%, including but not limited to about 25%, 30%, 35%, 40%, 45%, 50%, 55%, 60%, 65%, 70%, 75%, 80%, 85%, 90%, 95%, 100%, 150%, 200%, 300%, and 500%. In one particular embodiment, a significant increase in risk is at least 20%. In other embodiments, a significant increase in risk is at least 30%, at least 40%, at least 50%, at least 60%, at least 70%, at least 80%, at least 90% and at least 100%. Other cutoffs or ranges as deemed suitable by the person skilled in the art to characterize the invention are however also contemplated, and those are also within scope of the present invention. In certain embodiments, a significant increase in risk is characterized by a p-value, such as a p-value of less than 0.05, less than 0.01, less than 0.001, less than 0.0001, less than 0.00001, less than 0.000001, less than 0.0000001, less than 0.00000001, or less than 0.000000001.

An at-risk polymorphic marker or haplotype as described herein is one where at least one allele of at least one marker or haplotype is more frequently present in an individual at risk for the disease (or trait) (affected), or diagnosed with the disease, compared to the frequency of its presence in a comparison group (control), such that the presence of the marker or haplotype is indicative of susceptibility to the disease. The control group may in one embodiment be a population sample, i.e. a random sample from the general population. In another embodiment, the control group is represented by a group of individuals who are disease-free. Such disease-free controls may in one embodiment be characterized by the absence of one or more specific disease-associated symptoms. Alternatively, the disesae-free controls are those that have not been diagnosed with the disease. In another embodiment, the disease-free control group is characterized by the absence of one or more disease-specific risk factors. Such risk factors are in one embodiment at least one environmental risk factor. Representative environmental factors are natural products, minerals or other chemicals which are known to affect, or contemplated to affect, the risk of developing the specific disease or trait. Other environmental risk factors are risk factors related to lifestyle, including but not limited to food and drink habits, geographical location of main habitat, and occupational risk factors. In another embodiment, the risk factors comprise at least one additional genetic risk factor.

As an example of a simple test for correlation would be a Fisher-exact test on a two by two table. Given a cohort of chromosomes, the two by two table is constructed out of the number of chromosomes that include both of the markers or haplotypes, one of the markers or haplotypes but not the other and neither of the markers or haplotypes. Other statistical tests of association known to the skilled person are also contemplated and are also within scope of the invention.

The person skilled in the art will appreciate that for markers with two alleles present in the population being studied (such as SNPs), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

Thus, in other embodiments of the invention, an individual who is at a decreased susceptibility (i.e., at a decreased risk) for a disease or trait is an individual in whom at least one specific allele at one or more polymorphic marker or haplotype conferring decreased susceptibility for the disease or trait is identified. The marker alleles and/or haplotypes conferring decreased risk are also said to be protective. In one aspect, the protective marker or haplotype is one that confers a significant decreased risk (or susceptibility) of the disease or trait. In one embodiment, significant decreased risk is measured as a relative risk (or odds ratio) of less than 0.9, including but not limited to less than 0.9, less than 0.8, less than 0.7, less than 0.6, less than 0.5, less than 0.4, less than 0.3, less than 0.2 and less than 0.1. In one particular embodiment, significant decreased risk is less than 0.7. In another embodiment, significant decreased risk is less than 0.5. In yet another embodiment, significant decreased risk is less than 0.3. In another embodiment, the decrease in risk (or susceptibility) is at least 20%, including but not limited to at least 25%, at least 30%, at least 35%, at least 40%, at least 45%, at least 50%, at least 55%, at least 60%, at least 65%, at least 70%, at least 75%, at least 80%, at least 85%, at least 90%, at least 95% and at least 98%. In one particular embodiment, a significant decrease in risk is at least about 30%. In another embodiment, a significant decrease in risk is at least about 50%. In another embodiment, the decrease in risk is at least about 70%. Other cutoffs or ranges as deemed suitable by the person skilled in the art to characterize the invention are however also contemplated, and those are also within scope of the present invention.

A genetic variant associated with a disease or a trait can be used alone to predict the risk of the disease for a given genotype. For a biallelic marker, such as a SNP, there are 3 possible genotypes: homozygote for the at risk variant, heterozygote, and non carrier of the at risk variant. Risk associated with variants at multiple loci can be used to estimate overall risk. For multiple SNP variants, there are k possible genotypes k=3′×2P; where n is the number autosomal loci and p the number of gonosomal (sex chromosomal) loci. Overall risk assessment calculations for a plurality of risk variants usually assume that the relative risks of different genetic variants multiply, i.e. the overall risk (e.g., RR or OR) associated with a particular genotype combination is the product of the risk values for the genotype at each locus. If the risk presented is the relative risk for a person, or a specific genotype for a person, compared to a reference population with matched gender and ethnicity, then the combined risk is the product of the locus specific risk values and also corresponds to an overall risk estimate compared with the population. If the risk for a person is based on a comparison to non-carriers of the at risk allele, then the combined risk corresponds to an estimate that compares the person with a given combination of genotypes at all loci to a group of individuals who do not carry risk variants at any of those loci. The group of non-carriers of any at risk variant has the lowest estimated risk and has a combined risk, compared with itself (i.e., non-carriers) of 1.0, but has an overall risk, compare with the population, of less than 1.0. It should be noted that the group of non-carriers can potentially be very small, especially for large number of loci, and in that case, its relevance is correspondingly small.

The multiplicative model is a parsimonious model that usually fits the data of complex traits reasonably well. Deviations from multiplicity have been rarely described in the context of common variants for common diseases, and if reported are usually only suggestive since very large sample sizes are usually required to be able to demonstrate statistical interactions between loci.

By way of an example, let us consider seven variants disclosed herein to be associated with ECG measures and related diseases (rs6795970, rs7660702, rs132311, rs3807989, rs1733724, rs3825214 and rs365990). The total number of theoretical genotypic combinations for these seven SNP variants 3⁷=2187. Some of those genotypic combinations are very rare, but are still possible; therefore, all of these combinations should be considered for overall risk assessment.

It is likely that the multiplicative model applied in the case of multiple genetic variant will also be valid in conjugation with non-genetic risk variants assuming that the genetic variant does not clearly correlate with the “environmental” factor. In other words, genetic and non-genetic at-risk variants can be assessed under the multiplicative model to estimate combined risk, assuming that the non-genetic and genetic risk factors do not interact.

### Linkage Disequilibrium

The natural phenomenon of recombination, which occurs on average once for each chromosomal pair during each meiotic event, represents one way in which nature provides variations in sequence (and biological function by consequence). It has been discovered that recombination does not occur randomly in the genome; rather, there are large variations in the frequency of recombination rates, resulting in small regions of high recombination frequency (also called recombination hotspots) and larger regions of low recombination frequency, which are commonly referred to as Linkage Disequilibrium (LD) blocks (Myers, S. et al., Biochem Soc Trans 34:526-530 (2006); Jeffreys, A. J., et al., Nature Genet. 29:217-222 (2001); May, C. A., et al., Nature Genet. 31:272-275 (2002)).

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrence of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurrence of each allele or haplotype in the population. For populations of diploids, e.g., human populations, individuals will typically have two alleles or allelic combinations for each genetic element (e.g., a marker, haplotype or gene).

Many different measures have been proposed for assessing the strength of linkage disequilibrium (LD; reviewed in Devlin, B. & Risch, N., Genomics 29:311-22 (1995)). Most capture the strength of association between pairs of biallelic sites. Two important pairwise measures of LD are r² (sometimes denoted Δ²) and |D′| (Lewontin, R., Genetics 49:49-67 (1964); Hill, W. G. & Robertson, A. Theor. Appl. Genet. 22:226-231 (1968)). Both measures range from 0 (no disequilibrium) to 1 (‘complete’ disequilibrium), but their interpretation is slightly different. |D′| is defined in such a way that it is equal to 1 if just two or three of the possible haplotypes for two markers are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value between markers indicative of the markers being in linkage disequilibrium can be at least 0.1, such as at least 0.15, 0.20, 0.25, 0.30, 0.35, 0.40, 0.45, 0.50, 0.55, 0.60, 0.65, 0.70, 0.75, 0.80, 0.85, 0.90, 0.91, 0.92, 0.93, 0.94, 0.95, 0.96, 0.97, 0.98, or at least 0.99. In one preferred embodiment, the significant r² value can be at least 0.2. Alternatively, markers in linkage disequilibrium are characterized by values of |D′| of at least 0.2, such as 0.3, 0.4, 0.5, 0.6, 0.7, 0.8, 0.85, 0.9, 0.95, 0.96, 0.97, 0.98, or at least 0.99. Thus, linkage disequilibrium represents a correlation between alleles of distinct markers. In certain embodiments, linkage disequilibrium is defined in terms of values for both the r² and |D′| measures. In one such embodiment, a significant linkage disequilibrium is defined as r²>0.1 and/or |D′|>0.8, and markers fulfilling these criteria are said to be in linkage disequilibrium. In another embodiment, a significant linkage disequilibrium is defined as r²>0.2 and/or |D′|>0.9. Other combinations and permutations of values of r² and |D′| for determining linkage disequilibrium are also contemplated, and are also within the scope of the invention. Linkage disequilibrium can be determined in a single human population, as defined herein, or it can be determined in a collection of samples comprising individuals from more than one human population. In one embodiment of the invention, LD is determined in a sample from one or more of the HapMap populations (Caucasian, African (Yuroban), Japanese, Chinese), as defined (http://www.hapmap.org). In one such embodiment, LD is determined in the CEU population of the HapMap samples (Utah residents with ancestry from northern and western Europe). In another embodiment, LD is determined in the YR1 population of the HapMap samples (Yuroba in Ibadan, Nigeria). In another embodiment, LD is determined in the CHB population of the HapMap samples (Han Chinese from Beijing, China). In another embodiment, LD is determined in the JPT population of the HapMap samples (Japanese from Tokyo, Japan). In yet another embodiment, LD is determined in samples from the Icelandic population.

If all polymorphisms in the genome were independent at the population level (i.e., no LD), then every single one of them would need to be investigated in association studies, to assess all the different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Aced Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as regions flanked by recombination hotspots. As used herein, the terms “haplotype block” or “LD block” includes blocks defined by any of the above described characteristics, or other alternative methods used by the person skilled in the art to define such regions.

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. Markers shown herein to be associated with ECG measures and associated diseases (Atrial Fibrillation, Atrial Flutter and Stroke) are such tagging markers. If desired, neighboring haplotype blocks can be assessed concurrently, as there may also exist linkage disequilibrium among the haplotype blocks.

It has thus become apparent that for any given observed association to a polymorphic marker in the genome, additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the present invention. One or more causative (functional) variants or mutations may reside within the region found to be associating to the disease or trait. The functional variant may be another SNP, a tandem repeat polymorphism (such as a minisatellite or a microsatellite), a transposable element, or a copy number variation, such as an inversion, deletion or insertion. Such variants in LD with the variants described herein may confer a higher relative risk (RR) or odds ratio (OR) than observed for the tagging markers used to detect the association. The present invention thus refers to the markers used for detecting association to the disease, as described herein, as well as markers in linkage disequilibrium with the markers. Thus, in certain embodiments of the invention, markers that are in LD with the markers originally used to detect an association may be used as surrogate markers. The surrogate markers have in one embodiment relative risk (RR) and/or odds ratio (OR) values smaller than originally detected. In other embodiments, the surrogate markers have RR or OR values greater than those initially determined for the markers initially found to be associating with the disease. An example of such an embodiment would be a rare, or relatively rare (such as <10% allelic population frequency) variant in LD with a more common variant (>10% population frequency) initially found to be associating with the disease. Identifying and using such surrogate markers for detecting the association can be performed by routine methods well known to the person skilled in the art, and are therefore within the scope of the present invention.

### Determination of Haplotype Frequency

The frequencies of haplotypes in patient and control groups can be estimated using an expectation-maximization algorithm (Dempster A. et al., J. R. Stat. Soc. B, 39:1-38 (1977)). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistical significance.

To look for at-risk and protective markers and haplotypes within a susceptibility region, for example within an LD block, association of all possible combinations of genotyped markers within the region is studied. The combined patient and control groups can be randomly divided into two sets, equal in size to the original group of patients and controls. The marker and haplotype analysis is then repeated and the most significant p-value registered is determined. This randomization scheme can be repeated, for example, over 100 times to construct an empirical distribution of p-values. In a preferred embodiment, a p-value of <0.05 is indicative of a significant marker and/or haplotype association.

### Haplotype Analysis

One general approach to haplotype analysis involves using likelihood-based inference applied to NEsted MOdels (Gretarsdottir S., et al., Nat. Genet. 35:131-38 (2003)). The method is implemented in the program NEMO, which allows for many polymorphic markers, SNPs and microsatellites. The method and software are specifically designed for case-control studies where the purpose is to identify haplotype groups that confer different risks. It is also a tool for studying LD structures. In NEMO, maximum likelihood estimates, likelihood ratios and p-values are calculated directly, with the aid of the EM algorithm, for the observed data treating it as a missing-data problem.

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. The information measure for haplotype analysis is described in Nicolae and Kong (Technical Report 537, Department of Statistics, University of Statistics, University of Chicago; Biometrics, 60(2):368-75 (2004)) as a natural extension of information measures defined for linkage analysis, and is implemented in NEMO.

### Association Analysis

For single marker association to a disease, the Fisher exact test can be used to calculate two-sided p-values for each individual allele. Correcting for relatedness among patients can be done by extending a variance adjustment procedure previously described (Risch, N. & Teng, J. Genome Res., 8:1273-1288 (1998)) for sibships so that it can be applied to general familial relationships. The method of genomic controls (Devlin, B. & Roeder, K. Biometrics 55:997 (1999)) can also be used to adjust for the relatedness of the individuals and possible stratification.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

An association signal detected in one association study may be replicated in a second cohort, ideally from a different population (e.g., different region of same country, or a different country) of the same or different ethnicity. The advantage of replication studies is that the number of tests performed in the replication study is usually quite small, and hence the less stringent the statistical measure that needs to be applied. For example, for a genome-wide search for susceptibility variants for a particular disease or trait using 300,000 SNPs, a correction for the 300,000 tests performed (one for each SNP) can be performed. Since many SNPs on the arrays typically used are correlated (i.e., in LD), they are not independent. Thus, the correction is conservative. Nevertheless, applying this correction factor requires an observed P-value of less than 0.05/300,000 =1.7×10⁻⁷ for the signal to be considered significant applying this conservative test on results from a single study cohort. Obviously, signals found in a genome-wide association study with P-values less than this conservative threshold (i.e., more significant) are a measure of a true genetic effect, and replication in additional cohorts is not necessarily from a statistical point of view. Importantly, however, signals with P-values that are greater than this threshold may also be due to a true genetic effect. The sample size in the first study may not have been sufficiently large to provide an observed P-value that meets the conservative threshold for genome-wide significance, or the first study may not have reached genome-wide significance due to inherent fluctuations due to sampling. Since the correction factor depends on the number of statistical tests performed, if one signal (one SNP) from an initial study is replicated in a second case-control cohort, the appropriate statistical test for significance is that for a single statistical test, i.e., P-value less than 0.05. Replication studies in one or even several additional case-control cohorts have the added advantage of providing assessment of the association signal in additional populations, thus simultaneously confirming the initial finding and providing an assessment of the overall significance of the genetic variant(s) being tested in human populations in general.

The results from several case-control cohorts can also be combined to provide an overall assessment of the underlying effect. The methodology commonly used to combine results from multiple genetic association studies is the Mantel-Haenszel model (Mantel and Haenszel, J Natl Cancer Inst 22:719-48 (1959)). The model is designed to deal with the situation where association results from different populations, with each possibly having a different population frequency of the genetic variant, are combined. The model combines the results assuming that the effect of the variant on the risk of the disease, a measured by the OR or RR, is the same in all populations, while the frequency of the variant may differ between the populations. Combining the results from several populations has the added advantage that the overall power to detect a real underlying association signal is increased, due to the increased statistical power provided by the combined cohorts. Furthermore, any deficiencies in individual studies, for example due to unequal matching of cases and controls or population stratification will tend to balance out when results from multiple cohorts are combined, again providing a better estimate of the true underlying genetic effect.

### Methods of Determining Susceptibility to Electrocardiogram Measures, Atrial Fibrillation, Atrial Flutter and Stroke

The present inventors have for the first time shown that certain polymorphic variants are associated with electrocardiogram measures. Certain alleles at these variants correlate with increased electrocardiogram measures, including the PR, QRS and QT intervals, and heart rate. These variants have also been found to be associated with risk of sick sinus syndrome, atrioventricular block, pacemaker placement, as well as risk of developing Atrial Fibrillation, Atrial Flutter and/or Stroke. These polymorphic markers, as well as markers in linkage disequilibrium with these polymorphic markers, are contemplated to be useful as markers for determining susceptibility to any one or more, or any combination of, of these conditions. These markers are believed to be useful in a range of diagnostic applications, as described further herein.

Accordingly, in one aspect the invention provides a method of determining a susceptibility to a vascular condition selected from the group consisting of: an abnormal electrocardiogram measure, Atrial Fibrillation, Atrial Flutter, and Stroke, in a human individual, the method comprising: (i) obtaining sequence data about a human individual identifying at least one allele of at least one polymorphic marker, wherein different alleles of the at least one polymorphic marker are associated with different susceptibilities to the condition in humans, and (ii) determining a susceptibility to the condition from the sequence data, wherein the at least one polymorphic marker is selected from the group consisting of rs3825214, rs6795970, rs3807989, rs7660702, rs132311, rs1733724 and rs365990, and markers in linkage disequilibrium therewith.

Another aspect relates to a method of determining a susceptibility to a vascular condition selected from the group consisting of: an abnormal electrocardiogram measure, Atrial Fibrillation, Atrial Flutter, and Stroke, in a human individual, the method comprising analyzing sequence data about a human individual identifying at least one allele of at least one polymorphic marker, wherein different alleles of the at least one polymorphic marker are associated with different susceptibilities to the condition in humans, and determining a susceptibility to the condition from the sequence data, wherein the at least one polymorphic marker is selected from the group consisting of rs3825214, rs6795970, rs3807989, rs7660702, rs132311, rs1733724 and rs365990, and markers in linkage disequilibrium therewith.

Abnormal electrocardiogram measures, in the present context, are electrocardiogram measures that deviate from what is considered normal. The abnormal measure may be elevated, or increased, or the abnormal measure may be deflated or decreased.

In certain embodiments, the abnormal electrocardiogram measure is selected from the group consisting of an increased QRS interval, an increased PR interval, an increased QT interval, sick sinus syndrome and an increased heart rate. In certain embodiments, the abnormal electrocardiogram measure is selected from the group consisting of a decreased QRS interval, a decreased PR interval, a decreased QT interval, sick sinus syndrome and a decreased heart rate. In certain embodiments, the abnormal electrocardiogram measure is selected from the group consisting of an increased and/or decreased QRS interval, an increased and/or decreased PR interval, an increased and/or decreased QT interval, and an increased and/or decreased heart rate. Thus, certain alleles at polymorphic markers described herein are predictive of increased ECG measures, while other alleles at these markers are predictive of decreased ECG measures. Thus, the markers may be useful for detecting susceptibility to increased ECG measures, detecting a susceptibility to decreased ECG measures, or useful for detecting susceptibility to both increase and decreased ECG susceptibility.

In one preferred embodiment, the vascular condition is Atrial Fibrillation and the marker is selected from the group consisting of rs3825214 and rs3807989, and markers in linkage disequilibrium therewith. In another preferred embodiment, the vascular condition is Atrial Fibrillation and the marker is selected from the group consisting of the markers set forth in Table 15 and Table 17. In another preferred embodiment, the vascular condition is Atrial Fibrillation and the marker is selected from the group consisting of the markers set forth in Table 15. In another preferred embodiment, the vascular condition is Atrial Fibrillation and the marker is selected from the group consisting of the markers set forth in Table 17.

In another preferred embodiment, the vascular condition is Pacemaker placement and the marker is selected from the group consisting of rs6795970, and markers in linkage disequilibrium therewith. In another preferred embodiment, the vascular condition is Pacemaker placement and the marker is selected from the group consisting of the markers set forth in Table 20.

In another preferred embodiment, the vascular condition is abnormal heart rate, and the marker is selected from the group consisting of rs365990, and markers in linkage disequilibrium therewith. In another preferred embodiment, the vascular condition is abnormal heart rate, and the marker is selected from the group consisting of the markers set forth in Table 21. In certain embodiments, the abnormal heart rate is elevated heart rate.

In another embodiment, the electrocardiogram measure is QRS interval, and the at least one polymorphic marker is selected from the group consisting of rs3825214, rs6795970, rs1321311 and rs1733724, and markers in linkage disequilibrium therewith. In another embodiment, the electrocardiogram measure is QRS interval, and the at least one polymorphic marker is selected from the group consisting of the markers set forth in Table 13, Table 19, Table 23 and Table 24. In another embodiment, the electrocardiogram measure is QRS interval, and the at least one polymorphic marker is selected from the group consisting of the markers set forth in Table 13. In another embodiment, the electrocardiogram measure is QRS interval, and the at least one polymorphic marker is selected from the group consisting of the markers set forth in Table 19. In another embodiment, the electrocardiogram measure is QRS interval, and the at least one polymorphic marker is selected from the group consisting of the markers set forth in Table 23. In another embodiment, the electrocardiogram measure is QRS interval, and the at least one polymorphic marker is selected from the group consisting of the markers set forth in Table 24.

In another embodiment, the electrocardiogram measure is PR interval, and the at least one polymorphic marker is selected from the group consisting of rs3825214, rs3807989, rs6795970 and rs7660702, and markers in linkage disequilibrium therewith. In another embodiment, the electrocardiogram measure is PR interval, and the at least one polymorphic marker is selected from the group consisting of the markers set forth in Table 14, Table 16, Table 18 and Table 22. In another embodiment, the electrocardiogram measure is PR interval, and the at least one polymorphic marker is selected from the group consisting of the markers set forth in Table 14. In another embodiment, the electrocardiogram measure is PR interval, and the at least one polymorphic marker is selected from the group consisting of the markers set forth in Table 16. In another embodiment, the electrocardiogram measure is PR interval, and the at least one polymorphic marker is selected from the group consisting of the markers set forth in Table 18. In another embodiment, the electrocardiogram measure is PR interval, and the at least one polymorphic marker is selected from the group consisting of the markers set forth in Table 22.

In certain embodiments, the sequence data is nucleic acid sequence data. In certain other embodiments, the sequence data is amino acid sequence data (e.g., protein sequence data or polypeptide sequence data). It may be useful to obtain sequence data about more than more polymorphic marker. Certain embodiments therefore comprise obtaining sequence data (nucleic acid sequence data and/or polypeptide sequence data) about at least two polymorphic markers.

Nucleic acid sequence data may be obtained using techniques and methods known to the skilled person. For example, in one embodiment, obtaining nucleic acid sequence data comprises obtaining a biological sample from the human individual and analyzing sequence of the at least one polymorphic marker in the sample. Determination of the sequence of a polymorphism comprises a determination of the allele or alleles present at the polymorphic site in the genome of the individual. Alternatively, the sequence of the individual may be analyzed in a dataset that contains information about the sequence of the individual. In certain embodiments, the dataset is a genotype dataset. Analyzing the allele(s) present in the dataset for a polymorphic marker is a determination of the sequence of the polymorphic marker in the individual at the particular polymorphic marker. In certain other embodiments, the dataset is a sequence dataset comprising genomic sequence information about the individual. The sequence information may comprise the entire genomic sequence of the individual; alternatively, the sequence information comprises a portion of the genomic sequence of the individual. Preferably, the sequence information includes information about at least one polymorphic marker in the genome of the individual.

Sequence data may in certain embodiments be predetermined. In other words, the invention may be practiced by obtaining sequence information from a preexisting record and analyzing the sequence information. The preexisting record is suitably in a computer-readable format, but may also be in other formats, such as printed sequence information.

In one embodiment, the genotype dataset comprises a table, such as a look-up table, containing information about at least one risk measure of the vascular condition for the at least one polymorphic marker. For example, the look-up table may contain information about the relative risk (RR) and/or odds ratio (OR) for a particular polymorphic marker for the vascular condition.

Risk assessment for a particular polymorphic marker, or a plurality of markers, may be reported in any convenient manner known to the skilled person. For example, a risk assessment report for an individual may be generated and made available to the individual or a third party. The report may contain a personal identifier and at least one risk measure for at least one marker. The report may contain a risk measure for a combination of markers, as described herein. The report may be made available through a database or server, for example via a web interface. The report may also be provided in a printed format.

In certain embodiments, the sequence data is amino acid sequence data. In one embodiment, the amino acid sequence data identifies the presence or absence of an amino acid substitution in a protein selected from the group consisting of the human SCN10A protein and the human MYH6 protein. In one preferred embodiment, the amino acid substitution is a Valine to Alanine substitution at position 1073 (V1073A) of a human SCN10A protein. In another preferred embodiment, the amino acid substitution is an Alanine to Valine substitution at position 1101 (A1101V) of a human MYH6 protein.

As described further herein, surrogate markers in linkage disequilibrium with a marker of interest (e.g., a marker predictive of an ECG measure or a related phenotype, such as Atrial Fibrillation, Pacemaker placment) may also be used to practice the present invention. Surrogate markers are in linkage disequilibrium with the anchor marker by certain numerical values of a measure of linkage disequilibrium, such as D′ or r². In preferred embodiments of the invention, surrogate markers are selected from groups of markers correlated with an anchor marker (i.e., in linkage disequilibrium with the anchor marker) by certain values of r². In certain preferred embodiments, the surrogate markers are in LD with the anchor marker by values of r² of greater than 0.2. The surrogate markers may also be suitably selected based on other values of r², such as values of r² of greater than 0.3, greater than 0.4, greater than 0.5, greater than 0.6, greater than 0.7, greater than 0.8, greater than 0.9, and greater than 0.95. Exemplary surrogate markers are given in Tables 6-12 herein, together with values of the LD measures D′ and r², which can be used to selected surrogate markers using any suitable cutoff value of r² of 0.1 or 0.2 or greater. Further surrogate markers are given in Tables 13-24 herein, together with observed values of their association with electrocardiogram measures, Pacemaker placement and Atrial Fibrillation.

In certain embodiments, markers in linkage disequilibrium with rs6795970 are selected from the group consisting of rs6599240, rs11129800, rs11129801, rs11710006, rs11924846, rs9990137, rs6805187, rs7617547, rs6771157, rs4076737, rs12632942, rs7430477, rs6795970, rs6801957, rs7433306, rs6780103, rs6790396, rs6800541, rs7615140, rs6599250, rs6599251, rs7430451, rs6599254, rs6599255, rs12630795, rs6798015, rs6763876, rs6599256, rs7641844, rs7432804, rs7430439, rs7651106, rs6599257, rs7610489, rs7650384, rs4414778, and rs10212338, which are the markers listed in Table 6.

In certain embodiments, markers in linkage disequilibrium with rs7660702 are selected from the group consisting of rs7698203, rs6849659, rs2101134, rs10017047, rs12648692, rs13134382, rs17010599, rs7655100, rs7677064, rs10033273, rs7439720, rs900204, rs11731040, rs931195, rs17010632, rs1871864, rs1871865, rs1482085, rs11735639, rs4413396, rs13146939, rs13152150, rs13128115, rs12509904, rs12650494, rs10012090, rs7691602, rs7692808, rs7658797, rs17010697, rs343860, rs13108523, rs1482094, rs7676486, rs7660702, rs2062098, rs1482091, rs6813860, rs994285, rs343853, rs343849, rs3889735, rs2601855, rs2601857, rs7682971, rs10516755, rs1020584, rs13106553, rs12510813, rs12507272, rs13137008, rs13112493, rs4693735, rs12507198, rs13111662, rs11732231, rs11736641, rs11097071, rs7674888, rs1966862, rs12054628, rs17010839, rs11945319, rs6831420, rs7680588, rs17010851, rs17010857, rs4693736, rs13105921, rs17010887, rs17010892, rs17395020, rs17399123, rs10516756, rs1452681, rs9790823, rs7683733, rs7662174, rs7684607, rs13118915, rs17010925, rs12503243, rs7675429, rs7689056, and rs7693640, which are the markers listed in Table 7.

In certain embodiments, markers in linkage disequilibrium with rs1321311 are selected from the group consisting of rs6457931, rs12207916, rs1321313, rs4713994, rs1321311, rs1321310, rs4331968, rs9470361, rs6930671, rs11969445, rs9470366, rs6936993, rs9470367, rs7756236, rs9462207, rs9368950, rs9462208, rs9462209, rs9462210, rs10807170, rs4713996, rs9394368, rs4713999, rs4711457, rs6930083, rs4714001, rs1321309, rs733590, rs2395655, rs3176352, rs12207548, rs12191972, rs7767246, rs6937605, rs7762245, which are the markers listed in Table 8.

In certain embodiments, markers in linkage disequilibrium with rs3807989 are selected from the group consisting of rs2157799, rs721994, rs1728723, rs2049902, rs11772856, rs1858810, rs7781492, rs10464649, rs12706089, rs7782281, rs4727831, rs768108, rs717957, rs1883049, rs6959099, rs6975771, rs6976316, rs6954077, rs728690, rs10228178, rs2402081, rs2270188, rs10271007, rs4730743, rs4727833, rs2109513, rs6466579, rs3919515, rs975028, rs2215448, rs2742125, rs3779512, rs9649394, rs1474510, rs3807986, rs6466584, rs6466585, rs1476833, rs976739, rs3807989, rs3801995, rs3815412, rs11773845, rs9886215, rs9886219, rs2109516, rs3757732, rs3757733, rs7804372, rs729949, rs3807990, rs3807992, rs3807994, rs6466587, rs6466588, rs1049314, rs8713, rs6867, rs1049337, rs6961215, rs6961388, rs10280730, rs10232369, rs6959106, rs7802124, rs7802438, rs1860588, rs2052106, rs11979486, rs10273326, rs6466589, rs7795356, rs2109517, rs2056865, rs2191503, rs4727835, rs7800573, rs6955302, rs6978354, which are the markers listed in Table 9.

In certain embodiments, markers in linkage disequilibrium with rs1733724 are selected from the group consisting of rs1149782, rs1149781, rs1194673, rs1149776, rs1149775, rs1149772, rs1149769, rs1194671, rs1194670, rs1194669, rs1194668, rs6480837, rs1209265, rs1194664, rs1194663, rs1660760, rs12355839, rs1194743, rs1733724, which are the markers listed in Table 10.

In certain embodiments, markers in linkage disequilibrium with rs3825214 are selected from the group consisting of rs6489952, rs1895593, rs7966567, rs8181608, rs10744818, rs8181683, rs8181627, rs10744819, rs6489953, rs10744820, rs1895587, rs9669457, rs6489955, rs7309910, rs7308120, rs2384409, rs2891503, rs7977083, rs1895597, rs7316919, rs6489956, rs883079, rs2113433, rs3825214, rs12367410, rs10507248, rs7955405, rs10744823, rs7312625, rs4767237, rs7135659, rs1895585, rs1946295, rs1946293, rs3825215, rs1895582, rs7964303, rs17731569, which are the markers listed in Table 11.

In certain embodiments, markers in linkage disequilibrium with rs365990 are selected from the group consisting of rs3811178, rs8022522, rs365990, rs445754, rs10149522, rs452036, rs412768, rs439735, rs388914, rs440466, rs2277474, rs7143356, rs12147570, rs2284651, rs7149517, rs2331979, rs3729833, rs765021, rs7140721, rs3729829, rs3729828, rs3729825, rs7159367, rs12894524, rs2277475, rs12147533, rs743567, rs7157716, rs2754163, which are the markers listed in Table 12.

Obviously, any particular marker is in linkage disequilibrium with itself. Markers in linkage disequilibrium with an anchor marker therefore include the anchor marker itself. Thus, even though a particular list, such as the foregoing list, of surrogate markers does not include the anchor marker itself, it should be understood that suitable surrogates of any particular anchor marker include the anchor marker itself. In one such embodiment, markers in linkage disequilibrium with rs365990 are selected from the group consisting of rs3811178, rs8022522, rs445754, rs10149522, rs452036, rs412768, rs439735, rs388914, rs440466, rs2277474, rs7143356, rs12147570, rs2284651, rs7149517, rs2331979, rs3729833, rs765021, rs7140721, rs3729829, rs3729828, rs3729825, rs7159367, rs12894524, rs2277475, rs12147533, rs743567, rs7157716, rs2754163. Comparable embodiments of surrogate markers based on the foregoing lists of markers are also contemplated and are within scope of the invention.

Particular alleles at the polymorphic markers disclosed herein are indicative of an increased or decreased susceptibility to a particular trait. For example, the G allele of rs3825214, the A allele of rs6795970, the A allele of rs3807989, and the T allele of rs7660702 are indicative of increased susceptibility to an increased PR interval. Marker alleles of surrogate markers are likewise indicative of increased susceptibility to an increased PR interval. Further, the T allele of rs132311, the T allele of rs1733724 and the G allele of rs365990 are indicative of susceptibility to an increased QRS interval, as are marker alleles in linkage disequilibrium with these alleles. The G allele of marker rs365990 is indicative of increased heart rate in humans, as are its surrogate marker alleles, and the G allele of rs3825214 and its surrogate markers is indicative of increased susceptibility to an increased QT interval. The risk of advanced atrioventricular block (AVB) is increased in individuals with the G allele of rs3825214 or its surrogates. Risk of having a pacemaker placed is increased in individuals with the G allele of rs3825214 or its surrogates. Furthermore, individuals with the G allele of rs3825214 are at increased susceptibility to a condition selected from the group consisting of: increased PR interval, increased QRS interval, increased QT interval, atrioventricular block, and pacemaker placement. Risk of the ECG related disorders Atrial Fibrillation and Atrial Flutter is also affected by the variants described herein. Thus, the presence of at least one allele selected from the group consisting of: the A allele of rs3825214 and the G allele of rs3807989, is indicative of increased susceptibility to Atrial Fibrillation or Atrial Flutter. Surrogate marker alleles of these alleles are also indicative of risk of these diseases.

Alleles that are correlated with particular risk alleles are themselves predicted risk alleles. Thus, by way of example, the alleles recited in Tables 6-12 herein as being correlated with particular risk variants are predicted risk alleles for the particular marker.

The present invention also provides certain risk genes that are predictive of whether certain humans are at increased risk of certain vascular conditions, i.e. abnormal ECG measures, Atrial Fibrillation, Atrial Flutter and/or Stroke. Thus, certain embodiments of the invention provide methods of determining susceptibility to one or more of these conditions by evaluating markers associated with a gene selected from the group consisting of: the human TBX5 gene, the human SCN10A gene, the human CAV1 gene, the human ARHGAP24 gene, the human CDKN1A gene and the human MYH6 gene. The assaying may in certain embodiments involve obtaining sequence data about a human individual identifying certain alleles at one or more of these markers, as further discussed herein.

In certain embodiments, the at least one marker associated with the human TBX5 gene is selected from the group consisting of rs3825214, and markers in linkage disequilibrium therewith; markers associated with the human SCN10A gene are selected from the group consisting of rs6795970, and markers in linkage disequilibrium therewith; markers associated with the human CAV1 gene are selected from the group consisting of rs3807989, and markers in linkage disequilibrium therewith; markers associated with the human ARHGAP24 gene are selected from the group consisting of rs7660702, and markers in linkage disequilibrium therewith; markers associated with the human CDKN1A gene are selected from the group consisting of rs132311, and markers in linkage disequilibrium therewith; and markers associated with the human MYH6 gene are selected from the group consisting of rs365990, and markers in linkage disequilibrium therewith.

Determination of the absence of at least one of the at-risk alleles recited above is indicative of a decreased risk of the condition for the human individual. As a consequence, in certain embodiments, the analyzing comprises determining the presence or absence of at least one at-risk allele of the polymorphic marker for the condition. In one preferred embodiment, the determination of the presence of a particular at-risk susceptibility allele is indicative of increased risk of the condition for the individual. Individuals who are homozygous for at-risk alleles are at particularly high risk. Thus, in certain embodiments determination of the presence of two alleles of one or more of the above-recited risk alleles is indicative of particularly high risk (susceptibility) of the condition.

Alternatively, the allele that is detected can be the allele of the complementary strand of DNA, such that the nucleic acid sequence data includes the identification of at least one allele which is complementary to any of the alleles of the polymorphic markers referenced above.

In certain embodiments, the nucleic acid sequence data is obtained from a biological sample containing nucleic acid from the human individual. The nucleic acids sequence may suitably be obtained using a method that comprises at least one procedure selected from (i) amplification of nucleic acid from the biological sample; (ii) hybridization assay using a nucleic acid probe and nucleic acid from the biological sample; and (iii) hybridization assay using a nucleic acid probe and nucleic acid obtained by amplification of the biological sample. The nucleic acid sequence data may also be obtained from a preexisting record. For example, the preexisting record may comprise a genotype dataset for at least one polymorphic marker. In certain embodiments, the determining comprises comparing the sequence data to a database containing correlation data between the at least one polymorphic marker and susceptibility to the condition.

It is contemplated that in certain embodiments of the invention, it may be convenient to prepare a report of results of risk assessment. Thus, certain embodiments of the methods of the invention comprise a further step of preparing a report containing results from the determination, wherein said report is written in a computer readable medium, printed on paper, or displayed on a visual display. In certain embodiments, it may be convenient to report results of susceptibility to at least one entity selected from the group consisting of the individual, a guardian of the individual, a genetic service provider, a physician, a medical organization, and a medical insurer.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

### Risk Calculations

The creation of a model to calculate the overall genetic risk involves two steps: i) conversion of odds-ratios for a single genetic variant into relative risk and ii) combination of risk from multiple variants in different genetic loci into a single relative risk value.

Deriving Risk from Odds-Ratios

Most gene discovery studies for complex diseases that have been published to date in authoritative journals have employed a case-control design because of their retrospective setup. These studies sample and genotype a selected set of cases (people who have the specified disease condition) and control individuals. The interest is in genetic variants (alleles) which frequency in cases and controls differ significantly.

The results are typically reported in odds ratios, that is the ratio between the fraction (probability) with the risk variant (carriers) versus the non-risk variant (non-carriers) in the groups of affected versus the controls, i.e. expressed in terms of probabilities conditional on the affection status:

OR=(Pr(c|A)/Pr(nc|A))/(Pr(c|C)/Pr(nc|C))

Sometimes it is however the absolute risk for the disease that we are interested in, i.e. the fraction of those individuals carrying the risk variant who get the disease or in other words the probability of getting the disease. This number cannot be directly measured in case-control studies, in part, because the ratio of cases versus controls is typically not the same as that in the general population. However, under certain assumption, we can estimate the risk from the odds ratio.

It is well known that under the rare disease assumption, the relative risk of a disease can be approximated by the odds ratio. This assumption may however not hold for many common diseases. Still, it turns out that the risk of one genotype variant relative to another can be estimated from the odds ratio expressed above. The calculation is particularly simple under the assumption of random population controls where the controls are random samples from the same population as the cases, including affected people rather than being strictly unaffected individuals. To increase sample size and power, many of the large genome-wide association and replication studies use controls that were neither age-matched with the cases, nor were they carefully scrutinized to ensure that they did not have the disease at the time of the study. Hence, while not exactly, they often approximate a random sample from the general population. It is noted that this assumption is rarely expected to be satisfied exactly, but the risk estimates are usually robust to moderate deviations from this assumption.

Calculations show that for the dominant and the recessive models, where we have a risk variant carrier, “c”, and a non-carrier, “nc”, the odds ratio of individuals is the same as the risk ratio between these variants:

OR=Pr(A|c)/Pr(A|nc)=r

And likewise for the multiplicative model, where the risk is the product of the risk associated with the two allele copies, the allelic odds ratio equals the risk factor:

OR=Pr(A|aa)/Pr(A|ab)=Pr(A|ab)/Pr(A|bb)=r

Here “a” denotes the risk allele and “b” the non-risk allele. The factor “r” is therefore the relative risk between the allele types.

For many of the studies published in the last few years, reporting common variants associated with complex diseases, the multiplicative model has been found to summarize the effect adequately and most often provide a fit to the data superior to alternative models such as the dominant and recessive models.

### The Risk Relative to the Average Population Risk

It is most convenient to represent the risk of a genetic variant relative to the average population since it makes it easier to communicate the lifetime risk for developing the disease compared with the baseline population risk. For example, in the multiplicative model we can calculate the relative population risk for variant “aa” as:

RR(aa)=Pr(A|aa)/Pr(A)=(Pr(A|aa)/Pr(A|bb))/(Pr(A)/Pr(A|bb))=r²/(Pr(aa)r²+Pr(ab)r+Pr(bb))=r²/(p²r²+2pqr+q²)=r²/R

Here “p” and “q” are the allele frequencies of “a” and “b” respectively. Likewise, we get that RR(ab)=r/R and RR(bb)=1/R. The allele frequency estimates may be obtained from the publications that report the odds-ratios and from the HapMap database. Note that in the case where we do not know the genotypes of an individual, the relative genetic risk for that test or marker is simply equal to one.

As an example, for Atrial Fibrillation risk, allele A of marker rs3825214 has an allelic OR of 1.14 and a frequency (p) around 0.80 in Caucasian populations. The genotype relative risk compared to genotype GG are estimated based on the multiplicative model.

For AA it is 1.14×1.14=1.30; for AG it is simply the OR 1.14, and for GG it is 1.0 by definition.

The frequency of allele G is q=1−p=1−0.80=0.20. Population frequency of each of the three possible genotypes at this marker is:

Pr(AA)=p²=0.64, Pr(AG)=2pq=0.32, and Pr(GG)=q²=0.04

The average population risk relative to genotype GG (which is defined to have a risk of one) is:

R=0.64×1.30+0.32×1.14+0.04×1=1.24

Therefore, the risk relative to the general population (RR) for individuals who have one of the following genotypes at this marker is:

RR(AA)=1.30/1.24=1.05, RR(AG)=1.14/1.24=0.92, RR(GG)=1/1.24=0.81.

Combining the Risk from Multiple Markers

When genotypes of many SNP variants are used to estimate the risk for an individual a multiplicative model for risk can generally be assumed. This means that the combined genetic risk relative to the population is calculated as the product of the corresponding estimates for individual markers, e.g. for two markers g1 and g2:

RR(g1,g2)=RR(g1)RR(g2)

The underlying assumption is that the risk factors occur and behave independently, i.e. that the joint conditional probabilities can be represented as products:

Pr(A|g1,g2)=Pr(A|g1)Pr(A|g2)/Pr(A) and Pr(g1,g2)=Pr(g1)Pr(g2)

Obvious violations to this assumption are markers that are closely spaced on the genome, i.e. in linkage disequilibrium, such that the concurrence of two or more risk alleles is correlated. In such cases, we can use so called haplotype modeling where the odds-ratios are defined for all allele combinations of the correlated SNPs.

As is in most situations where a statistical model is utilized, the model applied is not expected to be exactly true since it is not based on an underlying bio-physical model. However, the multiplicative model has so far been found to fit the data adequately, i.e. no significant deviations are detected for many common diseases for which many risk variants have been discovered.

As an example, an individual who has the following genotypes at 4 hypothetical markers associated with a particular disease along with the risk relative to the population at each marker:

| Marker | Genotype | Calculated risk |
|---|---|---|
| M1 | CC | 1.03 |
| M2 | GG | 1.30 |
| M3 | AG | 0.88 |
| M4 | TT | 1.54 |

Combined, the overall risk relative to the population for this individual is:

1.03×1.30×0.88×1.54=1.81.

### Adjusted Life-Time Risk

The lifetime risk of an individual is derived by multiplying the overall genetic risk relative to the population with the average life-time risk of the disease in the general population of the same ethnicity and gender and in the region of the individual's geographical origin. As there are usually several epidemiologic studies to choose from when defining the general population risk, we will pick studies that are well-powered for the disease definition that has been used for the genetic variants.

For example, if the overall genetic risk relative to the population for a particular disease or trait is 1.8, and if the average life-time risk of the disease is 20%, then the adjusted lifetime risk is 20%×1.8=36%.

Note that since the average RR for a population is one, this multiplication model provides the same average adjusted life-time risk of the disease. Furthermore, since the actual life-time risk cannot exceed 100%, there must be an upper limit to the genetic RR.

### Risk Assessment

As described herein, certain polymorphic markers and haplotypes comprising such markers are found to be useful for risk assessment of certain vascular conditions, including abnormal electrocardiogram measures, Atrial Fibrillation, Atrial Flutter and Stroke. Risk assessment can involve the use of the markers for determining a susceptibility to a particular condition. Particular alleles of certain polymorphic markers are found more frequently in individuals with the condition, than in individuals without the condition. Therefore, these marker alleles have predictive value for detecting the condition, or a susceptibility to the condition, in an individual. Markers in linkage disequilibrium with risk variants (or protective variants) can be used as surrogates for these markers (and/or haplotypes). Such surrogate markers can for example be located within a particular haplotype block or LD block. Such surrogate markers can also sometimes be located outside the physical boundaries of such a haplotype block or LD block, either in close vicinity of the LD block/haplotype block, but possibly also located in a more distant genomic location.

Long-distance LD can for example arise if particular genomic regions (e.g., genes) are in a functional relationship. For example, if two genes encode proteins that play a role in a shared metabolic pathway, then particular variants in one gene may have a direct impact on observed variants for the other gene. Let us consider the case where a variant in one gene leads to increased expression of the gene product. To counteract this effect and preserve overall flux of the particular pathway, this variant may have led to selection of one (or more) variants at a second gene that confers decreased expression levels of that gene. These two genes may be located in different genomic locations, possibly on different chromosomes, but variants within the genes are in apparent LD, not because of their shared physical location within a region of high LD, but rather due to evolutionary forces. Such LD is also contemplated and within scope of the present invention. The skilled person will appreciate that many other scenarios of functional gene-gene interaction are possible, and the particular example discussed here represents only one such possible scenario.

Markers with values of r² equal to 1 are perfect surrogates for the at-risk variants (anchor variants), i.e. genotypes for one marker perfectly predicts genotypes for the other. Markers with smaller values of r² than 1 (e.g., markers with values of r² to the marker of 0.2-1.0) can also be surrogates for the at-risk variant, or alternatively represent variants with relative risk values as high as or possibly even higher than the at-risk variant. In certain preferred embodiments, markers with values of r² greater than 0.2 to the at-risk anchor variant are useful surrogate markers. The at-risk variant identified may not be the functional variant itself, but is in this instance in linkage disequilibrium with the true functional variant. The functional variant may be a SNP, but may also for example be a tandem repeat, such as a minisatellite or a microsatellite, a transposable element (e.g., an Alu element), or a structural alteration, such as a deletion, insertion or inversion (sometimes also called copy number variations, or CNVs). The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation identify and genotype surrogate markers in linkage disequilibrium with the markers and/or haplotypes as described herein. The tagging or surrogate markers in LD with the at-risk variants detected also have predictive value.

The present invention can in certain embodiments be practiced by assessing a sample comprising genomic DNA from an individual for the presence of certain variants described herein. Such assessment typically steps that detect the presence or absence of at least one allele of at least one polymorphic marker, using methods well known to the skilled person and further described herein, and based on the outcome of such assessment, determine whether the individual from whom the sample is derived is at increased or decreased risk (i.e., increased or decreased susceptibility) of a particular condition. Detecting particular alleles of polymorphic markers can in certain embodiments be done by obtaining nucleic acid sequence data about a particular human individual, that identifies at least one allele of at least one polymorphic marker. Different alleles of the at least one marker are associated with different susceptibility to the disease in humans. Obtaining nucleic acid sequence data can comprise nucleic acid sequence at a single nucleotide position, which is sufficient to identify alleles at SNPs. The nucleic acid sequence data can also comprise sequence at any other number of nucleotide positions, in particular for genetic markers that comprise multiple nucleotide positions, and can be anywhere from two to hundreds of thousands, possibly even millions, of nucleotides (in particular, in the case of copy number variations (CNVs)).

In certain embodiments, the invention can be practiced utilizing a dataset comprising information about the genotype status of at least one polymorphic marker associated with a disease (or markers in linkage disequilibrium with at least one marker associated with the disease). In other words, a dataset containing information about such genetic status, for example in the form of genotype counts at a certain polymorphic marker, or a plurality of markers (e.g., an indication of the presence or absence of certain at-risk alleles), or actual genotypes for one or more markers, can be queried for the presence or absence of certain at-risk alleles at certain polymorphic markers shown by the present inventors to be associated with the disease. A positive result for a variant (e.g., marker allele) associated with the disease, is indicative of the individual from which the dataset is derived is at increased susceptibility (increased risk) of the disease.

In certain embodiments of the invention, a polymorphic marker is correlated to a disease by referencing genotype data for the polymorphic marker to a database, such as a look-up table, that comprises correlation data between at least one allele of the polymorphism and the disease. In some embodiments, the table comprises a correlation for one polymorphism. In other embodiments, the table comprises a correlation for a plurality of polymorphisms. In both scenarios, by referencing to a look-up table that gives an indication of a correlation between a marker and the disease, a risk for the disease, or a susceptibility to the disease, can be identified in the individual from whom the sample is derived. In some embodiments, the correlation is reported as a statistical measure. The statistical measure may be reported as a risk measure, such as a relative risk (RR), an absolute risk (AR) or an odds ratio (OR).

Risk markers may be useful for risk assessment and diagnostic purposes, either alone or in combination. Results of disease risk assessment based on the markers described herein can also be combined with data for other genetic markers or risk factors for the disease, to establish overall risk. Thus, even in cases where the increase in risk by individual markers is relatively modest, e.g. on the order of 10-30%, the association may have significant implications when combined with other risk markers. Thus, relatively common variants may have significant contribution to the overall risk (Population Attributable Risk is high), or combination of markers can be used to define groups of individual who, based on the combined risk of the markers, is at significant combined risk of developing the disease or condition.

Thus, in certain embodiments of the invention, a plurality of variants (genetic markers, biomarkers and/or haplotypes) is used for overall risk assessment. These variants are in one embodiment selected from the variants as disclosed herein. Other embodiments include the use of the variants of the present invention in combination with other variants known to be useful for diagnosing a susceptibility to vascular conditions that include one or more electrocardiogram measure, Atrial Fibrillation, Atrial Flutter and/or Stroke. In such embodiments, the genotype status of a plurality of markers and/or haplotypes is determined in an individual, and the status of the individual compared with the population frequency of the associated variants, or the frequency of the variants in clinically healthy subjects, such as age-matched and sex-matched subjects. Methods known in the art, such as multivariate analyses or joint risk analyses, such as those described herein, or other methods known to the skilled person, may subsequently be used to determine the overall risk conferred based on the genotype status at the multiple loci. Assessment of risk based on such analysis may subsequently be used in the methods, uses and kits of the invention, as described herein.

### Study Population

In a general sense, the methods and kits described herein can be utilized from samples containing nucleic acid material (DNA or RNA) from any source and from any individual, or from genotype or sequence data derived from such samples. In preferred embodiments, the individual is a human individual. The individual can be an adult, child, or fetus. The nucleic acid source may be any sample comprising nucleic acid material, including biological samples, or a sample comprising nucleic acid material derived therefrom. The present invention also provides for assessing markers and/or haplotypes in individuals who are members of a target population. Such a target population is in one embodiment a population or group of individuals at risk of developing a particular condition, based on other genetic factors, biomarkers, biophysical parameters (e.g., weight, BMD, blood pressure), or general health and/or lifestyle parameters (e.g., history of the condition or related condition, previous diagnosis of the condition, family history of the condition).

The invention provides for embodiments that include individuals from specific age subgroups, such as those over the age of 40, over age of 45, or over age of 50, 55, 60, 65, 70, 75, 80, or 85. Other embodiments of the invention pertain to other age groups, such as individuals aged less than 85, such as less than age 80, less than age 75, or less than age 70, 65, 60, 55, 50, 45, 40, 35, or age 30. Other embodiments relate to individuals with age at onset of the condition in any of the age ranges described in the above. It is also contemplated that a range of ages may be relevant in certain embodiments, such as age at onset at more than age 45 but less than age 60. Other age ranges are however also contemplated, including all age ranges bracketed by the age values listed in the above. The invention furthermore relates to individuals of either gender, males or females.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Sulem, P., et al. Nat Genet May 17, 2009 (Epub ahead of print); Rafnar, T., et al. Nat Genet. 41:221-7 (2009); Gretarsdottir, S., et al. Ann Neurol 64:402-9 (2008); Stacey, S, N., et al. Nat Genet. 40:1313-18 (2008); Gudbjartsson, D. F., et al. Nat Genet. 40:886-91 (2008); Styrkarsdottir, U., et al. N Engl J Med 358:2355-65 (2008); Thorgeirsson, T., et al. Nature 452:638-42 (2008); Gudmundsson, 3., et al. Nat. Genet. 40:281-3 (2008); Stacey, S, N., et al., Nat. Genet. 39:865-69 (2007); Helgadottir, A., et al., Science 316:1491-93 (2007); Steinthorsdottir, V., et al., Nat. Genet. 39:770-75 (2007); Gudmundsson, 3., et al., Nat. Genet. 39:631-37 (2007); Frayling, T M, Nature Reviews Genet. 8:657-662 (2007); Amundadottir, L. T., et al., Nat. Genet. 38:652-58 (2006); Grant, S. F., et al., Nat. Genet. 38:320-23 (2006)). Thus, genetic findings in the Icelandic population have in general been replicated in other populations, including populations from Africa and Asia.

It is thus believed that the markers described herein will show similar association profiles in other human populations. Particular embodiments comprising individual human populations are thus also contemplated and within the scope of the invention. Such embodiments relate to human subjects that are from one or more human population including, but not limited to, Caucasian populations, European populations, American populations, Eurasian populations, Asian populations, Central/South Asian populations, East Asian populations, Middle Eastern populations, African populations, Hispanic populations, and Oceanian populations. European populations include, but are not limited to, Swedish, Norwegian, Finnish, Russian, Danish, Icelandic, Irish, Kelt, English, Scottish, Dutch, Belgian, French, German, Spanish, Portuguese, Italian, Polish, Bulgarian, Slavic, Serbian, Bosnian, Czech, Greek and Turkish populations.

The racial contribution in individual subjects may also be determined by genetic analysis. Genetic analysis of ancestry may be carried out using unlinked microsatellite markers such as those set out in Smith et al. (Am J Hum Genet. 74, 1001-13 (2004)).

In certain embodiments, the invention relates to markers and/or haplotypes identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply the methods available and as thought herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Utility of Genetic Testing

The person skilled in the art will appreciate and understand that the risk variants described herein in general do not, by themselves, provide an absolute identification of individuals who will develop a particular condition. The variants described herein do however indicate increased and/or decreased likelihood that individuals carrying the at-risk or protective variants of the invention will develop the condition. The present inventors have discovered that certain variants confer risk of developing certain vascular condition (e.g., abnormal ECG measures, Atrial Fibrillation, Atrial Flutter, Stroke), as supported by the results presented in the Exemplification herein. This information is extremely valuable in itself, as outlined in more detail in the below, as it can be used to, for example, initiate preventive measures at an early stage, perform regular physical exams to monitor the progress and/or appearance of symptoms, or to schedule exams at a regular interval to identify early symptoms, so as to be able to apply treatment at an early stage.

Genetic testing may be useful for selecting appropriate work-up for individuals presenting with subtle cardiac symptoms. A genetic test that identifies an individual at-risk for abnormal ECG and/or at-risk for Atrial Fibrillation, Atrial Flutter and/or stroke may be used to select the appropriate work-up in the clinic. Thus, individuals who carry one or more genetic risk factors for an abnormal ECG measure and/or Atrial Fibrillation, Atrial Flutter and/or stroke, using any one, or a combination of, the markers described herein, would undergo a more thorough work-up. Thus, genetic testing may be used to determine the aggressiveness of the clinical work-up of individual who present with vague or unclear initial symptoms.

Genetic testing may also be useful for therapy choice. It is known that calcium and beta blockers may predispose to increased PR interval in humans. Thus, individuals determined to be at increased genetic risk for increased PR interval using any one or a combination of the markers described herein may be given alternative therapy upon presentation of Atrial Fibrillation and/or Atrial Flutter, to minimize the adverse reaction of an increased PR interval caused by calcium and beta blockers.

Heart blocks can usually be diagnosed using ECG. Symtpoms associated with heart block depend on the severity of the conduction disturbance and may range from a lack of apparent symptoms to syncope (fainting) and life-threatening collapse. For individuals with intermittent heart-block, symptoms may be more subtle, and hence the diagnosis is not straightforward. Genetic testing can be used to identify those individuals at increased risk of heart block, (e.g., individuals determined as being at elevated risk of heart block using any one, or a combination of, the markers described herein) who may then be chosen for a more extensive clinical work-up to identify underlying heart block.

The present invention relates to risk assessment for vascular conditions such as abnormal ECG measures, cardiac arrhythmia (e.g., atrial fibrillation or atrial flutter) and/or stroke, including determining whether an individual is at risk for developing cardiac arrhythmia (e.g., atrial fibrillation or atrial flutter) and/or stroke. The markers of the present invention can be used alone or in combination, as well as in combination with other factors, including other genetic risk factors or biomarkers, for risk assessment of an individual for these conditions. Many factors known to affect the predisposition of an individual towards vascular conditions are known to the person skilled in the art and can be utilized in such assessment. These include, but are not limited to, age, gender, smoking status, physical activity, waist-to-hip circumference ratio, family history of cardiac arrhythmia or an abnormal ECG measure (in particular atrial fibrillation and/or atrial flutter) and/or stroke, previously diagnosed cardiac arrhythmia (e.g., atrial fibrillation or atrial flutter), abnormal ECG measure and/or stroke, obesity, hypertriglyceridemia, low HDL cholesterol, hypertension, elevated blood pressure, cholesterol levels, HDL cholesterol, LDL cholesterol, triglycerides, apolipoprotein AI and B levels, fibrinogen, ferritin, C-reactive protein and leukotriene levels. Particular biomarkers that have been associated with Atrial fibrillation/Atrial flutter and stroke are discussed in Allard et al. (Clin Chem 51:2043-2051 (2005) and Becker (J Thromb Thrombolys 19:71-75 (2005)). These include, but are not limited to, fibrin D-dimer, prothrombin activation fragment 1.2 (F1.2), thrombin-antithrombin III complexes (TAT), fibrinopeptide A (FPA), lipoprotein-associated phospholipase A2 (Ip-PLA2), beta-thromboglobulin, platelet factor 4, P-selectin, von Willebrand Factor, pro-natriuretic peptide (BNP), matrix metalloproteinase-9 (MMP-9), PARK7, nucleoside diphosphate kinase (NDKA), tau, neuron-specific enolase, B-type neurotrophic growth factor, astroglial protein S-100b, glial fibrillary acidic protein, C-reactive protein, seum amyloid A, marix metalloproteinase-9, vascular and intracellular cell adhesion molecules, tumor necrosis factor alpha, and interleukins, including interleukin-1, -6, and -8). Circulating progenitor cells have also been implicated as being useful biomarkers for AF. In particular embodiments, more than one biomarker is determined for an individual, and combined with results of a determination of at least one polymorphic marker as described herein. Preferably, biomarker is measured in plasma or serum from the individual.

Alternatively, the biomarker is determined in other suitable tissues containing measurable amounts of the biomarker, and such embodiments are also within scope of the invention.

Methods known in the art can be used for overall risk assessment, including multivariate analyses or logistic regression.

Atrial fibrillation is a disease of great significance both to the individual patient and to the health care system as a whole. It can be a permanent condition but may also be paroxysmal and recurrent in which case it can be very challenging to diagnose. The most devastating complication of atrial fibrillation and atrial flutter is the occurrence of debilitating stroke. Importantly the risk of stroke is equal in permanent and paroxysmal atrial fibrillation. It has repeatedly been shown that therapy with warfarin anticoagulation can significantly reduce the risk of first or further episodes of stroke in the setting of atrial fibrillation. Therefor, anticoagulation with warfarin is standard therapy for almost all patients with atrial fibrillation for stroke-prevention, whether they have the permanent or paroxysmal type. The only patients for whom warfarin is not strongly recommended are those younger than 65 years old who are considered low-risk, i.e., they have no organic heart disease, including, neither hypertension no coronary artery disease, no previous history of stroke or transient ischemic attacks and no diabetes. This group has a lower risk of stroke and stroke-prevention with aspirin is recommended.

Due to the nature of paroxysmal atrial fibrillation, it can be very difficult to diagnose. When the patient seeks medical attention due to disease-related symptoms, such as palpitations, chest pain, shortness of breath, dizziness, heart failure, transient ischemic attacks or even stroke, normal heart rhythm may already be restored precluding diagnosis of the arrhythmia. In these cases cardiac rhythm monitoring is frequently applied in the attempt to diagnose the condition. The cardiac rhythm is commonly monitored continuously for 24 to 48 hours. Unfortunately atrial fibrillation episodes are unpredictable and frequently missed by this approach. The opportunity to diagnose the arrhythmia, institute recommended therapy, and possibly prevent a debilitating first or recurrent stroke may be missed with devastating results to the patient. Prolonged and more complex cardiac rhythm monitoring measures are available and applied occasionally when the suspicion of atrial fibrillation is very strong. These tests are expensive, the diagnostic yield with current approach is often low, and they are used sparingly for this indication. In these circumstances additional risk stratification with genetic testing may be extremely helpful. Understanding that the individual in question carries either an at-risk or a protective genetic variant can be an invaluable contribution to diagnostic and/or treatment decision making. This way, in some cases, unnecessary testing and therapy may be avoided, and in other cases, with the help of more aggressive diagnostic approach, the arrhythmia may be diagnosed and/or proper therapy initiated and later complications of disease diminished.

### How Genetic Testing May Directly Affect Choice of Treatment

When individuals present with their first (diagnosed) episode of paroxysmal atrial fibrillation and either spontaneously convert to sinus rhythm or undergo electrical or chemical cardioversion less than 48 hours into the episode, the decision to initiate, or not to initiate, anticoagulation therapy, is individualized based on the risk profile of the patient in question and the managing physicians preference. This can be a difficult choice to make since committing the patient to anticoagulation therapy has a major impact on the patients life. Often the choice is made to withhold anticoagulation in such a situation and this may be of no significant consequence to the patient. On the other hand the patient may later develop a stroke and the opportunity of prevention may thus have been missed. In such circumstances, knowing that the patient is a carrier of the at-risk variant may be of great significance and support initiation of anticoagulation treatment.

Individuals who are diagnosed with atrial fibrillation under the age of 65 and are otherwise considered low risk for stroke, i.e. have no organic heart disease, no hypertension, no diabetes and no previous history of stroke, are generally treated with aspirin only for stroke-prevention and not anticoagulation. If such a patient is found to be carrier for any one, or a combination of, the at-risk variants described herein, this could be considered support for initiating anticoagulation earlier than otherwise recommended. This would be a reasonable consideration since the results of stroke from atrial fibrillation can be devastating.

Ischemic stroke is generally classified into five subtypes based on suspected cause; large artery atherosclerosis, small artery occlusion, cardioembolism (majority due to atrial fibrillation), stroke of other determined cause and stroke of undetermined cause (either no cause found or more than one plausible cause). Importantly, stroke due to cardioembolism has the highest recurrence, is most disabling and is associated with the lowest survival. It is therefore imperative not to overlook atrial fibrillation as the major cause of stroke, particularly since treatment measures vary based on the subtype. Therefore, if an individual is diagnosed with stroke or a transient ischemic attack and a plausible cause is not identified despite standard work-up, knowing that the patient is a carrier of the at-risk variant may be of great value and support either initiation of anticoagulation treatment or more aggressive diagnostic testing in the attempt to diagnose atrial fibrillation.

Furthermore, the markers of the present invention can be used to increase power and effectiveness of clinical trials. Thus, individuals who are carriers of at least one at-risk variant of the present invention, i.e. individuals who are carriers of at least one allele of at least one polymorphic marker conferring increased risk of developing cardiac arrhythmia (e.g., atrial fibrillation or atrial flutter) and/or stroke may be more likely to respond to a particular treatment modality, e.g., as described in the above. In one embodiment, individuals who carry at-risk variants for gene(s) in a pathway and/or metabolic network for which a particular treatment (e.g., small molecule drug) is targeting, are more likely to be responders to the treatment. In another embodiment, individuals who carry at-risk variants for a gene, which expression and/or function is altered by the at-risk variant, are more likely to be responders to a treatment modality targeting that gene, its expression or its gene product. This application can improve the safety of clinical trials, but can also enhance the chance that a clinical trial will demonstrate statistically significant efficacy, which may be limited to a certain sub-group of the population. Thus, one possible outcome of such a trial is that carriers of certain genetic variants, e.g., the markers and haplotypes of the present invention, are statistically significantly likely to show positive response to the therapeutic agent, i.e. experience alleviation of symptoms associated with cardiac arrhythmia (e.g., atrial fibrillation or atrial flutter) and/or stroke when taking the therapeutic agent or drug as prescribed.

In a further aspect, the markers and haplotypes of the present invention can be used for targeting the selection of pharmaceutical agents for specific individuals. Personalized selection of treatment modalities, lifestyle changes or combination of the two, can be realized by the utilization of the at-risk variants of the present invention. Thus, the knowledge of an individual's status for particular markers of the present invention, can be useful for selection of treatment options that target genes or gene products affected by the at-risk variants of the invention. Certain combinations of variants may be suitable for one selection of treatment options, while other gene variant combinations may target other treatment options. Such combination of variant may include one variant, two variants, three variants, or four or more variants, as needed to determine with clinically reliable accuracy the selection of treatment module.

### Diagnostic and Screening Methods

In certain embodiments, the present invention pertains to methods of diagnosing, or aiding in the diagnosis of, certain vascular conditions or a susceptibility to the conditions, by detecting particular alleles at genetic markers that appear more frequently in subjects with the conditions or subjects who are susceptible to the conditions. In a particular embodiment, the invention is a method of determining a susceptibility to these conditions by detecting at least one allele of at least one polymorphic marker. In certain other embodiments, the invention relates to a method of determining a susceptibility to these conditions by detecting at least one allele of at least one polymorphic marker. The present invention describes methods whereby detection of particular alleles of particular markers or haplotypes is indicative of a susceptibility to these vascular conditions.

The present invention pertains in some embodiments to methods of clinical applications of diagnosis, e.g., diagnosis performed by a medical professional. In other embodiments, the invention pertains to methods of diagnosis or methods of determination of a susceptibility performed by a layman. The layman can be the customer of a genotyping service. The layman may also be a genotype service provider, who performs genotype analysis on a DNA sample from an individual, in order to provide service related to genetic risk factors for particular traits or diseases, based on the genotype status of the individual (i.e., the customer). Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively little cost. The resulting genotype information, which can be made available to the individual, can be compared to information about disease or trait risk associated with various SNPs, including information from public literature and scientific publications. The diagnostic application of disease-associated alleles as described herein, can thus for example be performed by the individual, through analysis of his/her genotype data, by a health professional based on results of a clinical test, or by a third party, including the genotype service provider. The third party may also be service provider who interprets genotype information from the customer to provide service related to specific genetic risk factors, including the genetic markers described herein. In other words, the diagnosis or determination of a susceptibility of genetic risk can be made by health professionals, genetic counselors, third parties providing genotyping service, third parties providing risk assessment service or by the layman (e.g., the individual), based on information about the genotype status of an individual and knowledge about the risk conferred by particular genetic risk factors (e.g., particular SNPs). In the present context, the term “diagnosing”, “diagnose a susceptibility” and “determine a susceptibility” is meant to refer to any available diagnostic method, including those mentioned above.

In certain embodiments, a sample containing genomic DNA from an individual is collected. Such sample can for example be a buccal swab, a saliva sample, a blood sample, or other suitable samples containing genomic DNA, as described further herein. The genomic DNA is then analyzed using any common technique available to the skilled person, such as high-throughput array technologies. Results from such genotyping are stored in a convenient data storage unit, such as a data carrier, including computer databases, data storage disks, or by other convenient data storage means. In certain embodiments, the computer database is an object database, a relational database or a post-relational database. The genotype data is subsequently analyzed for the presence of certain variants known to be susceptibility variants for a particular human conditions, such as the genetic variants described herein. Genotype data can be retrieved from the data storage unit using any convenient data query method. Calculating risk conferred by a particular genotype for the individual can be based on comparing the genotype of the individual to previously determined risk (expressed as a relative risk (RR) or and odds ratio (OR), for example) for the genotype, for example for an heterozygous carrier of an at-risk variant for a particular disease or trait. The calculated risk for the individual can be the relative risk for a person, or for a specific genotype of a person, compared to the average population with matched gender and ethnicity. The average population risk can be expressed as a weighted average of the risks of different genotypes, using results from a reference population, and the appropriate calculations to calculate the risk of a genotype group relative to the population can then be performed. Alternatively, the risk for an individual is based on a comparison of particular genotypes, for example heterozygous carriers of an at-risk allele of a marker compared with non-carriers of the at-risk allele. Using the population average may in certain embodiments be more convenient, since it provides a measure which is easy to interpret for the user, i.e. a measure that gives the risk for the individual, based on his/her genotype, compared with the average in the population. The calculated risk estimated can be made available to the customer via a website, preferably a secure website.

In certain embodiments, a service provider will include in the provided service all of the steps of isolating genomic DNA from a sample provided by the customer, performing genotyping of the isolated DNA, calculating genetic risk based on the genotype data, and report the risk to the customer. In some other embodiments, the service provider will include in the service the interpretation of genotype data for the individual, i.e., risk estimates for particular genetic variants based on the genotype data for the individual. In some other embodiments, the service provider may include service that includes genotyping service and interpretation of the genotype data, starting from a sample of isolated DNA from the individual (the customer).

Overall risk for multiple risk variants can be performed using standard methodology. For example, assuming a multiplicative model, i.e. assuming that the risk of individual risk variants multiply to establish the overall effect, allows for a straight-forward calculation of the overall risk for multiple markers.

In addition, in certain other embodiments, the present invention pertains to methods of determining a decreased susceptibility to particular vascular conditions, by detecting particular genetic marker alleles or haplotypes that appear less frequently in subjects with the conditions than in individuals that do not have the conditions, or in the general population.

As described and exemplified herein, particular marker alleles or haplotypes are associated with risk of certain vascular conditions. In one embodiment, the marker allele or haplotype is one that confers a significant risk or susceptibility to the condition. In another embodiment, the invention relates to a method of determining a susceptibility to the condition in a human individual, the method comprising determining the presence or absence of at least one allele of at least one polymorphic marker in a nucleic acid sample obtained from the individual. In another embodiment, the invention pertains to methods of determining a susceptibility to the vascular condition in a human individual, by screening for at least one marker allele or haplotype as described herein. In another embodiment, the marker allele or haplotype is more frequently present in a subject having, or who is susceptible to, the vascular condition (affected), as compared to the frequency of its presence in a healthy subject (control, such as population controls). In certain embodiments, the significance of association of the at least one marker allele or haplotype is characterized by a p value<0.05. In other embodiments, the significance of association is characterized by smaller p-values, such as <0.01, <0.001, <0.0001, <0.00001, <0.000001, <0.0000001, <0.00000001 or <0.000000001.

Determining risk or susceptibility in certain embodiments includes steps of obtaining sequence data that identifies at least one allele of at least one polymorphic marker. In other words, the sequence data identifies the nucleotide that is present at a particular site in the genome of the individual, thus identifying a particular allele at that site. The sequence information may optionally be represented as digital genetic profile data. Such genetic profile data may for example be in the form of allelic counts at particular polymorphic sites, in the form of the allelic identity at the particular sites or in other convenient form. The data is then suitably transformed so as to obtain a risk measure. The transformation may suitably performed on a processor, such as a computer processor on a computer system. The transformation typically involves an assessment of genetic risk associated with the allelic identity at one or more polymorphic sites (i.e., genotypes at the particular sites). Such risk assessment utilizes risk measures obtained by performing a comparison of the genetic composition of individuals with the particular condition (affecteds) to a reference group (controls) for the particular polymorphic site. The present inventors have identified certain risk markers for vascular conditions using such an approach. Risk for an individual involves comparing the individual's genotype to the risk conferred by the genotype based on a risk analysis of affecteds as compared with controls, and calculating a genetic risk for the individual based on the estimated risk for his/her genotype. The risk assessment may be based on assessment of a single polymorphic site. Alternatively, the risk assessment involves an analysis of multiple polymorphic sites, as further described herein. Results of risk assessment for an individual are then reported to the individual or a third party using any convenient method or a convenient output device. The output device is in one embodiment located on a computer server, which can be accessed remotely by the user, preferably using user-restricted access. The output device may also be a printer, which delivers a printed report which is then forwarded to the user or a third party.

In these embodiments, determination of the presence of the at least one marker allele or haplotype is indicative of a susceptibility to the particular vascular condition. The diagnostic methods involve determining whether particular alleles or haplotypes that are associated with risk of the condition are present in particular individuals, and calculate a risk measure for the individual based on the result of such determination. For multiple markers, methods of determining overall risk can be used, as described further herein. The detection of particular genetic marker alleles can be performed by a variety of methods described herein and/or known in the art. For example, genetic markers can be detected at the nucleic acid level (e.g., by direct nucleotide sequencing, or by other genotyping means known to the skilled in the art) or at the amino acid level if the genetic marker affects the coding sequence of a protein (e.g., by protein sequencing or by immunoassays using antibodies that recognize such a protein). Marker alleles correspond to fragments of a genomic segments (e.g., genes) associated with a condition (disease or trait). Such fragments encompass the DNA sequence of the polymorphic marker in question, but may also include DNA segments in strong LD (linkage disequilibrium) with the marker. In one embodiment, such fragments comprises segments in LD with the marker or haplotype as determined by a value of r² greater than 0.2 and/or |D′|>0.8).

In one embodiment, determination of a susceptibility can be accomplished using hybridization methods. (see Current Protocols in Molecular Biology, Ausubel, F. et al., eds., John Wiley & Sons, including all supplements). The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample. The invention can also be reduced to practice using any convenient genotyping method, including commercially available technologies and methods for genotyping particular polymorphic markers.

To determine a susceptibility to a condition, a hybridization sample can be formed by contacting the test sample, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 180, 250 or 500 nucleotides in length that is sufficient to specifically hybridize under stringent conditions to appropriate mRNA or genomic DNA. In certain embodiments, the oligonucleotide is from about 15 to about 100 nucleotides in length. In certain other embodiments, the oligonucleotide is from about 20 to about 50 nucleotides in length. The nucleic acid probe can comprise all or a portion of the nucleotide sequence of a particular LD block, e.g., any one of LD block C03, LD block C04, LD block C06, LD block C07, LD block C10, LD block C12 and LD block C14, as described herein, optionally comprising at least one allele of a marker described herein, or the probe can be the complementary sequence of such a sequence. In a particular embodiment, the nucleic acid probe is a portion of the nucleotide sequence of any one of LD block C03, LD block C04, LD block C06, LD block C07, LD block C10, LD block C12 and LD block C14, as described herein, optionally comprising at least one allele of a marker described herein, or at least one allele of one polymorphic marker or haplotype comprising at least one polymorphic marker described herein, or the probe can be the complementary sequence of such a sequence. Other suitable probes for use in the diagnostic assays of the invention are described herein. Hybridization can be performed by methods well known to the person skilled in the art (see, e.g., Current Protocols in Molecular Biology, Ausubel, F. et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe. The process can be repeated for any markers of the present invention, or markers that make up a haplotype, or multiple probes can be used concurrently to detect more than one marker alleles at a time. It is also possible to design a single probe containing more than one marker alleles of a particular haplotype (e.g., a probe containing alleles complementary to 2, 3, 4, 5 or all of the markers that make up a particular haplotype). Detection of the particular markers of the haplotype in the sample is indicative that the source of the sample has the particular haplotype.

In one preferred embodiment, a method utilizing a detection oligonucleotide probe comprising a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection. In such an embodiment, the amplified DNA serves as the template for the detection probe and the enhancer probe.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

Alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P., et al., Bioconjug. Chem. 5:3-7 (1994)). The PNA probe can be designed to specifically hybridize to a molecule in a sample suspected of containing one or more of the marker alleles or haplotypes that are associated with a particular vascular condition as described herein.

In one embodiment of the invention, a test sample containing genomic DNA obtained from the subject is collected and the polymerase chain reaction (PCR) is used to amplify a fragment comprising one or more markers or haplotypes of the present invention. As described herein, identification of a particular marker allele or haplotype can be accomplished using a variety of methods (e.g., sequence analysis, analysis by restriction digestion, specific hybridization, single stranded conformation polymorphism assays (SSCP), electrophoretic analysis, etc.). In another embodiment, diagnosis is accomplished by expression analysis, for example by using quantitative PCR (kinetic thermal cycling). This technique can, for example, utilize commercially available technologies, such as TaqMan® (Applied Biosystems, Foster City, Calif.). The technique can assess the presence of an alteration in the expression or composition of a polypeptide or splicing variant(s). Further, the expression of the variant(s) can be quantified as physically or functionally different.

In another embodiment of the methods of the invention, analysis by restriction digestion can be used to detect a particular allele if the allele results in the creation or elimination of a restriction site relative to a reference sequence. Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Sequence analysis can also be used to detect specific alleles or haplotypes. Therefore, in one embodiment, determination of the presence or absence of a particular marker alleles or haplotypes comprises sequence analysis of a test sample of DNA or RNA obtained from a subject or individual. PCR or other appropriate methods can be used to amplify a portion of a nucleic acid that contains a polymorphic marker or haplotype, and the presence of specific alleles can then be detected directly by sequencing the polymorphic site (or multiple polymorphic sites in a haplotype) of the genomic DNA in the sample.

In another embodiment, arrays of oligonucleotide probes that are complementary to target nucleic acid sequence segments from a subject, can be used to identify particular alleles at polymorphic sites. For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier, F. F., et al. Adv Biochem Eng Biotechnol 109:433-53 (2008); Hoheisel, J. D., Nat Rev Genet. 7:200-10 (2006); Fan, J. B., et al. Methods Enzymol 410:57-73 (2006); Raqoussis, J. & Elvidge, G., Expert Rev Mol Diagn 6:145-52 (2006); Mockler, T. C., et al Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Other methods of nucleic acid analysis that are available to those skilled in the art can be used to detect a particular allele at a polymorphic site. Representative methods include, for example, direct manual sequencing (Church and Gilbert, Proc. Natl. Acad. Sci. USA, 81: 1991-1995 (1988); Sanger, F., et al., Proc. Natl. Acad. Sci. USA, 74:5463-5467 (1977); Beavis, et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Natl. Acad. Sci. USA, 86:2766-2770 (1989)), restriction enzyme analysis (Flavell, R., et al., Cell, 15:25-41 (1978); Geever, R., et al., Proc. Natl. Acad. Sci. USA, 78:5081-5085 (1981)); heteroduplex analysis; chemical mismatch cleavage (CMC) (Cotton, R., et al., Proc. Natl. Acad. Sci. USA, 85:4397-4401 (1985)); RNase protection assays (Myers, R., et al., Science, 230:1242-1246 (1985); use of polypeptides that recognize nucleotide mismatches, such as E. coli mutS protein; and allele-specific PCR.

In another embodiment of the invention, determination of a susceptibility to a condition can be made by examining expression and/or composition of a polypeptide encoded by a nucleic acid associated with the condition, in those instances where the genetic marker(s) or haplotype(s) of the present invention result in a change in the composition or expression of the polypeptide. Thus, determination of a susceptibility to the condition can be made by examining expression and/or composition such polypeptides. The markers described herein that show association to vascular conditions may also affect expression of nearby genes (e.g., any one of the TBX5, SCN10A, CAV1, ARHGAP24, CDKN1A and MYH genes). It is well known that regulatory element affecting gene expression may be located far away, even as far as tenths or hundreds of kilobases away, from the promoter region of a gene. By assaying for the presence or absence of at least one allele of at least one polymorphic marker of the present invention, it is thus possible to assess the expression level of such nearby genes. Possible mechanisms affecting these genes include, e.g., effects on transcription, effects on RNA splicing, alterations in relative amounts of alternative splice forms of mRNA, effects on RNA stability, effects on transport from the nucleus to cytoplasm, and effects on the efficiency and accuracy of translation.

A variety of methods can be used for detecting protein expression levels, including enzyme linked immunosorbent assays (ELISA), Western blots, immunoprecipitations and immunofluorescence. A test sample from a subject is assessed for the presence of an alteration in the expression and/or an alteration in composition of the polypeptide encoded by a particular nucleic acid. An alteration in expression of a polypeptide encoded by the nucleic acid can be, for example, an alteration in the quantitative polypeptide expression (i.e., the amount of polypeptide produced). An alteration in the composition of a polypeptide encoded by the nucleic acid is an alteration in the qualitative polypeptide expression (e.g., expression of a mutant polypeptide or of a different splicing variant). In one embodiment, determination of a susceptibility is made by detecting a particular splicing variant, or a particular pattern of splicing variants.

Both such alterations (quantitative and qualitative) can also be present. An “alteration” in the polypeptide expression or composition, as used herein, refers to an alteration in expression or composition in a test sample, as compared to the expression or composition of the polypeptide in a control sample. A control sample is a sample that corresponds to the test sample (e.g., is from the same type of cells), and is from a subject who is not affected by, and/or who does not have a susceptibility to, the particular condition. In one embodiment, the control sample is from a subject that does not possess a marker allele or haplotype associated with the condition, as described herein. Similarly, the presence of one or more different splicing variants in the test sample, or the presence of significantly different amounts of different splicing variants in the test sample, as compared with the control sample, can be indicative of a susceptibility to the condition. An alteration in the expression or composition of the polypeptide in the test sample, as compared with the control sample, can be indicative of a specific allele in the instance where the allele alters a splice site relative to the reference in the control sample. Various means of examining expression or composition of a polypeptide encoded by a nucleic acid are known to the person skilled in the art and can be used, including spectroscopy, colorimetry, electrophoresis, isoelectric focusing, and immunoassays (e.g., David et al., U.S. Pat. No. 4,376,110) such as immunoblotting (see, e.g., Current Protocols in Molecular Biology, particularly chapter 10, supra).

For example, in one embodiment, an antibody (e.g., an antibody with a detectable label) that is capable of binding to a polypeptide encoded by a nucleic acid associated with a vascular condition, as described herein, can be used. Antibodies can be polyclonal or monoclonal. An intact antibody, or a fragment thereof (e.g., Fv, Fab, Fab′, F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a labeled secondary antibody (e.g., a fluorescently-labeled secondary antibody) and end-labeling of a DNA probe with biotin such that it can be detected with fluorescently-labeled streptavidin.

In one embodiment of this method, the level or amount of a polypeptide in a test sample is compared with the level or amount of the polypeptide in a control sample. A level or amount of the polypeptide in the test sample that is higher or lower than the level or amount of the polypeptide in the control sample, such that the difference is statistically significant, is indicative of an alteration in the expression of the polypeptide encoded by the nucleic acid, and is diagnostic for a particular allele or haplotype responsible for causing the difference in expression. Alternatively, the composition of the polypeptide in a test sample is compared with the composition of the polypeptide in a control sample. In another embodiment, both the level or amount and the composition of the polypeptide can be assessed in the test sample and in the control sample.

In another embodiment, determination of a susceptibility is made by detecting at least one marker or haplotype of the present invention, in combination with an additional protein-based, RNA-based or DNA-based assay.

### Kits

Kits useful in the methods of the invention comprise components useful in any of the methods described herein, including for example, primers for nucleic acid amplification, hybridization probes, restriction enzymes (e.g., for RFLP analysis), allele-specific oligonucleotides, antibodies, means for amplification of nucleic acids, means for analyzing the nucleic acid sequence of a nucleic acids, means for analyzing the amino acid sequence of a polypeptide, etc. The kits can for example include necessary buffers, nucleic acid primers for amplifying nucleic acids, and reagents for allele-specific detection of the fragments amplified using such primers and necessary enzymes (e.g., DNA polymerase). Additionally, kits can provide reagents for assays to be used in combination with the methods of the present invention, e.g., reagents for use with other diagnostic assays.

In one embodiment, the invention pertains to a kit for assaying a sample from a subject to detect a susceptibility to a vascular condition selected from the group consisting of: an abnormal electrocardiogram measure, Atrial Fibrillation, Atrial Flutter, and Stroke in a subject, wherein the kit comprises reagents necessary for selectively detecting at least one allele of at least one polymorphism of the present invention in the genome of the individual. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism associated with risk of the vascular condition. In embodiment, the polymorphism is selected from the group consisting of rs3825214, rs6795970, rs3807989, rs7660702, rs132311, rs1733724 and rs365990, and markers in linkage disequilibrium therewith. In yet another embodiment the fragment is at least 20 base pairs in size. Such oligonucleotides or nucleic acids (e.g., oligonucleotide primers) can be designed using portions of the nucleic acid sequence flanking polymorphisms (e.g., SNPs or microsatellites). In another embodiment, the kit comprises one or more labeled nucleic acids capable of allele-specific detection of one or more specific polymorphic markers or haplotypes, and reagents for detection of the label. Suitable labels include, e.g., a radioisotope, a fluorescent label, an enzyme label, an enzyme co-factor label, a magnetic label, a spin label, an epitope label.

In particular embodiments, the polymorphic marker or haplotype to be detected by the reagents of the kit comprises one or more markers, two or more markers, three or more markers, four or more markers or five or more markers selected from the group consisting of rs3825214, rs6795970, rs3807989, rs7660702, rs132311, rs1733724 and rs365990, and markers in linkage disequilibrium therewith. In another embodiment, the marker or haplotype to be detected comprises at least one marker from the group of markers in strong linkage disequilibrium, as defined by values of r² greater than 0.2, to at least one of the group of markers rs3825214, rs6795970, rs3807989, rs7660702, rs132311, rs1733724 and rs365990. In another embodiment, the marker or haplotype to be detected is selected from the group consisting of rs3825214, rs6795970, rs3807989, rs7660702, rs132311, rs1733724 and rs365990.

In a preferred embodiment, the DNA template containing the SNP polymorphism is amplified by Polymerase Chain Reaction (PCR) prior to detection, and primers for such amplification are included in the reagent kit. In such an embodiment, the amplified DNA serves as the template for the detection probe and the enhancer probe.

In one embodiment, the DNA template is amplified by means of Whole Genome Amplification (WGA) methods, prior to assessment for the presence of specific polymorphic markers as described herein. Standard methods well known to the skilled person for performing WGA may be utilized, and are within scope of the invention. In one such embodiment, reagents for performing WGA are included in the reagent kit.

In certain embodiments, determination of the presence of a particular marker allele or haplotype is indicative of a susceptibility (increased susceptibility or decreased susceptibility) to the vascular condition. In another embodiment, determination of the presence of the marker allele or haplotype is indicative of response to a therapeutic agent for the vascular condition. In another embodiment, the presence of the marker allele or haplotype is indicative of prognosis of the vascular condition. In yet another embodiment, the presence of the marker allele or haplotype is indicative of progress of treatment of the condition. Such treatment may include intervention by surgery, medication or by other means (e.g., lifestyle changes).

In a further aspect of the present invention, a pharmaceutical pack (kit) is provided, the pack comprising a therapeutic agent and a set of instructions for administration of the therapeutic agent to humans diagnostically tested for one or more variants of the present invention, as disclosed herein. The therapeutic agent can be a small molecule drug, an antibody, a peptide, an antisense or rnai molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention is instructed to take a prescribed dose of the therapeutic agent. In another embodiment, an individual identified as a non-carrier of at least one variant of the present invention is instructed to take a prescribed dose of the therapeutic agent.

In certain embodiments, the kit further comprises a set of instructions for using the reagents comprising the kit. In certain embodiments, the kit further comprises a collection of data comprising correlation data between the polymorphic markers assessed by the kit and susceptibility to prostate cancer and/or colorectal cancer.

### Therapeutic Agents

Treatment of Atrial Fibrillation and Atrial flutter is generally directed by two main objectives: (i) to prevent stroke and (ii) to treat symptoms.

### (i) Stroke Prevention

Anticoagulation is the therapy of choice for stroke prevention in atrial fibrillation and is indicated for the majority of patients with this arrhythmia. The only patients for whom anticoagulation is not strongly recommended are those younger than 65 years old who are considered low-risk, i.e., they have no organic heart disease, no hypertension, no previous history of stroke or transient ischemic attacks and no diabetes. This group as a whole has a lower risk of stroke and stroke prevention with aspirin is generally recommended. For all other patients, anticoagulation is indicated whether the atrial fibrillation is permanent, recurrent paroxysmal or recurrent persistent. It cannot be generalized how patients who present with their first episode of paroxysmal atrial fibrillation should be treated and the decision needs to be individualized for each patient. Anticoagulation is also indicated even when the patient with atrial fibrillation is felt to be maintained in sinus rhythm with antiarrhythmic therapy (rhythm controlled) since this type of therapy does not affect stroke risk.

Anticoagulants.

Anticoagulation is recommended in atrial fibrillation, as detailed above, for prevention of cardioembolism and stroke. The most widely studied oral anticoagulant is warfarin and this medication is universally recommended for chronic oral anticoagulation in atrial fibrillation. Warfarin has few side effects aside from the risk of bleeding but requires regular and careful monitoring of blood values during therapy (to measure the effect of the anticoagulation). The oral anticoagulant ximelagatran showed promise in stroke prevention in patients with atrial fibrillation and had the advantage of not requiring regular monitoring like warfarin. Ximelagatran was found however to cause unexplained liver injury and was withdrawn from the market in 2006. Several agents are available for intravenous and/or subcutaneous therapy, including heparin and the low molecular weight heparins (e.g. enoxaparin, dalteparin, tinzaparin, ardeparin, nadroparin and reviparin). These medications are recommended when rapid initiation of anticoagulation is necessary or if oral anticoagulation therapy has to be interrupted in high risk patients or for longer than one week in other patients for example due to a series of procedures. Other parenteral anticoagulants are available but not specifically recommended as therapy in atrial fibrillation; e.g., the factor Xa inhibitors fondaparinux and idraparinux, the thrombin-inhibitors lepirudin, bivalirudin and argatroban as well as danaparoid.

### (ii) Symptom Control.

Medical and surgical therapy applied to control symptoms of atrial fibrillation is tailored to the individual patient and consists of heart rate and/or rhythm control with medications, radiofrequency ablation and/or surgery.

Antiarrhythmic Medications.

In general terms, antiarrhythmic agents are used to suppress abnormal rhythms of the heart that are characteristic of cardiac arrhythmias, including atrial fibrillation and atrial flutter. One classification of antiarrhythmic agents is the Vaughan Williams classification, in which five main categories of antiarrhythmic agents are defined. Class I agents are fast sodium channel blockers and are subclassified based on kinetics and strenght of blockade as well as their effect on repolarization. Class Ia includes disopyramide, moricizine, procainamide and quinidine. Class Ib agents are lidocaine, mexiletine, tocamide, and phenyloin. Class Ic agents are encamide, flecamide, propafenone, ajmaline, cibenzoline and detajmium. Class II agents are beta blockers, they block the effects of catecholamines at beta-adrenergic receptors. Examples of beta blockers are esmolol, propranolol, metoprolol, alprenolol, atenolol, carvedilol, bisoprolol, acebutolol, nadolol, pindolol, labetalol, oxprenotol, penbutolol, timolol, betaxolol, cartelol, sotalol and levobunolol. Class III agents have mixed properties but are collectively potassium channel blockers and prolong repolarization. Medications in this category are amiodarone, azimilide, bretylium, dofetilide, tedisamil, ibutilide, sematilide, sotalol, N-acetyl procainamide, nifekalant hydrochloride, vernakalant and ambasilide. Class IV agents are calcium channel blockers and include verapamil, mibefradil and diltiazem. Finally, class V consists of miscellaneous antiarrhythmics and includes digoxin and adenosine.

Heart Rate Control,

Pharmacologic measures for maintenance of heart rate control include beta blockers, calcium channel blockers and digoxin. All these medications slow the electrical conduction through the atrioventricular node and slow the ventricular rate response to the rapid atrial fibrillation. Some antiarrhythmics used primarily for rhythm control (see below) also slow the atrioventricular node conduction rate and thus the ventricular heart rate response. These include some class III and Ic medications such as amiodarone, sotalol and flecamide.

Cardioversion.

Cardioversion of the heart rhythm from atrial fibrillation or atrial flutter to sinus rhythm can be achieved electrically, with synchronized direct-current cardioversion, or with medications such as ibutilide, amiodarone, procainamide, propafenone and flecamide.

### Heart Rhythm Control

Medications used for maintenance of sinus rhythm, i.e. rhythm control, include mainly antiarrhythmic medications from classes III, Ia and Ic. Examples are sotalol, amiodarone and dofetilide from class III, disopyramide, procainamide and quinidine from class Ia and flecinide and propafenone from class Ic. Treatment with these antiarrhythmic medications is complicated, can be hazardous, and should be directed by physicians specifically trained to use these medications. Many of the antiarrhythmics have serious side effects and should only be used in specific populations. For example, class Ic medications should not be used in patients with coronary artery disease and even if they can suppress atrial fibrillation, they can actually promote rapid ventricular response in atrial flutter. Class Ia medications can be used as last resort in patients without structural heart diseases. Sotalol (as most class III antiarrhythmics) can cause significant prolongation of the QT interval, specifically in patients with renal failure, and promote serious ventricular arrhythmias. Both sotalol and dofetilide as well as the Ia medications need to be initiated on an inpatient basis to monitore the QT interval. Although amiodarone is usually well tolerated and is widely used, amiodarone has many serious side effects with long-term therapy.

The variants (markers and/or haplotypes) disclosed herein can be useful in the identification of novel therapeutic targets for cardiac arrhythmia, in particular Atrial Fibrillation and Atrial Flutter. For example, genes containing, or in linkage disequilibrium with, one or more of these variants, or their products, as well as genes or their products that are directly or indirectly regulated by or interact with these variant genes or their products, can be targeted for the development of therapeutic agents. Therapeutic agents may comprise one or more of, for example, small non-protein and non-nucleic acid molecules, proteins, peptides, protein fragments, nucleic acids (DNA, RNA), PNA (peptide nucleic acids), or their derivatives or mimetics which can modulate the function and/or levels of the target genes or their gene products.

The nucleic acids and/or variants described herein, or nucleic acids comprising their complementary sequence, may be used as antisense constructs to control gene expression in cells, tissues or organs. The methodology associated with antisense techniques is well known to the skilled artisan, and is for example described and reviewed in AntisenseDrug Technology: Principles, Strategies, and Applications, Crooke, ed., Marcel Dekker Inc., New York (2001). In general, antisense agents (antisense oligonucleotides) are comprised of single stranded oligonucleotides (RNA or DNA) that are capable of binding to a complimentary nucleotide segment. By binding the appropriate target sequence, an RNA-RNA, DNA-DNA or RNA-DNA duplex is formed. The antisense oligonucleotides are complementary to the sense or coding strand of a gene. It is also possible to form a triple helix, where the antisense oligonucleotide binds to duplex DNA.

Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Layery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer. Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug.Dev. 12:215-24 (2002).

In certain embodiments, the antisense agent is an oligonucleotide that is capable of binding to a particular nucleotide segment. In certain embodiments, the nucleotide segment comprises the nucleotide sequence, or a fragment of the nucleotide sequence, of a gene selected from the group consisting of the human TBX5 gene, the human SCN10A gene, the human CAV1 gene, the human ARHGAP24 gene, the human CDKN1A gene, and the human MYH6 gene. In certain other embodiments, the antisense nucleotide is capable of binding to a nucleotide segment of as set forth in any one of SEQ ID NO:1-3623. Antisense nucleotides are suitably in the range of 5-400 nucleotides in length, including 5-200 nucleotides, 5-100 nucleotides, 10-50 nucleotides, and 10-30 nucleotides. In certain preferred embodiments, the antisense nucleotides is from 14-50 nucleotides in length, including 14-40 nucleotides and 14-30 nucleotides.

The variants described herein can also be used for the selection and design of antisense reagents that are specific for particular variants (e.g., any one of the variants disclosed herein, e.g., any one of the variants as set forth in SEQ ID NO:1-3623). Using information about the variants described herein, antisense oligonucleotides or other antisense molecules that specifically target mRNA molecules that contain one or more variants of the invention can be designed. In this manner, expression of mRNA molecules that contain one or more variant of the present invention (i.e. certain marker alleles and/or haplotypes) can be inhibited or blocked. In one embodiment, the antisense molecules are designed to specifically bind a particular allelic form (i.e., one or several variants (alleles and/or haplotypes)) of the target nucleic acid, thereby inhibiting translation of a product originating from this specific allele or haplotype, but which do not bind other or alternate variants at the specific polymorphic sites of the target nucleic acid molecule. As antisense molecules can be used to inactivate mRNA so as to inhibit gene expression, and thus protein expression, the molecules can be used for disease treatment. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants presented herein can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Layery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2: E31-6 (2000), and Hunter, Curr. Biol. 9: R440-442 (1999).

A genetic defect leading to increased predisposition or risk for development of a disease, or a defect causing the disease, may be corrected permanently by administering to a subject carrying the defect a nucleic acid fragment that incorporates a repair sequence that supplies the normal/wild-type nucleotide(s) at the site of the genetic defect. Such site-specific repair sequence may concompass an RNA/DNA oligonucleotide that operates to promote endogenous repair of a subject's genomic DNA. The administration of the repair sequence may be performed by an appropriate vehicle, such as a complex with polyethelenimine, encapsulated in anionic liposomes, a viral vector such as an adenovirus vector, or other pharmaceutical compositions suitable for promoting intracellular uptake of the adminstered nucleic acid. The genetic defect may then be overcome, since the chimeric oligonucleotides induce the incorporation of the normal sequence into the genome of the subject, leading to expression of the normal/wild-type gene product. The replacement is propagated, thus rendering a permanent repair and alleviation of the symptoms associated with the disease or condition.

The present invention provides methods for identifying compounds or agents that can be used to treat a disease characterized by an abnormal ECG measure, including Atrial Fibrillation and Atrial Flutter, or Stroke. Thus, the variants of the invention are useful as targets for the identification and/or development of therapeutic agents. In certain embodiments, such methods include assaying the ability of an agent or compound to modulate the activity and/or expression of a nucleic acid that includes at least one of the variants (markers and/or haplotypes) of the present invention, or the encoded product of the nucleic acid (e.g., an encoded product of one or more of the human TBX5, SCN10A, CAV1, ARHGAP24, CDKN1A and MYH6 genes). This in turn can be used to identify agents or compounds that inhibit or alter the undesired activity or expression of the encoded nucleic acid product. Assays for performing such experiments can be performed in cell-based systems or in cell-free systems, as known to the skilled person. Cell-based systems include cells naturally expressing the nucleic acid molecules of interest, or recombinant cells that have been genetically modified so as to express a certain desired nucleic acid molecule.

Variant gene expression in a patient can be assessed by expression of a variant-containing nucleic acid sequence (for example, a gene containing at least one variant of the present invention, which can be transcribed into RNA containing the at least one variant, and in turn translated into protein), or by altered expression of a normal/wild-type nucleic acid sequence due to variants affecting the level or pattern of expression of the normal transcripts, for example variants in the regulatory or control region of the gene. Assays for gene expression include direct nucleic acid assays (mRNA), assays for expressed protein levels, or assays of collateral compounds involved in a pathway, for example a signal pathway. Furthermore, the expression of genes that are up- or down-regulated in response to the signal pathway can also be assayed. One embodiment includes operably linking a reporter gene, such as luciferase, to the regulatory region of the gene(s) of interest.

Modulators of gene expression can in one embodiment be identified when a cell is contacted with a candidate compound or agent, and the expression of mRNA is determined. The expression level of mRNA in the presence of the candidate compound or agent is compared to the expression level in the absence of the compound or agent. Based on this comparison, candidate compounds or agents for treating the disease can be identified as those modulating the gene expression of the variant gene. When expression of mRNA or the encoded protein is statistically significantly greater in the presence of the candidate compound or agent than in its absence, then the candidate compound or agent is identified as a stimulator or up-regulator of expression of the nucleic acid. When nucleic acid expression or protein level is statistically significantly less in the presence of the candidate compound or agent than in its absence, then the candidate compound is identified as an inhibitor or down-regulator of the nucleic acid expression.

The invention further provides methods of treatment using a compound identified through drug (compound and/or agent) screening as a gene modulator (i.e. stimulator and/or inhibitor of gene expression).

### Methods of Assessing Probability of Response to Therapeutic Agents, Methods of Monitoring Progress of Treatment and Methods of Treatment

As is known in the art, individuals can have differential responses to a particular therapy (e.g., a therapeutic agent or therapeutic method). Pharmacogenomics addresses the issue of how genetic variations (e.g., the variants (markers and/or haplotypes) of the present invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the present invention), or therapeutic failure of the drug. Therefore, the variants of the present invention may determine the manner in which a therapeutic agent and/or method acts on the body, or the way in which the body metabolizes the therapeutic agent.

Accordingly, in one embodiment, the presence of a particular allele at a polymorphic site is indicative of a different response, e.g. a different response rate, to a particular treatment modality. This means that a patient diagnosed with a disease, and carrying a certain allele at a polymorphic would respond better to, or worse to, a specific therapeutic, drug and/or other therapy used to treat the disease. Therefore, the presence or absence of the marker allele or haplotype could aid in deciding what treatment should be used for a the patient. For example, for a newly diagnosed patient, the presence of a marker or haplotype of the present invention may be assessed (e.g., through testing DNA derived from a blood sample, as described herein). If the patient is positive for a marker allele or haplotype (that is, at least one specific allele of the marker, or haplotype, is present), then the physician recommends one particular therapy, while if the patient is negative for the at least one allele of a marker, or a haplotype, then a different course of therapy may be recommended (which may include recommending that no immediate therapy, other than serial monitoring for progression of the disease, be performed). Thus, the patient's carrier status could be used to help determine whether a particular treatment modality should be administered. The value lies within the possibilities of being able to diagnose the disease at an early stage, to select the most appropriate treatment, and provide information to the clinician about prognosis/aggressiveness of the disease in order to be able to apply the most appropriate treatment.

Thus, one aspect of the invention relates to methods of assessing probability of response to a therapeutic agent for preventing, treating and/or ameliorating symptoms associated with a vascular condition selected from the group consisting of: an abnormal electrocardiogram measure, Atrial Fibrillation, Atrial Flutter, and Stroke, by determining whether certain variants found to correlate with risk of these conditions are present in the genome of the individual, as described in more detail herein. In one embodiment, the method comprises obtaining sequence data about a human individual identifying at least one allele of at least one polymorphic marker selected from the group consisting of rs3825214, rs6795970, rs3807989, rs7660702, rs132311, rs1733724 and rs365990, and markers in linkage disequilibrium therewith, wherein different alleles of the at least one polymorphic marker are associated with different probabilities of response to the therapeutic agent in humans, and determining the probability of a positive response to the therapeutic agent from the sequence data. The therapeutic agent may be any therapeutic agent that is useful for treating, or ameliorating symptoms of, any of the above mentioned conditions.

In one embodiment, the therapeutic agent is an agent for treating or controlling abnormal heart rate, and the polymorphic marker is selected from the group consisting of rs365990, and markers in linkage disequilibrium therewith. In another embodiment, the therapeutic agent is an agent for treating Atrial Fibrillation, and the polymorphic marker is selected from the group consisting of rs3825214 and rs3807989, and markers in linkage disequilibrium therewith.

The present invention also relates to methods of monitoring progress or effectiveness of a treatment for any of these conditions. This can be done based on the genotype and/or haplotype status of the markers and haplotypes of the present invention, i.e., by assessing the absence or presence of at least one allele of at least one polymorphic marker as disclosed herein, or by monitoring expression of genes that are associated with the variants (markers and haplotypes) of the present invention. The risk gene mrna or the encoded polypeptide can be measured in a tissue sample (e.g., a peripheral blood sample, or a biopsy sample). Expression levels and/or mrna levels can thus be determined before and during treatment to monitor its effectiveness. Alternatively, or concomitantly, the genotype and/or haplotype status of at least one risk variant for the condition as presented herein is determined before and during treatment to monitor its effectiveness.

Alternatively, biological networks or metabolic pathways related to the markers and haplotypes of the present invention can be monitored by determining mRNA and/or polypeptide levels. This can be done for example, by monitoring expression levels or polypeptides for several genes belonging to the network and/or pathway, in samples taken before and during treatment. Alternatively, metabolites belonging to the biological network or metabolic pathway can be determined before and during treatment. Effectiveness of the treatment is determined by comparing observed changes in expression levels/metabolite levels during treatment to corresponding data from healthy subjects.

In a further aspect, the markers of the present invention can be used to increase power and effectiveness of clinical trials. Thus, individuals who are carriers of at least one at-risk variant of the present invention may be more likely to respond favorably to a particular treatment modality. In one embodiment, individuals who carry at-risk variants for gene(s) in a pathway and/or metabolic network for which a particular treatment (e.g., small molecule drug) is targeting, are more likely to be responders to the treatment. In another embodiment, individuals who carry at-risk variants for a gene, which expression and/or function is altered by the at-risk variant, are more likely to be responders to a treatment modality targeting that gene, its expression or its gene product. This application can improve the safety of clinical trials, but can also enhance the chance that a clinical trial will demonstrate statistically significant efficacy, which may be limited to a certain sub-group of the population. Thus, one possible outcome of such a trial is that carriers of certain genetic variants are statistically significantly likely to show positive response to the therapeutic agent, i.e. experience alleviation of symptoms associated with the condition when taking the therapeutic agent or drug as prescribed.

In a further aspect, the markers and haplotypes of the present invention can be used for targeting the selection of pharmaceutical agents for specific individuals. Personalized selection of treatment modalities, lifestyle changes or combination of lifestyle changes and administration of particular treatment, can be realized by the utilization of the at-risk variants of the present invention. Thus, the knowledge of an individual's status for particular markers of the present invention, can be useful for selection of treatment options that target genes or gene products affected by the at-risk variants of the invention. Certain combinations of variants may be suitable for one selection of treatment options, while other gene variant combinations may target other treatment options. Such combination of variant may include one variant, two variants, three variants, or four or more variants, as needed to determine with clinically reliable accuracy the selection of treatment module.

One such aspect relates to the use of a therapeutic agent in the preparation of a medicament for treating a condition selected from the group consisting of: an abnormal electrocardiogram measure, Atrial Fibrillation, Atrial Flutter, and Stroke in a human individual that has been tested for the presence of at least one allele of at least one polymorphic marker selected from the group consisting of rs3825214, rs6795970, rs3807989, rs7660702, rs132311, rs1733724 and rs365990, and markers in linkage disequilibrium therewith. In one embodiment, determination of the presence of at least one at-risk allele of the at least one marker is indicative that the human individual is suitable for administration of the therapeutic agent. In certain embodiments, the risk allele is an allele that increases risk of an increased ECG interval and/or increased risk of Atrial Fibrillation, Atrial Flutter and/or Stroke. The therapeutic agent may be suitably selected from the therapeutic agents as described herein.

Another aspect relates to a method of treating a condition selected from the group consisting of: an abnormal electrocardiogram measure, Atrial Fibrillation, Atrial Flutter, and Stroke, the method comprising: (a) selecting a human individual that has been tested for the presence of at least one allele of at least one polymorphic marker selected from the group consisting of rs3825214, rs6795970, rs3807989, rs7660702, rs132311, rs1733724 and rs365990, and markers in linkage disequilibrium therewith; and (b) administering to the individual a therapeutically effective amount of the therapeutic agent.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

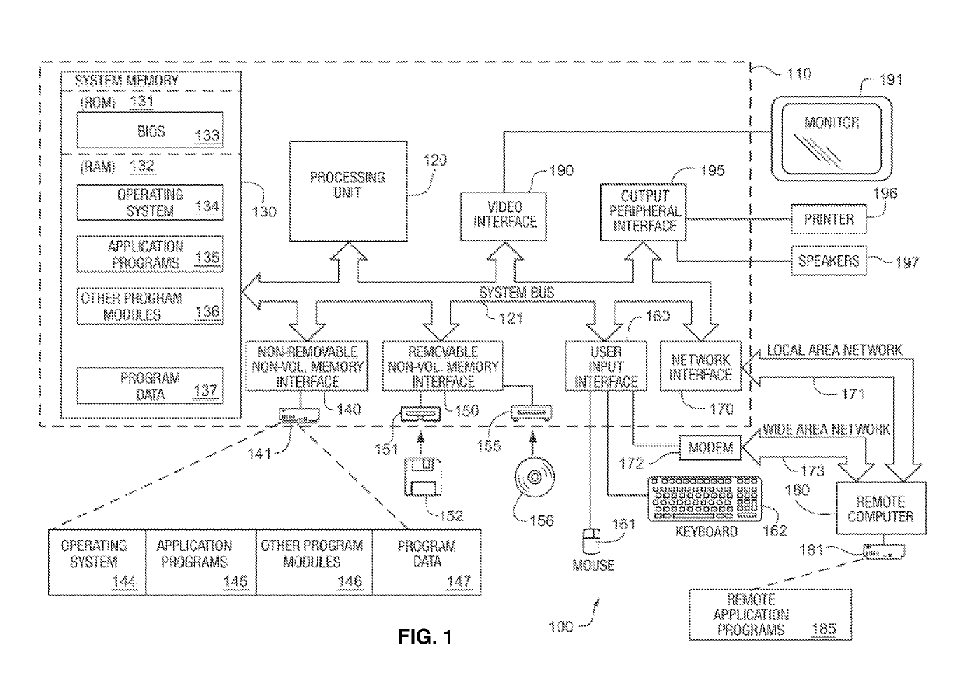

FIG. 1 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method or apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 1, an exemplary system for implementing the steps of the claimed method and system includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (USA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 1 illustrates operating system 134, application programs 135, other program modules 136, and program data 137.

The computer 110 may also include other removable/non-removable, volatile/nonvolatile computer storage media. By way of example only, FIG. 1 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 1, provide storage of computer readable instructions, data structures, program modules and other data for the computer 110. In FIG. 1, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 1. The logical connections depicted in FIG. 1 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 1 illustrates remote application programs 185 as residing on memory device 181. It will be appreciated that the network connections shown are exemplary and other means of establishing a communications link between the computers may be used.

Although the forgoing text sets forth a detailed description of numerous different embodiments of the invention, it should be understood that the scope of the invention is defined by the words of the claims set forth at the end of this patent. The detailed description is to be construed as exemplary only and does not describe every possibly embodiment of the invention because describing every possible embodiment would be impractical, if not impossible. Numerous alternative embodiments could be implemented, using either current technology or technology developed after the filing date of this patent, which would still fall within the scope of the claims defining the invention.

While the risk evaluation system and method, and other elements, have been described as preferably being implemented in software, they may be implemented in hardware, firmware, etc., and may be implemented by any other processor. Thus, the elements described herein may be implemented in a standard multi-purpose CPU or on specifically designed hardware or firmware such as an application-specific integrated circuit (ASIC) or other hard-wired device as desired, including, but not limited to, the computer 110 of FIG. 1. When implemented in software, the software routine may be stored in any computer readable memory such as on a magnetic disk, a laser disk, or other storage medium, in a RAM or ROM of a computer or processor, in any database, etc. Likewise, this software may be delivered to a user or a diagnostic system via any known or desired delivery method including, for example, on a computer readable disk or other transportable computer storage mechanism or over a communication channel such as a telephone line, the internet, wireless communication, etc. (which are viewed as being the same as or interchangeable with providing such software via a transportable storage medium).

Thus, many modifications and variations may be made in the techniques and structures described and illustrated herein without departing from the spirit and scope of the present invention. Thus, it should be understood that the methods and apparatus described herein are illustrative only and are not limiting upon the scope of the invention.

Accordingly, the invention relates to computer-implemented applications using the polymorphic markers described herein, and genotype and/or disease-association data derived therefrom. Such applications can be useful for storing, manipulating or otherwise analyzing genotype data that is useful in the methods of the invention. One example pertains to storing genotype information derived from an individual on readable media, so as to be able to provide the genotype information to a third party (e.g., the individual, a guardian of the individual, a health care provider or genetic analysis service provider), or for deriving information from the genotype data, e.g., by comparing the genotype data to information about genetic risk factors contributing to increased susceptibility to the disease, and reporting results based on such comparison.

In general terms, computer-readable media has capabilities of storing (i) identifier information for at least one polymorphic marker or a haplotype, as described herein; (ii) an indicator of the frequency of at least one allele of said at least one marker, or the frequency of a haplotype, in individuals with the disease; and an indicator of the frequency of at least one allele of said at least one marker, or the frequency of a haplotype, in a reference population. The reference population can be a disease-free population of individuals. Alternatively, the reference population is a random sample from the general population, and is thus representative of the population at large. The frequency indicator may be a calculated frequency, a count of alleles and/or haplotype copies, or normalized or otherwise manipulated values of the actual frequencies that are suitable for the particular medium.

The markers and haplotypes described herein are in certain embodiments useful for interpretation and/or analysis of genotype data. Thus in certain embodiments, determination of the presence of an at-risk allele for a vascular condition, as described herein, or determination of the presence of an allele at a polymorphic marker in LD with any such risk allele, is indicative of the individual from whom the genotype data originates is at increased risk of the condition. In one such embodiment, genotype data is generated for at least one polymorphic marker shown herein to be associated with risk of the condition, or a marker in linkage disequilibrium therewith. The genotype data is subsequently made available to a third party, such as the individual from whom the data originates, his/her guardian or representative, a physician or health care worker, genetic counsellor, or insurance agent, for example via a user interface accessible over the internet, together with an interpretation of the genotype data, e.g., in the form of a risk measure (such as an absolute risk (AR), risk ratio (RR) or odds ratio (OR)) for the disease. In another embodiment, at-risk markers identified in a genotype dataset derived from an individual are assessed and results from the assessment of the risk conferred by the presence of such at-risk variants in the dataset are made available to the third party, for example via a secure web interface, or by other communication means. The results of such risk assessment can be reported in numeric form (e.g., by risk values, such as absolute risk, relative risk, and/or an odds ratio, or by a percentage increase in risk compared with a reference), by graphical means, or by other means suitable to illustrate the risk to the individual from whom the genotype data is derived.

One computer-implemented aspect relates to a system for generating a risk assessment report for a vascular condition (e.g., an abnormal electrocardiogram measure, Atrial Fibrillation, Atrial Flutter, and/or Stroke). The system suitably comprises (a) a memory configured to store sequence data for at least one human subject, the sequence data identifying at least one allele of at least one polymorphic marker, wherein different alleles of the marker are associated with different susceptibilities to the condition in humans; and (b) a processor configured to (i) receive information identifying the at least one allele of the at least one polymorphic marker; (ii) transform said information into a risk measure of the condition for the human subject; (iii) generate a risk assessment report based on the received information; and (iv) provide the risk assessment report on an output device. The sequence data may be a dataset for particular polymorphic markers. The sequence data may also be continuous sequence data from the subject, such as complete genomic sequence data from the individual. The identification of particular alleles at particular polymorphic sites (markers) can be used for determining risk, be transforming the allelic data (genotype data) into a suitable risk measure. The risk measure can be any convenient risk measure, as is described in more detail in the foregoing, including for example a lifetime risk measure (e.g., a percentage absolute risk, a relative risk value compared with an average person from the population, a relative risk value compared with individuals who do not carry the particular at-risk variant, etc.).

### Nucleic Acids and Polypeptides

The nucleic acids and polypeptides described herein can be used in methods and kits of the present invention. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S, and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). Another example of an algorithm is BLAT (Kent, W. J. Genome Res. 12:656-64 (2002)). Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988). In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, the nucleotide sequence of LD Block C03, LD Block CO₄, LD Block C06, LD Block C07, LD Block C10, LD Block C12 or LD Block C14, or a nucleotide sequence comprising, or consisting of, the complement of the nucleotide sequence of LD Block C03, LD Block C04, LD Block C06, LD Block C07, LD Block C10, LD Block C12 or LD Block C14, wherein the nucleotide sequence comprises at least one polymorphic marker as described herein. The nucleic acid fragments of the invention may suitably be at least about 15, at least about 18, 20, 23 or 25 nucleotides, and can be 30, 40, 50, 100, 200, 500, 1000, 10,000 or more nucleotides in length. In certain embodiments, the nucleotides are less than 1000, less than 500, less than 400, less than 300, less than 200, less than 100, or less than 50 nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled, fluorescently labeled) and used as a probe for screening a cDNA library derived from human cells. The cDNA can be derived from mRNA and contained in a suitable vector. Corresponding clones can be isolated, DNA obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

### Antibodies

The invention also provides antibodies which bind to an epitope comprising either a variant amino acid sequence (e.g., comprising an amino acid substitution) encoded by a variant allele or the reference amino acid sequence encoded by the corresponding non-variant or wild-type allele. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies may also be useful in pharmacogenomic analysis. In such embodiments, antibodies against variant proteins encoded by nucleic acids according to the invention, such as variant proteins that are encoded by nucleic acids that contain at least one polymorpic marker of the invention, can be used to identify individuals that require modified treatment modalities.

Antibodies can furthermore be useful for assessing expression of variant proteins in disease states, or in an individual with a predisposition to a disease related to the function of the protein, such as one or more of the vascular conditions disclosed herein (e.g., abnormal ECG measures, Atrial Fibrillation, Atrial Flutter, Stroke). Antibodies specific for a variant protein of the present invention that is encoded by a nucleic acid that comprises at least one polymorphic marker or haplotype as described herein can be used to screen for the presence of the variant protein, for example to screen for a predisposition to a vascular condition as described herein, as indicated by the presence of the variant protein.

Antibodies can be used in other methods. Thus, antibodies are useful as diagnostic tools for evaluating proteins, such as variant proteins of the invention, in conjunction with analysis by electrophoretic mobility, isoelectric point, tryptic or other protease digest, or for use in other physical assays known to those skilled in the art. Antibodies may also be used in tissue typing. In one such embodiment, a specific variant protein has been correlated with expression in a specific tissue type, and antibodies specific for the variant protein can then be used to identify the specific tissue type.

Subcellular localization of proteins, including variant proteins, can also be determined using antibodies, and can be applied to assess aberrant subcellular localization of the protein in cells in various tissues. Such use can be applied in genetic testing, but also in monitoring a particular treatment modality. In the case where treatment is aimed at correcting the expression level or presence of the variant protein or aberrant tissue distribution or developmental expression of the variant protein, antibodies specific for the variant protein or fragments thereof can be used to monitor therapeutic efficacy.

Antibodies are further useful for inhibiting variant protein function, for example by blocking the binding of a variant protein to a binding molecule or partner. Such uses can also be applied in a therapeutic context in which treatment involves inhibiting a variant protein's function. An antibody can be for example be used to block or competitively inhibit binding, thereby modulating (i.e., agonizing or antagonizing) the activity of the protein. Antibodies can be prepared against specific protein fragments containing sites required for specific function or against an intact protein that is associated with a cell or cell membrane. For administration in vivo, an antibody may be linked with an additional therapeutic payload, such as radionuclide, an enzyme, an immunogenic epitope, or a cytotoxic agent, including bacterial toxins (diphtheria or plant toxins, such as ricin). The in vivo half-life of an antibody or a fragment thereof may be increased by pegylation through conjugation to polyethylene glycol.

The present invention further relates to kits for using antibodies in the methods described herein. This includes, but is not limited to, kits for detecting the presence of a variant protein in a test sample. One preferred embodiment comprises antibodies such as a labelled or labelable antibody and a compound or agent for detecting variant proteins in a biological sample, means for determining the amount or the presence and/or absence of variant protein in the sample, and means for comparing the amount of variant protein in the sample with a standard, as well as instructions for use of the kit.

The present invention will now be exemplified by the following non-limiting examples.

### Example 1

### Several Common Variants Modulate Heart Rate, PR Interval and QRS Duration and Affect Risk of Cardiac Arrhythmias

To search for sequence variants that associate with HR, PR interval, QRS duration and QT interval in a population of European origin, we performed a GWAS on ten thousand Icelanders using the Illumine HumanHap300 and HumanHapCNV370 bead chips (Table 2). We then attempted to replicate the observed associations in additional ten thousand Icelanders (Table 2). All subjects had ECG data from the Landspitali University Hospital in Reykjavik, Iceland (see Methods). Individuals with AF, PM and/or defibrillator implants were excluded from PR interval, QRS duration and QT interval scans. Individuals with prolonged QRS interval (>120 ms) were also excluded from the QT interval analysis. The analysis was performed by regressing the measured parameters, adjusted for birth cohort, age at measurement and sex, on SNP allele counts. The QT interval was additionally adjusted for heart rate. The PR interval, QRS duration, QT interval and heart rate information were obtained for the same individuals, allowing us to calculate their correlations. The strongest observed correlation between ECG parameters was between the QRS duration and the HR adjusted QT interval (correlation=0.44, see Table 3 for all pair-wise correlations) whereas the correlation between PR vs QRS and PR vs QT was much weaker (0.09 and 0.06, respectively).

Due to the known correlation between the various ECG parameters and arrhythmias, we systematically tested all ECG parameter-associated SNPs in AF, SSS, advanced (second and third degree) atrioventricular block (AVB) and a PM population (see descriptions of sample sets in methods below).

### Methods

The study was approved by the Data Protection Commission of Iceland and the National Bioethics Committee of Iceland. Written informed consent was obtained from all patients and controls. Personal identifiers associated with medical information and blood samples were encrypted with a third-party encryption system as provided by the Data Protection Commission of Iceland.

### ECG Study Sample

This analysis included all ECGs obtained and digitally stored at the Landspitali University Hospital, Reykjavik, the largest medical center in Iceland, from 2004 to 2008. The ECGs were digitally recorded with the Philips PageWriter Trim III and PageWriter 200 cardiographs and stored in the Philips TraceMasterVue ECG Management System. These were ECGs obtained in all hospital departments, from inpatients and outpatients, representing unselected general medical and surgical patients. Digitally measured ECG waveforms and parameters were extracted from the database for analysis. The Philips PageWriter Trim III QT interval measurement algorithm has been previously described and shown to fulfill industrial ECG measurement accuracy standards⁶⁴. The Philips PR interval and QRS complex measurements have been shown to fulfill industrial accuracy standards⁶⁵. Individuals with atrial fibrillation, pacemakers and/or defibrillators implants and prolonged QRS interval (>120 ms), indicating abnormal conduction, were excluded.

### Icelandic Atrial Fibrillation Sample

This study sample included patients diagnosed with AF and/or atrial flutter (AFL) (International Classification of Diseases (ICD) 10 code 148 and ICD 9 code 427.3) at Landspitali University Hospital in Reykjavik, the only tertiary referral centre in Iceland, and at Akureyri Regional Hospital, the second largest hospital in Iceland, from 1987 to 2008. All diagnoses were confirmed with a 12-lead ECG. The AF/AFL-free controls used in this study consisted of controls randomly selected from the Icelandic genealogical database and individuals from other ongoing related, but not cardiovascular, genetic studies at deCODE. Controls with first-degree relatives (siblings, parents or offspring) with AF/AFI, or a first-degree control relative, were excluded from the analysis.

### Icelandic Sick Sinus Syndrome Sample

This sample set included patients that received the discharge diagnosis of SSS (International Classification of Diseases (ICD) 10 code 149.5 and ICD 9 code 427.8) at Landspitali University Hospital in Reykjavik, from 1987 to 2008. All diagnoses were confirmed with a 12-lead ECG. The SSS-free controls used in this study consisted of controls randomly selected from the Icelandic genealogical database and individuals from other ongoing related, but not cardiovascular, genetic studies at deCODE. Controls with first-degree relatives (siblings, parents or offspring) with SSS, or a first-degree control relative, were excluded from the analysis.

### Icelandic Atrioventricular Block Sample

This sample set included all patients that received the discharge diagnosis of second and/or third degree AVB (International Classification of Diseases (ICD) 10 codes 144.1, 144.2 and 144.3 and ICD 9 codes 426.0 and 426.1) at Landspitali University Hospital in Reykjavik, from 1987 to 2008. All diagnoses were confirmed with a 12-lead ECG. The AVB-free controls used in this study consisted of controls randomly selected from the Icelandic genealogical database and individuals from other ongoing related, but not cardiovascular, genetic studies at deCODE. Controls with first-degree relatives (siblings, parents or offspring) with AVB, or a first-degree control relative, were excluded from the analysis.

### Icelandic Pacemaker Population Sample

This study included all patients that received a permanent pacemaker (PM) implantation at the Landspitali University Hospital in Reykjavik, also from 1987 to 2008. The causes of pacemaker implantation break down as follows: SSS=676, AVB=263, AF=240, other=41. All diagnoses were confirmed with a 12-lead ECG. The PM-free controls used in this study consisted of controls randomly selected from the Icelandic genealogical database and individuals from other ongoing related, but not cardiovascular, genetic studies at deCODE. Controls with first-degree relatives (siblings, parents or offspring) with PM, or a first-degree control relative, were excluded from the analysis.

Norwegian Atrial Fibrillation Sample from the Tromsø Study

The Tromsø Study AF population has been described previously {Gudbjartsson, 2009 #452}. Briefly, the Tromsø Study is a population-based prospective study with repeated health surveys in the municipality of Tromsø, Norway. The population is being followed-up on an individual level with registration and validation of diseases and death and an endpoint registry has been established for CVD. For the current project, one sex- and age matched control was selected for each case of AF from the population based Tromsø 4 survey. Participants in the Tromsø Study gave informed, written consent. The study was approved by the Regional Committee for Medical Research Ethics.

### Illumina Genome-Wide Genotyping

All Icelandic discovery samples were assayed with the Illumina HumanHap300 or HumanHapCNV370 bead chips (Illumina, SanDiego, Calif., USA), containing 317,503 and 370,404 haplotype tagging SNPs derived from phase I of the International HapMap project. Only SNPs present on both chips were included in the analysis and SNPs were excluded if they had (a) yield lower than 95% in cases or controls, (b) minor allele frequency less than 1% in the population, or (c) showed significant deviation from Hardy-Weinberg equilibrium in the controls (P<0.001). Any samples with a call rate below 98% were excluded from the analysis. The final analysis included, 306,060 SNPs.

### Single SNP Genotyping

Replication single SNP genotyping was carried out by deCODE genetics in Reykjavik, Iceland, applying the Centaurus (Kutyavin et al. 2006) (Nanogen) platform. The quality of each Centaurus SNP assay was evaluated by genotyping each assay on the CEU samples and comparing the results with the HapMap data. All assays had mismatch rate<0.5%. Additionally, all markers were re-genotyped on more than 10°/0 of samples typed with the Illumina platform resulting in an observed mismatch in less than 0.5% of samples.

### Quantitative Trait Analysis

All ECG measurements were adjusted for sex, year of birth and age at measurement after log transformation. In addition, the QT interval duration was adjusted for heart rate. After adjustment, the residuals ECG measurements were standardized using quantile-quantile standardization. For individuals with multiple ECG measurements, the mean standardized residual value was used. Drugs are known to influence many ECG variables, including specifically the heart rate, PR interval and QT interval. We were not able to adjust for drugs in our analysis as detailed drug information was not available for our sample set. However, Pfeufer et al in their GWAS on the QT interval¹⁷, found that accounting for QT-prolonging drugs explained only 0.25%-0.51% of the QT variance in their individual studies, and did not adjust for drugs in their meta-analysis.

For each SNP, a classical linear regression, using the genotype as an additive covariate and the standardized blood measurement as a response, was fit to test for association. The test statistics from the GWAS were scaled by the method of genomic control⁶⁶ obtained by comparing the observed median of all χ2-test statistic to the value predicted by theory (0.675²). The estimated inflation factors were 1.10, 1.16, 1.16 and 1.09 for the QT interval, PR interval, QRS complex and heart rate, respectively.

For the SNPs previously reported to associate with QT interval duration, that were not present on the Illumine chips, expected allele counts were obtained using the IMPUTE software⁶⁷, using the HapMap CEU samples as a training set⁵⁰. The test for association was then performed using the expected allele counts as covariates. The imputation information was estimated by the ratio of the observed variance of allele counts and the predicted variance of allele counts from the observed allele frequencies under the assumption of Hardy-Weinberg equilibrium.

### Heritability Estimation

The heritability of ECG measurements was estimated as twice the correlation between sibling pairs. The standardized residual measurements described above were used for the estimation of correlation.

### Case-Control Association Analysis

Association with disease phenotypes, such as AF, CVB and PM, was tested with a likelihood procedure described by Gretarsdottir, S. et al⁶⁸.

### Results

### Heritability of ECG Measures

We estimated the heritability of each of the four ECG variables assessed in this study based on twelve thousand Icelandic sibling pairs. Our analysis revealed estimates similar to those previously reported in other populations. The estimated heritability was 18% for HR, 40% for the PR interval, 33% for the QRS complex duration and 30% for the QT interval.

Several Sequence Variants Associate with PR Interval and QRS Duration

From the GWAS of PR interval, QRS complex duration and HR in ten thousand Icelanders, SNPs from seven regions were chosen for replication in additional ten thousand Icelanders with ECG information (Table 2).

The GWAS on the PR interval and QRS complex yielded GWS signals (P<1.6×10⁻⁷) for one locus common to the PR interval and the QRS complex and three PR interval specific loci. In addition to these loci, signals from two additional loci near GWS for QRS were tested in additional ten thousand replication samples. In the combined analysis of the discovery and replication samples we observed significant association between the PR interval and QRS complex and several loci (Table 2).

The combined analysis showed GWS association between TBX5 on chromosome 12q24.1 and the PR interval, QRS duration and QT interval and GWS association between SCN10A on 3p22.2 and both the PR interval and QRS duration. Two loci showed GWS association with the PR interval only, ARHGAP24 on 4q22.1 and CAV1 on 7q31, and another two loci to the QRS duration only, on chromosomes 6p21.31 (near CDKN1A) and 10q21.1 (near DKK1). Note that the QRS association at 10q21.1 borders on GWS (P=1.6×10-7) after adjustment for the four ECG parameters being tested (GWS threshold adjusting for four traits tested: P<4×10-8), although the six other primary associations to ECG parameters satisfy this more stringent threshold (Table 2).

The TBX5 variant, rs3825214[G] (freq=0.22), that associates with prolongation of the PR interval (combined P=3.3×10⁻¹²), QRS duration (combined P=3.0×10⁻¹³) and QT interval (combined P=9.5×10⁻⁸) in our data is located in the last intron of the TBX5 gene. As discussed before, the QRS duration and QT interval are correlated (correlation=0.44 in our data), and the association of rs3825214 with QT interval is weaker after conditioning on QRS duration but still significant (P=0.0065). No other genes share LD block with rs3825214. TBX5 encodes a transcription factor and plays a key role in cardiac development. Mutations in this gene cause limb and cardiac malformation in the Holt-Oram syndrome (HOS)^{20,21} with the main structural cardiac abnormalities being atrial and ventricular septal defects. Conduction disorders are frequently present (also in the absence of structural defects) and may affect the SN and AVN as well as the bundle branches, ranging in severity from asymptomatic conduction disturbances to SCD due to heart block²². TBX5 is widely expressed in the AVN and ventricular bundle branches in mice and is critical for development of the murine cardiac conduction system²³. TbxS has also been shown to regulate the connexin 40 gene in mice²⁴, a gap junction protein associated with electrical conduction. Interestingly, TbxS interacts with another transcription factor, Mef2c, to activate expression of the MYH6 gene²⁵ that associates with HR in our data. Most recently, a large GWAS reported association between diastolic blood pressure (DBP) and a common variant, rs2384550, close to TBX3 and T8X5²⁶. There is no correlation between this variant and the TBX5 variant reported here (r²=0.0018, D=0.058 in the Icelandic data) and similarly, the DBP variant did not associate with any of the four ECG variables.

The association between SCN10A and the PR interval and QRS duration was compelling in our data. The strongest association observed (combined P=9.5×10⁻⁵⁹ for prolonged PR interval, P=3.5×10⁻⁹ for prolonged QRS duration) was with rs6795970[A] (freq=0.36), representing a missense mutation, V1073A. SCN10A encodes a tetrodotoxin (TTX) resistant voltage-gated sodium channel (α-subunit), implicated in pain perception and modulation, that has previously been found primarily expressed in small sensory neurons in dorsal root ganglia^{27,28}. The most closely related human sodium channel gene is the cardiac voltage-gated sodium channel, SCN5A, with 70.4% similarity to SCN10A²⁷ and located next to SCN10A on chromosome 3. In contrast to SCNA5, the SCN10A gene has not previously been linked to cardiac function. Mutations in SCN5A have been demonstrated in several cardiac disorders including long QT syndrome, Brugada syndrome, SSS and AF²⁹, and recently, common variants in SCN5A were associated with the QT interval in two large GWAS^{16,17}.

We observed a strong association between prolongation of the PR interval and a common intronic variant in ARHGAP24, rs7660702[T] (freq=0.74, combined P=2.5×10⁻¹⁷). No other genes share the LD block with ARHGAP24 and this variant. ARHGAP24 encodes one of the Rho GTPase-activating proteins (RhoGAPs)³⁹, modulators of the Rho family of small GTPases (members of the Ras superfamily), binary molecular switches that are turned on and off in response to a variety of extracellular stimuli³¹. These GTPases are implicated in almost every fundamental cellular process and are crucially involved in the regulation of cell cytoskeletal organization, cell maturation and transcription³¹. The RhoGAPs accelerate the low intrinsic GTP hydrolysis rate of most Rho family members, converting their substrates to a GDP-bound inactive state. ARHGAP24 has specifically been shown to be involved in regulation of angiogenesis, actin remodeling and cell polarity^{32,33} but has not been linked to myocardial depolarization before. Abnormal expression of RhoGAP proteins has been observed in certain cancers but current knowledge of the normal biological function of most RhoGAPs is rudimentary.

The CAV1 variant, rs3807989[A] (freq=0.40), is an intronic variant that showed GWS association with prolongation of the PR interval (combined P=7.4×10⁻¹³) and secondary association with prolonged QRS duration (combined P=0.00011). The only other gene in the same LD block is CAV2. The caveolins are a family of highly conserved integral membrane proteins involved in dynamic and regulatory processes occurring at the plasma membrane, including vesicular trafficking and signal transduction³⁴. They act as scaffolding proteins and provide a framework for organization of specific caveolin-interacting lipids and regulation of lipid-modified signaling molecules³⁵. CAV-1 is involved in the regulation of the nitric oxide (NO) pathway^{36,37}, and CAV-1 knockout mice develop a dramatic increase in systemic NO levels, pulmonary hypertension, dilated cardiomyopathy and decreased lifespan³⁸.

Two common variants associated with prolongation of the QRS duration only, rs1321311[T] on chromosome 6p21.31 (freq=0.21, combined P=2.7×10⁻¹⁰), and rs1733724[T] on 10q21.1 (freq=0.21, combined P=6.5×10⁻⁸). The SNP rs1321311 shares LD block with one gene, CDKN1A, an important mediator of p53-dependent cell cycle arrest and thus a key player in cellular response to DNA damage and tumor growth suppression³⁹. The closest genes to rs1733724 include DKK1, CSTF2T, PRKG1 and MBL2. Further investigation is required to uncover the biological pathways connecting these two loci to QRS duration.

### Association Between Heart Rate and the MYH6 Gene on Chromosome 14q11.2-q13

We observed GWS association between the non-synonymous variant rs365990[G] (A1101V) in MYH6 (freq=0.34) and increased HR (combined P=9.4×10¹¹) with secondary association with shortened PR interval (combined P=1.8×10⁻⁵). The association with shortened PR interval remained significant after adjusting for the effect on HR (combined P=0.0027). Two different sarcomeric myosin heavy chain (MyHC) isoforms are expressed in the mammalian myocardium, α-MyHC (MYH6) and β-MyHC (MYH7)⁴⁰, with the two genes oriented in a head-to-tail tandem on chromosome 14⁴¹. Human hearts predominantly express the β isoform and little of the α isoform found there is primarily expressed in atrial tissue^{42,43}. The two isoforms have distinct properties as the α-MyHC exhibits markedly faster actin-activated ATPase activity⁴⁴ and actin filament sliding velocity⁴⁵ than β-MyHC, and myocytes containing only α-MyHC generate nearly three times greater peak normalized power than myocytes expressing exclusively β-MyHC⁴⁶. Despite the relatively small amount of α-MyHC expressed in the myocardium, a body of evidence suggests that MyHC isoform expression critically affects myocardial performance, such that a relatively small change in α-MyHC expression may significantly augment contractile capacity under stress⁴⁷. Mutations in MYH7 are a well known cause of hypertrophic cardiomyopathy, and recently, mutations in MYH6 have also been demonstrated in several cases of cardiomyopathy, both hypertrophic and dilated⁴⁸, and also in a family with dominantly inherited atrial septal defect (ASD)⁴⁹.

### Associations Between Several Loci and Cardiac Arrhythmias

The seven loci found to associate at a GWS level with ECG variables in the combined Icelandic data were next tested for association in Icelandic and Norwegian case-control samples of AF and Icelandic case-control samples of SSS, advanced AVB and an Icelandic PM population (see Tables 4 and 5 and descriptions of sample sets). We observed association between two loci, TBX5 and CAV1, and Atrial Fibrillation (N=4,304 cases and 46,508 controls, Table 5). For both variants, the allele that correlates with prolonged PR interval associates with less risk of AF; TBX5 (OR=0.88, P=4.0×10⁻⁵ for rs3825214[G]; risk allele rs3825214[A] with OR=1/0.88=1.14) and CAV1 (OR=0.92, P=0.00032 for rs3807989[A]; risk allele rs3807989[G] with OR=1/0.92=1.09).

For the TBX5 locus we also found association with advanced AVB (OR=1.27, P=0.0067 for rs3825214[G], N=359 cases, 48,994 controls) (Table 4). The allele that associates with prolonged PR interval carries an increased risk of advanced AVB. Finally, there was correlation between SCN10A and PM placement in the Icelandic PM sample set (OR=1.13, P=0.0029 for rs6795970[A], N=1,252 cases and 48,114 controls, Table 4). The same allele associates with prolonged PR/QRS duration and PM placement (See Tables 2 and 4). Further examination of the PM population according to the underlying arrhythmia did not reveal significant or stronger association with any one of the underlying diseases.

### Discussion

Through a large GWAS of Icelanders with ECG information we have discovered associations between several common sequence variants and HR, PR interval and QRS duration. Only one of the other genes identified, TBX5, has previously been implicated directly in cardiac conduction, although MYH6, the alpha-myosin cardiac heavy chain, and SCN10A, a sodium channel gene with marked similarity to the cardiac sodium channel, were good a priori candidates.

There are correlations between several of the ECG parameter SNPs and diseases, translating our findings to potential clinical relevance. We describe associations between common variants in CAV1 and AF, and TBX5 and both AF and advanced AVB. For both loci, the alleles that correlate with shorter PR interval predispose to AF. While the literature generally suggests a concordance between prolonged PR interval, representing delayed intra- and interatrial conduction, and risk of AF, familial syndromes have been described, including Lown-Ganong-Levine, exhibiting short PR, reflecting accelerated AV conduction, and increased risk of AF and other supraventricular tachycardias^{53,54}. Interestingly, a large family with atypical HOS, gain-of-function TBX5 mutation and paroxysmal AF was recently described⁵⁵. The observed associations with clinical syndromes require confirmation in additional cohorts.

While the associations we have identified explain only a small fraction of the variance of the ECG measures studied, the observed correlations with clinical disease yet again demonstrate how studies of intermediate traits may lead to discovery of clinically pertinent biological pathways. This approach has previously been successful in the study of SCD through the QT interval¹⁵, lung cancer and peripheral arterial disease through smoking quantity⁵⁶, asthma and myocardial infarction through serum eosinophil counts⁵⁷ and risk of coronary artery disease through LDL serum concentration⁵⁸.

Two of the associated SNPs are coding non-synonymous variants, in SCN10A and MYH6, and others are in functionally relevant genes, including TBX5, suggesting causality. Functional studies are necessary to further elucidate the biological pathways that are represented by the observed common sequence variants and modulate cardiac conduction and clinical syndromes. Additionally, resequencing of the candidate genes may identify common and rare functional variants with greater impact on cardiac conduction.

### Accession Numbers

ARHGAP24: AK091196 and NM031305, CAV1: AF125348 and NM001753, CAV2: AF035752 and NM001233, CDKN1A: UO3106 and NM078467, CSTF2T: AB014589 and NM015235, DKK1: NM012242, MBL2: AF360991 and NM000242, MYH6: D00943, PRKG1: NM006258, TBX5: U89353 and NM080717, SCN5A: A3310893 and NM198056, SCN10A: AF117907 and NM006514.

TABLE 1
Shows an overview of the two Icelandic ECG study populations

| | | | | | Avg. | | |
|---|---|---|---|---|---|---|---|
| | | | | | number | | |
| | Popula- | | | Age^{a} | of measure- | Mean | |
| Trait | tion | Sex | N | (SD) | ments^{b} | (SD) | |
| QT | Discov- | Male | 4,466 | 66 (14) | 2.4 | 388 | ms |
| | ery | Female | 5,395 | 66 (16) | 2.0 | 385 | ms |
| | Replica- | Male | 4,171 | 60 (15) | 1.9 | 385 | ms |
| | tion | Female | 4,646 | 62 (15) | 1.7 | 388 | ms |
| PR | Discov- | Male | 4,803 | 66 (14) | 2.5 | 178 | ms |
| | ery | Female | 5,570 | 66 (16) | 2.0 | 168 | ms |
| | Replica- | Male | 4,400 | 60 (15) | 1.9 | 175 | ms |
| | tion | Female | 4,752 | 62 (15) | 1.7 | 167 | ms |
| QRS | Discov- | Male | 4,811 | 66 (14) | 2.5 | 99 | ms (17) |
| | ery | Female | 5,575 | 66 (16) | 2.0 | 90 | ms (14) |
| | Replica- | Male | 4,400 | 61 (15) | 1.9 | 97 | ms (15) |
| | tion | Female | 4,753 | 62 (15) | 1.7 | 89 | ms (12) |
| HR | Discov- | Male | 6,144 | 73 (16) | 3.1 | 73 | bpm |
| | ery | Female | 6,616 | 68 (16) | 2.4 | 76 | bpm |
| | Replica- | Male | 5,107 | 62 (15) | 2.3 | 72 | bpm |
| | tion | Female | 5,245 | 62 (16) | 1.9 | 73 | bpm |

^{a}Age at measurement.
^{b}Geometric mean of the number of measurements per individual.
SD = standard deviation, ms = milliseconds, bpm = beats per minute.

TABLE 2
Presenting association of common sequence variants with ECG measures for two Icelandic datasets and their combined values.

| | | | | | | | Discovery | | Follow-up | | Combined | |
|---|---|---|---|---|---|---|---|---|---|---|---|---|
| Closest | | | | | | Mea- | Ef- | P- | Ef- | P- | Effect^{b} | P- |
| gene | SNP | All | Chr | Position | Freq^{a} | sure | fect^{b} | value | fect^{b} | value | (95% CI) | val [symbol] |
| Missense | rs6795970 | A | 3 | 38,741,679 | 0.360 | PR | 15.06 | 6.2 · 10⁻³³ | 14.53 | 1.3 · 10⁻²⁷ | 14.81 (13.01, 16.60) | 9.5 · 10⁻ [symbol] |
| SCN10A | | | | | | QRS | 4.45 | 0.00026 | 6.02 | 1.7 · 10⁻⁶ | 5.17 (3.46, 6.89) | 3.5 · 10⁻ [symbol] |
| Intron | rs7660702 | T | 4 | 86,870,488 | 0.737 | PR | 8.93 | 1.3 · 10⁻¹⁰ | 7.97 | 2.9 · 10⁻⁸ | 8.46 (6.50, 10.42) | 2.5 · 10⁻ [symbol] |
| ARHGAP24 | | | | | | | | | | | | |
| Upstream | rs1321311 | T | 6 | 36,730,878 | 0.206 | QRS | 7.14 | 7.8 · 10⁻⁷ | 5.83 | 7.8 · 10⁻⁵ | 6.52 (4.50, 8.55) | 2.7 · 10⁻¹⁰ |
| CDKN1A | | | | | | | | | | | | |
| Intron | rs3807989 | A | 7 | 115,973,477 | 0.401 | PR | 6.60 | 7.1 · 10⁻⁸ | 6.19 | 2.0 · 10⁻⁶ | 6.40 (4.65, 8.15) | 7.4 · 10⁻¹³ |
| CAV1 | | | | | | QRS | 3.55 | 0.0026 | 3.02 | 0.014 | 3.30 (1.63, 4.97) | 0.00011 |
| Downstream | rs1733724 | T | 10 | 53,893,983 | 0.215 | QRS | 6.20 | 1.6 · 10⁻⁵ | 4.96 | 0.00099 | 5.62 (3.58, 7.66) | 6.5 · 10⁻⁸ |
| DKK1 | | | | | | | | | | | | |
| Intron | rs3825214 | G | 12 | 113,279,826 | 0.216 | QRS | 7.56 | 6.1 · 10⁻⁸ | 7.1 | 1.1 · 10⁻⁶ | 7.35 (5.37, 9.33) | 3.0 · 10⁻¹³ |
| TBX5 | | | | | | PR | 8.14 | 2.0 · 10⁻⁸ | 6.43 | 3.1 · 10⁻⁵ | 7.36 (5.29, 9.43) | 3.3 · 10⁻¹² |
| | | | | | | QT | 5.09 | 0.00069 | 6.82 | 2.7 · 10⁻⁵ | 5.88 (3.72, 8.03) | 9.5 · 10⁻⁸ |
| Missense | rs365990 | G | 14 | 22,931,651 | 0.341 | HR | 4.79 | 9.5 · 10⁻⁶ | 5.7 | 2.9 · 10⁻⁶ | 5.25 (3.66, 6.83) | 9.4 · 10⁻¹¹ |
| MYH6 | | | | | | PR | −4.17 | 0.001 | −3.73 | 0.0066 | −3.99 (−2.17, −5.82) | 1.8 · 10⁻⁵ |

Shown are the results for the discovery and follow-up samples and the two sample sets combined.
The sample sizes for the discovery/follow-up/combined samples for each ECG measure are as follows: PR interval 10,373/9,152/19,525; QRS complex 10,386/9,153/19,539; QT interval 9,861/8,817/18,678 and HR 12,760/10,352/23,112, respectively.
^{a}The reported allele frequencies are the frequencies in the combined sample sets.
^{b}Effects are given in percentage of standard deviation. The closest genes to the variants reported are shown for reference. [symbol]
indicates data missing or illegible when filed

TABLE 3
Shows correlation between all pairs of ECG measurements.

| Measure 1 | Measure 2 | Correlation |
|---|---|---|
| PR | QRS | 0.09 |
| PR | QT | 0.06 |
| QRS | QT | 0.44 |
| HR | PR | −0.23 |
| HR | QRS | −0.07 |
| HR | QT | 0.00 |

Note that the QT interval has been adjusted for HR.

TABLE 4
Presents association between ECG parameter loci and Icelandic case-control sample sets of atrial
fibrillation, pacemaker placement, sick sinus syndrome and advanced atrioventricular block.

| | | | | Atrial | | | | Sick sinus | | Advanced | |
|---|---|---|---|---|---|---|---|---|---|---|---|
| | | | | fibrillation | | Pacemaker | | syndrome | | AV blo [symbol] | |
| | | | | (N = 3,584) | | (N = 1,252) | | (N = 1,076) | | (N = 359) | |
| | Closest | | | OR | P- | OR | P- | OR | P- | OR | P- |
| SNP | gene^{a} | All | Chr | (95% CI) | value | (95% CI) | value | (95% CI) | value | (95% CI) | val [symbol] |
| rs6795970 | Missense | A | 3 | 0.97 (0.92, 1.03) | 0.31 | 1.13 (1.04, 1.23) | 0.0029 | 1.09 (1.00, 1.19) | 0.064 | 1.07 (0.91, 1.24) | 0.41 |
| | SCN10A | | | | | | | | | | |
| rs7660702 | Intron | T | 4 | 1.03 (0.98, 1.09) | 0.25 | 1.00 (0.91, 1.09) | 0.92 | 1.00 (0.90, 1.10) | 0.96 | 1.02 (0.86, 1.20) | 0.82 |
| | ARHGAP24 | | | | | | | | | | |
| rs1321311 | Upstream | T | 6 | 0.95 (0.89, 1.01) | 0.098 | 1.02 (0.93, 1.13) | 0.67 | 0.95 (0.85, 1.05) | 0.32 | 1.10 (0.92, 1.31) | 0.31 |
| | CDKN1A | | | | | | | | | | |
| rs3807989 | Intron | A | 7 | 0.93 (0.88, 0.98) | 0.0048 | 0.96 (0.89, 1.04) | 0.35 | 1.00 (0.91, 1.09) | 0.96 | 1.07 (0.93, 1.25) | 0.35 |
| | CAV1 | | | | | | | | | | |
| rs1733724 | Downstream | T | 10 | 1.04 (0.98, 1.10) | 0.23 | 1.07 (0.97, 1.18) | 0.15 | 1.09 (0.98, 1.21) | 0.10 | 1.08 (0.90, 1.29) | 0.39 |
| | DKK1 | | | | | | | | | | |
| rs3825214 | Intron | G | 12 | 0.88 (0.83, 0.94) | 8.5 · | 1.03 (0.94, 1.13) | 0.55 | 0.97 (0.88, 1.08) | 0.59 | 1.27 (1.07, 1.50) | 0.0067 |
| | TBX5 | | | | 10⁻⁵ | | | | | | |
| rs365990 | Missense | G | 14 | 1.04 (0.99, 1.10) | 0.13 | 0.94 (0.86, 1.02) | 0.13 | 0.94 (0.86, 1.03) | 0.18 | 0.93 (0.79, 1.08) | 0.33 |
| | MYH6 | | | | | | | | | | |

Shown is the marker name, the closest gene to the variant, risk allele, chromosome and odds ratio and P-value for all sample sets. [symbol]
indicates data missing or illegible when filed

TABLE 5
Shows association between ECG parameter loci in Icelandic and Norwegian AF case-control sample sets and combined results.

| | | | | Iceland | | Norway | | Combined | |
|---|---|---|---|---|---|---|---|---|---|
| | | | | N cases = 3,584, | | (N cases = 720, | | (N cases = 4,304, | |
| | | | | N controls = 45,783) | | N controls = 725) | | N controls = 46,508) | |
| SNP | Closest gene | All | Chr | OR (95% CI) | P-value | OR (95% CI) | P-value | OR (95% CI) | P-value |
| rs6795970 | Missense SCN10A | A | 3 | 0.97 (0.92, 1.03) | 0.31 | 0.97 (0.84, 1.14) | 0.74 | 0.97 (0.93, 1.02) | 0.29 |
| rs7660702 | Intron ARHGAP24 | T | 4 | 1.03 (0.98, 1.09) | 0.25 | 0.97 (0.83, 1.14) | 0.72 | 1.03 (0.97, 1.08) | 0.33 |
| rs1321311 | Upstream CDKN1A | T | 6 | 0.95 (0.89, 1.01) | 0.098 | 0.89 (0.75, 1.06) | 0.20 | 0.94 (0.89, 1.00) | 0.047 |
| rs3807989 | Intron CAV1 | A | 7 | 0.93 (0.88, 0.98) | 0.0048 | 0.80 (0.69, 0.93) | 0.0037 | 0.92 (0.87, 0.96) | 0.00032 |
| rs1733724 | Downstream DKK1 | T | 10 | 1.04 (0.98, 1.10) | 0.23 | 1.05 (0.88, 1.26) | 0.58 | 1.04 (0.98, 1.10) | 0.19 |
| rs3825214 | Intron TBX5 | G | 12 | 0.88 (0.83, 0.94) | 8.5 · 10⁻⁵ | 0.89 (0.75, 1.07) | 0.23 | 0.88 (0.83, 0.94) | 4.0 · 10⁻5 |
| rs365990 | Missense MYH6 | G | 14 | 1.04 (0.99, 1.10) | 0.13 | 1.00 (0.85, 1.18) | 0.98 | 1.04 (0.99, 1.09) | 0.14 |

Shown is the marker name, the closest gene to the variant, risk allele, chromosome. Odds ratio and P-value for separate and combined sample sets.

TABLE 6
List of surrogate markers to rs6795970 on Chromosome
3 with R² >0.2 in the HapMap CEU dataset.

| Marker | Correlated | Pos in NCBI | | | | Seq ID |
|---|---|---|---|---|---|---|
| Name | Allele | Build 36 | R² | D′ | P-value | NO: |
| rs6599240 | 1 | 38713721 | 0.51396 | 0.806534 | 3.46E−14 | 1 |
| rs11129800 | 4 | 38719374 | 0.541438 | 0.836706 | 2.87E−14 | 2 |
| rs11129801 | 3 | 38725379 | 0.235547 | 1 | 4.63E−10 | 3 |
| rs11710006 | 1 | 38727794 | 0.257268 | 1 | 5.97E−10 | 4 |
| rs11924846 | 2 | 38731570 | 1 | 1 | 1.81E−37 | 5 |
| rs9990137 | 1 | 38734469 | 0.443478 | 1 | 7.21E−17 | 6 |
| rs6805187 | 2 | 38735510 | 0.443478 | 1 | 1.16E−16 | 7 |
| rs7617547 | 3 | 38738504 | 0.342524 | 1 | 1.87E−13 | 8 |
| rs6771157 | 3 | 38738867 | 0.260745 | 1 | 1.09E−10 | 9 |
| rs4076737 | 2 | 38739786 | 1 | 1 | 1.01E−36 | 10 |
| rs12632942 | 1 | 38740002 | 0.25745 | 1 | 8.36E−11 | 11 |
| rs7430477 | 2 | 38740494 | 0.492754 | 1 | 3.30E−18 | 12 |
| rs6795970 | 1 | 38741679 | 1 | 1 | | 13 |
| rs6801957 | 4 | 38742319 | 0.93228 | 0.965876 | 3.05E−30 | 14 |
| rs7433306 | 2 | 38745643 | 0.966314 | 1 | 1.23E−34 | 15 |
| rs6780103 | 3 | 38746468 | 0.244908 | 1 | 2.62E−10 | 16 |
| rs6790396 | 2 | 38746929 | 0.781825 | 0.919395 | 5.18E−21 | 17 |
| rs6800541 | 2 | 38749836 | 0.932942 | 0.965889 | 1.31E−30 | 18 |
| rs7615140 | 4 | 38757030 | 0.365219 | 0.940648 | 1.80E−10 | 19 |
| rs6599250 | 4 | 38759033 | 0.932942 | 0.965889 | 1.31E−30 | 20 |
| rs6599251 | 3 | 38760813 | 0.783405 | 0.963023 | 1.90E−24 | 21 |
| rs7430451 | 2 | 38770499 | 0.350851 | 0.938908 | 5.04E−10 | 22 |
| rs6599254 | 1 | 38770559 | 0.932942 | 0.965889 | 1.31E−30 | 23 |
| rs6599255 | 1 | 38771419 | 0.778951 | 0.92858 | 7.83E−24 | 24 |
| rs12630795 | 1 | 38771989 | 0.214098 | 1 | 3.10E−09 | 25 |
| rs6798015 | 2 | 38773840 | 0.746427 | 0.925692 | 2.39E−22 | 26 |
| rs6763876 | 4 | 38775751 | 0.236944 | 0.919317 | 2.55E−07 | 27 |
| rs6599256 | 3 | 38776229 | 0.236944 | 0.919317 | 2.55E−07 | 28 |
| rs7641844 | 1 | 38777255 | 0.222485 | 0.905737 | 2.96E−06 | 29 |
| rs7432804 | 1 | 38778513 | 0.236944 | 0.919317 | 2.55E−07 | 30 |
| rs7430439 | 3 | 38778643 | 0.235513 | 0.510992 | 2.09E−06 | 31 |
| rs7651106 | 4 | 38779345 | 0.5233 | 0.950002 | 4.03E−15 | 32 |
| rs6599257 | 2 | 38779592 | 0.5233 | 0.950002 | 4.03E−15 | 33 |
| rs7610489 | 1 | 38781482 | 0.538271 | 0.950007 | 5.01E−15 | 34 |
| rs7650384 | 2 | 38781515 | 0.395362 | 0.936308 | 2.95E−10 | 35 |
| rs4414778 | 4 | 38787169 | 0.229284 | 0.91387 | 8.01E−07 | 36 |
| rs10212338 | 1 | 38787654 | 0.522914 | 0.949651 | 8.10E−15 | 37 |

The table includes, for each SNP, the correlating allele with allele A of rs6795970, position in NCBI build 36, and r², D′ and P-value for the test of correlation between the two markers.

TABLE 7
List of surrogate markers to rs7660702 on Chromosome
4, with R² >0.2 in the HapMap CEU dataset.

| Marker | Correlated | Pos in NCBI | | | | Seq ID |
|---|---|---|---|---|---|---|
| Name | Allele | Build 36 | R² | D′ | P-value | NO: |
| rs7698203 | 4 | 86823753 | 0.201953 | 0.871243 | 5.12E−06 | 38 |
| rs6849659 | 1 | 86829480 | 0.638204 | 0.944142 | 1.25E−16 | 39 |
| rs2101134 | 1 | 86831373 | 0.772296 | 1 | 9.61E−24 | 40 |
| rs10017047 | 3 | 86834415 | 0.456462 | 0.930433 | 2.01E−12 | 41 |
| rs12648692 | 1 | 86837105 | 0.220999 | 1 | 4.21E−07 | 42 |
| rs13134382 | 3 | 86837827 | 0.207596 | 0.6802 | 3.16E−06 | 43 |
| rs17010599 | 4 | 86838704 | 0.239669 | 1 | 1.01E−07 | 44 |
| rs7655100 | 1 | 86839892 | 0.816682 | 1 | 6.15E−26 | 45 |
| rs7677064 | 4 | 86839908 | 0.475018 | 0.932274 | 5.87E−13 | 46 |
| rs10033273 | 2 | 86840097 | 0.457043 | 0.931024 | 1.00E−12 | 47 |
| rs7439720 | 1 | 86840878 | 0.457043 | 0.931024 | 1.00E−12 | 48 |
| rs900204 | 3 | 86841290 | 0.223745 | 0.693487 | 9.57E−07 | 49 |
| rs11731040 | 2 | 86841367 | 0.239669 | 1 | 1.01E−07 | 50 |
| rs931195 | 1 | 86843465 | 0.457043 | 0.931024 | 1.00E−12 | 51 |
| rs17010632 | 1 | 86844920 | 0.457043 | 0.931024 | 1.00E−12 | 52 |
| rs1871864 | 1 | 86848126 | 0.441842 | 0.926246 | 8.42E−12 | 53 |
| rs1871865 | 3 | 86848557 | 0.457043 | 0.931024 | 1.00E−12 | 54 |
| rs1482085 | 3 | 86849536 | 0.810964 | 1 | 5.18E−25 | 55 |
| rs11735639 | 4 | 86850942 | 0.457043 | 0.931024 | 1.00E−12 | 56 |
| rs4413396 | 3 | 86851795 | 0.457043 | 0.931024 | 1.00E−12 | 57 |
| rs13146939 | 1 | 86852284 | 0.459761 | 0.927606 | 5.12E−12 | 58 |
| rs13152150 | 1 | 86852331 | 0.457043 | 0.931024 | 1.00E−12 | 59 |
| rs13128115 | 1 | 86852508 | 0.456175 | 0.929484 | 1.42E−12 | 60 |
| rs12509904 | 3 | 86854591 | 0.22264 | 0.690722 | 1.08E−06 | 61 |
| rs12650494 | 2 | 86856334 | 0.239669 | 1 | 1.10E−07 | 62 |
| rs10012090 | 3 | 86857950 | 1 | 1 | 2.84E−32 | 63 |
| rs7691602 | 1 | 86859569 | 0.213759 | 1 | 5.01E−07 | 64 |
| rs7692808 | 3 | 86860173 | 1 | 1 | 1.48E−32 | 65 |
| rs7658797 | 2 | 86861738 | 1 | 1 | 9.56E−29 | 66 |
| rs17010697 | 1 | 86862179 | 0.49595 | 1 | 1.01E−14 | 67 |
| rs343860 | 3 | 86863360 | 1 | 1 | 1.48E−32 | 68 |
| rs13108523 | 4 | 86867448 | 0.348199 | 1 | 1.20E−10 | 69 |
| rs1482094 | 3 | 86869043 | 1 | 1 | 1.48E−32 | 70 |
| rs7676486 | 2 | 86869655 | 0.348199 | 1 | 1.20E−10 | 71 |
| rs7660702 | 4 | 86870488 | 1 | 1 | | 72 |
| rs2062098 | 3 | 86871272 | 0.783784 | 1 | 6.93E−22 | 73 |
| rs1482091 | 2 | 86872385 | 0.239669 | 1 | 1.01E−07 | 74 |
| rs6813860 | 2 | 86874152 | 0.348199 | 1 | 1.20E−10 | 75 |
| rs994285 | 2 | 86878138 | 1 | 1 | 2.75E−31 | 76 |
| rs343853 | 1 | 86880195 | 0.213759 | 1 | 5.01E−07 | 77 |
| rs343849 | 1 | 86882079 | 1 | 1 | 2.05E−32 | 78 |
| rs3889735 | 4 | 86882860 | 0.348199 | 1 | 1.20E−10 | 79 |
| rs2601855 | 4 | 86883964 | 1 | 1 | 1.48E−32 | 80 |
| rs2601857 | 4 | 86884123 | 0.213759 | 1 | 5.01E−07 | 81 |
| rs7682971 | 4 | 86884320 | 0.239669 | 1 | 1.01E−07 | 82 |
| rs10516755 | 3 | 86885065 | 1 | 1 | 1.48E−32 | 83 |
| rs1020584 | 1 | 86888074 | 0.455202 | 1 | 2.47E−12 | 84 |
| rs13106553 | 3 | 86888786 | 0.348199 | 1 | 1.20E−10 | 85 |
| rs12510813 | 1 | 86892745 | 0.625117 | 1 | 1.90E−18 | 86 |
| rs12507272 | 4 | 86892912 | 0.625117 | 1 | 2.36E−18 | 87 |
| rs13137008 | 4 | 86893223 | 1 | 1 | 1.48E−32 | 88 |
| rs13112493 | 1 | 86895103 | 0.49595 | 1 | 2.44E−14 | 89 |
| rs4693735 | 4 | 86896131 | 0.348199 | 1 | 1.20E−10 | 90 |
| rs12507198 | 3 | 86901712 | 0.348199 | 1 | 1.75E−10 | 91 |
| rs13111662 | 4 | 86902145 | 1 | 1 | 5.28E−31 | 92 |
| rs11732231 | 2 | 86902584 | 0.810964 | 1 | 5.18E−25 | 93 |
| rs11736641 | 3 | 86902753 | 1 | 1 | 2.05E−32 | 94 |
| rs11097071 | 1 | 86904360 | 0.360568 | 1 | 7.62E−11 | 95 |
| rs7674888 | 3 | 86904770 | 0.49595 | 1 | 1.01E−14 | 96 |
| rs1966862 | 1 | 86907085 | 0.348199 | 1 | 1.20E−10 | 97 |
| rs12054628 | 4 | 86907793 | 0.49595 | 1 | 1.01E−14 | 98 |
| rs17010839 | 4 | 86910281 | 0.247863 | 1 | 9.07E−08 | 99 |
| rs11945319 | 3 | 86910619 | 0.34607 | 1 | 5.44E−10 | 100 |
| rs6831420 | 3 | 86911689 | 0.468186 | 1 | 2.37E−13 | 101 |
| rs7680588 | 2 | 86912777 | 0.49595 | 1 | 1.01E−14 | 102 |
| rs17010851 | 1 | 86914746 | 0.348199 | 1 | 1.36E−10 | 103 |
| rs17010857 | 3 | 86915002 | 0.348199 | 1 | 1.20E−10 | 104 |
| rs4693736 | 3 | 86915344 | 0.295644 | 1 | 6.85E−09 | 105 |
| rs13105921 | 1 | 86918750 | 1 | 1 | 1.48E−32 | 106 |
| rs17010887 | 2 | 86920745 | 0.213759 | 1 | 5.43E−07 | 107 |
| rs17010892 | 2 | 86920909 | 0.213759 | 1 | 5.43E−07 | 108 |
| rs17395020 | 4 | 86921136 | 0.261778 | 1 | 5.24E−11 | 109 |
| rs17399123 | 4 | 86921194 | 0.270936 | 1 | 4.77E−11 | 110 |
| rs10516756 | 4 | 86923000 | 0.213759 | 1 | 5.01E−07 | 111 |
| rs1452681 | 4 | 86924688 | 0.270936 | 1 | 2.75E−11 | 112 |
| rs9790823 | 2 | 86927744 | 0.217904 | 1 | 6.85E−07 | 113 |
| rs7683733 | 2 | 86928771 | 0.220422 | 0.479314 | 3.56E−06 | 114 |
| rs7662174 | 4 | 86928923 | 0.220422 | 0.479314 | 3.56E−06 | 115 |
| rs7684607 | 1 | 86929302 | 0.225814 | 0.50849 | 2.51E−06 | 116 |
| rs13118915 | 2 | 86930744 | 0.220422 | 0.479314 | 3.56E−06 | 117 |
| rs17010925 | 4 | 86932148 | 0.39058 | 0.860672 | 1.62E−10 | 118 |
| rs12503243 | 4 | 86934428 | 0.213759 | 1 | 5.01E−07 | 119 |
| rs7675429 | 2 | 86940109 | 0.201953 | 0.871243 | 5.12E−06 | 120 |
| rs7689056 | 3 | 86942197 | 0.201953 | 0.871243 | 5.12E−06 | 121 |
| rs7693640 | 2 | 86942405 | 0.201953 | 0.871243 | 5.12E−06 | 122 |

The table includes, for each SNP, the correlating allele with allele T of rs7660702, position in NCBI build 36, and r², D′ and P-value for the test of correlation between the two markers.

TABLE 8
List of surrogate markers to rs1321311 on Chromosome
6 with R² >0.2 in the HapMap CEU dataset.

| Marker | Correlated | Pos in NCBI | | | | Seq ID |
|---|---|---|---|---|---|---|
| name | Allele | Build 36 | R² | D′ | P-value | NO: |
| rs6457931 | 3 | 36721790 | 0.543871 | 0.952077 | 3.77E−17 | 123 |
| rs12207916 | 2 | 36725630 | 0.787201 | 0.958333 | 3.45E−24 | 124 |
| rs1321313 | 1 | 36726799 | 0.959677 | 1 | 7.68E−31 | 125 |
| rs4713994 | 4 | 36729511 | 0.375 | 1 | 1.67E−14 | 126 |
| rs1321311 | 4 | 36730878 | 1 | 1 | | 127 |
| rs1321310 | 3 | 36731102 | 0.960784 | 1 | 2.28E−31 | 128 |
| rs4331968 | 4 | 36731221 | 0.960784 | 1 | 2.28E−31 | 129 |
| rs9470361 | 1 | 36731357 | 0.879061 | 0.958567 | 3.91E−26 | 130 |
| rs6930671 | 2 | 36733250 | 0.563404 | 0.952505 | 2.21E−17 | 131 |
| rs11969445 | 2 | 36733360 | 0.563404 | 0.952505 | 2.21E−17 | 132 |
| rs9470366 | 1 | 36733540 | 0.848485 | 1 | 1.26E−26 | 133 |
| rs6936993 | 2 | 36734300 | 0.584107 | 0.953211 | 6.15E−18 | 134 |
| rs9470367 | 3 | 36734910 | 0.337685 | 0.938491 | 3.55E−11 | 135 |
| rs7756236 | 3 | 36735031 | 0.922481 | 1 | 1.37E−29 | 136 |
| rs9462207 | 2 | 36735577 | 0.922481 | 1 | 1.37E−29 | 137 |
| rs9368950 | 4 | 36735850 | 0.337685 | 0.938491 | 3.55E−11 | 138 |
| rs9462208 | 2 | 36735868 | 0.563404 | 0.952505 | 2.21E−17 | 139 |
| rs9462209 | 3 | 36736020 | 0.558936 | 0.951008 | 1.86E−16 | 140 |
| rs9462210 | 1 | 36736931 | 0.920319 | 1 | 4.60E−29 | 141 |
| rs10807170 | 2 | 36737422 | 0.525322 | 0.906625 | 2.75E−16 | 142 |
| rs4713996 | 2 | 36737692 | 0.521913 | 0.906391 | 3.85E−16 | 143 |
| rs9394368 | 2 | 36738503 | 0.560693 | 0.908894 | 4.21E−17 | 144 |
| rs4713999 | 1 | 36741047 | 0.520598 | 0.905258 | 7.59E−16 | 145 |
| rs4711457 | 4 | 36741138 | 0.49595 | 0.901374 | 8.21E−15 | 146 |
| rs6930083 | 1 | 36742134 | 0.542469 | 0.907759 | 1.11E−16 | 147 |
| rs4714001 | 3 | 36746153 | 0.466434 | 0.89957 | 3.47E−14 | 148 |
| rs1321309 | 2 | 36746614 | 0.333627 | 0.83924 | 8.26E−11 | 149 |
| rs733590 | 2 | 36753181 | 0.547811 | 0.907176 | 2.26E−16 | 150 |
| rs2395655 | 3 | 36753674 | 0.549019 | 0.908175 | 5.67E−17 | 151 |
| rs3176352 | 3 | 36760317 | 0.222202 | 0.546647 | 2.35E−06 | 152 |
| rs12207548 | 4 | 36764234 | 0.26932 | 0.601822 | 1.00E−07 | 153 |
| rs12191972 | 2 | 36766720 | 0.289736 | 0.668157 | 6.01E−08 | 154 |
| rs7767246 | 3 | 36767193 | 0.312694 | 0.679409 | 8.45E−09 | 155 |
| rs6937605 | 4 | 36767910 | 0.363382 | 0.769281 | 4.12E−10 | 156 |
| rs7762245 | 1 | 36824207 | 0.201313 | 0.484629 | 6.59E−06 | 157 |

The table includes, for each SNP, the correlating allele with allele T of rs1321311, position in NCBI build 36, and r², D′ and P-value for the test of correlation between the two markers.

TABLE 9
List of surrogate markers to rs3807989 on Chromosome
7, with R² >0.2 in the HapMap CEU dataset.

| Marker | Correlated | Pos in NCBI | | | | Seq ID |
|---|---|---|---|---|---|---|
| Name | Allele | Build 36 | R² | D′ | P-value | NO: |
| rs2157799 | 4 | 115791226 | 0.253596 | 0.633413 | 1.50E−06 | 158 |
| rs721994 | 1 | 115805024 | 0.250393 | 0.685905 | 3.21E−06 | 159 |
| rs1728723 | 3 | 115805415 | 0.235497 | 0.671188 | 8.71E−06 | 160 |
| rs2049902 | 2 | 115816159 | 0.226304 | 0.566185 | 4.86E−06 | 161 |
| rs11772856 | 2 | 115822080 | 0.248563 | 0.604284 | 1.60E−06 | 162 |
| rs1858810 | 2 | 115846095 | 0.297303 | 0.546642 | 1.21E−06 | 163 |
| rs7781492 | 3 | 115857211 | 0.323984 | 0.570206 | 1.18E−07 | 164 |
| rs10464649 | 2 | 115860803 | 0.353195 | 0.605573 | 2.72E−09 | 165 |
| rs12706089 | 4 | 115871603 | 0.346706 | 0.588817 | 2.39E−09 | 166 |
| rs7782281 | 3 | 115874582 | 0.241009 | 0.823196 | 5.52E−06 | 167 |
| rs4727831 | 1 | 115881219 | 0.363424 | 0.611386 | 9.31E−10 | 168 |
| rs768108 | 4 | 115895894 | 0.346706 | 0.588817 | 2.39E−09 | 169 |
| rs717957 | 3 | 115900343 | 0.296077 | 0.611789 | 4.46E−08 | 170 |
| rs1883049 | 3 | 115905619 | 0.223085 | 1 | 7.03E−09 | 171 |
| rs6959099 | 1 | 115906716 | 0.223085 | 1 | 7.03E−09 | 172 |
| rs6975771 | 1 | 115910081 | 0.223085 | 1 | 7.03E−09 | 173 |
| rs6976316 | 3 | 115910179 | 0.296077 | 0.611789 | 4.46E−08 | 174 |
| rs6954077 | 3 | 115916389 | 0.305707 | 0.61506 | 4.44E−07 | 175 |
| rs728690 | 4 | 115919122 | 0.208646 | 1 | 2.09E−08 | 176 |
| rs10228178 | 3 | 115919447 | 0.294818 | 0.608492 | 3.88E−08 | 177 |
| rs2402081 | 2 | 115924531 | 0.213336 | 1 | 4.29E−08 | 178 |
| rs2270188 | 3 | 115927760 | 0.389009 | 0.749679 | 1.03E−10 | 179 |
| rs10271007 | 1 | 115933085 | 0.369378 | 0.729328 | 2.85E−09 | 180 |
| rs4730743 | 1 | 115933193 | 0.388598 | 0.742622 | 4.13E−10 | 181 |
| rs4727833 | 2 | 115935144 | 0.389009 | 0.749679 | 1.03E−10 | 182 |
| rs2109513 | 4 | 115937344 | 0.22815 | 1 | 5.39E−09 | 183 |
| rs6466579 | 4 | 115938391 | 0.389009 | 0.749679 | 1.03E−10 | 184 |
| rs3919515 | 3 | 115939020 | 0.385928 | 0.745217 | 1.73E−10 | 185 |
| rs975028 | 1 | 115944363 | 0.223085 | 1 | 7.03E−09 | 186 |
| rs2215448 | 1 | 115951188 | 0.203338 | 1 | 3.85E−08 | 187 |
| rs2742125 | 4 | 115952699 | 0.21142 | 1 | 2.31E−08 | 188 |
| rs3779512 | 4 | 115958299 | 0.242436 | 0.536345 | 3.53E−07 | 189 |
| rs9649394 | 1 | 115958746 | 0.221316 | 0.501891 | 1.42E−06 | 190 |
| rs1474510 | 1 | 115964693 | 0.223085 | 1 | 9.82E−09 | 191 |
| rs3807986 | 3 | 115965061 | 0.388538 | 0.939359 | 5.00E−11 | 192 |
| rs6466584 | 3 | 115967192 | 0.223085 | 1 | 7.03E−09 | 193 |
| rs6466585 | 3 | 115967220 | 0.223085 | 1 | 7.03E−09 | 194 |
| rs1476833 | 4 | 115968711 | 0.223085 | 1 | 8.31E−09 | 195 |
| rs976739 | 1 | 115971767 | 0.237811 | 1 | 2.32E−09 | 196 |
| rs3807989 | 1 | 115973477 | 1 | 1 | | 197 |
| rs3801995 | 4 | 115977833 | 0.43423 | 0.944245 | 7.89E−13 | 198 |
| rs3815412 | 2 | 115977929 | 0.499781 | 1 | 8.27E−18 | 199 |
| rs11773845 | 2 | 115978537 | 1 | 1 | 1.05E−37 | 200 |
| rs9886215 | 3 | 115978887 | 0.290501 | 1 | 5.25E−11 | 201 |
| rs9886219 | 4 | 115979104 | 0.283789 | 1 | 7.44E−11 | 202 |
| rs2109516 | 3 | 115979305 | 0.259675 | 0.912736 | 1.05E−07 | 203 |
| rs3757732 | 1 | 115980941 | 0.445619 | 0.943658 | 5.07E−13 | 204 |
| rs3757733 | 1 | 115980965 | 0.495249 | 1 | 1.61E−17 | 205 |
| rs7804372 | 1 | 115981464 | 0.499781 | 1 | 8.27E−18 | 206 |
| rs729949 | 1 | 115982141 | 0.499781 | 1 | 8.27E−18 | 207 |
| rs3807990 | 4 | 115983999 | 0.499781 | 1 | 8.27E−18 | 208 |
| rs3807992 | 1 | 115984481 | 0.499781 | 1 | 8.27E−18 | 209 |
| rs3807994 | 1 | 115984815 | 0.499781 | 1 | 8.27E−18 | 210 |
| rs6466587 | 3 | 115985237 | 0.283789 | 1 | 7.44E−11 | 211 |
| rs6466588 | 4 | 115985326 | 0.499781 | 1 | 8.27E−18 | 212 |
| rs1049314 | 1 | 115986931 | 0.283789 | 1 | 9.15E−11 | 213 |
| rs8713 | 2 | 115987033 | 0.283789 | 1 | 7.44E−11 | 214 |
| rs6867 | 1 | 115987759 | 0.283789 | 1 | 7.44E−11 | 215 |
| rs1049337 | 2 | 115987823 | 0.263682 | 1 | 6.03E−11 | 216 |
| rs6961215 | 4 | 115989766 | 0.283789 | 1 | 7.44E−11 | 217 |
| rs6961388 | 3 | 115989973 | 0.283789 | 1 | 9.15E−11 | 218 |
| rs10280730 | 4 | 115990409 | 0.283789 | 1 | 7.44E−11 | 219 |
| rs10232369 | 1 | 115990559 | 0.283789 | 1 | 7.44E−11 | 220 |
| rs6959106 | 2 | 115991294 | 0.237811 | 1 | 2.32E−09 | 221 |
| rs7802124 | 2 | 115993150 | 0.237811 | 1 | 2.32E−09 | 222 |
| rs7802438 | 1 | 115993436 | 0.237811 | 1 | 2.32E−09 | 223 |
| rs1860588 | 2 | 115993722 | 0.223085 | 1 | 7.03E−09 | 224 |
| rs2052106 | 1 | 115993901 | 0.585826 | 1 | 1.27E−20 | 225 |
| rs11979486 | 3 | 115994130 | 0.216124 | 1 | 2.15E−08 | 226 |
| rs10273326 | 2 | 115996769 | 0.237811 | 1 | 2.32E−09 | 227 |
| rs6466589 | 3 | 115999209 | 0.334457 | 0.932019 | 7.80E−10 | 228 |
| rs7795356 | 4 | 116004265 | 0.299863 | 0.926059 | 6.90E−09 | 229 |
| rs2109517 | 3 | 116004893 | 0.503765 | 0.947548 | 2.52E−14 | 230 |
| rs2056865 | 3 | 116007768 | 0.521994 | 0.908758 | 1.70E−15 | 231 |
| rs2191503 | 4 | 116009428 | 0.299863 | 0.926059 | 6.90E−09 | 232 |
| rs4727835 | 1 | 116011385 | 0.268267 | 0.915579 | 3.98E−07 | 233 |
| rs7800573 | 3 | 116011874 | 0.305266 | 0.870485 | 8.55E−09 | 234 |
| rs6955302 | 4 | 116012940 | 0.430781 | 0.882852 | 4.22E−11 | 235 |
| rs6978354 | 3 | 116013658 | 0.293672 | 0.736242 | 2.13E−08 | 236 |

The table includes, for each SNP, the correlating allele with allele A of rs3807989, position in NCBI build 36, and r², D′ and P-value for the test of correlation between the two markers.

TABLE 10
List of surrogate markers to rs1733724 on Chromosome
10, with R² >0.2 in the HapMap CEU dataset.

| Marker | Correlated | Pos in NCBI | | | | Seq ID |
|---|---|---|---|---|---|---|
| Name | Allele | Build 36 | R² | D′ | P-value | NO: |
| rs1149782 | 4 | 53808502 | 0.214229 | 0.790176 | 4.09E−07 | 237 |
| rs1149781 | 4 | 53809582 | 0.273357 | 0.807508 | 2.06E−08 | 238 |
| rs1194673 | 2 | 53811658 | 0.234785 | 0.614501 | 6.96E−07 | 239 |
| rs1149776 | 4 | 53812481 | 0.218988 | 0.597389 | 2.74E−06 | 240 |
| rs1149775 | 3 | 53812501 | 0.210797 | 0.782266 | 8.10E−07 | 241 |
| rs1149772 | 4 | 53815310 | 0.23734 | 0.615503 | 5.87E−07 | 242 |
| rs1149769 | 3 | 53816103 | 0.239771 | 0.626724 | 1.50E−06 | 243 |
| rs1194671 | 3 | 53817795 | 0.23734 | 0.615503 | 5.87E−07 | 244 |
| rs1194670 | 4 | 53818050 | 0.23388 | 0.611 | 1.11E−06 | 245 |
| rs1194669 | 4 | 53818295 | 0.214229 | 0.790176 | 4.09E−07 | 246 |
| rs1194668 | 2 | 53818632 | 0.224627 | 0.610352 | 1.14E−06 | 247 |
| rs6480837 | 1 | 53819249 | 0.22959 | 0.612413 | 9.83E−07 | 248 |
| rs1209265 | 3 | 53819777 | 0.214229 | 0.790176 | 4.09E−07 | 249 |
| rs1194664 | 3 | 53820821 | 0.23734 | 0.615503 | 5.87E−07 | 250 |
| rs1194663 | 2 | 53821101 | 0.214229 | 0.790176 | 4.09E−07 | 251 |
| rs1660760 | 4 | 53824626 | 0.214229 | 0.790176 | 4.09E−07 | 252 |
| rs12355839 | 2 | 53877702 | 0.290323 | 1 | 1.62E−11 | 253 |
| rs1194743 | 1 | 53882603 | 0.95092 | 1 | 1.37E−26 | 254 |
| rs1733724 | 4 | 53893983 | 1 | 1 | | 255 |

The table includes, for each SNP, the correlating allele with allele T of rs1733724, position in NCBI build 36, and r², D′ and P-value for the test of correlation between the two markers.

TABLE 11
List of surrogate markers to rs3825214 on Chromosome
12, with R² >0.2 in the HapMap CEU dataset.

| Marker | Correlated | Pos in NCBI | | | | Seq ID |
|---|---|---|---|---|---|---|
| Name | Allele | Build 36 | R² | D′ | P-value | NO: |
| rs6489952 | 3 | 113243156 | 0.583962 | 0.923097 | 3.70E−13 | 256 |
| rs1895593 | 2 | 113245198 | 0.786096 | 1 | 3.86E−19 | 257 |
| rs7966567 | 2 | 113245863 | 0.786096 | 1 | 2.78E−19 | 258 |
| rs8181608 | 1 | 113245966 | 0.786096 | 1 | 2.78E−19 | 259 |
| rs10744818 | 4 | 113246068 | 0.786096 | 1 | 2.78E−19 | 260 |
| rs8181683 | 2 | 113246101 | 0.786096 | 1 | 2.78E−19 | 261 |
| rs8181627 | 4 | 113246147 | 0.786096 | 1 | 2.78E−19 | 262 |
| rs10744819 | 1 | 113246213 | 0.786096 | 1 | 3.86E−19 | 263 |
| rs6489953 | 2 | 113249145 | 0.837984 | 1 | 9.19E−21 | 264 |
| rs10744820 | 1 | 113252510 | 0.786096 | 1 | 3.28E−19 | 265 |
| rs1895587 | 1 | 113253912 | 0.786096 | 1 | 2.78E−19 | 266 |
| rs9669457 | 1 | 113260665 | 0.449812 | 0.762707 | 5.75E−11 | 267 |
| rs6489955 | 3 | 113269662 | 0.786096 | 1 | 2.78E−19 | 268 |
| rs7309910 | 2 | 113270637 | 0.786096 | 1 | 2.78E−19 | 269 |
| rs7308120 | 4 | 113273429 | 0.78955 | 0.888566 | 7.45E−19 | 270 |
| rs2384409 | 1 | 113273861 | 0.78955 | 0.888566 | 7.45E−19 | 271 |
| rs2891503 | 1 | 113274193 | 0.653082 | 0.830546 | 1.54E−15 | 272 |
| rs7977083 | 1 | 113274883 | 0.705085 | 0.88612 | 4.59E−17 | 273 |
| rs1895597 | 1 | 113275267 | 0.897959 | 1 | 1.14E−23 | 274 |
| rs7316919 | 1 | 113275838 | 0.745481 | 0.887356 | 6.79E−18 | 275 |
| rs6489956 | 4 | 113276619 | 0.789135 | 0.888333 | 3.02E−18 | 276 |
| rs883079 | 3 | 113277623 | 0.673469 | 1 | 4.72E−19 | 277 |
| rs2113433 | 1 | 113278440 | 0.78955 | 0.888566 | 7.45E−19 | 278 |
| rs3825214 | 3 | 113279826 | 1 | 1 | | 279 |
| rs12367410 | 4 | 113281071 | 1 | 1 | 2.10E−24 | 280 |
| rs10507248 | 3 | 113281476 | 0.737609 | 1 | 2.64E−20 | 281 |
| rs7955405 | 1 | 113281689 | 0.729997 | 1 | 5.76E−19 | 282 |
| rs10744823 | 2 | 113282465 | 1 | 1 | 1.10E−26 | 283 |
| rs7312625 | 3 | 113284357 | 0.737609 | 1 | 2.64E−20 | 284 |
| rs4767237 | 1 | 113285196 | 0.678528 | 0.938483 | 3.21E−16 | 285 |
| rs7135659 | 3 | 113286155 | 0.619607 | 0.87392 | 1.02E−13 | 286 |
| rs1895585 | 4 | 113286521 | 0.633616 | 0.883563 | 1.16E−15 | 287 |
| rs1946295 | 4 | 113286744 | 0.633616 | 0.883563 | 1.16E−15 | 288 |
| rs1946293 | 2 | 113287143 | 0.682713 | 0.938566 | 2.20E−16 | 289 |
| rs3825215 | 3 | 113289281 | 0.624683 | 0.877949 | 7.18E−15 | 290 |
| rs1895582 | 2 | 113291418 | 0.633616 | 0.883563 | 1.16E−15 | 291 |
| rs7964303 | 4 | 113298669 | 0.631578 | 0.883482 | 1.40E−15 | 292 |
| rs17731569 | 3 | 113312090 | 0.384817 | 0.644375 | 4.22E−09 | 293 |

The table includes, for each SNP, the correlating allele with allele G of rs3825214, position in NCBI build 36, and r², D′ and P-value for the test of correlation between the two markers.

TABLE 12
List of surrogate markers to rs365990 on Chromosome
14, with R² >0.2 in the HapMap CEU dataset.

| Marker | Correlated | Pos in NCBI | | | | Seq ID |
|---|---|---|---|---|---|---|
| Name | Allele | Build 36 | R2 | D′ | P-value | NO: |
| rs3811178 | 4 | 22915084 | 0.265259 | 0.576202 | 6.24E−07 | 294 |
| rs8022522 | 1 | 22927191 | 0.356 | 0.702714 | 1.30E−09 | 295 |
| rs365990 | 3 | 22931651 | 1 | 1 | | 296 |
| rs445754 | 4 | 22933642 | 0.6 | 1 | 6.92E−19 | 297 |
| rs10149522 | 2 | 22934361 | 0.201835 | 1 | 2.67E−07 | 298 |
| rs452036 | 1 | 22935725 | 0.962963 | 1 | 1.45E−32 | 299 |
| rs412768 | 2 | 22936553 | 0.823529 | 1 | 9.01E−27 | 300 |
| rs439735 | 1 | 22938125 | 0.572447 | 0.947708 | 1.00E−16 | 301 |
| rs388914 | 4 | 22942932 | 0.572447 | 0.947708 | 1.00E−16 | 302 |
| rs440466 | 2 | 22943957 | 0.853694 | 0.924846 | 8.41E−26 | 303 |
| rs2277474 | 4 | 22944363 | 0.530018 | 0.897793 | 5.13E−15 | 304 |
| rs7143356 | 2 | 22950923 | 0.718976 | 0.849564 | 4.79E−21 | 305 |
| rs12147570 | 4 | 22951760 | 0.340672 | 0.922866 | 3.61E−10 | 306 |
| rs2284651 | 3 | 22951984 | 0.718588 | 0.849337 | 9.60E−21 | 307 |
| rs7149517 | 3 | 22952026 | 0.720551 | 0.849665 | 3.35E−21 | 308 |
| rs2331979 | 3 | 22952695 | 0.720551 | 0.849665 | 3.35E−21 | 309 |
| rs3729833 | 1 | 22953024 | 0.340234 | 0.921912 | 4.29E−10 | 310 |
| rs765021 | 1 | 22953439 | 0.722119 | 0.849776 | 1.55E−21 | 311 |
| rs7140721 | 1 | 22955534 | 0.722119 | 0.849776 | 1.55E−21 | 312 |
| rs3729829 | 4 | 22955727 | 0.722119 | 0.849776 | 1.55E−21 | 313 |
| rs3729828 | 4 | 22955837 | 0.716937 | 0.849216 | 2.08E−20 | 314 |
| rs3729825 | 1 | 22956104 | 0.340672 | 0.922866 | 3.61E−10 | 315 |
| rs7159367 | 2 | 22957485 | 0.722119 | 0.849776 | 1.55E−21 | 316 |
| rs12894524 | 4 | 22957880 | 0.722119 | 0.849776 | 1.55E−21 | 317 |
| rs2277475 | 4 | 22958505 | 0.722119 | 0.849776 | 1.55E−21 | 318 |
| rs12147533 | 3 | 22960531 | 0.340672 | 0.922866 | 3.61E−10 | 319 |
| rs743567 | 2 | 22960822 | 0.688975 | 0.845857 | 2.57E−20 | 320 |
| rs7157716 | 3 | 22962728 | 0.588928 | 0.826317 | 3.05E−16 | 321 |
| rs2754163 | 2 | 22967347 | 0.594419 | 0.83276 | 4.20E−17 | 322 |

The table includes, for each SNP, the correlating allele with allele G of rs365990, position in NCBI build 36, and r², D′ and P-value for the test of correlation between the two markers.

### REFERENCES

1. Palatini, P. & Julius, S. Elevated heart rate: a major risk factor for cardiovascular disease. Clin Exp Hypertens 26, 637-44 (2004).
2. Bjornsson, S., Thorgeirsson, G. et al. Samband hjartslattartidni, heilsufarsthatta, reyking a og danarmeina. Laeknabladid, 21-7 (1993).
3. Jouven, X. et al. Heart-rate profile during exercise as a predictor of sudden death. N Engl J Med 352, 1951-8 (2005).
4. Saksena, S., Camm J. A. Electrophysiological Disorders of the Heart, (Elsevier Churchill Livingstone, Philadelphia, 2004).
5. Cheng, S. et al. Long-term outcomes in individuals with prolonged PR interval or first-degree atrioventricular block. Jama 301, 2571-7 (2009).
6. Hesse, B., Diaz, L. A., Snader, C. E., Blackstone, E. H. & Lauer, M. S. Complete bundle branch block as an independent predictor of all-cause mortality: report of 7,073 patients referred for nuclear exercise testing. Am J Med 110, 253-9 (2001).
7. Desai, A. D. et al. Prognostic Significance of Quantitative QRS Duration. Am J Med 119, 600-6 (2006).
8. Newton-Cheh, C. et al. Genome-wide association study of electrocardiographic and heart rate variability traits: the Framingham Heart Study. BMC Med Genet. 8 Suppl 1, S7 (2007).
9. Hanson, B. et al. Genetic factors in the electrocardiogram and heart rate of twins reared apart and together. Am J Cardiol 63, 606-9 (1989).
10. Havlik, R. J., Garrison, R. J., Fabsitz, R. & Feinleib, M. Variability of heart rate, P-R, QRS and Q-T durations in twins. J Electrocardiol 13, 45-8 (1980).
11. Russell, M. W., Law, I., Sholinsky, P. & Fabsitz, R. R. Heritability of ECG measurements in adult male twins. J Electrocardiol 30 Suppl, 64-8 (1998).
12. Li, J. et al. Familial aggregation and heritability of electrocardiographic intervals and heart rate in a rural Chinese population. Ann Noninvasive Electrocardiol 14, 147-52 (2009).
13. Mutikainen, S. et al. Genetic influences on resting electrocardiographic variables in older women: a twin study. Ann Noninvasive Electrocardiol 14, 57-64 (2009).
14. Arking, D. E. et al. A common genetic variant in the NOS1 regulator NOS1AP modulates cardiac repolarization. Nat Genet. 38, 644-51 (2006).
15. Kao, W. H. et al. Genetic variations in nitric oxide synthase 1 adaptor protein are associated with sudden cardiac death in US white community-based populations. Circulation 119, 940-51 (2009).
16. Newton-Cheh, C. et al. Common variants at ten loci influence QT interval duration in the QTGEN Study. Nat Genet. 41, 399-406 (2009).
17. Pfeufer, A. et al. Common variants at ten loci modulate the QT interval duration in the QTSCD Study. Nat. Genet. 41, 407-14 (2009).
18. Cho, Y. S. et al. A large-scale genome-wide association study of Asian populations uncovers genetic factors influencing eight quantitative traits. Nat Genet. 41, 527-34 (2009).
19. Smith, J. G. et al. Genome-wide association study of electrocardiographic conduction measures in an isolated founder population: Kosrae. Heart Rhythm 6, 634-41 (2009).
20. Li, Q. Y. et al. Holt-Oram syndrome is caused by mutations in TBX5, a member of the Brachyury (T) gene family. Nat Genet. 15, 21-9 (1997).
21. Basson, C. T. et al. Mutations in human TBX5 [corrected] cause limb and cardiac malformation in Holt-Oram syndrome. Nat Genet. 15, 30-5 (1997).
22. Basson, C. T. et al. The clinical and genetic spectrum of the Holt-Oram syndrome (heart-hand syndrome). N Engl J Med 330, 885-91 (1994).
23. Moskowitz, I. P. et al. The T-Box transcription factor TbxS is required for the patterning and maturation of the murine cardiac conduction system. Development 131, 4107-16 (2004).
24. Bruneau, B. G. et al. A murine model of Holt-Oram syndrome defines roles of the T-box transcription factor Tbx5 in cardiogenesis and disease. Cell 106, 709-21 (2001).
25. Ghosh, T. K. et al. Physical interaction between TBX5 and MEF2C is required for early heart development. Mol Cell Biol 29, 2205-18 (2009).
26. Levy, D. et al. Genome-wide association study of blood pressure and hypertension. Nat Genet (2009).
27. Rabert, D. K. et al. A tetrodotoxin-resistant voltage-gated sodium channel from human dorsal root ganglia, hPN3/SCN10A. Pain 78, 107-14 (1998).
28. Sangameswaran, L. et al. Structure and function of a novel voltage-gated, tetrodotoxin-resistant sodium channel specific to sensory neurons. J Biol Chem 271, 5953-6 (1996).
29. Ruan, Y., Liu, N. & Priori, S. G. Sodium channel mutations and arrhythmias. Nat Rev Cardiol 6, 337-48 (2009).
30. Katoh, M. & Katoh, M. Identification and characterization of ARHGAP24 and ARHGAP25 genes in silico. Int J Mol Med 14, 333-8 (2004).
31. Moon, S. Y. & Zheng, Y. Rho GTPase-activating proteins in cell regulation. Trends Cell Biol 13, 13-22 (2003).
32. Kandpal, R. P. Rho GTPase activating proteins in cancer phenotypes. Curr Protein Pept Sci 7, 355-65 (2006).
33. Su, Z. J. et al. A vascular cell-restricted RhoGAP, p73RhoGAP, is a key regulator of angiogenesis. Proc Natl Acad Sci USA 101, 12212-7 (2004).
34. Engelman, J. A. et al. Molecular genetics of the caveolin gene family: implications for human cancers, diabetes, Alzheimer disease, and muscular dystrophy. Am J Hum Genet. 63, 1578-87 (1998).
35. Kurzchalia, T. V. & Parton, R. G. Membrane microdomains and caveolae. Curr Opin Cell Biol 11, 424-31 (1999).
36. Razani, B. et al. Caveolin-1 null mice are viable but show evidence of hyperproliferative and vascular abnormalities. J Biol Chem 276, 38121-38 (2001).
37. Drab, M. et al. Loss of caveolae, vascular dysfunction, and pulmonary defects in caveolin-1 gene-disrupted mice. Science 293, 2449-52 (2001).
38. Zhao, Y. Y. et al. Defects in caveolin-1 cause dilated cardiomyopathy and pulmonary hypertension in knockout mice. Proc Natl Acad Sci USA 99, 11375-80 (2002).
39. Zakut, R. & Givol, D. The tumor suppression function of p21Waf is contained in its N-terminal half (‘half-WAF’). Oncogene 11, 393-5 (1995).
40. Kurabayashi, M., Tsuchimochi, H., Komuro, I., Takaku, F. & Yazaki, Y. Molecular cloning and characterization of human cardiac alpha- and beta-form myosin heavy chain complementary DNA clones. Regulation of expression during development and pressure overload in human atrium. J Clin Invest 82, 524-31 (1988).
41. Mandavi, V., Chambers, A. P. & Nadal-Ginard, B. Cardiac alpha- and beta-myosin heavy chain genes are organized in tandem. Proc Natl Acad Sci USA 81, 2626-30 (1984).
42. Franco, D., Lamers, W. H. & Moorman, A. F. Patterns of expression in the developing myocardium: towards a morphologically integrated transcriptional model. Cardiovasc Res 38, 25-53 (1998).
43. Lompre, A. M., Nadal-Ginard, B. & Mandavi, V. Expression of the cardiac ventricular alpha- and beta-myosin heavy chain genes is developmentally and hormonally regulated. J Biol Chem 259, 6437-46 (1984).
44. Litten, R. Z., 3rd, Martin, B. J., Low, R. B. & Alpert, N. R. Altered myosin isozyme patterns from pressure-overloaded and thyrotoxic hypertrophied rabbit hearts. Circ Res 50, 856-64 (1982).
45. Harris, D. E., Work, S. S., Wright, R. K., Alpert, N. R. & Warshaw, D. M. Smooth, cardiac and skeletal muscle myosin force and motion generation assessed by cross-bridge mechanical interactions in vitro. J Muscle Res Cell Motil 15, 11-9 (1994).
46. Herron, T. J., Korte, F. S. & McDonald, K. S. Loaded shortening and power output in cardiac myocytes are dependent on myosin heavy chain isoform expression. Am J Physiol Heart Circ Physiol 281, H1217-22 (2001).
47. Herron, T. J. & McDonald, K. S. Small amounts of alpha-myosin heavy chain isoform expression significantly increase power output of rat cardiac myocyte fragments. Circ Res 90, 1150-2 (2002).
48. Carniel, E. et al. Alpha-myosin heavy chain: a sarcomeric gene associated with dilated and hypertrophic phenotypes of cardiomyopathy. Circulation 112, 54-9 (2005).
49. Ching, Y. H. et al. Mutation in myosin heavy chain 6 causes atrial septal defect. Nat Genet. 37, 423-8 (2005).
50. A haplotype map of the human genome. Nature 437, 1299-320 (2005).
51. Kong, A. et al. Detection of sharing by descent, long-range phasing and haplotype imputation. Nat Genet (2008).
52. Maier, S. K. et al. Distinct subcellular localization of different sodium channel alpha and beta subunits in single ventricular myocytes from mouse heart. Circulation 109, 1421-7 (2004).
53. Brodsky, M., Wu, D., Denes, P. & Rosen, K. M. Familial atrial tachyarrhythmia with short PR interval. Arch Intern Med 137, 165-9 (1977).
54. Lown, B., Ganong, W. F. & Levine, S. A. The syndrome of short P-R interval, normal QRS complex and paroxysmal rapid heart action. Circulation 5, 693-706 (1952).
55. Postma, A. V. et al. A gain-of-function TBX5 mutation is associated with atypical Holt-Oram syndrome and paroxysmal atrial fibrillation. Circ Res 102, 1433-42 (2008).
56. Thorgeirsson, T. E. et al. A variant associated with nicotine dependence, lung cancer and peripheral arterial disease. Nature 452, 638-42 (2008).
57. Gudbjartsson, D. F. et al. Sequence variants affecting eosinophil numbers associate with asthma and myocardial infarction. Nat Genet. 41, 342-7 (2009).
58. Willer, C. J. et al. Newly identified loci that influence lipid concentrations and risk of coronary artery disease. Nat Genet. 40, 161-9 (2008).
59. Gouas, L. et al. Association of KCNQ1, KCNE1, KCNH2 and SCN5A polymorphisms with QTc interval length in a healthy population. Eur J Hum Genet. 13, 1213-22 (2005).
60. Newton-Cheh, C. et al. Common genetic variation in KCNH2 is associated with QT interval duration: the Framingham Heart Study. Circulation 116, 1128-36 (2007).
61. Pfeufer, A. et al. Common variants in myocardial ion channel genes modify the QT interval in the general population: results from the KORA study. Circ Res 96, 693-701 (2005).
62. Friedlander, Y. et al. Possible association of the human KCNE1 (minK) gene and QT interval in healthy subjects: evidence from association and linkage analyses in Israeli families. Ann Hum Genet. 69, 645-56 (2005).
63. Akyol, M. et al. The common non-synonymous variant G38S of the KCNE1-(minK)-gene is not associated to QT interval in Central European Caucasians: results from the KORA study. Eur Heart J 28, 305-9 (2007).
64. Zhou, S. H., Helfenbein, E. D., Lindauer, J. M., Gregg, R. E. & Feild, D. Q. Philips QT interval measurement algorithms for diagnostic, ambulatory, and patient monitoring ECG applications. Ann Noninvasive Electrocardiol 14 Suppl 1, S3-8 (2009).
65. James Lindauer, R. G., Eric Helfenbein, Min Shao, Sophia Zhou. Global QT measurements in the Philips 12-lead algorithm. Journal of Electrocardiology, 90 (2005).
66. Devlin, B., Bacanu, S. A. & Roeder, K. Genomic Control to the extreme. Nat Genet. 36, 1129-30; author reply 1131 (2004).
67. Marchini, J., Howie, B., Myers, S., McVean, G. & Donnelly, P. A new multipoint method for genome-wide association studies by imputation of genotypes. Nat Genet. 39, 906-13 (2007).
68. Gretarsdottir, S. et al. The gene encoding phosphodiesterase 4D confers risk of ischemic stroke. Nat Genet. 35, 131-8 (2003).

### Example 2

Association of further markers in regions identified as harboring variants associated with ECG measures and related phenotypes was performed.

Data for SNPs was imputed based on whole genome sequencing of 84 Icelanders using the IMPUTE model (Marchini, 3., Howie, B., Myers, S., McVean, G. & Donnelly, P. A new multipoint method for genome-wide association studies by imputation of genotypes. Nat Genet. 39, 906-13 (2007)), using long range phasing (Kong, A., et al. Nat Genet. 40:1068-75 (2008)). Quantitative traits were regressed on expected allele counts using classical linear regression. Disease phenotypes were analyzed using logistic regression where affection status was treated as the response variable and the expected genotype as a explanatory variable.

The results below show that large number of variants in the identified regions are indeed associating with ECG phenotypes, atrial fibrillation and pacemaker placement. This is to be expected, due to the extensive LD in the respective regions.

TABLE 13
Association results for QRS interval in a 2 Mb
region flanking rs3825214 on chromosome 12.

| | | | | | effect | other | SEQ ID |
|---|---|---|---|---|---|---|---|
| marker | p-value | effect | freq | position | allele | allele | NO: |
| rs1895597 | 1.96E−08 | −0.073865 | 0.769214 | 113,275,267 | A | T | 274 |
| rs2891503 | 2.31E−08 | 0.074026 | 0.238778 | 113,274,193 | A | G | 272 |
| rs3825214 | 2.78E−08 | −0.073117 | 0.781334 | 113,279,826 | A | G | 279 |
| rs10744823 | 2.80E−08 | 0.074731 | 0.216396 | 113,282,465 | C | T | 283 |
| rs12367410 | 2.85E−08 | −0.073818 | 0.783762 | 113,281,071 | C | T | 280 |
| rs2113433 | 3.95E−08 | −0.072419 | 0.777774 | 113,278,440 | G | T | 278 |
| rs6489956 | 4.01E−08 | −0.07261 | 0.778493 | 113,276,619 | C | T | 276 |
| rs7316919 | 4.05E−08 | 0.072705 | 0.221127 | 113,275,838 | A | C | 275 |
| rs2384409 | 4.31E−08 | 0.073008 | 0.219567 | 113,273,861 | A | G | 271 |
| rs2384408 | 4.64E−08 | 0.073329 | 0.215815 | 113,273,733 | A | G | 3353 |
| rs7308120 | 4.69E−08 | −0.073366 | 0.784397 | 113,273,429 | C | T | 270 |
| rs7977083 | 1.24E−07 | 0.067698 | 0.25662 | 113,274,883 | A | G | 273 |
| rs1862909 | 1.51E−07 | 0.072489 | 0.200957 | 113,271,488 | A | G | 3354 |
| rs7309910 | 1.68E−07 | 0.072367 | 0.200136 | 113,270,637 | C | G | 269 |
| rs6489955 | 2.11E−07 | −0.071974 | 0.801216 | 113,269,662 | A | G | 268 |
| rs10744822 | 2.14E−07 | 0.072105 | 0.198608 | 113,268,423 | C | T | 3355 |
| rs10744824 | 2.64E−07 | −0.070628 | 0.799931 | 113,293,021 | A | G | 3356 |
| rs5015007 | 3.48E−07 | −0.070731 | 0.804004 | 113,289,466 | A | T | 3357 |
| rs7965033 | 5.06E−07 | −0.070557 | 0.808474 | 113,295,738 | C | T | 3358 |
| rs933748 | 1.39E−06 | 0.066658 | 0.205406 | 113,245,039 | C | T | 3359 |
| rs6489953 | 1.84E−06 | 0.066445 | 0.188781 | 113,249,145 | C | T | 264 |
| rs10774752 | 1.91E−06 | 0.066336 | 0.188509 | 113,252,695 | A | G | 3360 |
| rs7964836 | 1.93E−06 | −0.066304 | 0.811491 | 113,251,103 | C | G | 3361 |
| rs7307520 | 1.98E−06 | 0.066238 | 0.188515 | 113,249,082 | A | G | 3362 |
| rs11067054 | 2.58E−06 | 0.071644 | 0.16855 | 113,256,940 | A | G | 3363 |
| rs883079 | 2.71E−06 | 0.055535 | 0.304506 | 113,277,623 | C | T | 277 |
| rs2384407 | 2.92E−06 | −0.055678 | 0.697398 | 113,273,609 | A | G | 3364 |
| rs8181608 | 3.16E−06 | 0.065924 | 0.183954 | 113,245,966 | A | G | 259 |
| rs6489952 | 3.51E−06 | −0.065944 | 0.81572 | 113,243,156 | A | G | 256 |
| rs10744820 | 3.74E−06 | 0.065004 | 0.184141 | 113,252,510 | A | G | 265 |
| rs1895593 | 3.95E−06 | −0.064969 | 0.81561 | 113,245,198 | A | G | 257 |
| rs1895587 | 3.96E−06 | −0.06486 | 0.815854 | 113,253,912 | C | T | 266 |
| rs10744819 | 4.02E−06 | 0.064795 | 0.184162 | 113,246,213 | A | G | 263 |
| rs8181627 | 4.02E−06 | −0.064792 | 0.815838 | 113,246,147 | C | T | 262 |
| rs8181683 | 4.03E−06 | 0.06479 | 0.184162 | 113,246,101 | C | T | 261 |
| rs10744818 | 4.03E−06 | −0.064789 | 0.815838 | 113,246,068 | C | T | 260 |
| rs7966567 | 4.03E−06 | 0.064789 | 0.184183 | 113,245,863 | C | T | 258 |
| rs7964303 | 4.98E−06 | −0.057261 | 0.708289 | 113,298,669 | G | T | 292 |
| rs10507248 | 5.79E−06 | 0.054386 | 0.292588 | 113,281,476 | G | T | 281 |
| rs7955405 | 5.96E−06 | 0.054366 | 0.292166 | 113,281,689 | A | G | 282 |
| chr12: 113277199 | 7.03E−06 | −0.532326 | 0.991346 | 113,277,199 | C | T | 3365 |
| rs2016047 | 8.56E−06 | −0.067207 | 0.777817 | 113,244,799 | G | T | 3366 |
| rs1946295 | 1.01E−05 | 0.05324 | 0.28992 | 113,286,744 | T | C | 288 |
| rs9669457 | 1.20E−05 | 0.05684 | 0.252844 | 113,260,665 | A | G | 267 |
| rs7135659 | 1.43E−05 | −0.052453 | 0.716493 | 113,286,155 | A | G | 286 |
| rs4767239 | 1.53E−05 | −0.061188 | 0.798894 | 113,300,931 | C | G | 3367 |
| rs1946293 | 1.65E−05 | −0.052049 | 0.717743 | 113,287,143 | A | G | 289 |
| rs7312625 | 2.08E−05 | −0.051807 | 0.718133 | 113,284,357 | A | G | 284 |
| rs1920591 | 2.35E−05 | −0.053467 | 0.345134 | 113,190,592 | A | G | 3368 |
| rs4767237 | 2.48E−05 | 0.051329 | 0.280263 | 113,285,196 | A | G | 285 |
| rs2016045 | 2.56E−05 | −0.063886 | 0.783388 | 113,244,750 | A | T | 3369 |
| rs3825215 | 3.08E−05 | −0.051016 | 0.720456 | 113,289,281 | C | G | 290 |
| rs1895583 | 3.37E−05 | 0.05089 | 0.275231 | 113,291,268 | A | G | 3370 |
| rs1895582 | 3.37E−05 | −0.050742 | 0.723842 | 113,291,418 | A | G | 291 |
| rs1895585 | 3.39E−05 | 0.050387 | 0.277734 | 113,286,521 | A | G | 287 |
| rs7961277 | 3.57E−05 | −0.047989 | 0.54415 | 113,298,462 | C | T | 3371 |
| chr12: 113294500 | 4.27E−05 | 0.059077 | 0.682865 | 113,294,500 | C | T | 3372 |
| rs1920596 | 5.40E−05 | −0.044024 | 0.497044 | 113,188,795 | A | T | 3373 |
| rs2701106 | 5.76E−05 | −0.044274 | 0.512179 | 113,181,930 | A | G | 3374 |
| rs1920597 | 5.82E−05 | 0.043909 | 0.501774 | 113,188,475 | A | G | 3375 |
| chr12: 113329460 | 6.08E−05 | −0.184802 | 0.975672 | 113,329,460 | G | T | 3376 |
| rs2555030 | 7.21E−05 | 0.057616 | 0.186758 | 113,301,715 | A | G | 3377 |
| chr12: 113202956 | 9.70E−05 | −0.156959 | 0.956652 | 113,202,956 | A | G | 3378 |
| rs10850391 | 1.31E−04 | −0.042162 | 0.563302 | 113,814,252 | C | T | 3379 |
| chr12: 113202737 | 1.39E−04 | 0.163653 | 0.038218 | 113,202,737 | A | G | 3380 |
| rs73201471 | 1.42E−04 | 0.061061 | 0.584984 | 113,256,837 | C | T | 3381 |
| rs10850315 | 1.50E−04 | 0.046007 | 0.2758 | 113,251,118 | G | T | 3382 |
| chr12: 113180871 | 1.70E−04 | 0.066415 | 0.184086 | 113,180,871 | A | T | 3383 |
| rs57155932 | 1.78E−04 | −0.051836 | 0.74495 | 113,211,470 | A | G | 3384 |
| rs10744815 | 2.85E−04 | −0.040812 | 0.423341 | 113,197,765 | A | C | 3385 |
| chr12: 113200986 | 3.75E−04 | 0.102708 | 0.067363 | 113,200,986 | A | G | 3386 |
| rs16943956 | 4.17E−04 | −0.100546 | 0.93096 | 113,195,145 | A | G | 3387 |
| rs10850302 | 4.27E−04 | 0.039772 | 0.603081 | 113,196,478 | C | T | 3388 |
| chr12: 113180727 | 4.51E−04 | −0.10063 | 0.928886 | 113,180,727 | C | T | 3389 |
| rs1920593 | 4.72E−04 | −0.046749 | 0.790005 | 113,189,926 | C | T | 3390 |
| chr12: 112810144 | 4.81E−04 | −0.104583 | 0.962169 | 112,810,144 | A | G | 3391 |
| rs1920595 | 4.84E−04 | −0.046674 | 0.789669 | 113,189,705 | A | C | 3392 |
| rs12813238 | 4.97E−04 | 0.03927 | 0.602051 | 113,206,207 | C | T | 3393 |
| chr12: 113362401 | 5.02E−04 | −0.129026 | 0.954173 | 113,362,401 | A | G | 3394 |
| rs10774750 | 5.32E−04 | 0.039176 | 0.582247 | 113,231,916 | A | C | 3395 |
| rs7303255 | 5.34E−04 | 0.038774 | 0.585161 | 113,203,974 | C | T | 3396 |
| rs1247933 | 5.64E−04 | 0.065619 | 0.19241 | 113,176,419 | A | G | 3397 |
| rs2162320 | 5.65E−04 | −0.049598 | 0.806317 | 113,191,172 | C | T | 3398 |
| rs17660551 | 6.00E−04 | −0.045934 | 0.787133 | 113,188,005 | A | C | 3399 |
| rs16943344 | 6.04E−04 | −0.100221 | 0.960292 | 112,804,599 | C | T | 3400 |
| rs17660485 | 6.14E−04 | −0.04585 | 0.786898 | 113,187,497 | C | G | 3401 |
| rs7138015 | 6.46E−04 | 0.045648 | 0.213642 | 113,186,261 | A | C | 3402 |
| chr12: 112823960 | 6.53E−04 | −0.106002 | 0.964422 | 112,823,960 | C | T | 3403 |
| rs58562176 | 6.80E−04 | −0.045439 | 0.785834 | 113,185,837 | C | T | 3404 |
| rs17660176 | 6.91E−04 | 0.070772 | 0.154323 | 113,177,009 | A | G | 3405 |
| rs6489926 | 7.09E−04 | 0.106083 | 0.035129 | 112,825,678 | C | T | 3406 |
| rs17660241 | 7.41E−04 | 0.045344 | 0.214841 | 113,179,284 | A | G | 3407 |
| chr12: 112790402 | 7.50E−04 | −0.096214 | 0.959028 | 112,790,402 | A | G | 3408 |
| rs3782445 | 7.56E−04 | −0.095967 | 0.958974 | 112,797,276 | C | T | 3409 |
| chr12: 112798862 | 7.62E−04 | −0.095924 | 0.958991 | 112,798,862 | C | T | 3410 |
| chr12: 113181795 | 7.77E−04 | −0.044947 | 0.784582 | 113,181,795 | A | G | 3411 |
| rs58872758 | 7.93E−04 | −0.04495 | 0.784238 | 113,180,030 | G | T | 3412 |
| chr12: 112784915 | 8.59E−04 | −0.095865 | 0.959301 | 112,784,915 | C | T | 3413 |
| rs11610607 | 8.65E−04 | 0.037903 | 0.639796 | 113,183,152 | A | G | 3414 |
| rs12426785 | 8.74E−04 | 0.037839 | 0.639772 | 113,185,981 | C | T | 3415 |
| rs11608388 | 8.81E−04 | −0.037792 | 0.360189 | 113,188,057 | A | C | 3416 |
| rs12425958 | 8.91E−04 | −0.095737 | 0.95937 | 112,784,122 | C | T | 3417 |
| chr12: 113579001 | 9.11E−04 | −0.144237 | 0.983231 | 113,579,001 | A | G | 3418 |
| rs12422921 | 9.71E−04 | −0.095491 | 0.959575 | 112,783,418 | G | T | 3419 |
| rs60029182 | 9.95E−04 | −0.046656 | 0.815531 | 113,227,756 | G | T | 3420 |
| rs57797354 | 1.01E−03 | 0.047947 | 0.171614 | 113,211,534 | C | T | 3421 |
| chr12: 113219987 | 1.01E−03 | 0.046316 | 0.192844 | 113,219,987 | A | C | 3422 |
| chr12: 13732445 | 1.04E−03 | −0.215436 | 0.013737 | 113,732,445 | A | G | 3423 |
| rs4767236 | 1.05E−03 | 0.046249 | 0.185644 | 113,221,039 | A | G | 3424 |
| rs12372752 | 1.06E−03 | −0.076544 | 0.168241 | 113,406,493 | A | G | 3425 |
| rs1078568 | 1.06E−03 | −0.046144 | 0.814344 | 113,220,504 | A | G | 3426 |
| rs1078567 | 1.07E−03 | −0.046107 | 0.814344 | 113,220,355 | A | G | 3427 |
| rs4259873 | 1.08E−03 | −0.04606 | 0.814508 | 113,219,162 | C | T | 3428 |
| rs4556590 | 1.09E−03 | 0.045974 | 0.185657 | 113,219,302 | C | T | 3429 |
| chr12: 113324458 | 1.10E−03 | −0.136702 | 0.973355 | 113,324,458 | A | C | 3430 |
| rs4540867 | 1.11E−03 | 0.045878 | 0.185657 | 113,218,077 | A | G | 3431 |
| rs753564 | 1.12E−03 | −0.045821 | 0.814343 | 113,217,328 | C | G | 3432 |
| chr12: 113383797 | 1.12E−03 | −0.141594 | 0.982428 | 113,383,797 | C | T | 3433 |
| rs4766729 | 1.15E−03 | 0.045175 | 0.190868 | 113,214,255 | C | G | 3434 |
| rs60302750 | 1.18E−03 | 0.045459 | 0.185243 | 113,209,985 | A | G | 3435 |
| rs1896018 | 1.19E−03 | −0.045544 | 0.815754 | 113,215,780 | A | G | 3436 |
| chr12: 113350212 | 1.20E−03 | 0.130523 | 0.027835 | 113,350,212 | A | C | 3437 |
| chr12: 113227545 | 1.21E−03 | −0.082289 | 0.919637 | 113,227,545 | C | T | 3438 |
| rs1896016 | 1.22E−03 | 0.04539 | 0.184476 | 113,215,558 | C | T | 3439 |
| rs1896015 | 1.22E−03 | 0.045384 | 0.184476 | 113,215,473 | A | T | 3440 |
| rs4261334 | 1.23E−03 | −0.045331 | 0.815524 | 113,214,723 | A | C | 3441 |
| rs4274232 | 1.23E−03 | 0.045323 | 0.184476 | 113,214,598 | C | T | 3442 |
| rs59777856 | 1.24E−03 | −0.045315 | 0.815591 | 113,213,862 | G | T | 3443 |
| chr12: 113213806 | 1.24E−03 | −0.04527 | 0.815523 | 113,213,806 | A | G | 3444 |
| chr12: 113268550 | 1.25E−03 | −0.077174 | 0.123983 | 113,268,550 | A | G | 3445 |
| rs60772998 | 1.25E−03 | 0.045237 | 0.184466 | 113,213,250 | A | G | 3446 |
| chr12: 113212688 | 1.26E−03 | 0.045214 | 0.184456 | 113,212,688 | A | G | 3447 |
| rs60268458 | 1.26E−03 | −0.045204 | 0.815552 | 113,212,437 | A | C | 3448 |
| rs1896014 | 1.28E−03 | 0.045086 | 0.184363 | 113,209,532 | A | G | 3449 |
| rs1896013 | 1.29E−03 | −0.045082 | 0.815639 | 113,209,450 | C | T | 3450 |
| rs1896012 | 1.29E−03 | 0.045081 | 0.18436 | 113,209,430 | C | T | 3451 |
| chr12: 113208133 | 1.30E−03 | −0.045009 | 0.815691 | 113,208,133 | A | T | 3452 |
| rs3782455 | 1.42E−03 | 0.087106 | 0.042204 | 112,843,648 | T | G | 3453 |
| rs16943340 | 1.45E−03 | −0.093704 | 0.960428 | 112,779,485 | C | T | 3454 |
| chr12: 112383295 | 1.45E−03 | −0.079289 | 0.852686 | 112,383,295 | C | T | 3455 |
| chr12: 113574398 | 1.61E−03 | 0.06362 | 0.238678 | 113,574,398 | A | G | 3456 |
| chr12: 113574393 | 1.63E−03 | 0.059167 | 0.282669 | 113,574,393 | A | G | 3457 |
| chr12: 113711753 | 1.69E−03 | 0.075603 | 0.943948 | 113,711,753 | C | T | 3458 |
| chr12: 112607596 | 1.78E−03 | 0.120073 | 0.027682 | 112,607,596 | A | C | 3459 |
| rs1458705 | 1.82E−03 | −0.054797 | 0.896153 | 112,938,219 | A | G | 3460 |
| chr12: 113566963 | 2.04E−03 | 0.122601 | 0.020445 | 113,566,963 | A | G | 3461 |
| rs10507243 | 2.16E−03 | 0.034082 | 0.575798 | 113,193,181 | A | G | 3462 |
| chr12: 112976915 | 2.23E−03 | −0.150199 | 0.976887 | 112,976,915 | C | T | 3463 |
| chr12: 113419930 | 2.34E−03 | 0.059491 | 0.309005 | 113,419,930 | G | T | 3464 |
| rs2052738 | 2.38E−03 | 0.033578 | 0.498154 | 112,537,633 | A | G | 3465 |
| rs7973374 | 2.40E−03 | 0.034208 | 0.609722 | 113,195,065 | C | T | 3466 |
| rs7977369 | 2.49E−03 | −0.0413 | 0.799173 | 113,196,032 | C | T | 3467 |
| chr12: 113205016 | 2.51E−03 | 0.041267 | 0.197894 | 113,205,016 | A | G | 3468 |
| rs2484589 | 2.52E−03 | −0.051541 | 0.145288 | 113,809,649 | A | G | 3469 |
| chr12: 112759378 | 2.52E−03 | 0.104504 | 0.030456 | 112,759,378 | C | G | 3470 |
| rs12579617 | 2.59E−03 | −0.033189 | 0.42287 | 113,210,458 | A | G | 3471 |
| chr12: 113198160 | 2.62E−03 | −0.04108 | 0.799802 | 113,198,160 | C | T | 3472 |
| chr12: 113451244 | 2.62E−03 | 0.12934 | 0.018602 | 113,451,244 | A | G | 3473 |
| rs12825923 | 2.64E−03 | −0.0331 | 0.422471 | 113,208,622 | A | G | 3474 |
| rs4766728 | 2.64E−03 | 0.04102 | 0.199688 | 113,199,628 | C | T | 3475 |
| rs7132243 | 2.64E−03 | 0.041016 | 0.199599 | 113,202,664 | C | T | 3476 |
| chr12: 113198279 | 2.65E−03 | −0.04099 | 0.80049 | 113,198,279 | A | C | 3477 |
| rs7315118 | 2.66E−03 | −0.033068 | 0.422231 | 113,206,867 | A | G | 3478 |
| rs11067028 | 2.66E−03 | 0.03306 | 0.577774 | 113,206,623 | A | G | 3479 |
| rs11608430 | 2.67E−03 | −0.033046 | 0.422225 | 113,205,886 | A | G | 3480 |
| rs10850305 | 2.67E−03 | −0.033046 | 0.422179 | 113,205,530 | C | T | 3481 |
| rs7314743 | 2.67E−03 | 0.033036 | 0.577841 | 113,203,030 | A | G | 3482 |
| rs11067027 | 2.68E−03 | 0.03303 | 0.577859 | 113,202,524 | G | T | 3483 |
| rs7973207 | 2.68E−03 | 0.033027 | 0.577899 | 113,198,429 | C | T | 3484 |
| rs10744816 | 2.68E−03 | −0.033024 | 0.422129 | 113,198,649 | C | T | 3485 |
| rs7314353 | 2.69E−03 | 0.033024 | 0.577922 | 113,202,776 | A | G | 3486 |
| chr12: 113700621 | 2.69E−03 | −0.101089 | 0.941078 | 113,700,621 | A | T | 3487 |
| rs55870641 | 2.78E−03 | 0.040753 | 0.198275 | 113,206,637 | C | T | 3488 |
| chr12: 113204921 | 2.80E−03 | 0.040728 | 0.198265 | 113,204,921 | C | T | 3489 |
| chr12: 113204801 | 2.80E−03 | −0.040728 | 0.801735 | 113,204,801 | G | T | 3490 |
| rs6489949 | 2.80E−03 | −0.040721 | 0.801738 | 113,202,536 | C | T | 3491 |
| rs58412031 | 2.80E−03 | −0.040713 | 0.801741 | 113,199,493 | C | T | 3492 |
| chr12: 112552749 | 2.87E−03 | 0.171391 | 0.014032 | 112,552,749 | A | G | 3493 |
| chr12: 113098529 | 2.87E−03 | 0.152842 | 0.023723 | 113,098,529 | A | G | 3494 |
| chr12: 113464954 | 2.92E−03 | 0.129382 | 0.019134 | 113,464,954 | A | G | 3495 |
| rs28423047 | 2.96E−03 | −0.04471 | 0.203897 | 113,279,211 | C | G | 3496 |
| rs10774783 | 2.98E−03 | −0.036781 | 0.596464 | 113,868,201 | A | G | 3497 |
| chr12: 112317197 | 3.14E−03 | 0.779193 | 0.003019 | 112,317,197 | A | C | 3498 |
| rs1895602 | 3.28E−03 | 0.034432 | 0.518661 | 113,319,811 | A | C | 3499 |
| rs16944547 | 3.32E−03 | 0.112397 | 0.020608 | 113,546,339 | C | T | 3500 |
| chr12: 113463839 | 3.33E−03 | −0.078777 | 0.943949 | 113,463,839 | C | G | 3501 |
| chr12: 113544303 | 3.40E−03 | −0.112012 | 0.979425 | 113,544,303 | G | T | 3502 |
| rs10774743 | 3.41E−03 | 0.032634 | 0.598604 | 113,209,047 | C | T | 3503 |
| rs10850304 | 3.43E−03 | 0.032629 | 0.599518 | 113,205,136 | C | T | 3504 |
| rs11614542 | 3.44E−03 | 0.0326 | 0.599156 | 113,205,721 | A | G | 3505 |
| rs10850303 | 3.46E−03 | 0.032595 | 0.599457 | 113,196,587 | G | T | 3506 |
| rs10850301 | 3.48E−03 | −0.032544 | 0.400884 | 113,196,041 | A | G | 3507 |
| rs7963168 | 3.49E−03 | −0.032537 | 0.400893 | 113,195,449 | C | T | 3508 |
| rs59448837 | 3.55E−03 | −0.111474 | 0.979438 | 113,542,021 | C | T | 3509 |
| rs16934281 | 3.58E−03 | −0.111396 | 0.979439 | 113,541,224 | A | G | 3510 |
| chr12: 113541135 | 3.58E−03 | −0.111388 | 0.97944 | 113,541,135 | C | G | 3511 |
| chr12: 113540897 | 3.59E−03 | 0.111347 | 0.020559 | 113,540,897 | C | G | 3512 |
| rs11067021 | 3.61E−03 | −0.032404 | 0.401154 | 113,194,097 | A | G | 3513 |
| rs7300648 | 3.66E−03 | 0.111107 | 0.020554 | 113,539,640 | A | G | 3514 |
| chr12: 113538985 | 3.68E−03 | 0.111068 | 0.020559 | 113,538,985 | G | T | 3515 |
| rs7297701 | 3.69E−03 | −0.032413 | 0.399684 | 113,202,792 | A | G | 3516 |
| chr12: 113819546 | 3.73E−03 | −0.423187 | 0.993509 | 113,819,546 | C | T | 3517 |
| rs1896010 | 3.74E−03 | −0.031884 | 0.424578 | 113,193,870 | A | G | 3518 |
| rs4261333 | 3.89E−03 | 0.031983 | 0.580364 | 113,214,722 | A | G | 3519 |
| rs11067288 | 3.91E−03 | −0.06834 | 0.057604 | 113,695,399 | C | T | 3520 |
| chr12: 113032676 | 3.93E−03 | 0.258423 | 0.994231 | 113,032,676 | A | C | 3521 |
| rs1895604 | 3.96E−03 | 0.033044 | 0.468775 | 113,318,062 | A | C | 3522 |
| chr12: 113869864 | 3.96E−03 | −0.04193 | 0.232371 | 113,869,864 | A | G | 3523 |
| chr12: 113814566 | 4.04E−03 | 0.140303 | 0.027063 | 113,814,566 | A | G | 3524 |
| chr12: 113466781 | 4.06E−03 | −0.12213 | 0.979558 | 113,466,781 | C | T | 3525 |
| chr12: 113852362 | 4.11E−03 | 0.057013 | 0.213377 | 113,852,362 | C | T | 3526 |
| rs1896004 | 4.13E−03 | −0.031946 | 0.419486 | 113,223,366 | C | G | 3527 |
| rs7298356 | 4.25E−03 | 0.031423 | 0.573747 | 113,192,523 | G | T | 3528 |
| rs1955098 | 4.27E−03 | 0.031735 | 0.580988 | 113,220,426 | A | G | 3529 |
| chr12: 113394225 | 4.57E−03 | −0.096614 | 0.972684 | 113,394,225 | C | T | 3530 |
| chr12: 113381615 | 4.62E−03 | 0.237749 | 0.993363 | 113,381,615 | C | T | 3531 |
| chr12: 113299763 | 4.93E−03 | −0.053551 | 0.696837 | 113,299,763 | A | T | 3532 |
| chr12: 112753344 | 5.19E−03 | −0.099736 | 0.97304 | 112,753,344 | A | C | 3533 |
| rs56940775 | 5.47E−03 | 0.099295 | 0.026953 | 112,752,159 | C | T | 3534 |
| rs11067037 | 5.54E−03 | −0.031465 | 0.398455 | 113,231,340 | C | T | 3535 |
| chr12: 112372775 | 5.60E−03 | 0.069842 | 0.234878 | 112,372,775 | A | C | 3536 |
| rs16934279 | 5.60E−03 | −0.067971 | 0.93889 | 113,378,758 | A | C | 3537 |
| rs725086 | 5.70E−03 | 0.031319 | 0.601982 | 113,230,122 | C | T | 3538 |
| rs7962399 | 5.74E−03 | 0.031255 | 0.602158 | 113,228,631 | A | G | 3539 |
| rs2114326 | 5.75E−03 | −0.031238 | 0.397763 | 113,228,027 | A | C | 3540 |
| chr12: 113325961 | 5.77E−03 | −0.103958 | 0.922456 | 113,325,961 | C | G | 3541 |
| rs17731569 | 5.77E−03 | −0.037684 | 0.792003 | 113,312,090 | A | G | 293 |
| rs4767108 | 5.80E−03 | 0.035836 | 0.278301 | 112,556,484 | G | T | 3542 |
| chr12: 112966339 | 5.81E−03 | 0.049553 | 0.141971 | 112,966,339 | A | G | 3543 |
| rs10850310 | 5.81E−03 | 0.031114 | 0.602596 | 113,221,288 | A | G | 3544 |
| rs1718375 | 5.85E−03 | 0.078002 | 0.960425 | 113,511,503 | A | G | 3545 |
| rs1895606 | 5.86E−03 | −0.030623 | 0.486339 | 113,317,767 | C | T | 3546 |
| chr12: 112751638 | 5.87E−03 | 0.098548 | 0.02653 | 112,751,638 | C | G | 3547 |
| rs3825209 | 5.97E−03 | 0.092773 | 0.027359 | 112,835,440 | C | T | 3548 |
| rs3782454 | 6.00E−03 | −0.092709 | 0.97266 | 112,835,598 | A | G | 3549 |
| chr12: 113489582 | 6.05E−03 | 0.230313 | 0.995232 | 113,489,582 | C | T | 3550 |
| chr12: 113489583 | 6.06E−03 | 0.232211 | 0.995297 | 113,489,583 | A | G | 3551 |
| rs1650059 | 6.19E−03 | −0.07694 | 0.039745 | 113,510,112 | C | T | 3552 |
| rs7487654 | 6.23E−03 | −0.058413 | 0.150034 | 113,796,605 | A | G | 3553 |
| rs1718376 | 6.28E−03 | −0.076815 | 0.039704 | 113,509,911 | A | T | 3554 |
| rs71442773 | 6.28E−03 | −0.064235 | 0.931593 | 113,379,331 | C | G | 3555 |
| rs7955377 | 6.29E−03 | −0.031113 | 0.396279 | 113,230,943 | A | G | 3556 |
| rs1718378 | 6.32E−03 | 0.07663 | 0.960202 | 113,508,466 | C | G | 3557 |
| rs7959528 | 6.33E−03 | −0.076626 | 0.039784 | 113,508,037 | C | T | 3558 |
| rs1718377 | 6.35E−03 | −0.076649 | 0.039744 | 113,508,620 | A | G | 3559 |
| rs1895605 | 6.41E−03 | −0.030285 | 0.487243 | 113,317,998 | A | G | 3560 |
| rs12229163 | 6.62E−03 | 0.062244 | 0.069726 | 113,384,812 | C | T | 3561 |
| rs1247936 | 6.75E−03 | 0.03045 | 0.551152 | 113,192,257 | C | T | 3562 |
| chr12: 112687164 | 6.77E−03 | 0.200237 | 0.009877 | 112,687,164 | C | G | 3563 |
| rs11067148 | 6.77E−03 | −0.049297 | 0.880403 | 113,426,887 | C | T | 3564 |
| rs10850293 | 6.78E−03 | 0.064074 | 0.942287 | 113,160,874 | C | T | 3565 |
| chr12: 113386769 | 6.82E−03 | 0.066033 | 0.060434 | 113,386,769 | A | C | 3566 |
| rs714253 | 6.89E−03 | −0.034332 | 0.75678 | 113,125,667 | A | G | 3567 |
| rs10507252 | 6.91E−03 | 0.059918 | 0.933461 | 113,690,748 | A | G | 3568 |
| chr12: 113724159 | 7.10E−03 | −0.079947 | 0.040181 | 113,724,159 | C | T | 3569 |
| chr12: 113152299 | 7.30E−03 | −0.062944 | 0.059861 | 113,152,299 | C | T | 3570 |
| rs12302924 | 7.34E−03 | 0.063048 | 0.940171 | 113,151,569 | C | T | 3571 |
| rs11831311 | 7.38E−03 | −0.063102 | 0.059728 | 113,150,948 | A | C | 3572 |
| rs10850298 | 7.44E−03 | −0.055233 | 0.811251 | 113,180,859 | A | T | 3573 |
| rs1896001 | 7.45E−03 | 0.031397 | 0.445075 | 113,213,388 | T | C | 3574 |
| chr12: 112950334 | 7.50E−03 | 0.049612 | 0.097315 | 112,950,334 | A | G | 3575 |
| rs1920566 | 7.59E−03 | −0.063089 | 0.059574 | 113,150,063 | A | G | 3576 |
| rs16944338 | 7.66E−03 | −0.06575 | 0.944481 | 113,379,111 | C | T | 3577 |
| rs11611982 | 7.76E−03 | 0.130258 | 0.015745 | 112,668,110 | A | G | 3578 |
| rs11066991 | 7.80E−03 | −0.033693 | 0.755546 | 113,134,878 | C | T | 3579 |
| chr12: 113692990 | 7.93E−03 | −0.060723 | 0.061791 | 113,692,990 | A | C | 3580 |
| rs11066711 | 7.96E−03 | −0.065863 | 0.935267 | 112,648,969 | C | T | 3581 |
| rs3843645 | 8.19E−03 | −0.06438 | 0.944741 | 113,382,729 | C | T | 3582 |
| rs11067127 | 8.26E−03 | 0.064058 | 0.055555 | 113,389,721 | C | G | 3583 |
| chr12: 113387553 | 8.32E−03 | 0.064027 | 0.055148 | 113,387,553 | A | G | 3584 |
| rs4767235 | 8.44E−03 | −0.03316 | 0.752781 | 113,122,292 | A | G | 3585 |
| rs34536114 | 8.52E−03 | 0.033041 | 0.245579 | 113,122,898 | C | T | 3586 |
| rs16943814 | 8.54E−03 | 0.032988 | 0.24591 | 113,125,101 | A | C | 3587 |
| rs11066982 | 8.54E−03 | −0.033136 | 0.75552 | 113,119,826 | C | T | 3588 |
| chr12: 113388455 | 8.55E−03 | −0.063766 | 0.944782 | 113,388,455 | C | T | 3589 |
| rs12367740 | 8.55E−03 | −0.032992 | 0.754122 | 113,126,183 | C | T | 3590 |
| chr12: 113393194 | 8.55E−03 | −0.063551 | 0.944933 | 113,393,194 | A | C | 3591 |
| rs11066980 | 8.57E−03 | 0.033471 | 0.237664 | 113,116,454 | A | G | 3592 |
| rs5025218 | 8.61E−03 | −0.06341 | 0.94497 | 113,395,228 | A | C | 3593 |
| chr12: 113582196 | 8.67E−03 | 0.218912 | 0.993241 | 113,582,196 | C | T | 3594 |
| rs12424926 | 8.68E−03 | −0.096857 | 0.977757 | 112,946,788 | C | T | 3595 |
| chr12: 113394267 | 8.70E−03 | 0.063364 | 0.055091 | 113,394,267 | C | T | 3596 |
| rs61930980 | 8.81E−03 | −0.032553 | 0.26388 | 113,258,868 | A | G | 3597 |
| rs10850321 | 8.88E−03 | 0.032474 | 0.735559 | 113,265,025 | C | G | 3598 |
| chr12: 112746178 | 8.88E−03 | 0.094921 | 0.024786 | 112,746,178 | C | T | 3599 |
| chr12: 112949950 | 8.89E−03 | −0.096737 | 0.97784 | 112,949,950 | A | G | 3600 |
| chr12: 112842125 | 8.90E−03 | 0.088354 | 0.026168 | 112,842,125 | A | C | 3601 |
| chr12: 113015483 | 8.92E−03 | 0.169795 | 0.986128 | 113,015,483 | A | G | 3602 |
| rs73394891 | 8.93E−03 | −0.062987 | 0.944938 | 113,399,694 | C | T | 3603 |
| rs11067089 | 8.94E−03 | −0.031207 | 0.355798 | 113,297,009 | C | T | 3604 |
| rs5025219 | 8.99E−03 | 0.063075 | 0.055156 | 113,395,083 | A | T | 3605 |
| chr12: 112842418 | 9.10E−03 | −0.088104 | 0.973895 | 112,842,418 | C | T | 3606 |
| chr12: 112842661 | 9.24E−03 | 0.087922 | 0.026058 | 112,842,661 | C | G | 3607 |
| rs513061 | 9.47E−03 | −0.041524 | 0.805637 | 113,589,060 | T | C | 3608 |
| chr12: 112742662 | 9.59E−03 | 0.078497 | 0.038839 | 112,742,662 | C | G | 3609 |
| rs7309593 | 9.67E−03 | 0.078636 | 0.038805 | 112,741,591 | G | T | 3610 |
| rs16944342 | 9.77E−03 | −0.06206 | 0.941217 | 113,381,253 | A | G | 3611 |
| chr12: 112860534 | 9.82E−03 | −0.087219 | 0.974138 | 112,860,534 | C | T | 3612 |
| chr12: 112858391 | 9.82E−03 | 0.087218 | 0.025862 | 112,858,391 | C | T | 3613 |
| chr12: 112861860 | 9.82E−03 | −0.087217 | 0.974138 | 112,861,860 | C | T | 3614 |
| rs12422551 | 9.83E−03 | 0.08719 | 0.025864 | 112,849,732 | C | T | 3615 |
| chr12: 112743374 | 9.86E−03 | −0.077657 | 0.960862 | 112,743,374 | C | G | 3616 |
| rs2290800 | 9.87E−03 | −0.08822 | 0.974302 | 112,888,766 | T | G | 3617 |
| rs2290793 | 9.89E−03 | −0.087205 | 0.974148 | 112,868,747 | C | T | 3618 |
| rs1380004 | 9.93E−03 | 0.087189 | 0.025846 | 112,875,147 | C | T | 3619 |
| chr12: 112878896 | 9.93E−03 | 0.087189 | 0.025845 | 112,878,896 | A | G | 3620 |
| rs11833922 | 9.96E−03 | 0.0291 | 0.452354 | 113,869,352 | A | G | 3621 |
| rs3825211 | 9.99E−03 | 0.087161 | 0.025836 | 112,881,080 | C | T | 3622 |
| rs3825212 | 9.99E−03 | 0.087158 | 0.025836 | 112,881,124 | A | T | 3623 |

Shown is marker identity, p-value of the association, magnitude of effect (in units of fractions of standard deviations), frequency of effect allele, position in NCBI Build 36, identity of effect allele, identity of other allele, SEQ ID for the marker. It should be noted that a positive value for an effect represents an increase in the interval conferred by the effect allele, while a negative value for the effect represents a decrease (i.e. the allele correlated with the negative effect is predictive of a decreased value of the measure).

TABLE 14
Association results for PR interval in a 2 Mb region flanking rs3825214 on
chromosome 12. Shown is marker identity, p-value of the association, magnitude of effect (in
units of fractions of standard deviations), frequency of effect allele, position in NCBI Build 36,
identity of effect allele, identity of other allele, SEQ ID for the marker. It should be noted that a
positive value for an effect represents a predicted increase in the interval conferred by the effect
allele, while a negative value for the effect represents a decrease (i.e. the allele correlated with
the negative effect is predictive of a decreased value of the measure).

| | | | | | effect | other | |
|---|---|---|---|---|---|---|---|
| marker | p-value | effect | freq | position | allele | allele | SEQ ID NO: |
| rs10850371 | 7.93E−07 | 0.057551 | 0.430437 | 113,652,609 | C | T | 2819 |
| rs2134219 | 1.03E−06 | 0.056266 | 0.530922 | 113,663,910 | C | G | 2820 |
| rs10850373 | 1.42E−06 | 0.057378 | 0.421858 | 113,653,862 | A | G | 2821 |
| rs10744832 | 3.50E−06 | −0.056243 | 0.630348 | 113,662,838 | C | T | 2822 |
| rs10774763 | 3.86E−06 | 0.05622 | 0.361557 | 113,652,756 | A | G | 2823 |
| rs10774766 | 4.13E−06 | −0.055091 | 0.635961 | 113,656,237 | C | T | 2824 |
| chr12: 113306769 | 4.41E−06 | −0.353152 | 0.980677 | 113,306,769 | C | G | 2825 |
| rs7979951 | 4.48E−06 | −0.057551 | 0.344101 | 113,669,320 | A | T | 2826 |
| rs11067268 | 4.71E−06 | −0.055006 | 0.639191 | 113,654,584 | C | T | 2827 |
| rs12302975 | 5.03E−06 | 0.054949 | 0.369425 | 113,658,331 | C | T | 2828 |
| rs2062716 | 6.26E−06 | −0.051529 | 0.579814 | 113,658,640 | C | T | 2829 |
| rs10774764 | 7.06E−06 | −0.051814 | 0.590562 | 113,652,823 | A | G | 2830 |
| rs2173957 | 8.09E−06 | −0.053367 | 0.598238 | 113,665,488 | A | G | 2831 |
| rs2173958 | 9.12E−06 | 0.052694 | 0.402674 | 113,663,629 | G | T | 2832 |
| rs2062715 | 9.68E−06 | −0.051711 | 0.589196 | 113,661,010 | A | G | 2833 |
| rs10850372 | 1.16E−05 | 0.052171 | 0.393652 | 113,652,726 | C | T | 2834 |
| chr12: 113325961 | 1.18E−05 | −0.168788 | 0.922456 | 113,325,961 | C | G | 2835 |
| rs2891503 | 1.25E−05 | 0.05932 | 0.238778 | 113,274,193 | A | G | 272 |
| rs10219718 | 1.33E−05 | −0.052296 | 0.586101 | 113,662,422 | C | T | 2836 |
| rs7134874 | 1.36E−05 | −0.051355 | 0.589231 | 113,664,540 | G | T | 2837 |
| rs3759174 | 1.39E−05 | −0.068535 | 0.717644 | 113,608,121 | C | G | 2838 |
| rs2384409 | 1.42E−05 | 0.059245 | 0.219567 | 113,273,861 | A | G | 271 |
| rs7316919 | 1.44E−05 | 0.058835 | 0.221127 | 113,275,838 | A | C | 275 |
| rs6489956 | 1.45E−05 | −0.058731 | 0.778493 | 113,276,619 | C | T | 276 |
| rs2113433 | 1.47E−05 | −0.058497 | 0.777774 | 113,278,440 | G | T | 278 |
| rs1920591 | 1.52E−05 | −0.056069 | 0.345134 | 113,190,592 | A | G | 2839 |
| rs1895597 | 1.63E−05 | −0.058075 | 0.769214 | 113,275,267 | A | T | 274 |
| rs7964303 | 1.91E−05 | −0.05495 | 0.708289 | 113,298,669 | G | T | 292 |
| rs7977083 | 2.00E−05 | 0.055968 | 0.25662 | 113,274,883 | A | G | 273 |
| rs12302135 | 2.16E−05 | −0.052937 | 0.697445 | 113,191,009 | C | T | 2840 |
| rs4767249 | 2.28E−05 | −0.048413 | 0.453856 | 113,660,063 | A | G | 2841 |
| rs3825214 | 2.34E−05 | −0.057024 | 0.781334 | 113,279,826 | A | G | 279 |
| chr12: 113577245 | 2.57E−05 | −0.082063 | 0.187876 | 113,577,245 | C | T | 2842 |
| rs2062717 | 2.62E−05 | −0.047311 | 0.448219 | 113,658,476 | A | G | 2843 |
| rs2062713 | 2.71E−05 | 0.047498 | 0.558064 | 113,660,935 | C | T | 2844 |
| rs2062714 | 2.72E−05 | 0.047474 | 0.558245 | 113,660,989 | G | T | 2845 |
| rs11067267 | 2.76E−05 | 0.047568 | 0.543058 | 113,654,410 | G | T | 2846 |
| rs12424875 | 3.23E−05 | 0.049172 | 0.523314 | 113,671,713 | C | T | 2847 |
| rs2701106 | 3.27E−05 | −0.046855 | 0.512179 | 113,181,930 | A | G | 2848 |
| rs7308120 | 3.32E−05 | −0.057068 | 0.784397 | 113,273,429 | C | T | 270 |
| rs2384408 | 3.35E−05 | 0.056996 | 0.215815 | 113,273,733 | A | G | 2849 |
| rs12367410 | 3.37E−05 | −0.056488 | 0.783762 | 113,281,071 | C | T | 280 |
| rs10774765 | 3.51E−05 | 0.047576 | 0.54067 | 113,653,064 | G | T | 2850 |
| rs883079 | 3.51E−05 | 0.050168 | 0.304506 | 113,277,623 | C | T | 277 |
| rs2384407 | 3.72E−05 | −0.050296 | 0.697398 | 113,273,609 | A | G | 2851 |
| chr12: 113315810 | 3.76E−05 | −0.106639 | 0.877438 | 113,315,810 | A | C | 2852 |
| rs2134220 | 4.00E−05 | −0.048422 | 0.384636 | 113,663,704 | A | G | 2853 |
| rs12314828 | 4.91E−05 | −0.053796 | 0.517481 | 113,652,522 | A | G | 2854 |
| rs35023333 | 5.07E−05 | −0.109808 | 0.938187 | 113,574,784 | C | T | 2855 |
| rs2555030 | 5.11E−05 | 0.060222 | 0.186758 | 113,301,715 | A | G | 2856 |
| rs10744823 | 5.25E−05 | 0.05575 | 0.216396 | 113,282,465 | C | T | 283 |
| rs10431392 | 6.47E−05 | −0.093687 | 0.915649 | 113,588,026 | A | G | 2857 |
| rs10850377 | 6.68E−05 | −0.046908 | 0.363447 | 113,685,819 | A | G | 2858 |
| rs4767250 | 6.98E−05 | 0.050771 | 0.644904 | 113,667,311 | G | T | 2859 |
| rs11067277 | 7.04E−05 | 0.048444 | 0.561052 | 113,666,342 | C | T | 2860 |
| rs11608388 | 7.31E−05 | −0.046214 | 0.360189 | 113,188,057 | A | C | 2861 |
| rs11610607 | 7.31E−05 | 0.046277 | 0.639796 | 113,183,152 | A | G | 2862 |
| rs12426785 | 7.31E−05 | 0.046236 | 0.639772 | 113,185,981 | C | T | 2863 |
| rs10744833 | 7.36E−05 | −0.045996 | 0.483158 | 113,672,466 | A | G | 2864 |
| chr12: 113422460 | 7.38E−05 | −0.289538 | 0.98279 | 113,422,460 | A | G | 2865 |
| rs7311915 | 7.52E−05 | 0.046374 | 0.365692 | 113,855,525 | C | G | 2866 |
| rs4767253 | 7.59E−05 | −0.054737 | 0.720724 | 113,682,079 | C | G | 2867 |
| rs1862909 | 8.14E−05 | 0.055677 | 0.200957 | 113,271,488 | A | G | 2868 |
| rs11067275 | 8.41E−05 | 0.051696 | 0.281985 | 113,665,093 | C | T | 2869 |
| rs1946295 | 8.74E−05 | 0.048428 | 0.28992 | 113,286,744 | T | C | 288 |
| rs7309910 | 8.77E−05 | 0.055548 | 0.200136 | 113,270,637 | C | G | 269 |
| chr12: 113684980 | 9.06E−05 | −0.045989 | 0.370117 | 113,684,980 | C | T | 2870 |
| rs12228985 | 9.38E−05 | 0.104954 | 0.060947 | 113,585,467 | A | T | 2871 |
| chr12: 113593897 | 9.53E−05 | −0.106402 | 0.940196 | 113,593,897 | C | T | 2872 |
| chr12: 113237864 | 9.67E−05 | 0.124114 | 0.966735 | 113,237,864 | G | T | 2873 |
| rs10507248 | 1.02E−04 | 0.047755 | 0.292588 | 113,281,476 | G | T | 281 |
| rs6489955 | 1.03E−04 | −0.05514 | 0.801216 | 113,269,662 | A | G | 268 |
| rs10744822 | 1.05E−04 | 0.05521 | 0.198608 | 113,268,423 | C | T | 2874 |
| rs7955405 | 1.08E−04 | 0.047605 | 0.292166 | 113,281,689 | A | G | 282 |
| rs11067280 | 1.17E−04 | −0.047051 | 0.331227 | 113,674,984 | A | G | 2875 |
| rs10850409 | 1.35E−04 | 0.045963 | 0.347359 | 113,866,123 | A | G | 2876 |
| rs12422933 | 1.38E−04 | −0.059331 | 0.351224 | 113,591,071 | A | C | 2877 |
| rs7138531 | 1.42E−04 | −0.045916 | 0.422417 | 113,650,674 | A | G | 2878 |
| rs17731569 | 1.42E−04 | −0.053115 | 0.792003 | 113,312,090 | A | G | 293 |
| rs11067279 | 1.45E−04 | 0.046653 | 0.662866 | 113,670,422 | A | C | 2879 |
| rs10850378 | 1.48E−04 | 0.044575 | 0.648136 | 113,688,002 | C | T | 2880 |
| rs7313200 | 1.55E−04 | −0.04565 | 0.421676 | 113,650,579 | C | T | 2881 |
| rs3741695 | 1.61E−04 | −0.100976 | 0.93865 | 113,593,282 | C | T | 2882 |
| rs61931191 | 1.62E−04 | 0.044482 | 0.64752 | 113,688,814 | G | T | 2883 |
| rs11615965 | 1.62E−04 | 0.04448 | 0.647464 | 113,688,840 | C | T | 2884 |
| chr12: 113689107 | 1.69E−04 | −0.044878 | 0.349154 | 113,689,107 | A | G | 2885 |
| rs12370593 | 1.72E−04 | 0.045785 | 0.663827 | 113,673,744 | C | T | 2886 |
| chr12: 113234638 | 1.72E−04 | −0.781926 | 0.991611 | 113,234,638 | G | T | 2887 |
| rs3858619 | 1.73E−04 | 0.05489 | 0.737932 | 113,412,658 | A | C | 2888 |
| chr12: 113237275 | 1.73E−04 | −0.11057 | 0.0398 | 113,237,275 | C | T | 2889 |
| rs7301677 | 1.77E−04 | −0.044212 | 0.655658 | 113,865,530 | C | T | 2890 |
| rs7132327 | 1.77E−04 | 0.044212 | 0.344351 | 113,865,454 | C | T | 2891 |
| rs11067283 | 1.79E−04 | −0.04569 | 0.333909 | 113,680,274 | A | T | 2892 |
| rs55724378 | 1.81E−04 | −0.045192 | 0.569477 | 113,682,288 | A | C | 2893 |
| rs1920597 | 1.87E−04 | 0.041824 | 0.501774 | 113,188,475 | A | G | 2894 |
| chr12: 113264239 | 1.87E−04 | −0.111434 | 0.039341 | 113,264,239 | A | G | 2895 |
| rs1920596 | 1.98E−04 | −0.041593 | 0.497044 | 113,188,795 | A | T | 2896 |
| rs10850406 | 2.04E−04 | 0.043879 | 0.36438 | 113,852,223 | A | G | 2897 |
| rs10850381 | 2.07E−04 | 0.043804 | 0.646232 | 113,690,335 | G | T | 2898 |
| rs7966748 | 2.08E−04 | 0.045767 | 0.491781 | 113,856,605 | A | T | 2899 |
| rs7980129 | 2.17E−04 | 0.043419 | 0.352789 | 113,856,935 | G | T | 2900 |
| rs7966651 | 2.20E−04 | −0.043376 | 0.647102 | 113,856,341 | C | T | 2901 |
| rs10744836 | 2.22E−04 | −0.043345 | 0.647026 | 113,856,125 | G | T | 2902 |
| rs7311039 | 2.29E−04 | −0.04322 | 0.64673 | 113,855,591 | C | G | 2903 |
| rs4767282 | 2.30E−04 | 0.043205 | 0.353305 | 113,853,421 | C | G | 2904 |
| chr12: 113274919 | 2.30E−04 | −0.112154 | 0.039098 | 113,274,919 | A | C | 2905 |
| rs2384555 | 2.31E−04 | −0.043195 | 0.646673 | 113,852,040 | C | T | 2906 |
| rs1896356 | 2.31E−04 | 0.043192 | 0.353333 | 113,851,669 | C | T | 2907 |
| chr12: 113408476 | 2.31E−04 | −0.072643 | 0.088533 | 113,408,476 | A | G | 2908 |
| rs10850405 | 2.31E−04 | 0.043188 | 0.353343 | 113,851,052 | A | T | 2909 |
| rs10850404 | 2.31E−04 | −0.043185 | 0.646651 | 113,850,668 | G | T | 2910 |
| rs7980361 | 2.33E−04 | −0.043162 | 0.6466 | 113,847,494 | C | T | 2911 |
| rs7976673 | 2.33E−04 | 0.043151 | 0.353424 | 113,846,997 | A | T | 2912 |
| rs10444497 | 2.34E−04 | −0.043241 | 0.645207 | 113,855,029 | C | G | 2913 |
| rs4767239 | 2.35E−04 | −0.053278 | 0.798894 | 113,300,931 | C | G | 2914 |
| rs10774767 | 2.38E−04 | −0.04579 | 0.615057 | 113,660,418 | C | T | 2915 |
| chr12: 113315599 | 2.39E−04 | −0.363621 | 0.982587 | 113,315,599 | C | T | 2916 |
| rs9630280 | 2.40E−04 | −0.043113 | 0.646362 | 113,846,140 | C | T | 2917 |
| rs1896358 | 2.48E−04 | 0.043072 | 0.353846 | 113,845,574 | C | T | 2918 |
| rs10444496 | 2.57E−04 | −0.050502 | 0.748433 | 113,677,561 | C | T | 2919 |
| rs12582045 | 2.59E−04 | 0.071809 | 0.91072 | 113,410,458 | C | T | 2920 |
| rs7966951 | 2.59E−04 | 0.043086 | 0.344541 | 113,849,594 | A | G | 2921 |
| rs3914956 | 2.60E−04 | −0.043075 | 0.655445 | 113,848,134 | A | T | 2922 |
| rs7487962 | 2.66E−04 | 0.043029 | 0.344747 | 113,846,313 | A | G | 2923 |
| rs7132593 | 2.72E−04 | −0.043028 | 0.646446 | 113,845,249 | A | G | 2924 |
| rs6416327 | 2.74E−04 | −0.043001 | 0.65507 | 113,845,747 | G | T | 2925 |
| rs10507250 | 2.82E−04 | −0.074567 | 0.093037 | 113,380,179 | T | C | 2926 |
| rs10744824 | 2.94E−04 | −0.050879 | 0.799931 | 113,293,021 | A | G | 2927 |
| rs7312625 | 3.08E−04 | −0.044974 | 0.718133 | 113,284,357 | A | G | 284 |
| rs11067265 | 3.25E−04 | 0.043095 | 0.576768 | 113,651,372 | C | T | 2928 |
| rs2162320 | 3.67E−04 | −0.052608 | 0.806317 | 113,191,172 | C | T | 2929 |
| rs1920593 | 3.79E−04 | −0.048752 | 0.790005 | 113,189,926 | C | T | 2930 |
| rs11067278 | 3.96E−04 | 0.043651 | 0.339824 | 113,668,436 | C | G | 2931 |
| rs1920595 | 3.97E−04 | −0.048605 | 0.789669 | 113,189,705 | A | C | 2932 |
| rs10774768 | 4.12E−04 | −0.040966 | 0.489835 | 113,677,577 | C | T | 2933 |
| rs4767237 | 4.13E−04 | 0.044027 | 0.280263 | 113,285,196 | A | G | 285 |
| rs7132580 | 4.25E−04 | −0.041522 | 0.64212 | 113,845,207 | A | G | 2934 |
| rs7978143 | 4.33E−04 | 0.053319 | 0.823835 | 113,421,296 | A | G | 2935 |
| rs5015007 | 4.37E−04 | −0.050007 | 0.804004 | 113,289,466 | A | T | 2936 |
| rs1981946 | 4.45E−04 | −0.112171 | 0.954945 | 113,601,413 | G | T | 2937 |
| chr12: 113287381 | 4.52E−04 | −0.115028 | 0.061441 | 113,287,381 | G | T | 2938 |
| rs1061651 | 4.57E−04 | −0.048588 | 0.251604 | 113,592,744 | C | T | 2939 |
| rs2891537 | 4.64E−04 | −0.041174 | 0.640651 | 113,843,335 | G | T | 2940 |
| rs1566643 | 4.73E−04 | 0.041402 | 0.417741 | 113,690,703 | A | G | 2941 |
| rs10774769 | 4.74E−04 | 0.04622 | 0.265593 | 113,683,120 | C | T | 2942 |
| rs12580721 | 4.94E−04 | 0.042685 | 0.337349 | 113,672,623 | A | C | 2943 |
| rs7135659 | 4.98E−04 | −0.043109 | 0.716493 | 113,286,155 | A | G | 286 |
| rs1896330 | 5.09E−04 | −0.040865 | 0.640324 | 113,841,386 | A | G | 2944 |
| rs12578160 | 5.24E−04 | 0.04245 | 0.335906 | 113,672,317 | A | G | 2945 |
| rs7300371 | 5.48E−04 | −0.042988 | 0.643799 | 113,855,564 | C | T | 2946 |
| rs7487237 | 5.67E−04 | 0.04046 | 0.360065 | 113,838,086 | A | G | 2947 |
| rs17660551 | 5.70E−04 | −0.047318 | 0.787133 | 113,188,005 | A | C | 2948 |
| rs17660485 | 5.90E−04 | −0.047183 | 0.786898 | 113,187,497 | C | G | 2949 |
| rs4118382 | 6.06E−04 | −0.057905 | 0.246322 | 113,149,947 | A | T | 2950 |
| rs11067262 | 6.17E−04 | −0.05331 | 0.749534 | 113,646,809 | G | T | 2951 |
| rs1946293 | 6.32E−04 | −0.042299 | 0.717743 | 113,287,143 | A | G | 289 |
| rs10850401 | 6.33E−04 | 0.040119 | 0.359619 | 113,836,292 | A | C | 2952 |
| rs1896347 | 6.38E−04 | 0.040098 | 0.359594 | 113,836,177 | G | T | 2953 |
| rs7138015 | 6.41E−04 | 0.046862 | 0.213642 | 113,186,261 | A | C | 2954 |
| chr12: 113203749 | 6.60E−04 | −0.673327 | 0.991193 | 113,203,749 | C | T | 2955 |
| rs1895585 | 6.62E−04 | 0.042375 | 0.277734 | 113,286,521 | A | G | 287 |
| chr12: 113374127 | 6.63E−04 | 0.073194 | 0.91537 | 113,374,127 | C | G | 2956 |
| chr12: 112944886 | 6.93E−04 | −0.04865 | 0.352462 | 112,944,886 | A | G | 2957 |
| rs58562176 | 6.95E−04 | −0.046538 | 0.785834 | 113,185,837 | C | T | 2958 |
| rs1124477 | 7.14E−04 | −0.039753 | 0.641287 | 113,833,880 | C | T | 2959 |
| rs10774752 | 7.15E−04 | 0.048264 | 0.188509 | 113,252,695 | A | G | 2960 |
| rs7964836 | 7.25E−04 | −0.04821 | 0.811491 | 113,251,103 | C | G | 2961 |
| rs8181608 | 7.27E−04 | 0.048933 | 0.183954 | 113,245,966 | A | G | 259 |
| rs7307520 | 7.39E−04 | 0.048132 | 0.188515 | 113,249,082 | A | G | 2962 |
| chr12: 113384018 | 7.40E−04 | 0.067741 | 0.913276 | 113,384,018 | C | T | 2963 |
| rs4547150 | 7.41E−04 | −0.045359 | 0.648302 | 113,649,565 | C | T | 2964 |
| rs6489953 | 7.43E−04 | 0.048118 | 0.188781 | 113,249,145 | C | T | 264 |
| rs10744820 | 7.50E−04 | 0.048488 | 0.184141 | 113,252,510 | A | G | 265 |
| rs1895587 | 7.55E−04 | −0.048479 | 0.815854 | 113,253,912 | C | T | 266 |
| rs11067266 | 7.60E−04 | −0.048251 | 0.756596 | 113,651,827 | G | T | 2965 |
| rs6489952 | 7.70E−04 | −0.048943 | 0.81572 | 113,243,156 | A | G | 256 |
| rs1247937 | 7.70E−04 | −0.039367 | 0.5612 | 113,192,294 | A | T | 2966 |
| rs7965033 | 7.84E−04 | −0.048307 | 0.808474 | 113,295,738 | C | T | 2967 |
| rs58872758 | 7.92E−04 | −0.046121 | 0.784238 | 113,180,030 | G | T | 2968 |
| chr12: 113181795 | 7.94E−04 | −0.046028 | 0.784582 | 113,181,795 | A | G | 2969 |
| rs10744819 | 7.99E−04 | 0.048241 | 0.184162 | 113,246,213 | A | G | 263 |
| rs8181627 | 7.99E−04 | −0.048238 | 0.815838 | 113,246,147 | C | T | 262 |
| rs8181683 | 8.00E−04 | 0.048236 | 0.184162 | 113,246,101 | C | T | 261 |
| rs10744818 | 8.00E−04 | −0.048234 | 0.815838 | 113,246,068 | C | T | 260 |
| rs7966567 | 8.03E−04 | 0.048224 | 0.184183 | 113,245,863 | C | T | 258 |
| rs3825215 | 8.09E−04 | −0.04199 | 0.720456 | 113,289,281 | C | G | 290 |
| rs1895593 | 8.10E−04 | −0.048276 | 0.81561 | 113,245,198 | A | G | 257 |
| rs17660241 | 8.36E−04 | 0.046061 | 0.214841 | 113,179,284 | A | G | 2970 |
| rs12231030 | 8.36E−04 | −0.099409 | 0.044054 | 113,295,169 | C | T | 2971 |
| rs11067098 | 8.57E−04 | −0.105933 | 0.038052 | 113,315,145 | A | G | 2972 |
| rs1896312 | 8.61E−04 | 0.039085 | 0.357458 | 113,830,807 | C | T | 2973 |
| rs933748 | 8.70E−04 | 0.047106 | 0.205406 | 113,245,039 | C | T | 2974 |
| rs2113437 | 8.73E−04 | 0.106956 | 0.962308 | 113,316,323 | C | T | 2975 |
| chr12: 113399882 | 8.79E−04 | −0.12697 | 0.032387 | 113,399,882 | A | G | 2976 |
| rs1895582 | 9.27E−04 | −0.041496 | 0.723842 | 113,291,418 | A | G | 291 |
| rs2555014 | 9.79E−04 | −0.036767 | 0.522542 | 113,161,874 | G | T | 2977 |
| chr12: 112607206 | 9.83E−04 | 0.265452 | 0.007079 | 112,607,206 | A | G | 2978 |
| chr12: 113180727 | 9.94E−04 | −0.09687 | 0.928886 | 113,180,727 | C | T | 2979 |
| chr12: 113200986 | 9.97E−04 | 0.097513 | 0.067363 | 113,200,986 | A | G | 2980 |
| rs17660176 | 9.98E−04 | 0.070649 | 0.154323 | 113,177,009 | A | G | 2981 |
| rs6489990 | 1.01E−03 | −0.040006 | 0.686155 | 113,828,768 | A | T | 2982 |
| rs1265496 | 1.04E−03 | −0.036537 | 0.522738 | 113,161,366 | C | T | 2983 |
| chr12: 113370640 | 1.04E−03 | 0.072302 | 0.78792 | 113,370,640 | A | C | 2984 |
| rs1895583 | 1.09E−03 | 0.041041 | 0.275231 | 113,291,268 | A | G | 2985 |
| chr12: 113556849 | 1.10E−03 | −0.081071 | 0.147128 | 113,556,849 | G | T | 2986 |
| rs16943956 | 1.10E−03 | −0.095432 | 0.93096 | 113,195,145 | A | G | 2987 |
| chr12: 112699391 | 1.11E−03 | −0.058052 | 0.19542 | 112,699,391 | A | T | 2988 |
| rs60121244 | 1.16E−03 | −0.0404 | 0.646003 | 113,683,398 | G | T | 2989 |
| rs10850346 | 1.19E−03 | −0.050954 | 0.146671 | 113,414,173 | C | T | 2990 |
| rs1270886 | 1.20E−03 | 0.036206 | 0.478379 | 113,160,853 | C | T | 2991 |
| rs16942762 | 1.23E−03 | 0.090859 | 0.068857 | 112,286,477 | A | G | 2992 |
| rs4639978 | 1.24E−03 | −0.044593 | 0.435705 | 114,210,500 | C | T | 2993 |
| rs12820329 | 1.25E−03 | −0.129388 | 0.961062 | 113,565,734 | A | C | 2994 |
| rs10850315 | 1.25E−03 | 0.0401 | 0.2758 | 113,251,118 | G | T | 2995 |
| rs4767255 | 1.26E−03 | −0.039128 | 0.63601 | 113,682,875 | C | G | 2996 |
| rs35383422 | 1.26E−03 | 0.045458 | 0.757185 | 113,576,401 | A | T | 2997 |
| chr12: 113808679 | 1.29E−03 | 0.103476 | 0.03599 | 113,808,679 | C | T | 2998 |
| rs10507249 | 1.30E−03 | 0.06255 | 0.893065 | 113,375,644 | C | G | 2999 |
| rs4767254 | 1.33E−03 | 0.038911 | 0.364132 | 113,682,775 | A | G | 3000 |
| rs11831276 | 1.39E−03 | −0.040582 | 0.643325 | 113,683,258 | G | T | 3001 |
| rs11067228 | 1.40E−03 | 0.036129 | 0.559691 | 113,578,643 | A | G | 3002 |
| rs4767236 | 1.45E−03 | 0.046046 | 0.185644 | 113,221,039 | A | G | 3003 |
| rs1078568 | 1.48E−03 | −0.045947 | 0.814344 | 113,220,504 | A | G | 3004 |
| rs10850370 | 1.48E−03 | 0.041849 | 0.359097 | 113,648,796 | A | G | 3005 |
| rs1078567 | 1.48E−03 | −0.045914 | 0.814344 | 113,220,355 | A | G | 3006 |
| rs4259873 | 1.50E−03 | −0.045856 | 0.814508 | 113,219,162 | C | T | 3007 |
| rs4556590 | 1.51E−03 | 0.045793 | 0.185657 | 113,219,302 | C | T | 3008 |
| rs4625524 | 1.52E−03 | −0.040231 | 0.680313 | 113,828,468 | A | G | 3009 |
| chr12: 113217529 | 1.53E−03 | −0.119633 | 0.02923 | 113,217,529 | C | T | 3010 |
| rs4540867 | 1.53E−03 | 0.045706 | 0.185657 | 113,218,077 | A | G | 3011 |
| rs753564 | 1.54E−03 | −0.045654 | 0.814343 | 113,217,328 | C | G | 3012 |
| rs12314827 | 1.58E−03 | −0.053328 | 0.260219 | 113,652,520 | A | G | 3013 |
| chr12: 113198160 | 1.61E−03 | −0.044145 | 0.799802 | 113,198,160 | C | T | 3014 |
| rs11067264 | 1.62E−03 | 0.04146 | 0.357983 | 113,648,407 | A | G | 3015 |
| rs4766728 | 1.62E−03 | 0.044074 | 0.199688 | 113,199,628 | C | T | 3016 |
| rs7132243 | 1.62E−03 | 0.044066 | 0.199599 | 113,202,664 | C | T | 3017 |
| chr12: 113562516 | 1.63E−03 | −0.080255 | 0.069418 | 113,562,516 | C | T | 3018 |
| rs7959283 | 1.63E−03 | 0.036599 | 0.365759 | 113,854,921 | A | T | 3019 |
| chr12: 113198279 | 1.63E−03 | −0.044035 | 0.80049 | 113,198,279 | A | C | 3020 |
| rs4122458 | 1.65E−03 | −0.036546 | 0.634149 | 113,848,337 | G | T | 3021 |
| rs60302750 | 1.65E−03 | 0.045191 | 0.185243 | 113,209,985 | A | G | 3022 |
| rs11067327 | 1.66E−03 | 0.036542 | 0.365858 | 113,847,826 | C | T | 3023 |
| rs7977151 | 1.66E−03 | 0.036537 | 0.365866 | 113,847,355 | A | G | 3024 |
| rs7979724 | 1.66E−03 | −0.036525 | 0.634113 | 113,846,967 | C | T | 3025 |
| rs2384554 | 1.67E−03 | −0.0365 | 0.63407 | 113,846,661 | G | T | 3026 |
| rs60029182 | 1.69E−03 | −0.045608 | 0.815531 | 113,227,756 | G | T | 3027 |
| chr12: 112655298 | 1.73E−03 | 0.377452 | 0.995095 | 112,655,298 | C | T | 3028 |
| chr12: 113375025 | 1.76E−03 | −0.064906 | 0.093572 | 113,375,025 | C | T | 3029 |
| rs57797354 | 1.79E−03 | 0.046704 | 0.171614 | 113,211,534 | C | T | 3030 |
| rs6489974 | 1.79E−03 | 0.03804 | 0.574558 | 113,640,913 | A | G | 3031 |
| rs1247926 | 1.79E−03 | 0.034777 | 0.492781 | 113,171,694 | C | T | 3032 |
| rs1976426 | 1.83E−03 | 0.037314 | 0.372629 | 113,688,911 | C | G | 3033 |
| chr12: 113009556 | 1.85E−03 | −0.137542 | 0.018095 | 113,009,556 | A | G | 3034 |
| rs1247927 | 1.86E−03 | 0.034665 | 0.492766 | 113,171,439 | C | T | 3035 |
| rs11067145 | 1.86E−03 | 0.048497 | 0.848802 | 113,417,288 | G | T | 3036 |
| rs2428243 | 1.89E−03 | 0.042594 | 0.270829 | 114,201,808 | C | T | 3037 |
| rs1247932 | 1.90E−03 | 0.044511 | 0.353284 | 113,177,514 | A | G | 3038 |
| rs60740080 | 1.90E−03 | −0.06875 | 0.077383 | 113,375,919 | C | G | 3039 |
| chr12: 113096076 | 1.90E−03 | 0.154969 | 0.985796 | 113,096,076 | A | T | 3040 |
| rs11067054 | 1.91E−03 | 0.048417 | 0.16855 | 113,256,940 | A | G | 3041 |
| rs1247928 | 1.92E−03 | 0.03457 | 0.492755 | 113,171,223 | C | T | 3042 |
| rs34474591 | 1.94E−03 | −0.087272 | 0.936175 | 112,299,900 | G | T | 3043 |
| rs34750359 | 1.95E−03 | 0.087222 | 0.063762 | 112,295,216 | C | T | 3044 |
| chr12: 113211476 | 1.95E−03 | 0.083888 | 0.105731 | 113,211,476 | A | G | 3045 |
| rs34340397 | 1.95E−03 | −0.087224 | 0.936202 | 112,297,447 | C | T | 3046 |
| rs71465900 | 1.96E−03 | −0.087158 | 0.93618 | 112,294,491 | A | G | 3047 |
| rs34863464 | 1.96E−03 | −0.087111 | 0.936184 | 112,282,616 | C | T | 3048 |
| rs35526182 | 1.96E−03 | −0.087129 | 0.936299 | 112,293,612 | A | G | 3049 |
| rs71465901 | 1.96E−03 | 0.08716 | 0.063703 | 112,297,266 | A | G | 3050 |
| rs1896018 | 1.96E−03 | −0.044598 | 0.815754 | 113,215,780 | A | G | 3051 |
| rs12824200 | 1.97E−03 | −0.087105 | 0.936239 | 112,293,645 | A | G | 3052 |
| chr12: 112290765 | 1.97E−03 | −0.08709 | 0.936344 | 112,290,765 | C | T | 3053 |
| rs12820985 | 1.97E−03 | −0.087045 | 0.933705 | 112,281,239 | A | C | 3054 |
| rs34991985 | 1.97E−03 | 0.087065 | 0.063662 | 112,290,476 | A | G | 3055 |
| rs35435518 | 1.98E−03 | −0.087823 | 0.936043 | 112,285,473 | A | G | 3056 |
| rs34593969 | 1.98E−03 | 0.087023 | 0.06374 | 112,286,571 | A | G | 3057 |
| rs1896016 | 1.99E−03 | 0.044477 | 0.184476 | 113,215,558 | C | T | 3058 |
| rs9668121 | 1.99E−03 | 0.086976 | 0.063688 | 112,283,095 | C | T | 3059 |
| rs1896015 | 1.99E−03 | 0.044472 | 0.184476 | 113,215,473 | A | T | 3060 |
| rs12815547 | 2.00E−03 | 0.086968 | 0.063729 | 112,284,300 | A | G | 3061 |
| rs71465902 | 2.00E−03 | −0.08706 | 0.935418 | 112,298,396 | A | G | 3062 |
| rs7973232 | 2.00E−03 | −0.086944 | 0.936313 | 112,282,788 | A | G | 3063 |
| rs7974117 | 2.00E−03 | −0.087647 | 0.936625 | 112,292,288 | C | T | 3064 |
| rs4261334 | 2.01E−03 | −0.044425 | 0.815524 | 113,214,723 | A | C | 3065 |
| rs4274232 | 2.01E−03 | 0.044417 | 0.184476 | 113,214,598 | C | T | 3066 |
| rs59777856 | 2.01E−03 | −0.044405 | 0.815591 | 113,213,862 | G | T | 3067 |
| rs71442754 | 2.02E−03 | 0.087418 | 0.063431 | 112,289,996 | G | T | 3068 |
| chr12: 113213806 | 2.02E−03 | −0.04437 | 0.815523 | 113,213,806 | A | G | 3069 |
| rs35586551 | 2.03E−03 | −0.086928 | 0.935016 | 112,293,845 | C | T | 3070 |
| rs55870641 | 2.03E−03 | 0.043102 | 0.198275 | 113,206,637 | C | T | 3071 |
| rs60772998 | 2.03E−03 | 0.044339 | 0.184466 | 113,213,250 | A | G | 3072 |
| chr12: 113212688 | 2.04E−03 | 0.044318 | 0.184456 | 113,212,688 | A | G | 3073 |
| chr12: 113204921 | 2.04E−03 | 0.043075 | 0.198265 | 113,204,921 | C | T | 3074 |
| chr12: 113204801 | 2.04E−03 | −0.043074 | 0.801735 | 113,204,801 | G | T | 3075 |
| rs60268458 | 2.04E−03 | −0.044308 | 0.815552 | 113,212,437 | A | C | 3076 |
| rs6489949 | 2.04E−03 | −0.043067 | 0.801738 | 113,202,536 | C | T | 3077 |
| rs34114640 | 2.04E−03 | 0.08688 | 0.065639 | 112,285,185 | C | T | 3078 |
| rs58412031 | 2.04E−03 | −0.043059 | 0.801741 | 113,199,493 | C | T | 3079 |
| rs1896014 | 2.08E−03 | 0.044196 | 0.184363 | 113,209,532 | A | G | 3080 |
| chr12: 113205016 | 2.08E−03 | 0.043082 | 0.197894 | 113,205,016 | A | G | 3081 |
| rs1896013 | 2.08E−03 | −0.044193 | 0.815639 | 113,209,450 | C | T | 3082 |
| rs1896012 | 2.08E−03 | 0.044192 | 0.18436 | 113,209,430 | C | T | 3083 |
| chr12: 113208133 | 2.11E−03 | −0.044123 | 0.815691 | 113,208,133 | A | T | 3084 |
| rs1955105 | 2.15E−03 | 0.0363 | 0.369981 | 113,829,165 | A | C | 3085 |
| rs4766741 | 2.20E−03 | 0.038223 | 0.648681 | 113,736,448 | C | G | 3086 |
| rs7301743 | 2.20E−03 | −0.036862 | 0.666866 | 113,828,944 | A | G | 3087 |
| chr12: 112944888 | 2.23E−03 | −0.049921 | 0.283068 | 112,944,888 | A | G | 3088 |
| rs17730547 | 2.24E−03 | 0.086305 | 0.95601 | 113,294,816 | A | C | 3089 |
| rs9669457 | 2.28E−03 | 0.040561 | 0.252844 | 113,260,665 | A | G | 267 |
| rs2253207 | 2.31E−03 | 0.033978 | 0.49268 | 113,169,820 | C | T | 3090 |
| chr12: 112454831 | 2.31E−03 | −0.367114 | 0.012141 | 112,454,831 | A | G | 3091 |
| rs1269789 | 2.35E−03 | 0.033926 | 0.492676 | 113,168,925 | C | T | 3092 |
| rs34388546 | 2.36E−03 | −0.039412 | 0.752233 | 113,167,597 | C | T | 3093 |
| rs1247938 | 2.39E−03 | 0.033869 | 0.49267 | 113,167,951 | A | G | 3094 |
| rs2252923 | 2.40E−03 | −0.033858 | 0.507334 | 113,167,703 | A | G | 3095 |
| rs2252924 | 2.40E−03 | 0.033857 | 0.492669 | 113,167,706 | A | C | 3096 |
| rs1247940 | 2.42E−03 | 0.033828 | 0.492666 | 113,167,034 | C | T | 3097 |
| rs73195096 | 2.44E−03 | −0.068472 | 0.086672 | 113,560,746 | G | T | 3098 |
| rs17676517 | 2.45E−03 | −0.061505 | 0.102807 | 113,372,692 | A | G | 3099 |
| rs1270885 | 2.47E−03 | −0.033763 | 0.507343 | 113,165,935 | A | G | 3100 |
| rs11067013 | 2.47E−03 | −0.039229 | 0.752236 | 113,165,410 | C | T | 3101 |
| rs10850403 | 2.49E−03 | −0.035085 | 0.619871 | 113,844,976 | C | T | 3102 |
| rs2701104 | 2.50E−03 | 0.035926 | 0.347812 | 113,188,039 | C | G | 3103 |
| rs12826247 | 2.59E−03 | 0.084699 | 0.065377 | 112,281,254 | G | T | 3104 |
| rs4766724 | 2.61E−03 | −0.040484 | 0.759374 | 113,092,421 | G | T | 3105 |
| rs7302926 | 2.66E−03 | −0.047583 | 0.173382 | 113,572,696 | C | T | 3106 |
| rs7967168 | 2.75E−03 | −0.034751 | 0.619504 | 113,843,589 | A | T | 3107 |
| chr12: 113179810 | 2.77E−03 | −0.309892 | 0.009514 | 113,179,810 | A | G | 3108 |
| rs2252414 | 2.79E−03 | 0.033351 | 0.49261 | 113,163,520 | A | G | 3109 |
| chr12: 112992048 | 2.80E−03 | 0.053073 | 0.117025 | 112,992,048 | A | G | 3110 |
| rs7980132 | 2.86E−03 | −0.038643 | 0.752337 | 113,163,056 | A | G | 3111 |
| rs16945315 | 2.86E−03 | 0.04065 | 0.227124 | 114,177,896 | A | G | 3112 |
| rs2555012 | 2.86E−03 | −0.03327 | 0.507465 | 113,163,108 | C | T | 3113 |
| rs2384552 | 2.88E−03 | 0.034578 | 0.380914 | 113,842,311 | A | G | 3114 |
| rs7972037 | 2.93E−03 | −0.035295 | 0.622833 | 113,854,909 | A | G | 3115 |
| chr12: 113850412 | 2.94E−03 | 0.036969 | 0.470248 | 113,850,412 | G | T | 3116 |
| rs7977369 | 2.95E−03 | −0.041605 | 0.799173 | 113,196,032 | C | T | 3117 |
| rs34888765 | 2.99E−03 | −0.082407 | 0.931539 | 112,298,867 | C | T | 3118 |
| rs1896329 | 3.01E−03 | 0.034425 | 0.380801 | 113,841,815 | C | T | 3119 |
| chr12: 113184391 | 3.02E−03 | −0.401927 | 0.008685 | 113,184,391 | C | T | 3120 |
| rs7306365 | 3.05E−03 | −0.036345 | 0.356827 | 112,954,373 | C | T | 3121 |
| rs1634149 | 3.09E−03 | 0.035243 | 0.346159 | 114,220,633 | C | T | 3122 |
| chr12: 113637935 | 3.13E−03 | −0.103347 | 0.968071 | 113,637,935 | G | T | 3123 |
| rs61928007 | 3.19E−03 | 0.072257 | 0.922843 | 114,034,895 | C | G | 3124 |
| chr12: 113219987 | 3.20E−03 | 0.042596 | 0.192844 | 113,219,987 | A | C | 3125 |
| chr12: 113186300 | 3.30E−03 | −0.381034 | 0.008961 | 113,186,300 | C | G | 3126 |
| rs4007267 | 3.31E−03 | −0.033369 | 0.522181 | 113,170,051 | A | T | 3127 |
| rs1732589 | 3.33E−03 | −0.0355 | 0.636913 | 114,213,421 | A | G | 3128 |
| chr12: 113557673 | 3.42E−03 | −0.04215 | 0.350636 | 113,557,673 | C | T | 3129 |
| rs873619 | 3.46E−03 | 0.038 | 0.695314 | 112,433,331 | A | C | 3130 |
| rs6489971 | 3.47E−03 | −0.040327 | 0.676737 | 113,636,006 | A | C | 3131 |
| rs11067307 | 3.50E−03 | −0.035287 | 0.464531 | 113,737,049 | C | T | 3132 |
| rs4766729 | 3.62E−03 | 0.041459 | 0.190868 | 113,214,255 | C | G | 3133 |
| chr12: 112682300 | 3.63E−03 | −1.575746 | 0.998811 | 112,682,300 | G | T | 3134 |
| rs2016045 | 3.66E−03 | −0.045209 | 0.783388 | 113,244,750 | A | T | 3135 |
| chr12: 113575316 | 3.69E−03 | 0.056696 | 0.730889 | 113,575,316 | G | T | 3136 |
| rs4766748 | 3.71E−03 | 0.03379 | 0.372258 | 113,838,562 | C | T | 3137 |
| chr12: 112468389 | 3.72E−03 | 0.548984 | 0.992532 | 112,468,389 | G | T | 3138 |
| rs7484657 | 3.75E−03 | 0.033738 | 0.37233 | 113,838,184 | C | T | 3139 |
| rs1732587 | 3.77E−03 | −0.034623 | 0.66244 | 114,220,761 | A | T | 3140 |
| rs34086925 | 3.83E−03 | 0.03855 | 0.328092 | 113,631,575 | C | T | 3141 |
| chr12: 113202737 | 3.83E−03 | 0.12765 | 0.038218 | 113,202,737 | A | G | 3142 |
| chr12: 112511570 | 3.89E−03 | −0.226443 | 0.009274 | 112,511,570 | C | T | 3143 |
| chr12: 112642481 | 3.99E−03 | −0.213403 | 0.990911 | 112,642,481 | C | T | 3144 |
| rs10744808 | 4.01E−03 | 0.037575 | 0.72037 | 113,036,293 | C | T | 3145 |
| chr12: 113194781 | 4.05E−03 | 0.355325 | 0.990991 | 113,194,781 | C | T | 3146 |
| rs16945140 | 4.06E−03 | −0.065331 | 0.065867 | 114,114,382 | C | T | 3147 |
| rs11609105 | 4.12E−03 | 0.03991 | 0.705504 | 113,586,865 | A | C | 3148 |
| chr12: 113229262 | 4.12E−03 | 0.220899 | 0.020002 | 113,229,262 | A | T | 3149 |
| rs12812747 | 4.26E−03 | 0.033385 | 0.627824 | 113,859,041 | A | G | 3150 |
| chr12: 113215006 | 4.33E−03 | 0.355226 | 0.99126 | 113,215,006 | C | T | 3151 |
| rs35514224 | 4.35E−03 | −0.033321 | 0.371508 | 113,857,662 | C | T | 3152 |
| rs2384556 | 4.40E−03 | −0.033242 | 0.371686 | 113,858,464 | G | T | 3153 |
| rs1896000 | 4.41E−03 | 0.034997 | 0.688649 | 113,212,961 | C | T | 3154 |
| rs11609455 | 4.43E−03 | 0.059049 | 0.154326 | 112,323,664 | A | C | 3155 |
| rs2217171 | 4.44E−03 | −0.033195 | 0.371374 | 113,857,935 | C | T | 3156 |
| rs12313095 | 4.46E−03 | −0.03467 | 0.403323 | 112,931,243 | A | G | 3157 |
| chr12: 112372775 | 4.50E−03 | 0.07368 | 0.234878 | 112,372,775 | A | C | 3158 |
| rs1896354 | 4.52E−03 | 0.033127 | 0.629139 | 113,857,348 | A | G | 3159 |
| rs759928 | 4.53E−03 | 0.038146 | 0.229675 | 113,094,894 | A | G | 3160 |
| rs7972041 | 4.54E−03 | −0.033897 | 0.616417 | 113,854,923 | A | G | 3161 |
| chr12: 113331181 | 4.54E−03 | 0.25804 | 0.005904 | 113,331,181 | A | G | 3162 |
| rs71467958 | 4.54E−03 | 0.038122 | 0.229688 | 113,095,188 | A | T | 3163 |
| rs4766727 | 4.57E−03 | 0.036018 | 0.261963 | 113,152,155 | C | G | 3164 |
| rs4766726 | 4.57E−03 | −0.036013 | 0.738014 | 113,152,122 | A | G | 3165 |
| rs1920948 | 4.58E−03 | 0.038337 | 0.215992 | 114,181,816 | A | G | 3166 |
| rs1920949 | 4.59E−03 | 0.038333 | 0.216015 | 114,181,565 | A | G | 3167 |
| chr12: 113394183 | 4.64E−03 | −0.071758 | 0.05537 | 113,394,183 | C | T | 3168 |
| rs4767232 | 4.75E−03 | −0.037887 | 0.770088 | 113,097,369 | G | T | 3169 |
| rs1155698 | 4.76E−03 | −0.034572 | 0.69916 | 114,195,031 | A | G | 3170 |
| chr12: 113193213 | 4.78E−03 | 0.451736 | 0.011129 | 113,193,213 | G | T | 3171 |
| rs1566646 | 4.82E−03 | 0.039476 | 0.773238 | 113,601,412 | G | T | 3172 |
| rs35715782 | 4.84E−03 | −0.034197 | 0.35286 | 113,857,692 | G | T | 3173 |
| rs1896002 | 4.93E−03 | 0.031627 | 0.510878 | 113,271,748 | A | C | 3174 |
| chr12: 112418434 | 4.94E−03 | 0.122361 | 0.065966 | 112,418,434 | G | T | 3175 |
| rs7296636 | 5.03E−03 | 0.038021 | 0.217508 | 114,178,676 | C | T | 3176 |
| rs11066975 | 5.12E−03 | 0.037394 | 0.229839 | 113,100,293 | A | G | 3177 |
| chr12: 114178455 | 5.12E−03 | −0.037957 | 0.782228 | 114,178,455 | C | T | 3178 |
| rs1248054 | 5.15E−03 | 0.040369 | 0.188486 | 113,335,534 | A | G | 3179 |
| rs2016047 | 5.17E−03 | −0.043217 | 0.777817 | 113,244,799 | G | T | 3180 |
| rs61928226 | 5.23E−03 | −0.037877 | 0.781917 | 114,178,194 | A | G | 3181 |
| rs16944707 | 5.23E−03 | 0.034502 | 0.705601 | 113,829,779 | C | G | 3182 |
| rs4767277 | 5.23E−03 | −0.034494 | 0.294526 | 113,830,279 | C | T | 3183 |
| chr12: 113202956 | 5.23E−03 | −0.115504 | 0.956652 | 113,202,956 | A | G | 3184 |
| rs4767278 | 5.24E−03 | 0.034492 | 0.705492 | 113,830,361 | C | G | 3185 |
| chr12: 114167248 | 5.27E−03 | 0.129598 | 0.980372 | 114,167,248 | C | G | 3186 |
| rs2125439 | 5.30E−03 | 0.038513 | 0.789759 | 112,958,281 | C | T | 3187 |
| chr12: 112280156 | 5.33E−03 | −0.086417 | 0.94159 | 112,280,156 | A | G | 3188 |
| chr12: 114040814 | 5.33E−03 | 0.188646 | 0.986913 | 114,040,814 | A | G | 3189 |
| rs1920594 | 5.38E−03 | −0.032271 | 0.623233 | 113,189,770 | C | G | 3190 |
| rs11067515 | 5.40E−03 | 0.032674 | 0.413298 | 114,212,015 | A | G | 3191 |
| chr12: 113333272 | 5.43E−03 | 0.374321 | 0.995492 | 113,333,272 | C | T | 3192 |
| chr12: 112987754 | 5.46E−03 | −0.049663 | 0.883368 | 112,987,754 | C | G | 3193 |
| rs11066980 | 5.46E−03 | 0.036338 | 0.237664 | 113,116,454 | A | G | 3194 |
| chr12: 113482681 | 5.51E−03 | 0.378105 | 0.99759 | 113,482,681 | C | T | 3195 |
| chr12: 112374980 | 5.59E−03 | 0.410257 | 0.991056 | 112,374,980 | C | T | 3196 |
| rs1248051 | 5.61E−03 | −0.039636 | 0.811002 | 113,339,312 | C | T | 3197 |
| rs6489968 | 5.66E−03 | −0.048467 | 0.825689 | 113,623,027 | A | G | 3198 |
| chr12: 113256876 | 5.70E−03 | 0.313909 | 0.990224 | 113,256,876 | C | T | 3199 |
| rs57155932 | 5.74E−03 | −0.039149 | 0.74495 | 113,211,470 | A | G | 3200 |
| rs898078 | 5.84E−03 | −0.033843 | 0.340578 | 112,947,970 | A | G | 3201 |
| rs11067508 | 5.93E−03 | −0.038227 | 0.780732 | 114,189,295 | A | T | 3202 |
| rs11832974 | 5.95E−03 | 0.069697 | 0.898906 | 112,530,277 | A | C | 3203 |
| rs12830928 | 5.96E−03 | −0.038312 | 0.257593 | 113,737,018 | A | G | 3204 |
| rs7309382 | 6.09E−03 | −0.032197 | 0.365942 | 113,850,565 | C | T | 3205 |
| chr12: 112488054 | 6.17E−03 | −0.205131 | 0.009159 | 112,488,054 | C | G | 3206 |
| rs12426457 | 6.18E−03 | −0.052662 | 0.215451 | 112,733,093 | A | G | 3207 |
| rs1061657 | 6.28E−03 | −0.037776 | 0.251718 | 113,592,519 | C | T | 3208 |
| chr12: 113943019 | 6.34E−03 | 0.351064 | 0.991292 | 113,943,019 | C | G | 3209 |
| rs11067036 | 6.48E−03 | −0.033305 | 0.31949 | 113,231,290 | C | T | 3210 |
| rs763824 | 6.50E−03 | 0.033556 | 0.705919 | 113,053,870 | A | G | 3211 |
| rs1001562 | 6.55E−03 | 0.033528 | 0.70594 | 113,058,173 | C | T | 3212 |
| rs2114866 | 6.55E−03 | 0.033527 | 0.70594 | 113,059,717 | C | G | 3213 |
| rs1634144 | 6.55E−03 | −0.031345 | 0.605914 | 114,214,175 | C | G | 3214 |
| rs1732590 | 6.56E−03 | 0.031344 | 0.394088 | 114,214,047 | A | G | 3215 |
| rs1634145 | 6.56E−03 | −0.031342 | 0.60594 | 114,214,386 | C | T | 3216 |
| rs1732592 | 6.61E−03 | −0.031321 | 0.606159 | 114,215,112 | A | G | 3217 |
| rs1882113 | 6.61E−03 | −0.033492 | 0.294193 | 113,052,199 | C | G | 3218 |
| rs1732593 | 6.61E−03 | 0.031319 | 0.393821 | 114,215,171 | A | G | 3219 |
| rs1634146 | 6.62E−03 | −0.031318 | 0.606195 | 114,215,218 | C | T | 3220 |
| rs7305509 | 6.62E−03 | 0.033485 | 0.705969 | 113,064,459 | A | G | 3221 |
| rs971870 | 6.66E−03 | −0.033461 | 0.294242 | 113,049,846 | A | G | 3222 |
| rs7298119 | 6.70E−03 | −0.065142 | 0.934548 | 113,564,175 | G | T | 3223 |
| rs12581666 | 6.74E−03 | −0.034857 | 0.282185 | 113,836,056 | C | T | 3224 |
| rs12832663 | 6.77E−03 | 0.037564 | 0.236313 | 113,099,855 | A | G | 3225 |
| chr12: 113569675 | 6.77E−03 | −0.119198 | 0.026238 | 113,569,675 | A | G | 3226 |
| chr12: 112531394 | 6.79E−03 | 0.06463 | 0.901994 | 112,531,394 | A | C | 3227 |
| rs4767231 | 6.81E−03 | 0.033569 | 0.706467 | 113,083,996 | A | G | 3228 |
| rs4767230 | 6.82E−03 | 0.033565 | 0.70649 | 113,083,644 | G | T | 3229 |
| rs7962531 | 6.85E−03 | 0.059306 | 0.071774 | 113,553,548 | C | T | 3230 |
| chr12: 112413804 | 6.89E−03 | 0.272093 | 0.986736 | 112,413,804 | A | G | 3231 |
| chr12: 114082036 | 6.99E−03 | −0.352625 | 0.005576 | 114,082,036 | A | T | 3232 |
| rs61931193 | 7.09E−03 | −0.032284 | 0.332147 | 113,695,844 | A | G | 3233 |
| chr12: 113287380 | 7.10E−03 | 0.070946 | 0.908922 | 113,287,380 | G | T | 3234 |
| chr12: 113576118 | 7.24E−03 | 0.072505 | 0.057179 | 113,576,118 | A | T | 3235 |
| chr12: 113556660 | 7.29E−03 | −0.059512 | 0.077719 | 113,556,660 | C | T | 3236 |
| rs73392140 | 7.33E−03 | −0.044305 | 0.289885 | 113,852,371 | C | T | 3237 |
| rs1465548 | 7.35E−03 | 0.034461 | 0.257969 | 113,141,212 | C | T | 3238 |
| chr12: 113929459 | 7.37E−03 | 0.517474 | 0.989102 | 113,929,459 | C | T | 3239 |
| chr12: 113616754 | 7.38E−03 | 0.093777 | 0.046599 | 113,616,754 | C | G | 3240 |
| chr12: 113241559 | 7.43E−03 | 0.160348 | 0.985956 | 113,241,559 | A | G | 3241 |
| rs11067402 | 7.44E−03 | −0.033166 | 0.714747 | 114,010,350 | A | G | 3242 |
| rs11067403 | 7.44E−03 | 0.033166 | 0.285251 | 114,010,367 | C | T | 3243 |
| rs11067217 | 7.44E−03 | 0.064629 | 0.065575 | 113,566,577 | C | T | 3244 |
| rs10850382 | 7.46E−03 | 0.031915 | 0.665792 | 113,698,931 | C | T | 3245 |
| rs4766752 | 7.47E−03 | −0.033144 | 0.714601 | 114,009,188 | C | T | 3246 |
| rs12581446 | 7.48E−03 | 0.033173 | 0.2851 | 114,010,692 | G | T | 3247 |
| chr12: 113547915 | 7.50E−03 | −0.058665 | 0.928038 | 113,547,915 | A | C | 3248 |
| rs7953486 | 7.56E−03 | −0.033181 | 0.715168 | 114,011,130 | C | T | 3249 |
| rs7967452 | 7.63E−03 | 0.033185 | 0.284636 | 114,011,588 | C | T | 3250 |
| rs1896001 | 7.63E−03 | 0.035078 | 0.699411 | 113,213,388 | C | T | 3251 |
| rs35569752 | 7.67E−03 | 0.078963 | 0.932331 | 114,041,599 | A | T | 3252 |
| chr12: 112435543 | 7.69E−03 | 0.35346 | 0.990075 | 112,435,543 | A | T | 3253 |
| rs4767256 | 7.70E−03 | 0.031817 | 0.66586 | 113,698,467 | C | T | 3254 |
| chr12: 113568742 | 7.70E−03 | 0.064154 | 0.066246 | 113,568,742 | A | G | 3255 |
| rs73400661 | 7.77E−03 | −0.058409 | 0.92797 | 113,546,449 | C | T | 3256 |
| rs12820517 | 7.78E−03 | 0.051769 | 0.902705 | 114,039,948 | C | T | 3257 |
| rs10850383 | 7.80E−03 | 0.031722 | 0.665794 | 113,699,301 | C | T | 3258 |
| rs2042849 | 7.83E−03 | −0.058698 | 0.925109 | 113,532,778 | C | T | 3259 |
| rs34627348 | 7.85E−03 | −0.118787 | 0.026355 | 113,601,720 | A | G | 3260 |
| rs60450122 | 7.86E−03 | −0.064349 | 0.933815 | 113,570,360 | A | G | 3261 |
| chr12: 113295165 | 7.93E−03 | −0.075389 | 0.046633 | 113,295,165 | C | T | 3262 |
| rs838327 | 7.95E−03 | 0.039245 | 0.793948 | 113,423,671 | A | G | 3263 |
| chr12: 113288017 | 7.95E−03 | −0.06071 | 0.103662 | 113,288,017 | A | C | 3264 |
| rs58768025 | 7.95E−03 | −0.057966 | 0.92513 | 113,535,163 | A | G | 3265 |
| rs2295233 | 7.96E−03 | 0.070707 | 0.943444 | 113,335,449 | C | T | 3266 |
| rs1563697 | 7.97E−03 | −0.031644 | 0.401944 | 112,933,180 | C | G | 3267 |
| rs73196908 | 7.99E−03 | 0.126582 | 0.973768 | 113,571,190 | A | G | 3268 |
| rs2019085 | 8.01E−03 | −0.058319 | 0.923796 | 113,532,714 | A | G | 3269 |
| rs11067002 | 8.02E−03 | 0.030139 | 0.418644 | 113,151,429 | A | T | 3270 |
| chr12: 113534904 | 8.05E−03 | 0.058026 | 0.075019 | 113,534,904 | A | T | 3271 |
| rs7961277 | 8.08E−03 | −0.031535 | 0.54415 | 113,298,462 | C | T | 3272 |
| rs12316683 | 8.12E−03 | −0.058069 | 0.924871 | 113,534,705 | C | T | 3273 |
| rs7968359 | 8.16E−03 | 0.03262 | 0.704555 | 113,045,972 | A | G | 3274 |
| rs73400615 | 8.16E−03 | 0.058127 | 0.075212 | 113,533,516 | A | G | 3275 |
| rs11067204 | 8.17E−03 | −0.058099 | 0.924789 | 113,534,431 | A | G | 3276 |
| rs11067205 | 8.17E−03 | −0.058095 | 0.924785 | 113,534,551 | C | T | 3277 |
| rs7978298 | 8.17E−03 | 0.058187 | 0.075137 | 113,533,149 | C | T | 3278 |
| rs12581626 | 8.17E−03 | −0.0581 | 0.924787 | 113,534,238 | C | G | 3279 |
| rs73400617 | 8.18E−03 | 0.058119 | 0.075205 | 113,533,559 | A | T | 3280 |
| rs7975100 | 8.18E−03 | −0.058103 | 0.92477 | 113,533,103 | A | C | 3281 |
| rs2042850 | 8.19E−03 | −0.058104 | 0.924758 | 113,532,557 | C | T | 3282 |
| chr12: 113532061 | 8.19E−03 | 0.058109 | 0.075237 | 113,532,061 | A | G | 3283 |
| rs12310902 | 8.19E−03 | −0.05811 | 0.924759 | 113,531,655 | C | T | 3284 |
| rs12309487 | 8.19E−03 | −0.058111 | 0.924757 | 113,531,476 | C | G | 3285 |
| rs12309422 | 8.19E−03 | −0.058111 | 0.924755 | 113,531,351 | C | G | 3286 |
| rs12309421 | 8.19E−03 | −0.058111 | 0.924755 | 113,531,346 | C | G | 3287 |
| rs12322489 | 8.19E−03 | 0.058112 | 0.075245 | 113,531,324 | C | G | 3288 |
| rs2042852 | 8.20E−03 | 0.058113 | 0.075248 | 113,531,045 | A | G | 3289 |
| rs2042853 | 8.20E−03 | 0.058113 | 0.075249 | 113,530,989 | C | T | 3290 |
| rs2042854 | 8.20E−03 | −0.058115 | 0.924745 | 113,530,733 | A | T | 3291 |
| rs12308436 | 8.21E−03 | −0.05779 | 0.925744 | 113,535,701 | C | G | 3292 |
| rs2555015 | 8.22E−03 | 0.029787 | 0.512111 | 113,158,157 | C | T | 3293 |
| rs11067417 | 8.28E−03 | −0.051067 | 0.096728 | 114,036,865 | G | T | 3294 |
| chr12: 112440357 | 8.32E−03 | −0.448687 | 0.004983 | 112,440,357 | C | T | 3295 |
| rs12308556 | 8.35E−03 | −0.057681 | 0.92604 | 113,535,930 | C | T | 3296 |
| rs12321753 | 8.39E−03 | 0.057646 | 0.073877 | 113,536,075 | C | G | 3297 |
| rs12321817 | 8.39E−03 | 0.057644 | 0.073873 | 113,536,180 | A | G | 3298 |
| rs7971904 | 8.39E−03 | 0.057643 | 0.073871 | 113,536,265 | A | G | 3299 |
| rs12317207 | 8.39E−03 | 0.057643 | 0.073871 | 113,536,277 | C | T | 3300 |
| rs12321869 | 8.39E−03 | 0.057642 | 0.073876 | 113,536,247 | C | G | 3301 |
| rs7972030 | 8.39E−03 | 0.057642 | 0.073869 | 113,536,403 | A | G | 3302 |
| rs7962277 | 8.39E−03 | 0.057642 | 0.073868 | 113,536,418 | A | T | 3303 |
| rs58705700 | 8.39E−03 | 0.036464 | 0.209816 | 114,177,031 | A | G | 3304 |
| rs12304158 | 8.39E−03 | 0.051252 | 0.903402 | 114,038,802 | A | G | 3305 |
| rs12310352 | 8.40E−03 | −0.057639 | 0.926138 | 113,536,717 | C | T | 3306 |
| rs73400631 | 8.40E−03 | −0.057639 | 0.926138 | 113,536,743 | A | G | 3307 |
| rs7975422 | 8.40E−03 | 0.057637 | 0.073863 | 113,536,858 | A | G | 3308 |
| rs16944528 | 8.40E−03 | −0.057637 | 0.926144 | 113,537,014 | G | T | 3309 |
| rs7965601 | 8.40E−03 | 0.057635 | 0.073866 | 113,536,894 | C | T | 3310 |
| rs12296971 | 8.40E−03 | 0.057633 | 0.073848 | 113,537,410 | A | G | 3311 |
| rs10850437 | 8.42E−03 | −0.031684 | 0.623524 | 114,021,234 | C | G | 3312 |
| rs61161138 | 8.44E−03 | −0.035519 | 0.774319 | 114,168,695 | A | C | 3313 |
| rs12298523 | 8.46E−03 | 0.057588 | 0.073772 | 113,537,749 | A | G | 3314 |
| rs12312330 | 8.47E−03 | −0.057577 | 0.926276 | 113,537,771 | C | T | 3315 |
| rs10774736 | 8.49E−03 | −0.033715 | 0.746485 | 113,112,523 | A | G | 3316 |
| rs3741698 | 8.50E−03 | 0.035207 | 0.733873 | 113,593,606 | C | G | 3317 |
| chr12: 113258682 | 8.52E−03 | −0.080182 | 0.036126 | 113,258,682 | A | G | 3318 |
| rs11067505 | 8.54E−03 | 0.037215 | 0.204254 | 114,189,009 | C | T | 3319 |
| rs7955248 | 8.54E−03 | −0.03242 | 0.295698 | 113,045,677 | A | G | 3320 |
| chr12: 112818361 | 8.54E−03 | −0.131106 | 0.014229 | 112,818,361 | A | G | 3321 |
| rs11067212 | 8.75E−03 | 0.057461 | 0.072253 | 113,545,050 | A | G | 3322 |
| rs10774762 | 8.80E−03 | 0.03511 | 0.319358 | 113,643,185 | A | G | 3323 |
| chr12: 113227545 | 8.85E−03 | −0.068039 | 0.919637 | 113,227,545 | C | T | 3324 |
| chr12: 112418264 | 8.86E−03 | −0.046352 | 0.217254 | 112,418,264 | A | T | 3325 |
| rs1025258 | 8.93E−03 | 0.033316 | 0.708119 | 113,090,264 | G | T | 3326 |
| rs1247936 | 9.05E−03 | 0.030061 | 0.551152 | 113,192,257 | C | T | 3327 |
| rs10774797 | 9.06E−03 | −0.03369 | 0.71439 | 114,154,969 | A | G | 3328 |
| chr12: 112568294 | 9.19E−03 | 0.161695 | 0.9881 | 112,568,294 | C | T | 3329 |
| rs1465550 | 9.21E−03 | −0.033505 | 0.747326 | 113,142,420 | C | T | 3330 |
| chr12: 114040639 | 9.22E−03 | −0.043277 | 0.672204 | 114,040,639 | A | T | 3331 |
| chr12: 113564871 | 9.25E−03 | −0.057932 | 0.082923 | 113,564,871 | A | G | 3332 |
| rs11066999 | 9.26E−03 | −0.033431 | 0.748 | 113,147,716 | G | T | 3333 |
| rs567223 | 9.27E−03 | −0.030864 | 0.459182 | 113,594,954 | G | T | 3334 |
| chr12: 114268624 | 9.31E−03 | 0.058195 | 0.093782 | 114,268,624 | A | G | 3335 |
| rs741636 | 9.32E−03 | −0.03192 | 0.297948 | 113,067,581 | A | G | 3336 |
| rs759922 | 9.32E−03 | 0.031919 | 0.702051 | 113,067,387 | C | T | 3337 |
| rs11066960 | 9.37E−03 | −0.031959 | 0.297768 | 113,068,849 | A | C | 3338 |
| chr12: 113292845 | 9.37E−03 | −0.161105 | 0.013438 | 113,292,845 | A | C | 3339 |
| rs11066997 | 9.48E−03 | −0.033301 | 0.748018 | 113,145,907 | C | T | 3340 |
| rs759926 | 9.52E−03 | 0.033732 | 0.690991 | 113,091,682 | A | C | 3341 |
| rs6489900 | 9.58E−03 | 0.031155 | 0.683037 | 112,439,970 | A | G | 3342 |
| rs12814627 | 9.60E−03 | −0.037011 | 0.254051 | 113,737,131 | G | T | 3343 |
| chr12: 113637997 | 9.62E−03 | 0.035857 | 0.362046 | 113,637,997 | A | G | 3344 |
| chr12: 112336025 | 9.64E−03 | 0.128967 | 0.978733 | 112,336,025 | C | T | 3345 |
| chr12: 112986437 | 9.65E−03 | −0.04027 | 0.714329 | 112,986,437 | A | G | 3346 |
| rs11066995 | 9.75E−03 | −0.033155 | 0.747967 | 113,144,364 | C | T | 3347 |
| chr12: 113235574 | 9.88E−03 | 0.049004 | 0.108484 | 113,235,574 | A | G | 3348 |
| chr12: 113348745 | 9.89E−03 | 0.166445 | 0.986234 | 113,348,745 | A | G | 3349 |
| chr12: 112601461 | 9.96E−03 | −0.086731 | 0.030454 | 112,601,461 | A | G | 3350 |
| chr12: 113180871 | 9.97E−03 | 0.046658 | 0.184086 | 113,180,871 | A | T | 3351 |
| rs11067299 | 9.98E−03 | −0.037622 | 0.186628 | 113,718,048 | T | C | 3352 |

TABLE 15
Association results for Atrial Fibrillation in a 2 Mb region flanking rs3825214 on
chromosome 12. Shown is marker identity, p-value of the association, odds ratio (OR),
frequency of effect allele in affecteds and controls, position in NCBI Build 36, identity of effect
allele, identity of other allele, and Seq ID for the marker. It should be noted that when reported
OR values are larger than unity, the effect allele is the at-risk allele, while, when reported OR
values are less than unity, the effect allele is the protective allele, and the other allele is the at-risk
allele. The OR value for the at-risk allele is in those cases equal to 1/OR for the protective (effect) allele.

| | | | | | | effect | other | SEQ ID |
|---|---|---|---|---|---|---|---|---|
| marker | p-value | OR | freq_aff | freq_ctl | position | allele | allele | NO: |
| rs1426434 | 2.65E−05 | 1.165855 | 0.261865 | 0.233036 | 114,150,745 | A | G | 2726 |
| rs7977083 | 1.29E−04 | 0.863749 | 0.232627 | 0.258198 | 113,274,883 | A | G | 273 |
| chr12: 113294500 | 2.58E−04 | 0.858966 | 0.662152 | 0.684227 | 113,294,500 | C | T | 2727 |
| rs1895597 | 2.81E−04 | 1.152996 | 0.791465 | 0.767752 | 113,275,267 | A | T | 274 |
| rs10744823 | 3.73E−04 | 0.866607 | 0.195153 | 0.217793 | 113,282,465 | C | T | 283 |
| rs12367410 | 3.95E−04 | 1.151327 | 0.805158 | 0.782355 | 113,281,071 | C | T | 280 |
| rs3825214 | 4.04E−04 | 1.14937 | 0.802918 | 0.779916 | 113,279,826 | A | G | 279 |
| rs73197386 | 4.26E−04 | 1.16576 | 0.17378 | 0.153608 | 113,237,330 | A | G | 2728 |
| rs2113433 | 4.39E−04 | 1.147987 | 0.79927 | 0.776361 | 113,278,440 | G | T | 278 |
| rs6489956 | 4.49E−04 | 1.148198 | 0.799879 | 0.777088 | 113,276,619 | C | T | 276 |
| rs7316919 | 4.58E−04 | 0.870884 | 0.199802 | 0.222528 | 113,275,838 | A | C | 275 |
| rs2384409 | 4.91E−04 | 0.87077 | 0.198486 | 0.220953 | 113,273,861 | A | G | 271 |
| chr12: 113225621 | 5.78E−04 | 0.817561 | 0.224107 | 0.239154 | 113,225,621 | A | G | 2729 |
| rs7964303 | 6.50E−04 | 1.135015 | 0.730224 | 0.706847 | 113,298,669 | G | T | 292 |
| rs10744824 | 7.59E−04 | 1.147924 | 0.819702 | 0.798632 | 113,293,021 | A | G | 2730 |
| rs2891503 | 8.51E−04 | 0.876751 | 0.218473 | 0.240113 | 113,274,193 | A | G | 272 |
| rs2384408 | 9.30E−04 | 0.876229 | 0.195917 | 0.217123 | 113,273,733 | A | G | 2731 |
| rs7308120 | 9.36E−04 | 1.141302 | 0.804266 | 0.783091 | 113,273,429 | C | T | 270 |
| rs35345 | 1.09E−03 | 0.890762 | 0.661001 | 0.684258 | 114,096,341 | A | G | 2732 |
| rs883079 | 1.10E−03 | 0.891688 | 0.282301 | 0.305965 | 113,277,623 | C | T | 277 |
| rs2384407 | 1.12E−03 | 1.122005 | 0.719434 | 0.695949 | 113,273,609 | A | G | 2733 |
| chr12: 112769351 | 1.32E−03 | 6.782825 | 0.003493 | 0.002281 | 112,769,351 | A | G | 2734 |
| rs9634170 | 1.40E−03 | 1.112225 | 0.387022 | 0.362823 | 114,116,167 | C | T | 2735 |
| rs1946295 | 1.43E−03 | 0.892221 | 0.268597 | 0.291322 | 113,286,744 | T | C | 288 |
| rs10507248 | 1.46E−03 | 0.892684 | 0.271268 | 0.293989 | 113,281,476 | G | T | 281 |
| rs7955405 | 1.47E−03 | 0.892653 | 0.270881 | 0.293565 | 113,281,689 | A | G | 282 |
| rs1895582 | 1.74E−03 | 1.120417 | 0.74445 | 0.722488 | 113,291,418 | A | G | 291 |
| rs12828095 | 1.75E−03 | 1.109689 | 0.368846 | 0.345107 | 114,128,897 | A | G | 2736 |
| rs5015007 | 1.82E−03 | 1.137928 | 0.822068 | 0.802817 | 113,289,466 | A | T | 2737 |
| rs9669457 | 1.86E−03 | 0.88762 | 0.233433 | 0.25412 | 113,260,665 | A | G | 267 |
| rs35335 | 1.88E−03 | 1.116341 | 0.307311 | 0.285163 | 114,101,059 | A | G | 2738 |
| rs35337 | 1.91E−03 | 0.895773 | 0.691688 | 0.713771 | 114,099,853 | A | G | 2739 |
| rs35343 | 1.92E−03 | 0.895888 | 0.692564 | 0.714642 | 114,097,827 | C | T | 2740 |
| rs35340 | 1.92E−03 | 0.895962 | 0.692654 | 0.714749 | 114,098,473 | A | G | 2741 |
| rs7135659 | 1.93E−03 | 1.11786 | 0.737119 | 0.715137 | 113,286,155 | A | G | 286 |
| rs1946293 | 1.94E−03 | 1.117715 | 0.738344 | 0.716389 | 113,287,143 | A | G | 289 |
| rs35342 | 1.94E−03 | 0.895972 | 0.692231 | 0.714295 | 114,098,017 | C | T | 2742 |
| rs35336 | 1.95E−03 | 1.115896 | 0.3073 | 0.285192 | 114,100,047 | A | C | 2743 |
| rs10774793 | 1.96E−03 | 1.116102 | 0.307166 | 0.285147 | 114,095,918 | C | T | 2744 |
| chr12: 112666241 | 2.02E−03 | 0.26925 | 0.002366 | 0.004555 | 112,666,241 | C | T | 2745 |
| rs1895583 | 2.03E−03 | 0.893819 | 0.254983 | 0.276562 | 113,291,268 | A | G | 2746 |
| rs3825215 | 2.04E−03 | 1.118504 | 0.740766 | 0.719121 | 113,289,281 | C | G | 290 |
| chr12: 113141748 | 2.16E−03 | 0.318177 | 0.004575 | 0.006972 | 113,141,748 | C | G | 2747 |
| chr12: 113083912 | 2.17E−03 | 3.020985 | 0.996946 | 0.994407 | 113,083,912 | C | T | 2748 |
| chr12: 113134372 | 2.20E−03 | 3.25286 | 0.996073 | 0.993725 | 113,134,372 | C | T | 2749 |
| chr12: 113125244 | 2.22E−03 | 0.316487 | 0.003699 | 0.006107 | 113,125,244 | C | T | 2750 |
| chr12: 112993658 | 2.23E−03 | 2.814843 | 0.996198 | 0.993532 | 112,993,658 | C | T | 2751 |
| chr12: 113477891 | 2.24E−03 | 1.470474 | 0.030776 | 0.024889 | 113,477,891 | A | G | 2752 |
| rs7312625 | 2.26E−03 | 1.116665 | 0.738341 | 0.716805 | 113,284,357 | A | G | 284 |
| rs12320320 | 2.26E−03 | 1.116802 | 0.30269 | 0.281482 | 113,294,066 | A | G | 2753 |
| chr12: 112932250 | 2.29E−03 | 8.706411 | 0.998712 | 0.997449 | 112,932,250 | C | T | 2754 |
| chr12: 113491653 | 2.31E−03 | 0.68392 | 0.968727 | 0.974693 | 113,491,653 | C | T | 2755 |
| rs4767237 | 2.40E−03 | 0.896097 | 0.260178 | 0.281583 | 113,285,196 | A | G | 285 |
| rs7959293 | 2.46E−03 | 0.897838 | 0.712871 | 0.7343 | 114,127,976 | A | G | 2756 |
| chr12: 113005908 | 2.66E−03 | 0.344874 | 0.003205 | 0.005724 | 113,005,908 | A | G | 2757 |
| rs1895585 | 2.69E−03 | 0.897424 | 0.25784 | 0.279041 | 113,286,521 | A | G | 287 |
| chr12: 113394183 | 2.75E−03 | 1.227535 | 0.065477 | 0.054705 | 113,394,183 | C | T | 2758 |
| rs58905048 | 2.80E−03 | 1.181896 | 0.90144 | 0.887654 | 114,268,601 | A | G | 2759 |
| chr12: 112866561 | 3.06E−03 | 7.360565 | 0.00305 | 0.002043 | 112,866,561 | C | T | 2760 |
| rs1862909 | 3.09E−03 | 0.885315 | 0.183715 | 0.20209 | 113,271,488 | A | G | 2761 |
| chr12: 112831844 | 3.19E−03 | 9.383192 | 0.002835 | 0.001934 | 112,831,844 | A | G | 2762 |
| rs61933020 | 3.24E−03 | 2.679617 | 0.996411 | 0.993828 | 112,971,280 | A | G | 2763 |
| rs16944123 | 3.25E−03 | 0.307865 | 0.002618 | 0.004759 | 113,290,599 | A | G | 2764 |
| rs7309910 | 3.27E−03 | 0.885733 | 0.183034 | 0.20126 | 113,270,637 | C | G | 269 |
| rs10744822 | 3.30E−03 | 0.885295 | 0.18162 | 0.199725 | 113,268,423 | C | T | 2765 |
| chr12: 112733905 | 3.48E−03 | 0.401928 | 0.003418 | 0.006121 | 112,733,905 | A | G | 2766 |
| rs11067138 | 3.48E−03 | 0.869066 | 0.867706 | 0.882858 | 113,404,753 | C | T | 2767 |
| chr12: 114104206 | 3.84E−03 | 0.819712 | 0.934105 | 0.944494 | 114,104,206 | C | T | 2768 |
| rs6489955 | 3.90E−03 | 1.126761 | 0.817956 | 0.800116 | 113,269,662 | A | G | 268 |
| chr12: 113809071 | 4.05E−03 | 0.748406 | 0.030972 | 0.038438 | 113,809,071 | C | T | 2769 |
| chr12: 113274191 | 4.31E−03 | 2.08051 | 0.990062 | 0.987043 | 113,274,191 | G | T | 2770 |
| rs7965033 | 4.40E−03 | 1.126544 | 0.824799 | 0.807401 | 113,295,738 | C | T | 2771 |
| rs6489959 | 4.48E−03 | 1.149067 | 0.135291 | 0.120785 | 113,376,841 | G | T | 2772 |
| chr12: 113462864 | 4.49E−03 | 1.782382 | 0.016547 | 0.013159 | 113,462,864 | A | G | 2773 |
| rs1426435 | 4.51E−03 | 0.899508 | 0.748918 | 0.768078 | 114,145,881 | C | T | 2774 |
| chr12: 112311600 | 4.89E−03 | 0.476216 | 0.018985 | 0.021888 | 112,311,600 | G | T | 2775 |
| rs6489953 | 4.95E−03 | 0.8898 | 0.17255 | 0.189848 | 113,249,145 | C | T | 264 |
| chr12: 112371400 | 5.03E−03 | 2.206676 | 0.982518 | 0.979799 | 112,371,400 | A | G | 2776 |
| chr12: 113254039 | 5.06E−03 | 0.83844 | 0.91383 | 0.92492 | 113,254,039 | G | T | 2777 |
| rs73201491 | 5.09E−03 | 1.104512 | 0.323052 | 0.30322 | 113,278,586 | C | T | 2778 |
| rs7307520 | 5.11E−03 | 0.890202 | 0.172343 | 0.189578 | 113,249,082 | A | G | 2779 |
| rs7964836 | 5.13E−03 | 1.123283 | 0.827657 | 0.810429 | 113,251,103 | C | G | 2780 |
| rs10774752 | 5.15E−03 | 0.890287 | 0.17235 | 0.189571 | 113,252,695 | A | G | 2781 |
| rs7296611 | 5.64E−03 | 1.100744 | 0.315313 | 0.295218 | 114,131,894 | A | G | 2782 |
| chr12: 113832847 | 5.79E−03 | 0.738365 | 0.029397 | 0.035978 | 113,832,847 | A | G | 2783 |
| chr12: 114118383 | 5.88E−03 | 1.130465 | 0.670569 | 0.654805 | 114,118,383 | C | T | 2784 |
| chr12: 113700621 | 5.94E−03 | 0.770496 | 0.934354 | 0.94152 | 113,700,621 | A | T | 2785 |
| chr12: 112339167 | 6.03E−03 | 0.472898 | 0.01782 | 0.020568 | 112,339,167 | G | T | 2786 |
| chr12: 114268788 | 6.09E−03 | 0.632556 | 0.98203 | 0.98596 | 114,268,788 | A | G | 2787 |
| rs61929720 | 6.16E−03 | 1.104605 | 0.266466 | 0.247506 | 114,125,242 | A | G | 2788 |
| rs7961277 | 6.20E−03 | 1.096646 | 0.563462 | 0.54288 | 113,298,462 | C | T | 2789 |
| rs7973825 | 6.36E−03 | 1.122321 | 0.175783 | 0.159615 | 114,134,543 | A | C | 2790 |
| rs1018243 | 6.36E−03 | 1.12234 | 0.175762 | 0.159604 | 114,134,749 | C | T | 2791 |
| rs1018244 | 6.41E−03 | 0.891039 | 0.824287 | 0.840419 | 114,134,875 | G | T | 2792 |
| chr12: 113206274 | 6.51E−03 | 0.458888 | 0.987356 | 0.989643 | 113,206,274 | C | T | 2793 |
| rs8181608 | 6.55E−03 | 0.892417 | 0.168585 | 0.185187 | 113,245,966 | A | G | 259 |
| rs7966567 | 6.55E−03 | 0.892418 | 0.168608 | 0.185207 | 113,245,863 | C | T | 258 |
| rs10744818 | 6.56E−03 | 1.120546 | 0.831415 | 0.814814 | 113,246,068 | C | T | 260 |
| rs8181683 | 6.56E−03 | 0.892423 | 0.168585 | 0.185186 | 113,246,101 | C | T | 261 |
| rs8181627 | 6.56E−03 | 1.120545 | 0.831415 | 0.814814 | 113,246,147 | C | T | 262 |
| rs10744819 | 6.56E−03 | 0.892425 | 0.168585 | 0.185186 | 113,246,213 | A | G | 263 |
| rs10744820 | 6.63E−03 | 0.892561 | 0.168585 | 0.185164 | 113,252,510 | A | G | 265 |
| rs1895593 | 6.65E−03 | 1.120509 | 0.831125 | 0.81459 | 113,245,198 | A | G | 257 |
| rs1895587 | 6.68E−03 | 1.120281 | 0.831391 | 0.814833 | 113,253,912 | C | T | 266 |
| chr12: 113703732 | 6.70E−03 | 0.354315 | 0.996078 | 0.997657 | 113,703,732 | C | T | 2794 |
| chr12: 114069962 | 6.86E−03 | 0.790866 | 0.061141 | 0.069218 | 114,069,962 | A | C | 2795 |
| rs6489952 | 6.98E−03 | 1.120898 | 0.830993 | 0.814716 | 113,243,156 | A | G | 256 |
| rs4766757 | 7.10E−03 | 0.891917 | 0.824826 | 0.840705 | 114,138,010 | C | T | 2796 |
| rs7968451 | 7.12E−03 | 0.891951 | 0.82484 | 0.840713 | 114,138,256 | C | T | 2797 |
| rs11610761 | 7.51E−03 | 1.180075 | 0.933105 | 0.921917 | 114,048,070 | C | T | 2798 |
| rs11067089 | 8.01E−03 | 1.095296 | 0.374055 | 0.354598 | 113,297,009 | C | T | 2799 |
| rs4767303 | 8.25E−03 | 0.908821 | 0.731486 | 0.749861 | 114,123,986 | C | T | 2800 |
| rs10850456 | 8.29E−03 | 0.916065 | 0.621727 | 0.641789 | 114,146,153 | A | T | 2801 |
| rs11612294 | 8.35E−03 | 0.864146 | 0.172083 | 0.184306 | 113,567,048 | A | T | 2802 |
| chr12: 113403495 | 8.42E−03 | 0.653525 | 0.969878 | 0.973862 | 113,403,495 | G | T | 2803 |
| chr12: 112404356 | 8.51E−03 | 2.094628 | 0.982872 | 0.980323 | 112,404,356 | C | T | 2804 |
| rs11067054 | 8.52E−03 | 0.887388 | 0.154649 | 0.169464 | 113,256,940 | A | G | 2805 |
| rs933748 | 8.63E−03 | 0.897778 | 0.190116 | 0.206411 | 113,245,039 | C | T | 2806 |
| rs56268591 | 9.02E−03 | 1.118593 | 0.184335 | 0.16909 | 114,152,846 | A | G | 2807 |
| chr12: 114020816 | 9.07E−03 | 0.674882 | 0.019125 | 0.023769 | 114,020,816 | C | T | 2808 |
| rs11615114 | 9.15E−03 | 0.620512 | 0.014453 | 0.018256 | 113,825,200 | A | G | 2809 |
| chr12: 113762881 | 9.31E−03 | 2.835002 | 0.003721 | 0.002263 | 113,762,881 | C | T | 2810 |
| rs3782464 | 9.43E−03 | 0.917343 | 0.563025 | 0.582748 | 113,288,963 | A | C | 2811 |
| rs4767305 | 9.45E−03 | 0.913795 | 0.684973 | 0.7038 | 114,133,134 | A | T | 2812 |
| rs4767306 | 9.46E−03 | 1.094329 | 0.315013 | 0.296188 | 114,133,139 | A | G | 2813 |
| rs10774795 | 9.50E−03 | 0.917108 | 0.632862 | 0.652506 | 114,137,570 | C | T | 2814 |
| chr12: 113808331 | 9.53E−03 | 2.792127 | 0.003641 | 0.002181 | 113,808,331 | A | G | 2815 |
| chr12: 113931316 | 9.74E−03 | 0.541059 | 0.011467 | 0.014391 | 113,931,316 | C | T | 2816 |
| rs4767239 | 9.78E−03 | 1.114927 | 0.813618 | 0.797927 | 113,300,931 | C | G | 2817 |
| rs10744846 | 9.80E−03 | 0.917515 | 0.632588 | 0.65217 | 114,138,628 | A | G | 2818 |

TABLE 16
Association results for PR interval in a 2 Mb region flanking rs3807989 on
chromosome 7. Shown is marker identity, p-value of the association, magnitude of effect (in
units of fractions of standard deviations), frequency of effect allele, position in NCBI Build
36, identity of effect allele, identity of other allele, SEQ ID for the marker. It should be noted
that a positive value for an effect represents an increase in the interval conferred by the
effect allele, while a negative value for the effect represents a decrease (i.e. the allele
correlated with the negative effect is predictive of a decreased value of the measure).

| | | | | | effect | other | SEQ ID |
|---|---|---|---|---|---|---|---|
| marker | p-value | effect | freq | position | allele | allele | NO: |
| rs3807989 | 3.09E−08 | 0.062818 | 0.404775 | 115,973,477 | A | G | 197 |
| rs11773845 | 3.11E−08 | −0.062813 | 0.595254 | 115,978,537 | A | C | 200 |
| rs1997572 | 3.12E−08 | 0.062835 | 0.40479 | 115,986,064 | A | G | 570 |
| rs1997571 | 3.13E−08 | −0.062831 | 0.595195 | 115,985,857 | A | G | 571 |
| rs10953822 | 4.94E−06 | 0.059099 | 0.249799 | 115,985,702 | C | G | 572 |
| rs3815412 | 4.95E−06 | 0.059043 | 0.249855 | 115,977,929 | C | T | 199 |
| rs9886216 | 4.95E−06 | −0.059043 | 0.750145 | 115,978,933 | A | G | 573 |
| rs9885998 | 4.95E−06 | 0.059043 | 0.249855 | 115,979,048 | A | G | 574 |
| rs3757732 | 4.95E−06 | 0.059043 | 0.249851 | 115,980,941 | A | C | 204 |
| rs3757733 | 4.95E−06 | 0.059043 | 0.24985 | 115,980,965 | A | T | 205 |
| rs7804372 | 4.95E−06 | 0.059043 | 0.249845 | 115,981,464 | A | T | 206 |
| rs7789117 | 4.95E−06 | −0.059043 | 0.750155 | 115,981,620 | C | T | 575 |
| rs729949 | 4.95E−06 | 0.059043 | 0.249844 | 115,982,141 | A | G | 207 |
| rs3807990 | 4.96E−06 | −0.059054 | 0.750109 | 115,983,999 | C | T | 208 |
| rs3807992 | 4.97E−06 | 0.059056 | 0.249904 | 115,984,481 | A | G | 209 |
| rs3807994 | 4.97E−06 | 0.059057 | 0.249905 | 115,984,815 | A | G | 210 |
| rs6466588 | 4.97E−06 | −0.059057 | 0.750094 | 115,985,326 | A | T | 212 |
| rs3801995 | 4.98E−06 | −0.059037 | 0.750178 | 115,977,833 | C | T | 198 |
| rs58718486 | 1.94E−05 | 0.052305 | 0.54813 | 115,916,238 | A | T | 576 |
| rs1476451 | 2.13E−05 | 0.049701 | 0.427803 | 115,856,547 | C | G | 577 |
| rs7810505 | 2.68E−05 | 0.047107 | 0.482705 | 115,898,314 | G | T | 578 |
| rs717957 | 2.68E−05 | −0.047101 | 0.517307 | 115,900,343 | C | G | 170 |
| rs28495552 | 2.68E−05 | 0.0471 | 0.482689 | 115,900,980 | C | G | 579 |
| rs926197 | 2.68E−05 | −0.047085 | 0.51734 | 115,905,566 | A | C | 580 |
| rs6976316 | 2.69E−05 | −0.047064 | 0.517371 | 115,910,179 | A | G | 174 |
| rs6953982 | 2.77E−05 | 0.047037 | 0.482172 | 115,916,526 | C | T | 581 |
| rs10228178 | 2.86E−05 | −0.046981 | 0.518163 | 115,919,447 | A | G | 177 |
| rs6975548 | 2.87E−05 | 0.046959 | 0.483103 | 115,913,401 | C | T | 582 |
| rs55883210 | 3.12E−05 | −0.046765 | 0.520851 | 115,947,760 | A | G | 583 |
| rs6954077 | 3.14E−05 | −0.047285 | 0.512207 | 115,916,389 | A | G | 175 |
| rs28557111 | 3.15E−05 | −0.046773 | 0.515849 | 115,903,500 | A | G | 584 |
| rs7795510 | 3.16E−05 | −0.046725 | 0.520871 | 115,944,197 | C | T | 585 |
| rs2109514 | 3.21E−05 | −0.046706 | 0.520633 | 115,947,197 | A | G | 586 |
| rs12531767 | 3.23E−05 | −0.047188 | 0.508548 | 115,916,241 | A | T | 587 |
| rs3919515 | 3.36E−05 | −0.046552 | 0.52099 | 115,939,020 | C | G | 185 |
| rs2270188 | 3.37E−05 | 0.046559 | 0.478718 | 115,927,760 | G | T | 179 |
| rs2402080 | 3.39E−05 | −0.046571 | 0.519518 | 115,922,252 | C | G | 588 |
| rs2270189 | 3.50E−05 | 0.046445 | 0.478871 | 115,927,852 | A | G | 589 |
| rs10271007 | 3.75E−05 | 0.046233 | 0.478751 | 115,933,085 | A | G | 180 |
| rs4730743 | 3.75E−05 | 0.046228 | 0.478748 | 115,933,193 | A | T | 181 |
| rs4727833 | 3.85E−05 | 0.046142 | 0.478703 | 115,935,144 | C | G | 182 |
| rs6980387 | 3.90E−05 | 0.046177 | 0.496888 | 115,914,118 | A | G | 590 |
| rs6466579 | 4.94E−05 | −0.046124 | 0.519502 | 115,938,391 | C | T | 184 |
| rs13223362 | 5.77E−05 | −0.045611 | 0.527322 | 115,924,242 | A | G | 591 |
| rs3807986 | 6.57E−05 | −0.051143 | 0.742199 | 115,965,061 | A | G | 192 |
| rs768108 | 7.86E−05 | 0.044788 | 0.427769 | 115,895,894 | A | G | 169 |
| rs9649392 | 7.91E−05 | −0.04477 | 0.572245 | 115,894,622 | A | G | 592 |
| rs11769417 | 8.04E−05 | 0.044719 | 0.427687 | 115,891,014 | A | G | 593 |
| rs1007751 | 8.13E−05 | 0.044347 | 0.474752 | 115,897,291 | A | T | 594 |
| rs28494601 | 8.14E−05 | 0.04468 | 0.427654 | 115,888,497 | A | C | 595 |
| rs35421698 | 8.21E−05 | −0.044653 | 0.572386 | 115,886,677 | C | T | 596 |
| rs10235658 | 8.23E−05 | 0.044644 | 0.427605 | 115,886,143 | C | T | 597 |
| rs10464649 | 8.85E−05 | 0.044377 | 0.430074 | 115,860,803 | C | T | 165 |
| rs13225166 | 8.87E−05 | −0.044373 | 0.569915 | 115,862,687 | C | T | 598 |
| rs7781492 | 8.96E−05 | −0.044346 | 0.569811 | 115,857,211 | C | G | 164 |
| rs12706089 | 9.15E−05 | −0.044286 | 0.569784 | 115,871,603 | C | T | 166 |
| rs4727831 | 9.41E−05 | 0.044532 | 0.423964 | 115,881,219 | A | G | 168 |
| rs987791 | 1.05E−04 | −0.076116 | 0.906193 | 115,921,937 | A | G | 599 |
| rs34123906 | 1.41E−04 | −0.080728 | 0.908622 | 115,986,905 | A | G | 600 |
| chr7: 115807875 | 2.03E−04 | 0.074809 | 0.85067 | 115,807,875 | A | T | 601 |
| rs55994026 | 2.20E−04 | −0.061232 | 0.346153 | 115,931,201 | A | G | 602 |
| rs12540549 | 2.26E−04 | −0.049432 | 0.529532 | 115,982,768 | A | G | 603 |
| chr7: 115982911 | 2.69E−04 | 0.067158 | 0.104774 | 115,982,911 | A | G | 604 |
| rs10261304 | 2.81E−04 | −0.04743 | 0.391013 | 115,943,213 | A | C | 605 |
| rs2056865 | 3.26E−04 | −0.043456 | 0.688606 | 116,007,768 | A | G | 231 |
| rs6978354 | 3.30E−04 | −0.045711 | 0.682245 | 116,013,658 | A | G | 236 |
| rs6955302 | 3.35E−04 | −0.044553 | 0.698159 | 116,012,940 | C | T | 235 |
| rs3807993 | 3.63E−04 | 0.062551 | 0.114223 | 115,984,808 | A | T | 606 |
| rs1049334 | 3.63E−04 | 0.062551 | 0.114226 | 115,987,616 | A | G | 607 |
| rs62469031 | 3.64E−04 | −0.062526 | 0.885797 | 115,983,077 | A | G | 608 |
| rs3807995 | 3.64E−04 | −0.062546 | 0.885775 | 115,985,212 | C | T | 609 |
| rs73206045 | 3.75E−04 | 0.066159 | 0.102339 | 115,890,043 | A | C | 610 |
| rs13246051 | 3.76E−04 | 0.043397 | 0.31462 | 116,010,773 | A | T | 611 |
| rs3801994 | 3.92E−04 | 0.062198 | 0.114253 | 115,977,705 | A | G | 612 |
| rs3801993 | 3.95E−04 | −0.06217 | 0.885746 | 115,977,618 | C | G | 613 |
| rs34678019 | 3.95E−04 | −0.062165 | 0.885746 | 115,976,726 | A | G | 614 |
| rs13242816 | 3.95E−04 | −0.062164 | 0.885746 | 115,976,612 | C | T | 615 |
| rs13243017 | 3.95E−04 | 0.062164 | 0.114254 | 115,976,607 | A | G | 616 |
| rs2052105 | 3.96E−04 | 0.062156 | 0.114254 | 115,975,215 | C | T | 617 |
| rs12668226 | 3.96E−04 | −0.062155 | 0.885746 | 115,974,926 | A | C | 618 |
| rs12672038 | 3.97E−04 | 0.062151 | 0.114254 | 115,974,342 | A | G | 619 |
| rs34979439 | 3.99E−04 | 0.069086 | 0.098271 | 115,833,967 | A | G | 620 |
| rs1858810 | 4.07E−04 | −0.040458 | 0.576707 | 115,846,095 | A | G | 163 |
| rs62471192 | 4.66E−04 | 0.06472 | 0.105528 | 115,891,690 | A | G | 621 |
| rs35866921 | 4.76E−04 | 0.065489 | 0.102714 | 115,891,694 | A | G | 622 |
| chr7: 115931213 | 4.83E−04 | 0.047327 | 0.48455 | 115,931,213 | A | G | 623 |
| rs34916272 | 4.84E−04 | 0.065591 | 0.102369 | 115,913,521 | C | T | 624 |
| rs73208106 | 4.90E−04 | 0.061876 | 0.112718 | 115,960,499 | A | G | 625 |
| rs13233553 | 4.91E−04 | −0.061842 | 0.887548 | 115,969,468 | C | G | 626 |
| rs12669209 | 4.95E−04 | 0.061872 | 0.112402 | 115,964,210 | A | G | 627 |
| rs3807988 | 4.97E−04 | −0.061833 | 0.887631 | 115,967,971 | A | C | 628 |
| rs55701446 | 5.13E−04 | −0.04934 | 0.81048 | 115,937,313 | G | T | 629 |
| rs56063232 | 5.15E−04 | −0.061883 | 0.888086 | 115,960,044 | G | T | 630 |
| rs55930206 | 5.21E−04 | −0.061927 | 0.888286 | 115,960,086 | C | T | 631 |
| rs3779513 | 5.22E−04 | 0.062018 | 0.111488 | 115,966,228 | C | T | 632 |
| rs36200236 | 5.35E−04 | 0.061735 | 0.110666 | 115,907,135 | C | T | 633 |
| rs34325296 | 5.41E−04 | −0.061425 | 0.888893 | 115,909,645 | C | T | 634 |
| rs35595472 | 5.41E−04 | 0.061425 | 0.111107 | 115,909,542 | A | G | 635 |
| rs35800921 | 5.41E−04 | 0.061424 | 0.111105 | 115,911,277 | A | G | 636 |
| rs6979182 | 5.41E−04 | 0.061424 | 0.111113 | 115,906,581 | G | T | 637 |
| rs34424131 | 5.41E−04 | 0.061424 | 0.111113 | 115,906,238 | A | G | 638 |
| rs36011826 | 5.41E−04 | 0.061424 | 0.111115 | 115,905,572 | A | G | 639 |
| rs35538349 | 5.42E−04 | −0.061423 | 0.888885 | 115,905,132 | C | T | 640 |
| rs35037267 | 5.42E−04 | −0.061423 | 0.888884 | 115,904,823 | A | T | 641 |
| rs56298491 | 5.42E−04 | −0.061423 | 0.888883 | 115,903,923 | C | T | 642 |
| rs34870046 | 5.42E−04 | 0.061422 | 0.111119 | 115,903,190 | A | G | 643 |
| rs34752288 | 5.42E−04 | 0.061422 | 0.11112 | 115,902,178 | G | T | 644 |
| rs62468973 | 5.42E−04 | −0.061423 | 0.888898 | 115,913,070 | G | T | 645 |
| rs17138714 | 5.42E−04 | −0.061421 | 0.888879 | 115,901,510 | C | T | 646 |
| rs34234085 | 5.42E−04 | 0.061421 | 0.111122 | 115,901,302 | A | G | 647 |
| rs13235720 | 5.42E−04 | 0.061421 | 0.111122 | 115,900,899 | C | T | 648 |
| rs13247987 | 5.42E−04 | −0.061421 | 0.888877 | 115,900,580 | A | C | 649 |
| rs62468976 | 5.42E−04 | −0.061422 | 0.8889 | 115,914,127 | C | G | 650 |
| rs62471202 | 5.42E−04 | 0.06142 | 0.111124 | 115,899,798 | A | G | 651 |
| rs62468978 | 5.42E−04 | −0.061422 | 0.8889 | 115,914,335 | A | G | 652 |
| rs55977427 | 5.42E−04 | −0.061419 | 0.888873 | 115,898,004 | C | T | 653 |
| rs7809755 | 5.42E−04 | −0.061419 | 0.888873 | 115,897,968 | C | T | 654 |
| rs55833477 | 5.42E−04 | −0.061418 | 0.888873 | 115,897,611 | C | G | 655 |
| rs35800776 | 5.42E−04 | 0.061418 | 0.111128 | 115,897,305 | A | C | 656 |
| rs67652573 | 5.42E−04 | 0.061418 | 0.111128 | 115,897,194 | C | T | 657 |
| rs35364754 | 5.42E−04 | 0.061418 | 0.111128 | 115,896,952 | G | T | 658 |
| rs2188243 | 5.42E−04 | 0.061418 | 0.111129 | 115,896,529 | A | G | 659 |
| rs17138708 | 5.42E−04 | 0.061417 | 0.111129 | 115,896,298 | G | T | 660 |
| rs17138706 | 5.42E−04 | −0.061417 | 0.888871 | 115,896,255 | A | G | 661 |
| rs56275709 | 5.42E−04 | −0.061417 | 0.88887 | 115,895,725 | A | G | 662 |
| rs62471198 | 5.42E−04 | 0.061416 | 0.111131 | 115,895,199 | C | T | 663 |
| rs34228114 | 5.42E−04 | −0.061415 | 0.888867 | 115,894,222 | C | T | 664 |
| rs35210394 | 5.42E−04 | −0.061413 | 0.888864 | 115,892,147 | C | G | 665 |
| rs35505552 | 5.42E−04 | 0.061413 | 0.111136 | 115,892,103 | A | T | 666 |
| rs12706090 | 5.42E−04 | −0.061412 | 0.888863 | 115,891,281 | C | T | 667 |
| rs56250718 | 5.42E−04 | −0.061412 | 0.888862 | 115,890,539 | A | G | 668 |
| rs55803834 | 5.42E−04 | −0.061411 | 0.888862 | 115,890,377 | C | T | 669 |
| rs4292624 | 5.42E−04 | −0.061411 | 0.888861 | 115,889,960 | A | C | 670 |
| rs62471189 | 5.43E−04 | 0.06141 | 0.111141 | 115,888,824 | A | G | 671 |
| rs56161415 | 5.43E−04 | −0.061409 | 0.888858 | 115,888,080 | G | T | 672 |
| rs55726675 | 5.43E−04 | 0.061408 | 0.111142 | 115,887,934 | A | G | 673 |
| rs62471187 | 5.43E−04 | 0.061408 | 0.111142 | 115,887,620 | A | G | 674 |
| rs13247015 | 5.43E−04 | −0.061401 | 0.888851 | 115,882,859 | A | C | 675 |
| rs62471184 | 5.44E−04 | 0.061394 | 0.111155 | 115,877,536 | A | G | 676 |
| rs67982517 | 5.44E−04 | −0.061388 | 0.888837 | 115,862,599 | A | G | 677 |
| rs34645128 | 5.44E−04 | 0.061388 | 0.111163 | 115,865,618 | A | T | 678 |
| rs34205980 | 5.44E−04 | 0.061388 | 0.111163 | 115,865,642 | A | T | 679 |
| rs56180538 | 5.44E−04 | −0.061388 | 0.888837 | 115,866,947 | C | T | 680 |
| rs12672717 | 5.49E−04 | 0.061408 | 0.110978 | 115,915,506 | A | G | 681 |
| rs2402078 | 5.53E−04 | −0.0614 | 0.889075 | 115,915,884 | A | C | 682 |
| rs2402079 | 5.53E−04 | 0.061399 | 0.110923 | 115,915,906 | A | T | 683 |
| rs66985071 | 5.63E−04 | −0.061368 | 0.889216 | 115,917,669 | C | T | 684 |
| rs12706091 | 5.65E−04 | 0.06136 | 0.110757 | 115,918,019 | C | G | 685 |
| rs3807987 | 5.75E−04 | 0.062025 | 0.110326 | 115,967,070 | A | G | 686 |
| rs34877271 | 5.78E−04 | 0.064553 | 0.10362 | 115,837,103 | A | G | 687 |
| rs2109517 | 5.81E−04 | −0.041996 | 0.69812 | 116,004,893 | A | G | 230 |
| rs34295495 | 5.86E−04 | 0.06102 | 0.111328 | 115,855,502 | A | C | 688 |
| rs1018859 | 6.12E−04 | −0.060787 | 0.888586 | 115,854,583 | C | T | 689 |
| rs2191498 | 6.31E−04 | 0.060975 | 0.110166 | 115,925,805 | T | C | 690 |
| rs10270072 | 6.32E−04 | −0.041866 | 0.698169 | 115,999,710 | A | C | 691 |
| rs36200748 | 6.46E−04 | −0.060604 | 0.888019 | 115,839,863 | C | T | 692 |
| rs17138747 | 6.49E−04 | 0.060866 | 0.109893 | 115,919,906 | A | G | 693 |
| rs36199027 | 6.54E−04 | 0.060552 | 0.112059 | 115,837,771 | C | T | 694 |
| rs56010123 | 6.54E−04 | −0.061707 | 0.890566 | 115,960,282 | C | T | 695 |
| rs35184471 | 6.55E−04 | 0.060827 | 0.109847 | 115,920,482 | A | G | 696 |
| rs9770220 | 6.62E−04 | 0.039194 | 0.41894 | 115,837,829 | A | G | 697 |
| rs17138750 | 6.63E−04 | −0.060765 | 0.890221 | 115,921,406 | A | G | 698 |
| rs987790 | 6.68E−04 | 0.060734 | 0.109745 | 115,921,869 | A | T | 699 |
| rs56382464 | 6.71E−04 | −0.060458 | 0.887669 | 115,828,064 | C | G | 700 |
| rs12706096 | 6.71E−04 | −0.041916 | 0.701462 | 116,001,878 | C | T | 701 |
| rs36125850 | 6.73E−04 | −0.060456 | 0.887609 | 115,827,154 | C | T | 702 |
| rs2024209 | 6.75E−04 | 0.060684 | 0.109693 | 115,922,572 | C | T | 703 |
| rs6968230 | 6.92E−04 | −0.060555 | 0.890433 | 115,924,285 | G | T | 704 |
| rs13226307 | 6.97E−04 | −0.06052 | 0.890465 | 115,924,721 | A | T | 705 |
| rs17138767 | 6.99E−04 | −0.060505 | 0.89048 | 115,924,913 | A | G | 706 |
| rs12669740 | 7.10E−04 | 0.060426 | 0.10945 | 115,925,812 | C | T | 707 |
| rs2191499 | 7.10E−04 | −0.060424 | 0.89055 | 115,925,835 | C | T | 708 |
| rs2191501 | 7.15E−04 | −0.060396 | 0.890561 | 115,926,067 | G | T | 709 |
| rs56001716 | 7.36E−04 | 0.0611 | 0.111903 | 115,887,896 | C | T | 710 |
| rs13235183 | 7.46E−04 | −0.060199 | 0.890629 | 115,927,385 | G | T | 711 |
| rs34936262 | 7.53E−04 | 0.060262 | 0.109657 | 115,947,258 | A | G | 712 |
| rs34652384 | 7.55E−04 | 0.060245 | 0.109635 | 115,944,247 | C | T | 713 |
| chr7: 115893706 | 7.55E−04 | 0.063481 | 0.113198 | 115,893,706 | A | T | 714 |
| rs35515812 | 7.56E−04 | −0.060232 | 0.890381 | 115,943,268 | C | G | 715 |
| rs7801950 | 7.58E−04 | −0.060214 | 0.890403 | 115,942,019 | C | T | 716 |
| rs34082946 | 7.59E−04 | 0.060206 | 0.109592 | 115,941,549 | A | G | 717 |
| rs35062002 | 7.65E−04 | 0.060083 | 0.109332 | 115,929,694 | A | G | 718 |
| rs7796429 | 7.74E−04 | 0.060024 | 0.109312 | 115,931,605 | C | T | 719 |
| chr7: 115982913 | 7.75E−04 | 0.061691 | 0.113359 | 115,982,913 | A | G | 720 |
| rs56309428 | 7.88E−04 | −0.059955 | 0.890675 | 115,937,373 | G | T | 721 |
| chr7: 116458967 | 7.92E−04 | −0.095774 | 0.05223 | 116,458,967 | C | T | 722 |
| rs2052106 | 8.00E−04 | 0.041662 | 0.284336 | 115,993,901 | A | G | 225 |
| rs55691296 | 8.38E−04 | −0.041453 | 0.716271 | 115,991,326 | A | G | 723 |
| rs13221364 | 8.74E−04 | −0.058173 | 0.88042 | 115,870,780 | C | T | 724 |
| rs12671606 | 1.01E−03 | 0.037543 | 0.44554 | 115,903,077 | G | T | 725 |
| rs7778733 | 1.18E−03 | 0.041339 | 0.724607 | 115,956,679 | A | C | 726 |
| rs10279056 | 1.19E−03 | 0.036248 | 0.457602 | 115,633,993 | A | G | 727 |
| chr7: 115941774 | 1.33E−03 | 0.074724 | 0.8607 | 115,941,774 | C | T | 728 |
| rs17587314 | 1.37E−03 | 0.046041 | 0.183577 | 115,919,879 | A | T | 729 |
| rs17138749 | 1.38E−03 | −0.046012 | 0.816486 | 115,920,334 | A | C | 730 |
| rs35774544 | 1.57E−03 | −0.122981 | 0.975299 | 115,906,674 | A | G | 731 |
| rs7811851 | 1.74E−03 | −0.036887 | 0.659672 | 115,943,967 | A | T | 732 |
| rs6466580 | 1.76E−03 | 0.036835 | 0.340258 | 115,941,962 | C | T | 733 |
| rs17516287 | 1.77E−03 | −0.036814 | 0.659726 | 115,941,422 | C | G | 734 |
| rs17588172 | 1.77E−03 | 0.036809 | 0.340226 | 115,941,251 | G | T | 735 |
| rs9640770 | 1.77E−03 | 0.039214 | 0.271981 | 115,897,149 | A | C | 736 |
| chr7: 115989137 | 1.81E−03 | 0.040261 | 0.417758 | 115,989,137 | G | T | 737 |
| rs28485381 | 1.82E−03 | −0.039127 | 0.727967 | 115,899,686 | C | T | 738 |
| rs12670840 | 1.89E−03 | −0.03893 | 0.727969 | 115,912,408 | C | T | 739 |
| rs1052990 | 1.92E−03 | −0.036714 | 0.660484 | 115,935,606 | T | G | 740 |
| rs12530912 | 2.02E−03 | −0.038735 | 0.728385 | 115,914,765 | T | G | 741 |
| rs73206052 | 2.06E−03 | −0.055838 | 0.893583 | 115,904,292 | G | T | 742 |
| rs28587043 | 2.08E−03 | 0.036249 | 0.339357 | 115,932,932 | A | G | 743 |
| rs12668473 | 2.08E−03 | −0.036247 | 0.660649 | 115,932,875 | C | T | 744 |
| chr7: 115588710 | 2.10E−03 | 0.067298 | 0.888747 | 115,588,710 | A | G | 745 |
| rs13229461 | 2.13E−03 | −0.036187 | 0.660839 | 115,931,038 | C | T | 746 |
| rs11980719 | 2.16E−03 | 0.036151 | 0.339058 | 115,930,044 | A | T | 747 |
| rs11983865 | 2.18E−03 | −0.036138 | 0.660973 | 115,929,698 | A | G | 748 |
| rs11983864 | 2.18E−03 | −0.036138 | 0.660973 | 115,929,696 | A | C | 749 |
| rs3779511 | 2.20E−03 | 0.036111 | 0.338952 | 115,929,014 | G | T | 750 |
| chr7: 115998079 | 2.23E−03 | −0.109345 | 0.039593 | 115,998,079 | C | T | 751 |
| chr7: 115950414 | 2.28E−03 | 0.075698 | 0.068932 | 115,950,414 | A | G | 752 |
| rs28613932 | 2.33E−03 | −0.034259 | 0.551984 | 115,633,516 | A | G | 753 |
| rs71564558 | 2.44E−03 | 0.125406 | 0.022055 | 115,895,488 | A | G | 754 |
| chr7: 115945331 | 2.48E−03 | 0.126766 | 0.021133 | 115,945,331 | A | G | 755 |
| rs13227232 | 2.49E−03 | −0.126581 | 0.978787 | 115,925,428 | C | G | 756 |
| rs7788491 | 2.53E−03 | 0.035248 | 0.395519 | 115,837,213 | C | T | 757 |
| rs35377909 | 2.64E−03 | −0.12459 | 0.978144 | 115,920,143 | A | G | 758 |
| rs13224179 | 2.67E−03 | 0.124482 | 0.021772 | 115,949,207 | A | C | 759 |
| rs13240764 | 2.74E−03 | 0.033696 | 0.440331 | 115,632,950 | C | T | 760 |
| rs62468956 | 2.74E−03 | 0.035483 | 0.386359 | 115,837,225 | A | G | 761 |
| rs71564555 | 3.00E−03 | 0.123686 | 0.022015 | 115,822,449 | C | T | 762 |
| chr7: 116043056 | 3.16E−03 | −0.198849 | 0.016856 | 116,043,056 | C | T | 763 |
| rs57669655 | 3.20E−03 | 0.035144 | 0.383399 | 115,837,228 | A | C | 764 |
| rs38860 | 3.23E−03 | 0.082706 | 0.046691 | 116,182,493 | C | T | 765 |
| rs193687 | 3.26E−03 | −0.082847 | 0.953927 | 116,219,242 | C | T | 766 |
| chr7: 116504377 | 3.37E−03 | −0.750072 | 0.997903 | 116,504,377 | A | G | 767 |
| chr7: 115950425 | 3.59E−03 | −0.039049 | 0.372423 | 115,950,425 | A | G | 768 |
| rs1049337 | 3.65E−03 | 0.037314 | 0.741937 | 115,987,823 | C | T | 216 |
| rs7788470 | 3.79E−03 | 0.036381 | 0.354316 | 115,837,158 | C | T | 769 |
| rs41788 | 3.81E−03 | −0.082849 | 0.961073 | 116,278,450 | A | C | 770 |
| rs13243307 | 3.81E−03 | 0.123656 | 0.021551 | 115,540,517 | A | C | 771 |
| rs71564553 | 3.91E−03 | 0.119675 | 0.02339 | 115,780,758 | A | G | 772 |
| chr7: 115786440 | 3.93E−03 | −0.119416 | 0.976671 | 115,786,440 | A | G | 773 |
| chr7: 115619988 | 3.98E−03 | 0.121874 | 0.022402 | 115,619,988 | A | G | 774 |
| chr7: 116633074 | 4.24E−03 | −0.124755 | 0.976069 | 116,633,074 | A | C | 775 |
| chr7: 115069465 | 4.49E−03 | −0.055797 | 0.111166 | 115,069,465 | A | G | 776 |
| rs41796 | 4.75E−03 | −0.081095 | 0.962321 | 116,297,475 | C | T | 777 |
| chr7: 115869733 | 4.81E−03 | 0.042863 | 0.678637 | 115,869,733 | C | T | 778 |
| rs437 | 4.94E−03 | 0.033163 | 0.343516 | 116,084,132 | C | T | 779 |
| chr7: 116078942 | 5.31E−03 | −0.528536 | 0.997313 | 116,078,942 | A | C | 780 |
| chr7: 115982816 | 5.34E−03 | −0.038456 | 0.655241 | 115,982,816 | A | G | 781 |
| chr7: 115672912 | 5.76E−03 | 0.100123 | 0.966158 | 115,672,912 | C | G | 782 |
| chr7: 116726785 | 6.09E−03 | 0.14397 | 0.017624 | 116,726,785 | C | T | 783 |
| rs6972100 | 6.25E−03 | 0.083117 | 0.04035 | 115,453,169 | A | C | 784 |
| chr7: 115466739 | 6.30E−03 | 0.069172 | 0.074031 | 115,466,739 | C | G | 785 |
| rs12672236 | 6.86E−03 | −0.046099 | 0.874044 | 115,898,605 | C | T | 786 |
| rs11764503 | 6.96E−03 | −0.031646 | 0.635626 | 116,535,234 | C | T | 787 |
| rs6466590 | 7.00E−03 | −0.04474 | 0.864214 | 116,012,071 | A | G | 788 |
| chr7: 115893708 | 7.17E−03 | −0.038615 | 0.694191 | 115,893,708 | A | T | 789 |
| chr7: 115682046 | 7.22E−03 | −0.045384 | 0.842724 | 115,682,046 | A | G | 790 |
| chr7: 116207887 | 7.55E−03 | 0.214222 | 0.992604 | 116,207,887 | C | T | 791 |
| chr7: 115636229 | 7.71E−03 | −0.070345 | 0.068434 | 115,636,229 | A | T | 792 |
| chr7: 115677922 | 7.75E−03 | −0.097729 | 0.031561 | 115,677,922 | G | T | 793 |
| chr7: 115950410 | 7.84E−03 | −0.044993 | 0.225192 | 115,950,410 | A | G | 794 |
| chr7: 115612346 | 7.98E−03 | −0.110753 | 0.973984 | 115,612,346 | G | T | 795 |
| chr7: 116394850 | 8.39E−03 | 0.072819 | 0.946993 | 116,394,850 | A | G | 796 |
| chr7: 116379652 | 8.43E−03 | −0.072633 | 0.053849 | 116,379,652 | C | T | 797 |
| chr7: 116075041 | 8.61E−03 | 0.524549 | 0.002601 | 116,075,041 | C | T | 798 |
| rs62470782 | 8.62E−03 | −0.103333 | 0.971191 | 116,318,743 | C | T | 799 |
| chr7: 115356605 | 8.94E−03 | 0.178653 | 0.985919 | 115,356,605 | C | T | 800 |
| rs4428611 | 9.07E−03 | −0.047607 | 0.891208 | 115,639,217 | A | G | 801 |
| chr7: 115082501 | 9.13E−03 | −0.053039 | 0.102469 | 115,082,501 | C | T | 802 |
| rs56327526 | 9.20E−03 | 0.065348 | 0.920229 | 115,190,808 | A | G | 803 |
| chr7: 115285478 | 9.26E−03 | 0.13353 | 0.964525 | 115,285,478 | A | T | 804 |
| chr7: 116623036 | 9.31E−03 | 0.130759 | 0.015401 | 116,623,036 | A | T | 805 |
| chr7: 115908127 | 9.50E−03 | 0.042879 | 0.794818 | 115,908,127 | G | T | 806 |
| rs2191502 | 9.51E−03 | 0.043602 | 0.130565 | 116,005,322 | T | C | 807 |

TABLE 17
Association results for Atrial Fibrillation in a 2 Mb region flanking rs3807989 on
chromosome 7. Shown is marker identity, p-value of the association, odds ratio (OR), frequency
of effect allele in affecteds and controls, position in NCBI Build 36, identity of effect allele,
identity of other allele, and Seq ID for the marker. It should be noted that when reported OR
values are larger than unity, the effect allele is the at-risk allele, while, when reported OR values
are less than unity, the effect allele is the protective allele, and the other allele is the at-risk allele.
The OR value for the at-risk allele is in those cases equal to 1/OR for the protective (effect) allele.

| | | | | | | | other | SEQ ID |
|---|---|---|---|---|---|---|---|---|
| marker | P-value | OR | freq_aff | freq_ctl | position | eff all | all | NO: |
| chr7: 116669969 | 0.000103865 | 0.431109 | 0.006424 | 0.011496 | 116,669,969 | C | T | 536 |
| chr7: 116605565 | 0.00103852 | 2.507544 | 0.995069 | 0.991686 | 116,605,565 | A | G | 537 |
| chr7: 116568306 | 0.00122287 | 2.487908 | 0.995156 | 0.991839 | 116,568,306 | C | G | 538 |
| chr7: 116656715 | 0.00165339 | 1.339189 | 0.96116 | 0.952258 | 116,656,715 | A | T | 539 |
| chr7: 116195414 | 0.00234327 | 0.184039 | 0.000932 | 0.002721 | 116,195,414 | A | G | 540 |
| chr7: 116624142 | 0.0026901 | 0.786535 | 0.054158 | 0.06394 | 116,624,142 | C | T | 541 |
| chr7: 116688711 | 0.00445245 | 0.196572 | 0.001645 | 0.003297 | 116,688,711 | A | T | 542 |
| chr7: 116636691 | 0.00458699 | 1.576514 | 0.985833 | 0.981066 | 116,636,691 | A | G | 543 |
| rs10277181 | 0.0055339 | 0.865636 | 0.883742 | 0.897048 | 116,724,756 | A | G | 544 |
| chr7: 116727904 | 0.00561554 | 1.136674 | 0.153842 | 0.138876 | 116,727,904 | A | T | 545 |
| chr7: 116220710 | 0.00567687 | 1.872547 | 0.99529 | 0.991888 | 116,220,710 | C | T | 546 |
| chr7: 116360303 | 0.00587002 | 1.70985 | 0.99351 | 0.989624 | 116,360,303 | C | T | 547 |
| rs10227271 | 0.00593221 | 0.879954 | 0.848899 | 0.863705 | 116,727,284 | A | G | 548 |
| rs12540549 | 0.00597803 | 1.111004 | 0.546679 | 0.52841 | 115,982,768 | A | G | 549 |
| rs61658621 | 0.0065635 | 0.860958 | 0.151562 | 0.164285 | 115,247,528 | A | G | 550 |
| chr7: 116071363 | 0.00680798 | 1.570063 | 0.98624 | 0.981912 | 116,071,363 | G | T | 551 |
| chr7: 116487098 | 0.00693011 | 0.75238 | 0.964433 | 0.970715 | 116,487,098 | A | C | 552 |
| rs10487362 | 0.00740228 | 1.150262 | 0.112521 | 0.099771 | 116,716,301 | A | G | 553 |
| chr7: 116083983 | 0.00746422 | 1.561322 | 0.985958 | 0.98167 | 116,083,983 | C | T | 554 |
| chr7: 115195734 | 0.00764368 | 0.847674 | 0.211875 | 0.222885 | 115,195,734 | G | T | 555 |
| rs2301633 | 0.0077128 | 1.131493 | 0.150258 | 0.13587 | 116,725,256 | C | T | 556 |
| chr7: 116026350 | 0.00784374 | 0.479735 | 0.004731 | 0.007423 | 116,026,350 | G | T | 557 |
| chr7: 115702904 | 0.00840518 | 0.576684 | 0.009924 | 0.01332 | 115,702,904 | A | C | 558 |
| chr7: 115797367 | 0.00847545 | 1.755545 | 0.992686 | 0.98933 | 115,797,367 | C | T | 559 |
| chr7: 115466016 | 0.00881763 | 1.68701 | 0.98832 | 0.984781 | 115,466,016 | A | T | 560 |
| chr7: 115799374 | 0.0091131 | 1.724806 | 0.992252 | 0.988857 | 115,799,374 | C | T | 561 |
| rs3779545 | 0.00918261 | 0.872595 | 0.888709 | 0.901169 | 116,722,799 | A | G | 562 |
| rs17139625 | 0.00930474 | 1.145687 | 0.110947 | 0.098522 | 116,722,714 | A | G | 563 |
| chr7: 115647806 | 0.00946261 | 0.319209 | 0.005014 | 0.006628 | 115,647,806 | A | G | 564 |
| chr7: 116241296 | 0.00969249 | 0.80707 | 0.936658 | 0.944422 | 116,241,296 | C | T | 565 |
| chr7: 116195913 | 0.00975066 | 1.263714 | 0.968856 | 0.961396 | 116,195,913 | C | T | 566 |
| rs3779546 | 0.00980563 | 1.144476 | 0.110457 | 0.098096 | 116,721,436 | A | G | 567 |
| chr7: 116238188 | 0.00983466 | 1.459092 | 0.985191 | 0.980488 | 116,238,188 | A | C | 568 |
| chr7: 116183965 | 0.00990869 | 2.097491 | 0.997633 | 0.995072 | 116,183,965 | A | G | 569 |

TABLE 18
Association results for PR interval in a 2 Mb region flanking rs6795970 on
chromosome 3. Shown is marker identity, p-value of the association, magnitude of effect (in
units of fractions of standard deviations), frequency of effect allele, position in NCBI Build 36,
identity of effect allele, identity of other allele, SEQ ID for the marker. It should be noted that a
positive value for an effect represents an increase in the interval conferred by the effect allele,
while a negative value for the effect represents a decrease (i.e. the allele correlated with the
negative effect is predictive of a decreased value of the measure).

| | | | | | effect | other | |
|---|---|---|---|---|---|---|---|
| marker | p-value | effect | freq | position | allele | allele | SEQ ID NO: |
| rs7433306 | 2.57E−32 | 0.138568 | 0.362944 | 38,745,643 | C | G | 15 |
| rs6795970 | 3.03E−32 | 0.138971 | 0.36095 | 38,741,679 | A | G | 13 |
| rs6800541 | 4.44E−32 | 0.13716 | 0.363841 | 38,749,836 | C | T | 18 |
| rs6783110 | 5.72E−32 | 0.138292 | 0.359181 | 38,727,939 | A | G | 1527 |
| rs11924846 | 6.13E−32 | 0.138135 | 0.359263 | 38,731,570 | C | T | 5 |
| rs9820042 | 6.43E−32 | 0.136896 | 0.363935 | 38,754,120 | C | T | 1528 |
| rs9844577 | 7.75E−32 | −0.136961 | 0.637157 | 38,763,732 | A | C | 1529 |
| rs4076737 | 9.43E−32 | 0.13723 | 0.359175 | 38,739,786 | G | T | 10 |
| rs6599250 | 1.03E−31 | −0.136503 | 0.635957 | 38,759,033 | C | T | 20 |
| rs6599254 | 1.13E−31 | 0.13662 | 0.36327 | 38,770,559 | A | G | 23 |
| rs6599251 | 5.22E−31 | 0.133801 | 0.383888 | 38,760,813 | G | T | 21 |
| rs6790396 | 5.27E−31 | 0.134898 | 0.369591 | 38,746,929 | C | G | 17 |
| rs6801957 | 1.02E−30 | −0.134169 | 0.635242 | 38,742,319 | C | T | 14 |
| rs10428132 | 1.10E−30 | −0.133513 | 0.629702 | 38,752,558 | G | T | 1530 |
| rs7433723 | 1.23E−30 | −0.133688 | 0.630913 | 38,759,961 | A | G | 1531 |
| rs6599255 | 2.54E−30 | 0.133142 | 0.3701 | 38,771,419 | A | C | 24 |
| rs11711941 | 4.23E−29 | −0.130768 | 0.607063 | 38,753,544 | C | T | 1532 |
| rs13098827 | 5.31E−26 | 0.127634 | 0.365009 | 38,735,947 | A | G | 1533 |
| rs6798015 | 3.91E−24 | 0.123609 | 0.323672 | 38,773,840 | C | T | 26 |
| rs6599257 | 1.57E−23 | −0.130896 | 0.724409 | 38,779,592 | T | C | 33 |
| rs7428232 | 7.90E−21 | −0.11759 | 0.544782 | 38,753,622 | C | T | 1534 |
| rs7611456 | 1.98E−20 | 0.118899 | 0.280545 | 38,788,469 | C | T | 1535 |
| rs10212338 | 5.77E−20 | 0.115154 | 0.279278 | 38,787,654 | A | G | 37 |
| rs7651106 | 6.16E−20 | −0.114367 | 0.720684 | 38,779,345 | C | T | 32 |
| rs56040630 | 6.62E−20 | −0.114569 | 0.720867 | 38,785,529 | C | T | 1536 |
| rs4417808 | 6.64E−20 | 0.114543 | 0.279116 | 38,785,241 | A | G | 1537 |
| rs59669930 | 7.26E−20 | 0.114176 | 0.27906 | 38,781,515 | C | T | 1538 |
| rs7610489 | 7.27E−20 | 0.114172 | 0.27906 | 38,781,482 | A | G | 34 |
| rs12497173 | 7.48E−20 | 0.11406 | 0.279043 | 38,780,394 | G | T | 1539 |
| rs4420805 | 1.72E−19 | −0.117522 | 0.720896 | 38,789,237 | A | C | 1540 |
| rs9874436 | 7.15E−19 | 0.099617 | 0.474006 | 38,750,328 | C | G | 1541 |
| rs9311197 | 1.28E−18 | 0.099179 | 0.459247 | 38,751,607 | A | G | 1542 |
| rs9809798 | 1.38E−18 | 0.099013 | 0.459673 | 38,748,809 | A | C | 1543 |
| rs7428167 | 1.74E−18 | −0.098711 | 0.540286 | 38,753,195 | C | T | 1544 |
| rs59856101 | 1.02E−16 | 0.112609 | 0.246351 | 38,794,198 | A | C | 1545 |
| rs73064540 | 1.13E−16 | 0.112813 | 0.246011 | 38,796,286 | A | T | 1546 |
| rs13091192 | 1.43E−16 | 0.094574 | 0.427403 | 38,709,756 | A | G | 1547 |
| rs7430477 | 1.64E−16 | 0.09248 | 0.52152 | 38,740,494 | C | T | 12 |
| rs6599240 | 2.05E−16 | 0.092723 | 0.435855 | 38,713,721 | A | G | 1 |
| chr3: 38801183 | 2.46E−16 | −0.112765 | 0.753603 | 38,801,183 | A | G | 1548 |
| rs11129800 | 2.54E−16 | −0.092615 | 0.563857 | 38,719,374 | C | T | 2 |
| rs62244104 | 4.93E−15 | 0.099652 | 0.343389 | 38,789,222 | A | C | 1549 |
| rs7430438 | 5.82E−15 | −0.090951 | 0.608505 | 38,778,622 | A | G | 1550 |
| rs7617547 | 7.47E−15 | −0.089718 | 0.399087 | 38,738,504 | C | G | 8 |
| rs7430439 | 1.87E−14 | −0.089516 | 0.624811 | 38,778,643 | A | G | 31 |
| rs10428168 | 2.68E−13 | −0.084045 | 0.399892 | 38,755,063 | C | T | 1551 |
| rs7430451 | 2.93E−11 | 0.078447 | 0.660228 | 38,770,499 | C | G | 22 |
| rs11129801 | 3.07E−11 | −0.081662 | 0.308514 | 38,725,379 | A | G | 3 |
| rs12638572 | 6.58E−11 | 0.077193 | 0.664935 | 38,762,801 | A | G | 1552 |
| rs7615140 | 6.94E−11 | −0.077216 | 0.333662 | 38,757,030 | C | T | 19 |
| rs6599253 | 7.83E−11 | 0.07684 | 0.665049 | 38,769,364 | A | C | 1553 |
| rs6784303 | 7.83E−11 | 0.076844 | 0.665051 | 38,769,919 | C | T | 1554 |
| rs7432787 | 8.75E−11 | 0.079791 | 0.459274 | 38,778,334 | A | T | 1555 |
| rs6805187 | 1.51E−10 | −0.075405 | 0.347023 | 38,735,510 | A | C | 7 |
| rs9990137 | 1.54E−10 | 0.075374 | 0.652707 | 38,734,469 | A | G | 6 |
| rs11129799 | 8.26E−10 | 0.084663 | 0.783824 | 38,717,233 | C | T | 1556 |
| rs6599248 | 2.63E−09 | 0.082036 | 0.785716 | 38,724,030 | A | T | 1557 |
| rs7373065 | 3.33E−09 | 0.252193 | 0.975596 | 38,685,319 | C | T | 1558 |
| rs7638275 | 4.56E−09 | −0.25519 | 0.022746 | 38,640,827 | A | G | 1559 |
| rs11926158 | 4.62E−09 | −0.074351 | 0.333793 | 38,798,319 | C | G | 1560 |
| rs6599220 | 6.73E−09 | 0.263995 | 0.981567 | 38,612,998 | C | T | 1561 |
| rs7373492 | 7.93E−09 | 0.247222 | 0.976569 | 38,662,373 | C | T | 1562 |
| rs12490478 | 7.94E−09 | −0.072833 | 0.346668 | 38,792,703 | G | T | 1563 |
| rs7374540 | 8.19E−09 | −0.066793 | 0.625127 | 38,609,146 | A | C | 1564 |
| rs6773331 | 8.93E−09 | −0.246139 | 0.02334 | 38,659,401 | A | T | 1565 |
| rs9878604 | 1.61E−08 | 0.071265 | 0.669519 | 38,801,665 | C | T | 1566 |
| rs7373862 | 2.06E−08 | −0.073119 | 0.748851 | 38,609,347 | A | G | 1567 |
| rs6599219 | 2.53E−08 | −0.0727 | 0.749036 | 38,612,714 | A | G | 1568 |
| rs6764249 | 2.62E−08 | 0.091001 | 0.846459 | 38,705,121 | T | C | 1569 |
| rs6804918 | 3.27E−08 | −0.071234 | 0.714411 | 38,573,960 | A | G | 1570 |
| rs7645178 | 3.29E−08 | −0.07068 | 0.712714 | 38,572,562 | A | G | 1571 |
| rs12053903 | 3.31E−08 | 0.069586 | 0.278414 | 38,568,397 | C | T | 1572 |
| rs7373779 | 3.33E−08 | 0.069645 | 0.278326 | 38,568,947 | C | T | 1573 |
| rs9825762 | 3.44E−08 | 0.073895 | 0.764542 | 38,741,342 | T | C | 1574 |
| chr3: 38762560 | 3.84E−08 | 0.0822 | 0.659245 | 38,762,560 | A | G | 1575 |
| chr3: 38778336 | 3.90E−08 | 0.07997 | 0.764108 | 38,778,336 | A | T | 1576 |
| rs9824157 | 4.31E−08 | 0.071431 | 0.254027 | 38,608,694 | C | T | 1577 |
| rs6793245 | 4.93E−08 | 0.070352 | 0.276953 | 38,574,041 | A | G | 1578 |
| rs3924120 | 5.18E−08 | −0.071046 | 0.746142 | 38,611,159 | A | G | 1579 |
| rs12636123 | 5.65E−08 | −0.0709 | 0.412242 | 38,762,595 | G | T | 1580 |
| rs9828737 | 7.04E−08 | −0.06674 | 0.293123 | 38,751,702 | C | T | 1581 |
| rs13096893 | 8.02E−08 | −0.065963 | 0.296355 | 38,747,868 | A | G | 1582 |
| chr3: 38717393 | 8.35E−08 | 0.123147 | 0.091492 | 38,717,393 | A | G | 1583 |
| rs1805126 | 8.42E−08 | −0.064788 | 0.679109 | 38,567,410 | T | C | 1584 |
| rs9836859 | 8.70E−08 | 0.065874 | 0.702976 | 38,746,906 | C | G | 1585 |
| rs11710462 | 9.01E−08 | −0.065814 | 0.297253 | 38,745,994 | A | C | 1586 |
| rs11710461 | 9.01E−08 | 0.065814 | 0.702747 | 38,745,993 | C | G | 1587 |
| rs9830687 | 9.26E−08 | −0.065587 | 0.295986 | 38,755,975 | A | G | 1588 |
| rs11129803 | 1.08E−07 | −0.070964 | 0.234378 | 38,727,972 | C | T | 1589 |
| rs6799257 | 1.08E−07 | 0.06541 | 0.701792 | 38,742,607 | A | G | 1590 |
| rs6599249 | 1.08E−07 | −0.070907 | 0.234374 | 38,733,384 | A | G | 1591 |
| rs9818087 | 1.08E−07 | −0.070936 | 0.234379 | 38,729,280 | C | T | 1592 |
| rs6777775 | 1.09E−07 | −0.062703 | 0.581722 | 38,796,125 | A | G | 1593 |
| rs11928905 | 1.10E−07 | −0.070879 | 0.234328 | 38,731,502 | C | T | 1594 |
| rs6794914 | 1.11E−07 | 0.070847 | 0.765667 | 38,732,239 | C | T | 1595 |
| rs13319504 | 1.11E−07 | 0.065121 | 0.704231 | 38,762,755 | C | T | 1596 |
| rs9844265 | 1.14E−07 | 0.065055 | 0.704272 | 38,763,510 | C | T | 1597 |
| rs7430861 | 1.15E−07 | 0.065055 | 0.70431 | 38,764,582 | A | C | 1598 |
| rs9816817 | 1.15E−07 | 0.065061 | 0.704327 | 38,765,380 | A | G | 1599 |
| rs6809264 | 1.47E−07 | 0.064962 | 0.706258 | 38,775,767 | A | C | 1600 |
| rs3935184 | 1.51E−07 | 0.067581 | 0.260777 | 38,613,206 | C | G | 1601 |
| rs9847662 | 1.56E−07 | 0.06489 | 0.706668 | 38,776,649 | A | G | 1602 |
| rs6780103 | 1.95E−07 | −0.063366 | 0.311179 | 38,746,468 | A | G | 16 |
| rs9874633 | 1.99E−07 | 0.063283 | 0.689095 | 38,746,998 | A | G | 1603 |
| chr3: 38568132 | 2.01E−07 | 0.089392 | 0.202766 | 38,568,132 | G | T | 1604 |
| rs11129805 | 2.05E−07 | 0.063242 | 0.688916 | 38,745,950 | A | T | 1605 |
| chr3: 38725868 | 2.25E−07 | −0.159607 | 0.039683 | 38,725,868 | C | T | 1606 |
| rs11129802 | 3.10E−07 | 0.09172 | 0.889621 | 38,725,440 | C | T | 1607 |
| rs7430191 | 3.62E−07 | 0.077789 | 0.823635 | 38,629,028 | C | T | 1608 |
| chr3: 38595050 | 4.15E−07 | −0.259531 | 0.981488 | 38,595,050 | A | G | 1609 |
| rs12638536 | 4.89E−07 | 0.074335 | 0.712806 | 38,762,592 | A | C | 1610 |
| rs11705730 | 4.94E−07 | 0.064996 | 0.61091 | 38,789,238 | A | C | 1611 |
| rs6791171 | 5.13E−07 | 0.069515 | 0.788617 | 38,741,705 | C | T | 1612 |
| chr3: 38777576 | 5.17E−07 | 0.157471 | 0.959603 | 38,777,576 | G | T | 1613 |
| rs62244074 | 5.63E−07 | −0.065158 | 0.252353 | 38,778,904 | A | G | 1614 |
| rs62244075 | 5.63E−07 | −0.065163 | 0.25241 | 38,778,938 | C | G | 1615 |
| chr3: 38780863 | 5.86E−07 | −0.065175 | 0.253144 | 38,780,863 | A | G | 1616 |
| rs62244077 | 5.87E−07 | 0.06518 | 0.746846 | 38,781,392 | A | G | 1617 |
| rs11129807 | 5.90E−07 | −0.065195 | 0.253152 | 38,782,421 | A | T | 1618 |
| chr3: 38782569 | 5.91E−07 | 0.065203 | 0.746893 | 38,782,569 | A | G | 1619 |
| rs12635859 | 5.95E−07 | 0.065198 | 0.746804 | 38,783,890 | A | G | 1620 |
| rs12635869 | 5.96E−07 | 0.065199 | 0.746802 | 38,783,971 | A | G | 1621 |
| rs62244078 | 5.99E−07 | −0.065205 | 0.253202 | 38,784,646 | C | T | 1622 |
| rs62244080 | 6.07E−07 | −0.065211 | 0.253282 | 38,786,821 | A | G | 1623 |
| rs62244079 | 6.08E−07 | −0.065241 | 0.253159 | 38,786,782 | C | T | 1624 |
| rs62244081 | 6.35E−07 | −0.065264 | 0.253389 | 38,788,013 | C | T | 1625 |
| rs62244103 | 6.62E−07 | −0.065334 | 0.253575 | 38,788,933 | C | G | 1626 |
| rs58454174 | 7.18E−07 | 0.071179 | 0.803661 | 38,590,537 | C | T | 1627 |
| rs6599239 | 7.19E−07 | 0.097216 | 0.906361 | 38,712,988 | A | G | 1628 |
| rs6599238 | 7.28E−07 | 0.097309 | 0.906287 | 38,711,909 | A | G | 1629 |
| rs6599237 | 7.38E−07 | −0.097401 | 0.093791 | 38,710,782 | C | T | 1630 |
| rs6599242 | 7.39E−07 | −0.097003 | 0.093542 | 38,714,849 | A | G | 1631 |
| rs11710077 | 7.94E−07 | 0.076884 | 0.824592 | 38,632,903 | A | T | 1632 |
| rs6422143 | 8.87E−07 | −0.096483 | 0.092894 | 38,722,167 | C | G | 1633 |
| rs6599243 | 9.29E−07 | −0.095971 | 0.09321 | 38,722,281 | C | T | 1634 |
| rs6599245 | 9.43E−07 | 0.095901 | 0.906812 | 38,722,765 | C | G | 1635 |
| rs6599247 | 9.48E−07 | 0.095881 | 0.906818 | 38,722,876 | C | T | 1636 |
| chr3: 38772763 | 9.99E−07 | 0.344763 | 0.014542 | 38,772,763 | A | G | 1637 |
| rs12636153 | 1.01E−06 | 0.065194 | 0.760858 | 38,744,301 | A | C | 1638 |
| chr3: 38783400 | 1.02E−06 | 0.155906 | 0.961109 | 38,783,400 | C | T | 1639 |
| rs7430283 | 1.11E−06 | −0.095183 | 0.092981 | 38,724,619 | C | G | 1640 |
| rs41312411 | 1.13E−06 | −0.087888 | 0.888794 | 38,596,241 | C | G | 1641 |
| chr3: 38791169 | 1.15E−06 | −0.156111 | 0.038776 | 38,791,169 | A | G | 1642 |
| rs13095477 | 1.15E−06 | 0.069278 | 0.804079 | 38,780,692 | G | T | 1643 |
| chr3: 38792243 | 1.17E−06 | −0.15616 | 0.038754 | 38,792,243 | C | T | 1644 |
| rs13071311 | 1.17E−06 | 0.069341 | 0.804099 | 38,784,058 | G | T | 1645 |
| rs9843500 | 1.24E−06 | 0.059568 | 0.303238 | 38,563,099 | C | G | 1646 |
| rs7429945 | 1.24E−06 | 0.058785 | 0.310687 | 38,566,693 | C | T | 1647 |
| rs62244070 | 1.32E−06 | 0.065003 | 0.764885 | 38,773,175 | C | T | 1648 |
| rs12630795 | 1.44E−06 | 0.064753 | 0.765737 | 38,771,989 | A | G | 25 |
| rs6799868 | 1.48E−06 | 0.065007 | 0.258119 | 38,576,560 | C | T | 1649 |
| rs13087440 | 1.63E−06 | −0.070181 | 0.192574 | 38,752,855 | A | T | 1650 |
| rs4073796 | 1.67E−06 | 0.058594 | 0.304242 | 38,565,853 | A | G | 1651 |
| rs4073797 | 1.67E−06 | −0.058594 | 0.695758 | 38,565,854 | A | T | 1652 |
| chr3: 38614051 | 1.69E−06 | −0.249132 | 0.982837 | 38,614,051 | C | T | 1653 |
| rs73070981 | 1.76E−06 | 0.084932 | 0.121417 | 38,606,842 | C | T | 1654 |
| chr3: 38388644 | 2.11E−06 | 0.449605 | 0.992966 | 38,388,644 | A | T | 1655 |
| rs11720166 | 2.14E−06 | −0.065273 | 0.739489 | 38,574,816 | C | G | 1656 |
| rs34786326 | 2.22E−06 | 0.063741 | 0.770275 | 38,744,872 | C | T | 1657 |
| rs7355944 | 2.28E−06 | −0.067628 | 0.194552 | 38,601,197 | A | G | 1658 |
| rs11708996 | 2.48E−06 | 0.083757 | 0.12072 | 38,608,927 | C | G | 1659 |
| chr3: 38770376 | 2.50E−06 | −0.295309 | 0.014192 | 38,770,376 | A | G | 1660 |
| rs7617919 | 2.53E−06 | −0.062982 | 0.229628 | 38,768,993 | A | G | 1661 |
| rs62242444 | 2.55E−06 | 0.062975 | 0.770208 | 38,752,237 | C | T | 1662 |
| rs62242448 | 2.56E−06 | −0.06296 | 0.229753 | 38,755,623 | A | C | 1663 |
| rs60554541 | 2.56E−06 | 0.06295 | 0.770269 | 38,757,476 | A | G | 1664 |
| rs6599252 | 2.57E−06 | −0.062923 | 0.229653 | 38,764,695 | A | T | 1665 |
| rs60969309 | 2.58E−06 | −0.06291 | 0.229666 | 38,763,008 | C | T | 1666 |
| rs12634001 | 2.58E−06 | −0.062909 | 0.229659 | 38,763,702 | A | G | 1667 |
| rs57326399 | 2.59E−06 | −0.063197 | 0.233081 | 38,743,304 | C | T | 1668 |
| rs59468016 | 2.59E−06 | −0.063183 | 0.233104 | 38,743,251 | A | G | 1669 |
| rs59858965 | 2.64E−06 | 0.063097 | 0.76677 | 38,742,965 | A | C | 1670 |
| rs7432804 | 2.72E−06 | 0.059354 | 0.732109 | 38,778,513 | A | G | 30 |
| rs4414778 | 2.75E−06 | −0.059711 | 0.269836 | 38,787,169 | C | T | 36 |
| rs7641844 | 2.82E−06 | 0.060199 | 0.736415 | 38,777,255 | A | G | 29 |
| rs6599256 | 3.10E−06 | 0.060531 | 0.739804 | 38,776,229 | G | T | 28 |
| rs6763876 | 3.35E−06 | −0.060577 | 0.258382 | 38,775,751 | C | T | 27 |
| rs62244105 | 3.38E−06 | 0.060756 | 0.741276 | 38,789,587 | A | G | 1671 |
| rs73070977 | 3.67E−06 | 0.083248 | 0.111239 | 38,606,435 | G | T | 1672 |
| rs3922843 | 3.79E−06 | −0.061674 | 0.251481 | 38,599,347 | A | G | 1673 |
| chr3: 38800367 | 4.13E−06 | −0.292346 | 0.013685 | 38,800,367 | C | T | 1674 |
| chr3: 38598527 | 4.29E−06 | −0.312534 | 0.009649 | 38,598,527 | C | G | 1675 |
| rs7638909 | 4.52E−06 | 0.062476 | 0.251338 | 38,569,977 | G | T | 1676 |
| rs11710498 | 5.01E−06 | 0.062734 | 0.233457 | 38,551,669 | C | T | 1677 |
| chr3: 38754327 | 5.61E−06 | 0.080343 | 0.835683 | 38,754,327 | C | T | 1678 |
| rs6772948 | 5.77E−06 | −0.069251 | 0.831966 | 38,590,320 | C | T | 1679 |
| rs62245110 | 6.14E−06 | −0.064517 | 0.195859 | 38,605,315 | A | C | 1680 |
| chr3: 38298531 | 7.26E−06 | −0.360365 | 0.006302 | 38,298,531 | A | G | 1681 |
| chr3: 38817911 | 8.79E−06 | 0.155759 | 0.968449 | 38,817,911 | C | T | 1682 |
| chr3: 38822386 | 8.92E−06 | 0.155698 | 0.968499 | 38,822,386 | A | G | 1683 |
| rs7645358 | 9.27E−06 | −0.087631 | 0.90866 | 38,602,829 | A | G | 1684 |
| chr3: 38721228 | 9.62E−06 | 0.228954 | 0.015825 | 38,721,228 | C | T | 1685 |
| rs11129795 | 9.86E−06 | 0.060513 | 0.223435 | 38,564,167 | A | G | 1686 |
| rs62239356 | 9.97E−06 | 0.060543 | 0.223882 | 38,562,097 | C | T | 1687 |
| rs11717455 | 9.98E−06 | 0.075758 | 0.854339 | 38,818,651 | T | C | 1688 |
| rs35231307 | 1.03E−05 | −0.060388 | 0.776282 | 38,564,412 | C | T | 1689 |
| rs41315485 | 1.04E−05 | −0.061345 | 0.767815 | 38,565,279 | A | G | 1690 |
| rs56283404 | 1.06E−05 | 0.060356 | 0.223353 | 38,561,710 | A | C | 1691 |
| rs12490047 | 1.07E−05 | −0.060336 | 0.776656 | 38,561,419 | C | G | 1692 |
| rs34519218 | 1.18E−05 | −0.085296 | 0.908353 | 38,599,846 | C | T | 1693 |
| rs55981643 | 1.20E−05 | −0.062353 | 0.195307 | 38,606,959 | A | G | 1694 |
| rs71323670 | 1.20E−05 | 0.085124 | 0.091768 | 38,600,158 | C | T | 1695 |
| rs6771881 | 1.26E−05 | −0.060009 | 0.776836 | 38,553,268 | A | G | 1696 |
| rs10154914 | 1.33E−05 | −0.061983 | 0.195228 | 38,607,634 | A | T | 1697 |
| rs12497866 | 1.38E−05 | −0.059784 | 0.776514 | 38,550,869 | C | T | 1698 |
| rs7432766 | 1.38E−05 | 0.084137 | 0.092183 | 38,601,932 | C | T | 1699 |
| rs1473804 | 1.39E−05 | −0.059756 | 0.776438 | 38,551,394 | C | T | 1700 |
| rs9311191 | 1.39E−05 | −0.061805 | 0.195189 | 38,608,114 | C | T | 1701 |
| rs61669000 | 1.43E−05 | 0.083864 | 0.092353 | 38,607,439 | C | T | 1702 |
| chr3: 38803994 | 1.52E−05 | 0.091157 | 0.870292 | 38,803,994 | C | T | 1703 |
| rs7373373 | 1.56E−05 | −0.082416 | 0.09272 | 38,732,380 | A | G | 1704 |
| rs7374599 | 1.56E−05 | −0.082416 | 0.09272 | 38,732,298 | C | T | 1705 |
| rs62242433 | 1.56E−05 | −0.082419 | 0.092713 | 38,732,525 | G | T | 1706 |
| chr3: 38731330 | 1.57E−05 | −0.082413 | 0.092708 | 38,731,330 | A | G | 1707 |
| rs62242432 | 1.57E−05 | −0.082413 | 0.092708 | 38,731,322 | C | T | 1708 |
| rs62242429 | 1.59E−05 | −0.082406 | 0.092674 | 38,728,334 | C | T | 1709 |
| rs62242399 | 1.59E−05 | −0.082406 | 0.092672 | 38,728,180 | A | G | 1710 |
| chr3: 38642561 | 1.60E−05 | −0.510884 | 0.006198 | 38,642,561 | A | G | 1711 |
| rs9311192 | 1.64E−05 | 0.061301 | 0.804968 | 38,609,615 | C | T | 1712 |
| rs9311194 | 1.69E−05 | −0.061218 | 0.195007 | 38,610,076 | A | G | 1713 |
| rs13084981 | 1.73E−05 | −0.083515 | 0.907089 | 38,621,003 | C | T | 1714 |
| rs34535972 | 1.73E−05 | −0.083492 | 0.907087 | 38,620,756 | A | G | 1715 |
| rs11721012 | 1.76E−05 | −0.061082 | 0.194968 | 38,610,878 | A | G | 1716 |
| rs1473805 | 1.81E−05 | −0.058949 | 0.775038 | 38,552,683 | A | G | 1717 |
| rs62242438 | 1.90E−05 | −0.056226 | 0.242986 | 38,741,352 | C | T | 1718 |
| rs62242436 | 1.91E−05 | 0.082365 | 0.907997 | 38,737,592 | C | T | 1719 |
| rs10212202 | 2.04E−05 | −0.060604 | 0.194835 | 38,613,394 | C | G | 1720 |
| rs12632942 | 2.06E−05 | 0.055927 | 0.758469 | 38,740,002 | A | G | 11 |
| rs61487238 | 2.09E−05 | 0.055925 | 0.758316 | 38,739,075 | C | T | 1721 |
| rs6771157 | 2.11E−05 | −0.055912 | 0.241736 | 38,738,867 | C | G | 9 |
| rs11710006 | 2.14E−05 | 0.056012 | 0.757762 | 38,727,794 | A | T | 4 |
| rs62242430 | 2.15E−05 | −0.055997 | 0.242208 | 38,729,180 | C | T | 1722 |
| rs62242435 | 2.15E−05 | −0.055964 | 0.242103 | 38,734,621 | A | G | 1723 |
| rs6781740 | 2.15E−05 | 0.055994 | 0.757796 | 38,728,981 | C | T | 1724 |
| chr3: 38701029 | 2.17E−05 | 0.625406 | 0.005272 | 38,701,029 | C | T | 1725 |
| rs61014925 | 2.18E−05 | 0.055954 | 0.757898 | 38,730,904 | A | G | 1726 |
| rs62242434 | 2.23E−05 | 0.055868 | 0.757732 | 38,734,533 | C | T | 1727 |
| rs55872442 | 2.25E−05 | 0.05819 | 0.226204 | 38,556,439 | A | G | 1728 |
| chr3: 38060947 | 2.40E−05 | −0.638075 | 0.006376 | 38,060,947 | A | G | 1729 |
| chr3: 38062521 | 2.42E−05 | −0.124313 | 0.041126 | 38,062,521 | A | C | 1730 |
| rs12635898 | 2.44E−05 | 0.110774 | 0.082952 | 38,601,069 | A | C | 1731 |
| rs9833775 | 2.65E−05 | 0.059706 | 0.805395 | 38,616,088 | C | T | 1732 |
| rs12491987 | 2.66E−05 | 0.083487 | 0.867744 | 38,624,048 | C | T | 1733 |
| chr3: 38128284 | 2.73E−05 | −0.12351 | 0.04117 | 38,128,284 | A | C | 1734 |
| rs55880069 | 2.87E−05 | 0.057291 | 0.227407 | 38,558,045 | C | T | 1735 |
| rs7374030 | 2.92E−05 | 0.085551 | 0.915396 | 38,772,932 | C | T | 1736 |
| rs7428779 | 2.96E−05 | −0.060177 | 0.189218 | 38,621,427 | T | C | 1737 |
| chr3: 39103060 | 3.27E−05 | 0.186406 | 0.977873 | 39,103,060 | A | G | 1738 |
| rs55849713 | 3.37E−05 | 0.080788 | 0.910167 | 38,769,902 | C | T | 1739 |
| rs12054245 | 3.43E−05 | 0.080419 | 0.909662 | 38,766,511 | C | T | 1740 |
| rs58802016 | 3.43E−05 | −0.080423 | 0.090277 | 38,767,581 | C | T | 1741 |
| chr3: 38764592 | 3.43E−05 | −0.080409 | 0.090448 | 38,764,592 | A | G | 1742 |
| rs7373595 | 3.43E−05 | −0.08044 | 0.090211 | 38,768,463 | A | T | 1743 |
| rs11129804 | 3.51E−05 | 0.077324 | 0.898155 | 38,741,829 | A | G | 1744 |
| chr3: 38265547 | 3.53E−05 | 0.63025 | 0.993638 | 38,265,547 | C | T | 1745 |
| rs62242453 | 3.55E−05 | 0.080268 | 0.909444 | 38,762,112 | C | T | 1746 |
| rs6762565 | 3.78E−05 | −0.056298 | 0.771542 | 38,557,195 | C | T | 1747 |
| rs41312433 | 3.87E−05 | 0.059273 | 0.810742 | 38,622,646 | G | T | 1748 |
| rs10865879 | 3.90E−05 | −0.056259 | 0.771705 | 38,552,366 | A | C | 1749 |
| chr3: 38751420 | 4.05E−05 | 0.080128 | 0.910483 | 38,751,420 | C | T | 1750 |
| rs62242443 | 4.11E−05 | −0.080032 | 0.089468 | 38,749,915 | C | T | 1751 |
| rs11129806 | 4.14E−05 | 0.080131 | 0.910627 | 38,757,741 | C | T | 1752 |
| rs6781009 | 4.15E−05 | 0.054922 | 0.236685 | 38,560,438 | C | T | 1753 |
| chr3: 38753692 | 4.15E−05 | 0.080047 | 0.910607 | 38,753,692 | C | T | 1754 |
| rs62242446 | 4.17E−05 | −0.080052 | 0.089364 | 38,755,194 | C | T | 1755 |
| rs62242447 | 4.17E−05 | −0.080053 | 0.089356 | 38,755,570 | C | T | 1756 |
| rs7426951 | 4.19E−05 | 0.08006 | 0.910678 | 38,757,297 | A | G | 1757 |
| rs3923697 | 4.19E−05 | −0.08006 | 0.089316 | 38,757,574 | A | G | 1758 |
| rs62242450 | 4.20E−05 | 0.080063 | 0.910695 | 38,758,165 | A | G | 1759 |
| rs6599236 | 4.36E−05 | 0.077024 | 0.886067 | 38,710,164 | A | G | 1760 |
| rs7374804 | 4.43E−05 | −0.07934 | 0.089935 | 38,743,338 | C | T | 1761 |
| chr3: 38765921 | 4.52E−05 | 0.208937 | 0.014526 | 38,765,921 | A | C | 1762 |
| chr3: 38297989 | 4.61E−05 | 0.681089 | 0.993441 | 38,297,989 | A | G | 1763 |
| rs11711602 | 4.68E−05 | 0.058089 | 0.809355 | 38,622,051 | C | T | 1764 |
| chr3: 39000860 | 4.71E−05 | 0.182537 | 0.977603 | 39,000,860 | A | G | 1765 |
| rs4420804 | 5.14E−05 | 0.045988 | 0.549449 | 38,787,712 | C | T | 1766 |
| rs11917835 | 5.20E−05 | −0.045809 | 0.451031 | 38,785,355 | A | G | 1767 |
| rs4676479 | 5.25E−05 | −0.045732 | 0.451168 | 38,783,037 | A | G | 1768 |
| rs7433352 | 5.32E−05 | 0.045629 | 0.548689 | 38,779,672 | A | C | 1769 |
| rs4676596 | 5.33E−05 | 0.045779 | 0.549266 | 38,785,701 | C | T | 1770 |
| chr3: 38742425 | 5.44E−05 | −0.11454 | 0.939981 | 38,742,425 | A | G | 1771 |
| rs6803189 | 5.48E−05 | −0.073395 | 0.105084 | 38,732,870 | C | T | 1772 |
| rs6802294 | 5.66E−05 | 0.073347 | 0.894998 | 38,734,826 | C | G | 1773 |
| chr3: 39231916 | 5.70E−05 | −0.177438 | 0.02171 | 39,231,916 | C | G | 1774 |
| chr3: 39249243 | 6.28E−05 | 0.175851 | 0.977845 | 39,249,243 | A | G | 1775 |
| chr3: 38979166 | 6.49E−05 | 0.180355 | 0.978204 | 38,979,166 | A | G | 1776 |
| rs7431144 | 6.56E−05 | 0.080016 | 0.904812 | 38,769,272 | T | C | 1777 |
| rs2364658 | 7.05E−05 | −0.046512 | 0.610214 | 37,764,386 | C | G | 1778 |
| rs9833086 | 7.09E−05 | −0.050013 | 0.695848 | 38,585,475 | A | G | 1779 |
| rs4395346 | 7.11E−05 | 0.049721 | 0.603072 | 38,826,088 | T | C | 1780 |
| rs4622847 | 7.12E−05 | −0.051223 | 0.291505 | 38,799,347 | A | G | 1781 |
| rs13085808 | 8.45E−05 | −0.066077 | 0.141233 | 38,804,274 | A | G | 1782 |
| chr3: 38779878 | 8.48E−05 | 0.199302 | 0.014225 | 38,779,878 | C | T | 1783 |
| rs73826324 | 9.62E−05 | 0.075615 | 0.904425 | 38,762,381 | A | G | 1784 |
| chr3: 38062226 | 9.64E−05 | 0.531336 | 0.99311 | 38,062,226 | C | T | 1785 |
| chr3: 38601173 | 9.80E−05 | −0.098198 | 0.079393 | 38,601,173 | A | G | 1786 |
| chr3: 38300452 | 1.03E−04 | 0.154628 | 0.965923 | 38,300,452 | A | G | 1787 |
| rs4130467 | 1.06E−04 | 0.0484 | 0.279216 | 38,586,708 | C | T | 1788 |
| rs9861242 | 1.10E−04 | 0.05323 | 0.218534 | 38,584,338 | A | G | 1789 |
| chr3: 37850981 | 1.14E−04 | 0.118446 | 0.960693 | 37,850,981 | C | T | 1790 |
| chr3: 38197176 | 1.17E−04 | 0.15315 | 0.966148 | 38,197,176 | C | T | 1791 |
| rs11713291 | 1.20E−04 | −0.075828 | 0.106248 | 38,061,467 | A | G | 1792 |
| rs11708345 | 1.21E−04 | 0.075717 | 0.893644 | 38,076,426 | C | T | 1793 |
| rs12631535 | 1.23E−04 | 0.054312 | 0.78803 | 38,706,121 | T | C | 1794 |
| rs4131778 | 1.29E−04 | −0.048078 | 0.721166 | 38,587,230 | A | T | 1795 |
| chr3: 38779651 | 1.37E−04 | 0.187341 | 0.982733 | 38,779,651 | C | T | 1796 |
| rs34418780 | 1.44E−04 | 0.083078 | 0.923539 | 38,909,550 | A | T | 1797 |
| chr3: 38048625 | 1.44E−04 | 0.550659 | 0.993517 | 38,048,625 | G | T | 1798 |
| rs60721494 | 1.49E−04 | 0.063956 | 0.866777 | 38,008,075 | G | T | 1799 |
| chr3: 38699887 | 1.51E−04 | −0.907248 | 0.996079 | 38,699,887 | C | T | 1800 |
| rs9845438 | 1.56E−04 | −0.057428 | 0.801105 | 38,575,460 | A | C | 1801 |
| chr3: 38678925 | 1.58E−04 | 0.203091 | 0.018207 | 38,678,925 | A | G | 1802 |
| rs28829975 | 1.72E−04 | −0.048831 | 0.709491 | 39,485,675 | A | G | 1803 |
| chr3: 38758496 | 1.80E−04 | −0.116817 | 0.044034 | 38,758,496 | C | T | 1804 |
| rs3922844 | 1.83E−04 | 0.044832 | 0.6762 | 38,599,257 | C | T | 1805 |
| rs73058503 | 1.89E−04 | −0.075511 | 0.910914 | 39,222,289 | A | G | 1806 |
| rs7620883 | 1.90E−04 | 0.050707 | 0.221489 | 38,589,484 | A | G | 1807 |
| rs9880327 | 1.98E−04 | −0.047752 | 0.265034 | 38,657,897 | A | G | 1808 |
| rs11711062 | 2.05E−04 | 0.332663 | 0.992064 | 38,728,736 | A | T | 1809 |
| rs9856387 | 2.05E−04 | 0.047761 | 0.735989 | 38,662,230 | C | T | 1810 |
| chr3: 38903936 | 2.07E−04 | −0.054206 | 0.41857 | 38,903,936 | G | T | 1811 |
| rs11712354 | 2.24E−04 | 0.060352 | 0.85916 | 38,810,892 | A | C | 1812 |
| rs11709828 | 2.26E−04 | −0.060307 | 0.140827 | 38,811,865 | C | T | 1813 |
| rs11721285 | 2.26E−04 | −0.060309 | 0.140817 | 38,811,576 | A | G | 1814 |
| chr3: 38812178 | 2.27E−04 | 0.060278 | 0.859196 | 38,812,178 | C | T | 1815 |
| rs9843296 | 2.30E−04 | −0.060009 | 0.141691 | 38,018,908 | C | T | 1816 |
| rs13075748 | 2.32E−04 | 0.060161 | 0.859242 | 38,814,393 | C | T | 1817 |
| rs62243862 | 2.37E−04 | −0.051902 | 0.201294 | 38,716,553 | C | T | 1818 |
| chr3: 38598253 | 2.38E−04 | 0.230967 | 0.977627 | 38,598,253 | C | T | 1819 |
| rs3922578 | 2.49E−04 | 0.06124 | 0.859413 | 38,836,126 | A | G | 1820 |
| rs6599244 | 2.51E−04 | 0.063679 | 0.875324 | 38,722,739 | A | G | 1821 |
| rs11718422 | 2.53E−04 | −0.059811 | 0.140555 | 38,834,190 | A | G | 1822 |
| rs11708296 | 2.53E−04 | 0.05977 | 0.85945 | 38,832,069 | A | G | 1823 |
| rs6804644 | 2.53E−04 | −0.059785 | 0.140552 | 38,833,051 | C | T | 1824 |
| rs6798701 | 2.53E−04 | 0.059772 | 0.859445 | 38,832,379 | A | G | 1825 |
| rs11713967 | 2.54E−04 | 0.059635 | 0.859446 | 38,830,277 | C | T | 1826 |
| rs11717588 | 2.54E−04 | 0.059631 | 0.859446 | 38,828,164 | C | T | 1827 |
| rs4528881 | 2.54E−04 | 0.059627 | 0.859448 | 38,823,410 | A | G | 1828 |
| chr3: 38823059 | 2.54E−04 | −0.059626 | 0.140552 | 38,823,059 | A | T | 1829 |
| rs13080911 | 2.54E−04 | 0.059624 | 0.859449 | 38,820,925 | A | G | 1830 |
| rs13061068 | 2.54E−04 | −0.059624 | 0.140551 | 38,820,915 | G | T | 1831 |
| rs35257385 | 2.54E−04 | −0.059624 | 0.140551 | 38,820,730 | A | G | 1832 |
| rs11713473 | 2.55E−04 | −0.059613 | 0.140543 | 38,818,967 | A | G | 1833 |
| rs11713400 | 2.55E−04 | 0.059612 | 0.859457 | 38,818,891 | C | T | 1834 |
| chr3: 38512241 | 2.59E−04 | 0.108613 | 0.046847 | 38,512,241 | A | G | 1835 |
| chr3: 39449599 | 2.64E−04 | 0.099382 | 0.061678 | 39,449,599 | C | T | 1836 |
| chr3: 38100896 | 2.65E−04 | −0.055714 | 0.709063 | 38,100,896 | A | G | 1837 |
| rs41314746 | 2.67E−04 | 0.234942 | 0.983996 | 38,650,719 | C | T | 1838 |
| rs7430323 | 2.72E−04 | 0.066142 | 0.894595 | 38,763,488 | A | G | 1839 |
| rs11719241 | 2.73E−04 | 0.059857 | 0.854074 | 38,810,088 | G | T | 1840 |
| rs3923696 | 2.74E−04 | −0.066616 | 0.10393 | 38,757,197 | A | T | 1841 |
| chr3: 38080491 | 2.81E−04 | −0.054471 | 0.647449 | 38,080,491 | C | T | 1842 |
| rs9842880 | 2.82E−04 | −0.058249 | 0.152469 | 38,027,617 | C | G | 1843 |
| rs58141279 | 2.83E−04 | −0.066461 | 0.103956 | 38,758,205 | C | G | 1844 |
| rs60847476 | 2.84E−04 | −0.066435 | 0.10396 | 38,758,369 | A | G | 1845 |
| rs6599233 | 2.84E−04 | 0.046062 | 0.721856 | 38,687,884 | C | T | 1846 |
| rs56206213 | 2.91E−04 | −0.065787 | 0.105383 | 38,765,835 | C | T | 1847 |
| chr3: 38819804 | 2.93E−04 | 0.072804 | 0.901423 | 38,819,804 | C | T | 1848 |
| chr3: 38774863 | 2.96E−04 | −0.181312 | 0.974528 | 38,774,863 | C | T | 1849 |
| rs7372391 | 2.96E−04 | −0.0606 | 0.137626 | 38,712,647 | C | T | 1850 |
| rs57803396 | 3.03E−04 | −0.065574 | 0.105355 | 38,767,092 | A | G | 1851 |
| rs56654680 | 3.05E−04 | 0.065526 | 0.894649 | 38,767,384 | A | T | 1852 |
| rs9758003 | 3.12E−04 | −0.05829 | 0.151993 | 38,028,210 | C | T | 1853 |
| chr3: 38709166 | 3.21E−04 | 0.059943 | 0.858517 | 38,709,166 | A | G | 1854 |
| rs9311175 | 3.30E−04 | −0.059065 | 0.141967 | 38,022,215 | C | G | 1855 |
| rs62242857 | 3.31E−04 | 0.059706 | 0.857718 | 38,708,421 | A | G | 1856 |
| rs3796387 | 3.31E−04 | 0.055572 | 0.171953 | 38,554,211 | A | G | 1857 |
| chr3: 38903943 | 3.33E−04 | 0.066871 | 0.825078 | 38,903,943 | A | G | 1858 |
| chr3: 38519917 | 3.35E−04 | 0.145876 | 0.02794 | 38,519,917 | A | C | 1859 |
| chr3: 38782784 | 3.36E−04 | −0.202091 | 0.979847 | 38,782,784 | A | C | 1860 |
| rs62244073 | 3.37E−04 | −0.058603 | 0.699527 | 38,778,361 | A | G | 1861 |
| chr3: 38016350 | 3.38E−04 | −0.058767 | 0.139859 | 38,016,350 | A | G | 1862 |
| rs12631918 | 3.51E−04 | −0.040934 | 0.466961 | 38,785,796 | C | T | 1863 |
| rs4676592 | 3.53E−04 | −0.053077 | 0.182793 | 38,839,941 | A | G | 1864 |
| rs62243861 | 3.63E−04 | −0.059798 | 0.136087 | 38,715,301 | A | G | 1865 |
| rs11714394 | 3.69E−04 | −0.059736 | 0.136057 | 38,715,416 | G | T | 1866 |
| rs62242859 | 3.73E−04 | 0.050368 | 0.800065 | 38,712,327 | C | T | 1867 |
| rs7432727 | 3.76E−04 | 0.040785 | 0.576485 | 38,774,400 | C | T | 1868 |
| chr3: 38661502 | 3.79E−04 | −0.091474 | 0.097432 | 38,661,502 | C | G | 1869 |
| rs4676594 | 3.93E−04 | 0.058723 | 0.861981 | 38,806,006 | G | T | 1870 |
| rs62241189 | 3.95E−04 | −0.07126 | 0.155663 | 38,581,750 | A | G | 1871 |
| rs3935472 | 3.97E−04 | 0.077452 | 0.097265 | 38,577,859 | A | G | 1872 |
| rs7648182 | 4.01E−04 | −0.055698 | 0.166108 | 38,010,512 | A | G | 1873 |
| rs7641519 | 4.06E−04 | 0.055302 | 0.843731 | 38,014,306 | C | T | 1874 |
| rs6809649 | 4.11E−04 | −0.055259 | 0.156261 | 38,013,986 | C | T | 1875 |
| rs62242814 | 4.27E−04 | −0.16577 | 0.021081 | 38,670,875 | A | G | 1876 |
| rs11715511 | 4.36E−04 | 0.055342 | 0.84243 | 38,001,918 | T | C | 1877 |
| rs12639182 | 4.40E−04 | 0.049774 | 0.800504 | 38,711,234 | C | T | 1878 |
| chr3: 38025163 | 4.49E−04 | −0.058079 | 0.13994 | 38,025,163 | A | G | 1879 |
| chr3: 37980243 | 4.84E−04 | 0.071709 | 0.916606 | 37,980,243 | A | G | 1880 |
| rs11715346 | 4.88E−04 | −0.058196 | 0.137128 | 38,817,026 | C | T | 1881 |
| rs9812912 | 4.91E−04 | 0.069795 | 0.845333 | 38,582,233 | C | T | 1882 |
| rs41276521 | 5.00E−04 | 0.071524 | 0.916647 | 37,985,831 | A | C | 1883 |
| rs11707277 | 5.27E−04 | −0.057375 | 0.139228 | 38,022,684 | A | G | 1884 |
| chr3: 38970944 | 5.28E−04 | −0.06503 | 0.170668 | 38,970,944 | G | T | 1885 |
| rs6808011 | 5.38E−04 | −0.054307 | 0.155257 | 38,011,918 | C | T | 1886 |
| rs59658035 | 5.43E−04 | 0.054273 | 0.844736 | 38,011,702 | A | G | 1887 |
| rs7611147 | 5.49E−04 | 0.054093 | 0.843805 | 38,006,376 | A | C | 1888 |
| chr3: 38659082 | 5.58E−04 | 0.189345 | 0.987583 | 38,659,082 | C | T | 1889 |
| rs62241530 | 5.60E−04 | −0.230905 | 0.012083 | 37,757,322 | A | G | 1890 |
| rs6776034 | 5.68E−04 | 0.085483 | 0.086042 | 38,754,193 | A | T | 1891 |
| rs62243860 | 5.75E−04 | −0.059308 | 0.124371 | 38,713,161 | C | T | 1892 |
| chr3: 39387876 | 6.05E−04 | −0.318357 | 0.987019 | 39,387,876 | C | T | 1893 |
| chr3: 39338381 | 6.24E−04 | 0.112552 | 0.036607 | 39,338,381 | A | G | 1894 |
| rs704941 | 6.29E−04 | 0.052198 | 0.188681 | 38,381,482 | A | G | 1895 |
| rs62242856 | 6.45E−04 | 0.058253 | 0.870202 | 38,708,250 | A | G | 1896 |
| rs11711097 | 6.48E−04 | 0.046274 | 0.21536 | 38,603,646 | A | G | 1897 |
| rs11922163 | 6.51E−04 | −0.053432 | 0.159536 | 38,003,855 | A | G | 1898 |
| rs9868464 | 6.54E−04 | 0.199174 | 0.988638 | 38,904,262 | C | T | 1899 |
| chr3: 38021473 | 6.57E−04 | 0.056401 | 0.861856 | 38,021,473 | C | T | 1900 |
| chr3: 38766563 | 6.69E−04 | −0.372385 | 0.988562 | 38,766,563 | C | T | 1901 |
| chr3: 38019729 | 6.70E−04 | −0.420892 | 0.006897 | 38,019,729 | A | G | 1902 |
| rs11715803 | 6.82E−04 | 0.053323 | 0.844785 | 38,006,024 | C | G | 1903 |
| rs6550514 | 6.97E−04 | 0.052991 | 0.838456 | 38,014,191 | C | T | 1904 |
| rs35710839 | 7.10E−04 | −0.126708 | 0.963133 | 38,720,820 | C | G | 1905 |
| chr3: 38518987 | 7.13E−04 | −0.404703 | 0.007668 | 38,518,987 | G | T | 1906 |
| rs11712625 | 7.15E−04 | 0.052935 | 0.843863 | 38,003,349 | C | T | 1907 |
| rs9875610 | 7.32E−04 | 0.040934 | 0.398496 | 38,805,121 | A | G | 1908 |
| rs9851724 | 7.39E−04 | 0.04346 | 0.718205 | 38,694,939 | T | C | 1909 |
| rs4131768 | 7.47E−04 | 0.042708 | 0.720193 | 38,670,178 | A | G | 1910 |
| rs56264852 | 7.55E−04 | 0.053016 | 0.845287 | 38,005,571 | G | T | 1911 |
| rs12637451 | 7.56E−04 | 0.064053 | 0.100708 | 39,222,662 | C | G | 1912 |
| rs7641182 | 7.57E−04 | 0.110356 | 0.035177 | 39,382,446 | C | T | 1913 |
| rs2201966 | 7.58E−04 | 0.110261 | 0.035135 | 39,381,029 | C | T | 1914 |
| rs6766291 | 7.59E−04 | −0.110662 | 0.964691 | 39,388,814 | C | T | 1915 |
| rs9813465 | 7.60E−04 | 0.064038 | 0.100596 | 39,222,999 | C | T | 1916 |
| rs7611087 | 7.61E−04 | −0.110315 | 0.964854 | 39,382,566 | A | T | 1917 |
| rs12632771 | 7.63E−04 | −0.063988 | 0.899441 | 39,223,856 | A | G | 1918 |
| rs6782237 | 7.64E−04 | 0.04263 | 0.720125 | 38,671,557 | C | G | 1919 |
| rs13320742 | 7.65E−04 | −0.110331 | 0.964864 | 39,383,818 | A | G | 1920 |
| rs13313922 | 7.80E−04 | −0.063871 | 0.899593 | 39,225,755 | C | T | 1921 |
| rs9809830 | 7.85E−04 | −0.063838 | 0.899629 | 39,226,145 | C | G | 1922 |
| rs9682325 | 7.87E−04 | 0.110195 | 0.03583 | 39,384,700 | C | T | 1923 |
| rs6764150 | 8.00E−04 | 0.094093 | 0.046719 | 39,383,318 | A | T | 1924 |
| chr3: 38920693 | 8.04E−04 | 0.195615 | 0.987696 | 38,920,693 | C | T | 1925 |
| rs6810361 | 8.06E−04 | 0.053876 | 0.164832 | 38,549,972 | C | T | 1926 |
| chr3: 38919280 | 8.06E−04 | −0.195565 | 0.012329 | 38,919,280 | A | G | 1927 |
| rs6599234 | 8.14E−04 | −0.041584 | 0.29089 | 38,690,304 | A | T | 1928 |
| chr3: 38965331 | 8.16E−04 | −0.195295 | 0.012403 | 38,965,331 | C | T | 1929 |
| chr3: 38938485 | 8.18E−04 | 0.195277 | 0.98763 | 38,938,485 | G | T | 1930 |
| rs12636775 | 8.21E−04 | −0.063622 | 0.899828 | 39,229,388 | A | G | 1931 |
| rs6599232 | 8.22E−04 | −0.042473 | 0.280398 | 38,674,764 | C | T | 1932 |
| chr3: 39120020 | 8.23E−04 | −0.195285 | 0.012477 | 39,120,020 | A | G | 1933 |
| chr3: 39090420 | 8.37E−04 | 0.194906 | 0.987549 | 39,090,420 | A | G | 1934 |
| rs73825452 | 8.46E−04 | −0.05237 | 0.155205 | 38,003,618 | A | G | 1935 |
| rs2133581 | 8.81E−04 | 0.112643 | 0.033776 | 39,381,427 | A | G | 1936 |
| rs11719260 | 9.00E−04 | 0.051861 | 0.843914 | 38,002,105 | A | G | 1937 |
| rs11711286 | 9.27E−04 | 0.051742 | 0.843912 | 38,001,656 | C | T | 1938 |
| rs73825449 | 9.32E−04 | −0.051726 | 0.156146 | 38,000,995 | A | G | 1939 |
| rs3922579 | 9.42E−04 | 0.053662 | 0.843232 | 38,836,277 | A | C | 1940 |
| rs61558743 | 9.48E−04 | −0.045376 | 0.79004 | 38,606,859 | A | G | 1941 |
| rs11709029 | 9.50E−04 | 0.05167 | 0.843762 | 37,999,424 | C | T | 1942 |
| rs11129775 | 9.50E−04 | 0.051669 | 0.843761 | 37,999,412 | C | T | 1943 |
| chr3: 39002755 | 9.53E−04 | 0.092386 | 0.954045 | 39,002,755 | C | T | 1944 |
| rs7430391 | 9.61E−04 | −0.041509 | 0.290387 | 38,698,971 | A | G | 1945 |
| rs73067126 | 9.69E−04 | 0.073902 | 0.144308 | 38,561,041 | A | C | 1946 |
| chr3: 39595718 | 9.75E−04 | −0.234545 | 0.982702 | 39,595,718 | C | T | 1947 |
| rs11923194 | 9.85E−04 | −0.049903 | 0.167844 | 38,870,675 | C | G | 1948 |
| rs10095 | 9.90E−04 | 0.051559 | 0.843857 | 37,998,557 | A | G | 1949 |
| rs6599246 | 9.94E−04 | −0.055277 | 0.13417 | 38,722,814 | C | G | 1950 |
| rs2018725 | 1.00E−03 | 0.038001 | 0.52228 | 39,448,228 | C | T | 1951 |
| rs9851710 | 1.00E−03 | 0.040816 | 0.709099 | 38,694,905 | A | C | 1952 |
| chr3: 38831238 | 1.00E−03 | 0.160441 | 0.024271 | 38,831,238 | A | G | 1953 |
| chr3: 38397851 | 1.03E−03 | −0.110487 | 0.958191 | 38,397,851 | C | T | 1954 |
| rs6790627 | 1.03E−03 | −0.055148 | 0.134265 | 38,723,837 | C | T | 1955 |
| rs9867831 | 1.04E−03 | 0.080174 | 0.059466 | 38,903,647 | A | G | 1956 |
| rs7633988 | 1.06E−03 | 0.041128 | 0.709848 | 38,698,221 | A | T | 1957 |
| rs7427574 | 1.06E−03 | −0.051991 | 0.824358 | 38,659,839 | C | G | 1958 |
| rs28474903 | 1.08E−03 | 0.082423 | 0.058013 | 38,846,457 | A | C | 1959 |
| rs631312 | 1.08E−03 | −0.041485 | 0.730434 | 39,483,972 | A | G | 1960 |
| rs6782263 | 1.09E−03 | 0.08008 | 0.059525 | 38,877,394 | C | G | 1961 |
| rs34710261 | 1.10E−03 | 0.068168 | 0.102071 | 38,089,066 | A | G | 1962 |
| rs11711336 | 1.11E−03 | 0.051101 | 0.844873 | 38,001,588 | G | T | 1963 |
| chr3: 38840481 | 1.11E−03 | 0.083336 | 0.057375 | 38,840,481 | A | G | 1964 |
| chr3: 38709564 | 1.12E−03 | 0.202018 | 0.014021 | 38,709,564 | C | T | 1965 |
| chr3: 37889067 | 1.13E−03 | 0.045642 | 0.55487 | 37,889,067 | C | T | 1966 |
| rs73064283 | 1.14E−03 | 0.08069 | 0.055656 | 39,052,990 | A | G | 1967 |
| rs73064286 | 1.14E−03 | 0.080672 | 0.055669 | 39,056,231 | C | G | 1968 |
| rs73066261 | 1.15E−03 | 0.080669 | 0.055773 | 39,077,609 | C | T | 1969 |
| chr3: 39532775 | 1.15E−03 | −0.235598 | 0.012477 | 39,532,775 | A | G | 1970 |
| rs13079576 | 1.15E−03 | 0.039522 | 0.518699 | 39,457,665 | A | G | 1971 |
| chr3: 37988767 | 1.17E−03 | −0.062697 | 0.098106 | 37,988,767 | A | G | 1972 |
| rs11717282 | 1.18E−03 | 0.053624 | 0.860575 | 37,994,928 | A | C | 1973 |
| rs17037286 | 1.18E−03 | −0.050454 | 0.159989 | 38,001,391 | A | G | 1974 |
| rs73066579 | 1.18E−03 | 0.079429 | 0.059944 | 38,879,661 | A | C | 1975 |
| rs9814913 | 1.19E−03 | −0.080406 | 0.943773 | 39,131,965 | C | T | 1976 |
| rs17852414 | 1.19E−03 | 0.080374 | 0.056005 | 39,110,555 | A | G | 1977 |
| rs13321595 | 1.21E−03 | −0.080281 | 0.943816 | 39,143,813 | C | T | 1978 |
| rs35581078 | 1.21E−03 | −0.080268 | 0.943805 | 39,145,374 | C | T | 1979 |
| chr3: 37934175 | 1.23E−03 | −0.19111 | 0.98842 | 37,934,175 | A | G | 1980 |
| rs66497952 | 1.24E−03 | 0.079053 | 0.060179 | 38,897,356 | C | T | 1981 |
| rs7625935 | 1.25E−03 | 0.078966 | 0.060232 | 38,896,416 | G | T | 1982 |
| chr3: 38773054 | 1.26E−03 | −0.1599 | 0.983573 | 38,773,054 | A | T | 1983 |
| rs28394020 | 1.26E−03 | 0.078933 | 0.060251 | 38,895,855 | A | G | 1984 |
| rs67630000 | 1.26E−03 | 0.078935 | 0.06025 | 38,888,937 | A | G | 1985 |
| rs67434693 | 1.26E−03 | −0.078935 | 0.93975 | 38,888,973 | A | G | 1986 |
| rs28504547 | 1.26E−03 | 0.078932 | 0.060252 | 38,889,554 | C | T | 1987 |
| chr3: 38893548 | 1.26E−03 | 0.078926 | 0.060278 | 38,893,548 | C | T | 1988 |
| chr3: 38893579 | 1.27E−03 | 0.078906 | 0.060267 | 38,893,579 | C | T | 1989 |
| chr3: 38894186 | 1.27E−03 | 0.078902 | 0.06027 | 38,894,186 | C | T | 1990 |
| chr3: 38894217 | 1.27E−03 | 0.078902 | 0.06027 | 38,894,217 | C | T | 1991 |
| rs35036319 | 1.29E−03 | −0.045424 | 0.216467 | 38,702,330 | A | G | 1992 |
| rs11719578 | 1.30E−03 | 0.050642 | 0.845121 | 37,997,143 | A | G | 1993 |
| chr3: 37996119 | 1.31E−03 | 0.053166 | 0.861381 | 37,996,119 | C | T | 1994 |
| chr3: 37919948 | 1.31E−03 | −0.190898 | 0.988438 | 37,919,948 | G | T | 1995 |
| chr3: 39454605 | 1.33E−03 | −0.194323 | 0.017293 | 39,454,605 | C | T | 1996 |
| rs73066514 | 1.33E−03 | −0.09033 | 0.947972 | 38,838,976 | C | T | 1997 |
| rs13079441 | 1.37E−03 | −0.042217 | 0.752695 | 37,751,837 | A | T | 1998 |
| rs4130468 | 1.37E−03 | −0.063422 | 0.910364 | 38,837,152 | C | T | 1999 |
| chr3: 39395225 | 1.39E−03 | 0.449711 | 0.996465 | 39,395,225 | G | T | 2000 |
| rs73065157 | 1.41E−03 | −0.21264 | 0.983137 | 39,691,747 | C | T | 2001 |
| chr3: 37879099 | 1.42E−03 | 0.190345 | 0.011522 | 37,879,099 | A | C | 2002 |
| chr3: 38499596 | 1.43E−03 | 0.393939 | 0.992846 | 38,499,596 | A | C | 2003 |
| rs55795043 | 1.44E−03 | −0.080077 | 0.056902 | 39,176,646 | A | C | 2004 |
| rs4315640 | 1.51E−03 | −0.049839 | 0.1528 | 38,898,704 | A | G | 2005 |
| rs7373157 | 1.53E−03 | 0.052127 | 0.137318 | 38,606,427 | G | T | 2006 |
| rs62242295 | 1.54E−03 | 0.180101 | 0.013167 | 38,935,550 | A | T | 2007 |
| rs1805124 | 1.60E−03 | 0.039802 | 0.731346 | 38,620,424 | A | G | 2008 |
| chr3: 38640584 | 1.62E−03 | 0.070504 | 0.908864 | 38,640,584 | A | G | 2009 |
| rs7429946 | 1.64E−03 | 0.054386 | 0.878805 | 38,722,526 | A | G | 2010 |
| rs6599230 | 1.65E−03 | 0.050478 | 0.836514 | 38,649,716 | C | T | 2011 |
| rs11129776 | 1.67E−03 | 0.0487 | 0.836656 | 38,003,679 | A | C | 2012 |
| rs9816693 | 1.68E−03 | −0.04827 | 0.168549 | 38,022,958 | C | G | 2013 |
| rs73063271 | 1.69E−03 | −0.097018 | 0.960026 | 38,442,253 | A | G | 2014 |
| chr3: 38721046 | 1.70E−03 | −0.104784 | 0.949736 | 38,721,046 | G | T | 2015 |
| rs7374040 | 1.71E−03 | 0.051582 | 0.137082 | 38,607,051 | C | G | 2016 |
| rs11715513 | 1.77E−03 | −0.054174 | 0.121375 | 38,725,387 | A | G | 2017 |
| rs71329572 | 1.78E−03 | −0.052792 | 0.770347 | 39,457,592 | G | T | 2018 |
| rs13075057 | 1.78E−03 | −0.036389 | 0.375907 | 38,819,780 | C | G | 2019 |
| rs7627421 | 1.79E−03 | −0.036374 | 0.375893 | 38,821,038 | A | T | 2020 |
| rs7627881 | 1.79E−03 | 0.036371 | 0.62411 | 38,822,156 | A | G | 2021 |
| rs9859214 | 1.79E−03 | −0.03637 | 0.375889 | 38,822,965 | A | T | 2022 |
| rs9860830 | 1.79E−03 | −0.036363 | 0.375881 | 38,826,952 | A | G | 2023 |
| rs4608627 | 1.79E−03 | 0.036363 | 0.624121 | 38,827,665 | A | G | 2024 |
| rs7625973 | 1.79E−03 | 0.036363 | 0.624122 | 38,828,437 | C | T | 2025 |
| rs9871453 | 1.79E−03 | −0.036363 | 0.375877 | 38,828,966 | A | G | 2026 |
| rs4274690 | 1.79E−03 | 0.036363 | 0.624123 | 38,829,106 | A | C | 2027 |
| rs4368436 | 1.79E−03 | −0.036364 | 0.375864 | 38,830,536 | C | T | 2028 |
| rs41311123 | 1.81E−03 | −0.155736 | 0.981773 | 38,576,669 | C | T | 2029 |
| chr3: 38966602 | 1.81E−03 | 0.060443 | 0.888848 | 38,966,602 | G | T | 2030 |
| rs62244110 | 1.82E−03 | −0.036608 | 0.619781 | 38,807,434 | G | T | 2031 |
| rs4676478 | 1.83E−03 | 0.036369 | 0.624329 | 38,831,749 | C | T | 2032 |
| rs6794328 | 1.83E−03 | 0.036369 | 0.624359 | 38,832,741 | C | T | 2033 |
| chr3: 38893880 | 1.84E−03 | −0.048841 | 0.150865 | 38,893,880 | C | T | 2034 |
| rs62244111 | 1.85E−03 | −0.03618 | 0.615751 | 38,807,690 | C | G | 2035 |
| rs13073816 | 1.85E−03 | 0.048834 | 0.849144 | 38,890,350 | A | T | 2036 |
| rs13096744 | 1.85E−03 | −0.048834 | 0.150856 | 38,890,339 | G | T | 2037 |
| rs35479964 | 1.85E−03 | 0.048829 | 0.849148 | 38,887,930 | C | T | 2038 |
| rs9815891 | 1.86E−03 | −0.036158 | 0.615745 | 38,808,001 | C | T | 2039 |
| chr3: 39238699 | 1.90E−03 | −0.181253 | 0.011791 | 39,238,699 | A | G | 2040 |
| rs9827941 | 1.90E−03 | −0.036454 | 0.373409 | 38,811,463 | A | T | 2041 |
| rs6599261 | 1.92E−03 | 0.036046 | 0.384143 | 38,809,846 | A | T | 2042 |
| rs13087030 | 1.94E−03 | −0.048626 | 0.150221 | 38,849,395 | A | G | 2043 |
| rs62244112 | 1.95E−03 | −0.035986 | 0.615677 | 38,812,037 | A | G | 2044 |
| rs9828912 | 1.95E−03 | 0.035984 | 0.38433 | 38,812,013 | A | T | 2045 |
| rs11720953 | 1.95E−03 | 0.053184 | 0.123087 | 39,301,088 | A | G | 2046 |
| rs73056420 | 1.96E−03 | 0.053115 | 0.123038 | 39,305,266 | A | C | 2047 |
| rs73065296 | 1.96E−03 | 0.096224 | 0.039373 | 38,540,985 | A | G | 2048 |
| rs3792526 | 1.97E−03 | −0.053249 | 0.855581 | 38,380,553 | C | G | 2049 |
| rs62244113 | 1.97E−03 | 0.035947 | 0.384361 | 38,812,742 | A | G | 2050 |
| rs9861437 | 1.99E−03 | −0.053804 | 0.877012 | 39,298,996 | A | G | 2051 |
| rs11129778 | 2.01E−03 | 0.066417 | 0.091582 | 38,109,442 | A | G | 2052 |
| rs12636576 | 2.02E−03 | −0.035852 | 0.615591 | 38,814,845 | A | G | 2053 |
| rs35674337 | 2.04E−03 | −0.048355 | 0.150225 | 38,853,885 | A | G | 2054 |
| rs6792467 | 2.05E−03 | 0.034885 | 0.419524 | 37,752,372 | C | T | 2055 |
| chr3: 37984105 | 2.05E−03 | −0.191517 | 0.978632 | 37,984,105 | C | T | 2056 |
| rs4453791 | 2.07E−03 | −0.048274 | 0.150222 | 38,857,523 | C | T | 2057 |
| rs62244134 | 2.07E−03 | −0.048272 | 0.150222 | 38,863,768 | A | G | 2058 |
| rs13094414 | 2.10E−03 | 0.048211 | 0.849638 | 38,905,572 | A | G | 2059 |
| rs11927309 | 2.12E−03 | 0.048177 | 0.849347 | 38,910,119 | C | T | 2060 |
| rs7619862 | 2.16E−03 | −0.039897 | 0.749704 | 37,765,593 | C | T | 2061 |
| chr3: 38619086 | 2.17E−03 | −0.163913 | 0.984965 | 38,619,086 | A | G | 2062 |
| rs62242262 | 2.19E−03 | −0.048072 | 0.151414 | 38,912,387 | A | C | 2063 |
| rs1768208 | 2.20E−03 | −0.038369 | 0.726172 | 39,498,007 | C | T | 2064 |
| chr3: 38700978 | 2.20E−03 | 0.176205 | 0.984077 | 38,700,978 | A | G | 2065 |
| rs4129279 | 2.21E−03 | 0.036319 | 0.626311 | 38,835,402 | A | C | 2066 |
| rs545397 | 2.24E−03 | −0.038724 | 0.725658 | 39,505,087 | C | T | 2067 |
| rs11712745 | 2.24E−03 | −0.039368 | 0.741612 | 37,754,649 | A | G | 2068 |
| rs616147 | 2.25E−03 | 0.038693 | 0.274407 | 39,509,485 | A | G | 2069 |
| chr3: 39455437 | 2.25E−03 | −0.077817 | 0.088406 | 39,455,437 | A | G | 2070 |
| rs1513219 | 2.27E−03 | −0.038683 | 0.725788 | 39,508,763 | C | T | 2071 |
| rs62244116 | 2.29E−03 | −0.081778 | 0.927946 | 38,820,551 | A | C | 2072 |
| rs11714074 | 2.29E−03 | 0.038757 | 0.736918 | 38,618,789 | C | T | 2073 |
| rs62241188 | 2.30E−03 | −0.05353 | 0.841749 | 38,578,073 | C | G | 2074 |
| rs59478900 | 2.31E−03 | 0.035362 | 0.384637 | 38,828,607 | A | G | 2075 |
| rs1708104 | 2.31E−03 | −0.038836 | 0.727303 | 39,509,746 | C | T | 2076 |
| rs7633317 | 2.31E−03 | 0.040121 | 0.246488 | 37,759,734 | T | G | 2077 |
| chr3: 39318395 | 2.31E−03 | 0.430757 | 0.996421 | 39,318,395 | C | T | 2078 |
| chr3: 37921729 | 2.32E−03 | 0.085237 | 0.944357 | 37,921,729 | C | T | 2079 |
| rs62244117 | 2.33E−03 | 0.035333 | 0.384486 | 38,827,497 | C | T | 2080 |
| chr3: 39115125 | 2.34E−03 | −0.140608 | 0.015723 | 39,115,125 | C | G | 2081 |
| rs1768190 | 2.34E−03 | −0.038224 | 0.725152 | 39,484,444 | C | T | 2082 |
| rs73058955 | 2.37E−03 | −0.08338 | 0.943334 | 37,961,430 | A | G | 2083 |
| chr3: 39086849 | 2.44E−03 | 0.139859 | 0.984128 | 39,086,849 | A | G | 2084 |
| rs2070492 | 2.44E−03 | 0.062006 | 0.100007 | 38,332,821 | T | C | 2085 |
| rs28810495 | 2.45E−03 | −0.038149 | 0.713853 | 39,485,659 | A | G | 2086 |
| chr3: 39022372 | 2.45E−03 | 0.102631 | 0.957007 | 39,022,372 | G | T | 2087 |
| chr3: 38343423 | 2.45E−03 | −0.184009 | 0.023826 | 38,343,423 | C | G | 2088 |
| chr3: 38901737 | 2.46E−03 | 0.208153 | 0.012008 | 38,901,737 | A | C | 2089 |
| rs62244119 | 2.47E−03 | 0.035467 | 0.385001 | 38,834,862 | A | C | 2090 |
| rs62242769 | 2.47E−03 | −0.038466 | 0.262457 | 38,619,266 | A | G | 2091 |
| rs62244120 | 2.49E−03 | −0.046285 | 0.160544 | 38,844,664 | C | G | 2092 |
| chr3: 38362018 | 2.51E−03 | −0.199713 | 0.987857 | 38,362,018 | G | T | 2093 |
| chr3: 37920290 | 2.52E−03 | 0.084852 | 0.945929 | 37,920,290 | A | G | 2094 |
| rs7428946 | 2.56E−03 | 0.065085 | 0.090761 | 38,058,767 | A | C | 2095 |
| chr3: 39021800 | 2.56E−03 | −0.111568 | 0.969653 | 39,021,800 | A | G | 2096 |
| rs73064265 | 2.57E−03 | −0.111518 | 0.969636 | 39,039,515 | C | T | 2097 |
| rs13097780 | 2.62E−03 | 0.046121 | 0.832343 | 38,658,025 | C | G | 2098 |
| chr3: 37965721 | 2.63E−03 | −0.161289 | 0.982585 | 37,965,721 | A | G | 2099 |
| rs17036845 | 2.63E−03 | 0.038754 | 0.262841 | 37,750,978 | A | C | 2100 |
| rs58228096 | 2.64E−03 | −0.038756 | 0.736175 | 37,749,308 | C | T | 2101 |
| chr3: 38628106 | 2.64E−03 | −0.163068 | 0.984458 | 38,628,106 | C | T | 2102 |
| chr3: 37910131 | 2.68E−03 | −0.084417 | 0.053575 | 37,910,131 | A | G | 2103 |
| rs2269350 | 2.73E−03 | −0.037674 | 0.294392 | 39,428,164 | A | G | 2104 |
| chr3: 38599504 | 2.73E−03 | 0.126959 | 0.965283 | 38,599,504 | C | T | 2105 |
| rs73825705 | 2.75E−03 | −0.068963 | 0.090062 | 38,634,252 | C | G | 2106 |
| rs73056401 | 2.76E−03 | −0.083354 | 0.952887 | 39,220,187 | A | T | 2107 |
| chr3: 38771537 | 2.76E−03 | 0.114105 | 0.028608 | 38,771,537 | A | G | 2108 |
| rs1118148 | 2.77E−03 | 0.038366 | 0.259901 | 37,753,485 | C | G | 2109 |
| rs2276866 | 2.77E−03 | −0.038362 | 0.740103 | 37,753,619 | A | T | 2110 |
| rs7433495 | 2.77E−03 | −0.055077 | 0.764993 | 39,555,967 | C | T | 2111 |
| rs11709059 | 2.78E−03 | 0.038357 | 0.259886 | 37,754,482 | G | T | 2112 |
| chr3: 38811822 | 2.78E−03 | 0.050979 | 0.801556 | 38,811,822 | G | T | 2113 |
| rs1402755 | 2.79E−03 | −0.038344 | 0.740137 | 37,756,282 | C | T | 2114 |
| rs6550504 | 2.82E−03 | −0.038395 | 0.735212 | 37,748,433 | C | T | 2115 |
| rs7613157 | 2.82E−03 | 0.038387 | 0.264901 | 37,748,375 | A | G | 2116 |
| chr3: 39519772 | 2.82E−03 | 0.210249 | 0.983785 | 39,519,772 | A | C | 2117 |
| rs3922580 | 2.84E−03 | 0.035799 | 0.625476 | 38,836,298 | C | T | 2118 |
| chr3: 38843435 | 2.92E−03 | 0.04713 | 0.853292 | 38,843,435 | C | T | 2119 |
| rs11919746 | 2.95E−03 | 0.04707 | 0.853193 | 38,846,900 | C | T | 2120 |
| rs9832895 | 2.97E−03 | 0.038315 | 0.563513 | 38,636,537 | C | T | 2121 |
| rs62244136 | 2.97E−03 | −0.114564 | 0.02482 | 38,865,438 | A | G | 2122 |
| rs784518 | 3.03E−03 | −0.037652 | 0.390642 | 39,161,042 | C | G | 2123 |
| chr3: 39000837 | 3.04E−03 | −0.134769 | 0.977692 | 39,000,837 | A | G | 2124 |
| chr3: 38182011 | 3.15E−03 | 0.066098 | 0.128366 | 38,182,011 | G | T | 2125 |
| rs13081054 | 3.17E−03 | −0.033817 | 0.486658 | 39,447,451 | A | G | 2126 |
| rs9857730 | 3.21E−03 | −0.043252 | 0.184284 | 38,026,945 | C | T | 2127 |
| rs4676613 | 3.26E−03 | −0.036393 | 0.285834 | 39,223,257 | C | G | 2128 |
| chr3: 38900648 | 3.28E−03 | 0.138139 | 0.022044 | 38,900,648 | C | T | 2129 |
| rs73056438 | 3.50E−03 | −0.059495 | 0.897729 | 38,646,478 | C | T | 2130 |
| rs196378 | 3.54E−03 | −0.035404 | 0.434786 | 38,392,349 | C | G | 2131 |
| chr3: 39281780 | 3.58E−03 | −0.231714 | 0.014006 | 39,281,780 | G | T | 2132 |
| chr3: 38758236 | 3.61E−03 | 0.16503 | 0.988793 | 38,758,236 | C | T | 2133 |
| rs169045 | 3.61E−03 | −0.035229 | 0.434819 | 38,394,586 | C | T | 2134 |
| chr3: 38437231 | 3.63E−03 | 0.149554 | 0.961836 | 38,437,231 | G | T | 2135 |
| chr3: 38548964 | 3.67E−03 | 0.146293 | 0.016497 | 38,548,964 | A | G | 2136 |
| rs13067055 | 3.69E−03 | 0.036067 | 0.306577 | 39,485,521 | A | G | 2137 |
| rs41315507 | 3.72E−03 | −0.08254 | 0.943966 | 38,572,871 | C | T | 2138 |
| chr3: 37944167 | 3.72E−03 | 0.076193 | 0.086811 | 37,944,167 | A | G | 2139 |
| chr3: 38318752 | 3.72E−03 | −0.04387 | 0.218405 | 38,318,752 | C | T | 2140 |
| rs34743428 | 3.76E−03 | −0.037067 | 0.650863 | 39,573,199 | A | T | 2141 |
| rs9819612 | 3.76E−03 | −0.034749 | 0.397194 | 38,834,980 | C | T | 2142 |
| rs73052961 | 3.78E−03 | −0.039215 | 0.774582 | 37,759,524 | A | G | 2143 |
| chr3: 39636115 | 3.82E−03 | 0.290626 | 0.990461 | 39,636,115 | A | G | 2144 |
| chr3: 38133126 | 3.82E−03 | 0.148964 | 0.012969 | 38,133,126 | A | G | 2145 |
| rs2284815 | 3.85E−03 | −0.050116 | 0.856069 | 38,389,678 | C | T | 2146 |
| rs6789468 | 3.86E−03 | 0.04686 | 0.851191 | 37,988,497 | A | G | 2147 |
| chr3: 39111314 | 3.90E−03 | 0.133923 | 0.021458 | 39,111,314 | A | T | 2148 |
| rs41312061 | 3.97E−03 | 0.137025 | 0.021433 | 38,659,631 | A | G | 2149 |
| chr3: 38408873 | 4.05E−03 | −0.146861 | 0.984239 | 38,408,873 | A | G | 2150 |
| rs9311172 | 4.05E−03 | 0.041383 | 0.81087 | 38,019,147 | C | G | 2151 |
| rs928807 | 4.09E−03 | −0.041278 | 0.188851 | 38,017,186 | G | T | 2152 |
| rs6769106 | 4.12E−03 | −0.041329 | 0.189053 | 38,019,973 | A | G | 2153 |
| rs6793254 | 4.14E−03 | 0.041316 | 0.810968 | 38,019,955 | A | G | 2154 |
| rs9843506 | 4.14E−03 | 0.04135 | 0.810854 | 38,021,027 | A | C | 2155 |
| rs9311173 | 4.14E−03 | −0.041334 | 0.189121 | 38,020,532 | A | G | 2156 |
| chr3: 38589666 | 4.14E−03 | −0.12489 | 0.03397 | 38,589,666 | A | G | 2157 |
| rs2070486 | 4.15E−03 | −0.049703 | 0.85603 | 38,389,795 | A | C | 2158 |
| rs9311176 | 4.16E−03 | 0.041384 | 0.810701 | 38,022,611 | A | C | 2159 |
| chr3: 39370781 | 4.19E−03 | −0.100309 | 0.968605 | 39,370,781 | A | G | 2160 |
| rs9876660 | 4.21E−03 | −0.049043 | 0.159312 | 38,639,719 | C | G | 2161 |
| chr3: 38844937 | 4.24E−03 | 0.109741 | 0.955277 | 38,844,937 | A | C | 2162 |
| chr3: 38017656 | 4.27E−03 | −0.175301 | 0.989781 | 38,017,656 | C | T | 2163 |
| rs2154776 | 4.34E−03 | −0.160913 | 0.984849 | 38,039,739 | A | C | 2164 |
| rs9311193 | 4.34E−03 | −0.034523 | 0.318703 | 38,609,663 | C | T | 2165 |
| rs2070487 | 4.36E−03 | 0.049528 | 0.142806 | 38,393,009 | A | G | 2166 |
| rs1573291 | 4.40E−03 | 0.04736 | 0.8652 | 37,984,753 | C | T | 2167 |
| chr3: 38431678 | 4.43E−03 | −0.338244 | 0.006759 | 38,431,678 | C | T | 2168 |
| rs9829569 | 4.50E−03 | 0.040937 | 0.811398 | 38,013,333 | A | G | 2169 |
| rs6776469 | 4.51E−03 | 0.049381 | 0.1469 | 38,394,012 | A | G | 2170 |
| rs28707243 | 4.53E−03 | −0.047467 | 0.68822 | 38,428,495 | C | T | 2171 |
| rs879444 | 4.58E−03 | 0.032363 | 0.505084 | 39,454,482 | C | T | 2172 |
| rs56990533 | 4.66E−03 | −0.08019 | 0.947176 | 38,569,611 | A | G | 2173 |
| rs1392283 | 4.67E−03 | 0.092036 | 0.040608 | 38,229,592 | C | T | 2174 |
| rs9823482 | 4.67E−03 | 0.04079 | 0.811498 | 38,011,811 | A | G | 2175 |
| rs41312437 | 4.69E−03 | 0.144416 | 0.014943 | 38,624,950 | A | G | 2176 |
| rs45505695 | 4.73E−03 | 0.080416 | 0.052743 | 38,573,403 | A | G | 2177 |
| rs73062884 | 4.75E−03 | 0.091895 | 0.040568 | 38,310,396 | A | G | 2178 |
| chr3: 39513170 | 4.76E−03 | −0.139775 | 0.022624 | 39,513,170 | C | T | 2179 |
| chr3: 38591624 | 4.77E−03 | 0.121504 | 0.966176 | 38,591,624 | G | T | 2180 |
| rs13096483 | 4.78E−03 | −0.03278 | 0.499713 | 39,449,429 | C | T | 2181 |
| rs169046 | 4.81E−03 | −0.034419 | 0.431862 | 38,392,371 | C | T | 2182 |
| rs73067159 | 4.82E−03 | −0.168738 | 0.982403 | 38,575,765 | C | T | 2183 |
| rs56157245 | 4.82E−03 | 0.048824 | 0.145961 | 38,395,386 | A | G | 2184 |
| rs73825594 | 4.83E−03 | 0.082195 | 0.052949 | 38,577,610 | A | G | 2185 |
| chr3: 38575105 | 4.83E−03 | 0.080374 | 0.052822 | 38,575,105 | A | G | 2186 |
| rs55707921 | 4.87E−03 | 0.048781 | 0.145685 | 38,395,400 | C | T | 2187 |
| chr3: 39309763 | 4.89E−03 | 0.314051 | 0.007787 | 39,309,763 | A | G | 2188 |
| rs34897882 | 4.89E−03 | 0.032072 | 0.504258 | 39,473,313 | C | T | 2189 |
| chr3: 38602671 | 4.93E−03 | −0.051406 | 0.235149 | 38,602,671 | C | G | 2190 |
| chr3: 38440647 | 4.99E−03 | 0.07772 | 0.924059 | 38,440,647 | A | G | 2191 |
| rs938183 | 5.10E−03 | −0.032782 | 0.504068 | 39,455,298 | A | G | 2192 |
| rs9872804 | 5.10E−03 | 0.040443 | 0.811712 | 38,008,466 | C | T | 2193 |
| rs56055643 | 5.11E−03 | −0.032248 | 0.481018 | 39,447,633 | A | G | 2194 |
| rs11719907 | 5.12E−03 | 0.046785 | 0.866258 | 37,984,704 | C | T | 2195 |
| rs13321209 | 5.13E−03 | −0.037265 | 0.764965 | 37,755,813 | A | G | 2196 |
| rs6786403 | 5.14E−03 | −0.086758 | 0.966861 | 38,198,086 | C | T | 2197 |
| rs7614714 | 5.14E−03 | −0.07094 | 0.93386 | 38,057,067 | C | T | 2198 |
| rs7625114 | 5.15E−03 | −0.086757 | 0.96687 | 38,189,717 | A | G | 2199 |
| chr3: 38494146 | 5.15E−03 | −0.236321 | 0.009346 | 38,494,146 | A | T | 2200 |
| chr3: 38804546 | 5.18E−03 | −0.104491 | 0.968816 | 38,804,546 | C | T | 2201 |
| rs71325510 | 5.24E−03 | 0.058795 | 0.869163 | 39,191,136 | A | G | 2202 |
| rs73056224 | 5.26E−03 | −0.043439 | 0.83993 | 39,338,624 | A | G | 2203 |
| chr3: 38152639 | 5.28E−03 | 0.086626 | 0.033054 | 38,152,639 | C | T | 2204 |
| rs3935183 | 5.29E−03 | −0.04227 | 0.831579 | 38,613,188 | A | G | 2205 |
| chr3: 39416948 | 5.30E−03 | −0.047145 | 0.702948 | 39,416,948 | C | T | 2206 |
| chr3: 39258463 | 5.30E−03 | 0.169154 | 0.015153 | 39,258,463 | A | C | 2207 |
| rs674243 | 5.31E−03 | −0.031932 | 0.496809 | 39,453,699 | C | T | 2208 |
| rs73054549 | 5.35E−03 | −0.042762 | 0.843494 | 38,631,999 | A | G | 2209 |
| rs11926412 | 5.36E−03 | 0.043314 | 0.1602 | 39,338,419 | C | T | 2210 |
| rs41312945 | 5.39E−03 | 0.042697 | 0.156833 | 38,631,849 | A | G | 2211 |
| rs62241769 | 5.39E−03 | 0.04769 | 0.18831 | 38,509,300 | C | T | 2212 |
| chr3: 38140553 | 5.39E−03 | −0.08647 | 0.966963 | 38,140,553 | A | G | 2213 |
| chr3: 38139868 | 5.40E−03 | 0.086459 | 0.033036 | 38,139,868 | C | T | 2214 |
| chr3: 38138454 | 5.42E−03 | −0.086438 | 0.966965 | 38,138,454 | C | T | 2215 |
| rs6773586 | 5.46E−03 | −0.042919 | 0.839158 | 39,326,461 | A | T | 2216 |
| chr3: 38680126 | 5.51E−03 | −0.118124 | 0.970366 | 38,680,126 | C | T | 2217 |
| rs4996738 | 5.56E−03 | −0.034949 | 0.304396 | 39,313,684 | G | T | 2218 |
| chr3: 39609933 | 5.56E−03 | 0.232073 | 0.98404 | 39,609,933 | G | T | 2219 |
| chr3: 39475164 | 5.58E−03 | −0.03148 | 0.499144 | 39,475,164 | C | T | 2220 |
| rs7651347 | 5.60E−03 | −0.042825 | 0.839876 | 39,315,054 | C | T | 2221 |
| rs73054554 | 5.66E−03 | −0.043339 | 0.845257 | 38,633,061 | A | T | 2222 |
| rs7644531 | 5.75E−03 | −0.039931 | 0.187983 | 38,005,937 | A | T | 2223 |
| rs9840828 | 5.79E−03 | 0.036732 | 0.234197 | 37,757,392 | G | T | 2224 |
| rs7632911 | 5.79E−03 | −0.039903 | 0.187967 | 38,005,903 | A | G | 2225 |
| chr3: 39340976 | 5.79E−03 | −0.22115 | 0.006301 | 39,340,976 | A | G | 2226 |
| rs11709075 | 5.82E−03 | −0.031301 | 0.499948 | 39,475,249 | A | G | 2227 |
| rs11709044 | 5.85E−03 | −0.031276 | 0.49996 | 39,475,204 | A | G | 2228 |
| chr3: 39423329 | 5.89E−03 | 0.498081 | 0.005121 | 39,423,329 | A | G | 2229 |
| rs12495623 | 5.89E−03 | −0.031424 | 0.496543 | 39,454,885 | G | T | 2230 |
| rs1009966 | 5.91E−03 | 0.031382 | 0.50367 | 39,448,595 | A | G | 2231 |
| rs11705761 | 5.91E−03 | −0.090316 | 0.963069 | 39,195,669 | C | T | 2232 |
| rs11129833 | 5.92E−03 | 0.031372 | 0.50373 | 39,451,385 | G | T | 2233 |
| rs11715770 | 5.96E−03 | −0.039777 | 0.187898 | 38,005,751 | A | G | 2234 |
| chr3: 39555969 | 5.96E−03 | −0.071786 | 0.894631 | 39,555,969 | C | T | 2235 |
| rs6773329 | 5.99E−03 | −0.038897 | 0.777762 | 38,689,480 | A | G | 2236 |
| rs73056206 | 6.01E−03 | 0.042395 | 0.160343 | 39,329,947 | A | G | 2237 |
| chr3: 38118773 | 6.01E−03 | 0.1428 | 0.013435 | 38,118,773 | G | T | 2238 |
| chr3: 39377371 | 6.05E−03 | 0.083243 | 0.037428 | 39,377,371 | A | G | 2239 |
| chr3: 38807868 | 6.05E−03 | −0.128737 | 0.980023 | 38,807,868 | C | G | 2240 |
| chr3: 39471903 | 6.07E−03 | 0.604999 | 0.004236 | 39,471,903 | A | G | 2241 |
| chr3: 39717722 | 6.08E−03 | −0.061197 | 0.109357 | 39,717,722 | C | G | 2242 |
| rs11129834 | 6.28E−03 | 0.03109 | 0.505621 | 39,463,350 | A | G | 2243 |
| rs12638676 | 6.35E−03 | 0.031291 | 0.487182 | 39,477,729 | A | G | 2244 |
| rs12495185 | 6.42E−03 | −0.031021 | 0.49789 | 39,447,794 | A | G | 2245 |
| rs9875443 | 6.43E−03 | −0.044538 | 0.840189 | 38,657,275 | A | T | 2246 |
| rs6770798 | 6.43E−03 | 0.030972 | 0.505437 | 39,464,883 | C | T | 2247 |
| rs6770797 | 6.44E−03 | −0.030971 | 0.494563 | 39,464,881 | A | C | 2248 |
| rs73064834 | 6.44E−03 | −0.077286 | 0.957144 | 38,333,860 | A | G | 2249 |
| rs2965067 | 6.53E−03 | 0.032689 | 0.329949 | 39,478,041 | A | G | 2250 |
| rs34629457 | 6.53E−03 | 0.030913 | 0.503328 | 39,466,571 | C | T | 2251 |
| rs13073538 | 6.54E−03 | 0.031271 | 0.512415 | 39,449,416 | A | G | 2252 |
| rs7638977 | 6.56E−03 | 0.030947 | 0.501917 | 39,448,105 | A | T | 2253 |
| rs1768209 | 6.66E−03 | 0.032582 | 0.330272 | 39,477,679 | C | G | 2254 |
| rs1707981 | 6.70E−03 | −0.032547 | 0.669638 | 39,477,510 | G | T | 2255 |
| rs11705945 | 6.74E−03 | 0.030829 | 0.502618 | 39,459,727 | A | G | 2256 |
| rs1392191 | 6.77E−03 | 0.101013 | 0.02616 | 39,391,157 | C | T | 2257 |
| chr3: 39387840 | 6.80E−03 | 0.100778 | 0.026052 | 39,387,840 | A | G | 2258 |
| rs2669845 | 6.81E−03 | −0.049088 | 0.885043 | 39,296,222 | C | T | 2259 |
| rs7374138 | 6.83E−03 | 0.051192 | 0.787241 | 38,580,736 | C | G | 2260 |
| rs2853704 | 6.84E−03 | 0.061662 | 0.926029 | 39,318,526 | C | T | 2261 |
| rs73060545 | 6.85E−03 | 0.049067 | 0.115018 | 39,295,602 | A | G | 2262 |
| rs2669843 | 6.85E−03 | −0.049068 | 0.884993 | 39,295,059 | A | G | 2263 |
| rs35310432 | 6.86E−03 | 0.030729 | 0.500997 | 39,449,962 | A | G | 2264 |
| chr3: 39389382 | 6.89E−03 | 0.100689 | 0.025983 | 39,389,382 | C | T | 2265 |
| chr3: 39388918 | 6.89E−03 | 0.100661 | 0.025971 | 39,388,918 | A | G | 2266 |
| chr3: 39384832 | 6.92E−03 | 0.100387 | 0.025908 | 39,384,832 | C | T | 2267 |
| rs34029841 | 6.92E−03 | −0.030698 | 0.497597 | 39,460,244 | C | G | 2268 |
| chr3: 39376174 | 6.94E−03 | 0.099931 | 0.02568 | 39,376,174 | A | G | 2269 |
| chr3: 39385505 | 6.94E−03 | 0.100375 | 0.025839 | 39,385,505 | C | T | 2270 |
| chr3: 39382184 | 6.95E−03 | 0.100187 | 0.025767 | 39,382,184 | C | T | 2271 |
| chr3: 39366736 | 6.95E−03 | 0.099744 | 0.025613 | 39,366,736 | A | G | 2272 |
| chr3: 39370532 | 6.95E−03 | 0.099806 | 0.025633 | 39,370,532 | A | G | 2273 |
| rs61732061 | 6.95E−03 | −0.099903 | 0.974336 | 39,376,069 | C | T | 2274 |
| chr3: 39365368 | 6.95E−03 | 0.099791 | 0.025568 | 39,365,368 | C | T | 2275 |
| chr3: 39373640 | 6.95E−03 | −0.09985 | 0.974354 | 39,373,640 | A | G | 2276 |
| chr3: 39366585 | 6.96E−03 | 0.099716 | 0.025598 | 39,366,585 | A | G | 2277 |
| chr3: 39366645 | 6.96E−03 | −0.099717 | 0.974402 | 39,366,645 | G | T | 2278 |
| chr3: 39366935 | 6.96E−03 | 0.099722 | 0.025599 | 39,366,935 | A | G | 2279 |
| rs7638752 | 6.96E−03 | 0.099763 | 0.025612 | 39,369,502 | C | G | 2280 |
| chr3: 39374205 | 6.96E−03 | −0.09984 | 0.974362 | 39,374,205 | C | T | 2281 |
| chr3: 39370419 | 6.97E−03 | 0.552067 | 0.002067 | 39,370,419 | A | G | 2282 |
| chr3: 39379661 | 6.97E−03 | 0.09997 | 0.02568 | 39,379,661 | C | T | 2283 |
| chr3: 39369972 | 6.98E−03 | 0.275896 | 0.004056 | 39,369,972 | A | G | 2284 |
| chr3: 39386312 | 6.99E−03 | 0.100332 | 0.025807 | 39,386,312 | A | G | 2285 |
| rs11129832 | 7.03E−03 | 0.030614 | 0.500396 | 39,447,529 | C | T | 2286 |
| rs2649749 | 7.03E−03 | 0.073029 | 0.951535 | 39,335,769 | A | T | 2287 |
| rs13059755 | 7.08E−03 | 0.030649 | 0.502918 | 39,459,515 | C | T | 2288 |
| rs36230797 | 7.15E−03 | 0.049122 | 0.114034 | 39,296,809 | C | T | 2289 |
| rs73062575 | 7.17E−03 | −0.10497 | 0.972279 | 38,741,764 | G | T | 2290 |
| rs2268750 | 7.30E−03 | −0.052614 | 0.910474 | 38,334,012 | G | T | 2291 |
| rs73064832 | 7.30E−03 | −0.052606 | 0.910494 | 38,333,800 | C | T | 2292 |
| rs12494100 | 7.31E−03 | −0.030448 | 0.499465 | 39,456,839 | A | G | 2293 |
| chr3: 38814087 | 7.38E−03 | −0.126012 | 0.968637 | 38,814,087 | A | G | 2294 |
| chr3: 38757371 | 7.40E−03 | 0.145848 | 0.013757 | 38,757,371 | A | G | 2295 |
| rs7640507 | 7.49E−03 | −0.04103 | 0.834847 | 39,324,614 | G | T | 2296 |
| chr3: 39386485 | 7.53E−03 | 0.100579 | 0.025535 | 39,386,485 | C | T | 2297 |
| chr3: 39383294 | 7.59E−03 | −0.275035 | 0.995359 | 39,383,294 | C | G | 2298 |
| chr3: 39360064 | 7.63E−03 | 0.09849 | 0.02571 | 39,360,064 | C | T | 2299 |
| chr3: 38658246 | 7.68E−03 | −0.059804 | 0.910479 | 38,658,246 | A | T | 2300 |
| rs4525803 | 7.68E−03 | 0.030199 | 0.501433 | 39,465,626 | G | T | 2301 |
| rs4676635 | 7.68E−03 | −0.098399 | 0.974281 | 39,359,820 | C | T | 2302 |
| chr3: 39573571 | 7.78E−03 | −0.607708 | 0.995555 | 39,573,571 | C | G | 2303 |
| rs36020975 | 7.79E−03 | −0.030123 | 0.499579 | 39,466,484 | A | G | 2304 |
| rs3914283 | 7.86E−03 | −0.030077 | 0.499603 | 39,466,288 | C | T | 2305 |
| chr3: 39261921 | 7.97E−03 | 0.093014 | 0.966877 | 39,261,921 | C | T | 2306 |
| chr3: 38352328 | 7.97E−03 | 0.042649 | 0.454071 | 38,352,328 | C | T | 2307 |
| rs73062845 | 8.02E−03 | −0.089827 | 0.961469 | 38,127,648 | A | G | 2308 |
| chr3: 39358213 | 8.05E−03 | −0.09779 | 0.974217 | 39,358,213 | C | T | 2309 |
| chr3: 38193667 | 8.15E−03 | 0.162806 | 0.011144 | 38,193,667 | C | G | 2310 |
| chr3: 39332286 | 8.15E−03 | 0.26837 | 0.003908 | 39,332,286 | A | G | 2311 |
| rs41312391 | 8.17E−03 | −0.045874 | 0.849766 | 38,573,610 | C | T | 2312 |
| chr3: 37857503 | 8.17E−03 | −0.20634 | 0.992799 | 37,857,503 | A | T | 2313 |
| chr3: 39357132 | 8.21E−03 | −0.098393 | 0.974454 | 39,357,132 | C | T | 2314 |
| chr3: 39357237 | 8.24E−03 | 0.097482 | 0.025888 | 39,357,237 | A | G | 2315 |
| rs73067106 | 8.24E−03 | −0.068304 | 0.925715 | 38,544,995 | G | T | 2316 |
| chr3: 38095026 | 8.33E−03 | −0.13734 | 0.987328 | 38,095,026 | C | T | 2317 |
| chr3: 39275596 | 8.47E−03 | 0.136636 | 0.013414 | 39,275,596 | A | C | 2318 |
| rs816510 | 8.47E−03 | −0.031342 | 0.663427 | 39,447,299 | G | T | 2319 |
| chr3: 37925422 | 8.73E−03 | 0.205016 | 0.007728 | 37,925,422 | A | G | 2320 |
| chr3: 39393491 | 8.79E−03 | 0.132059 | 0.018693 | 39,393,491 | A | G | 2321 |
| rs674192 | 8.80E−03 | 0.031084 | 0.333852 | 39,475,148 | A | G | 2322 |
| rs6599222 | 8.92E−03 | 0.040946 | 0.160447 | 38,623,066 | C | T | 2323 |
| rs55812368 | 8.96E−03 | −0.042091 | 0.829218 | 38,661,898 | C | G | 2324 |
| rs172111 | 8.97E−03 | −0.05969 | 0.929001 | 38,162,998 | C | T | 2325 |
| rs41315501 | 8.98E−03 | 0.087552 | 0.044081 | 38,572,062 | A | G | 2326 |
| chr3: 39404899 | 9.01E−03 | −0.129526 | 0.022635 | 39,404,899 | C | G | 2327 |
| chr3: 38496100 | 9.01E−03 | 0.079995 | 0.93265 | 38,496,100 | C | T | 2328 |
| chr3: 38098852 | 9.05E−03 | 0.088359 | 0.03965 | 38,098,852 | A | G | 2329 |
| chr3: 39280692 | 9.06E−03 | 0.145539 | 0.978436 | 39,280,692 | C | T | 2330 |
| rs2133578 | 9.16E−03 | −0.031613 | 0.341493 | 39,421,155 | C | T | 2331 |
| chr3: 39600305 | 9.23E−03 | 0.610006 | 0.005005 | 39,600,305 | C | T | 2332 |
| rs151618 | 9.27E−03 | −0.032599 | 0.493218 | 38,399,189 | T | C | 2333 |
| rs166631 | 9.32E−03 | 0.059314 | 0.071065 | 38,195,472 | A | G | 2334 |
| rs156261 | 9.33E−03 | 0.059271 | 0.07097 | 38,190,440 | A | G | 2335 |
| chr3: 39309140 | 9.33E−03 | 0.263445 | 0.003839 | 39,309,140 | C | T | 2336 |
| chr3: 38802401 | 9.35E−03 | 0.142818 | 0.013209 | 38,802,401 | A | T | 2337 |
| rs73056454 | 9.36E−03 | −0.041099 | 0.8348 | 38,656,360 | C | T | 2338 |
| chr3: 38872314 | 9.43E−03 | −0.056764 | 0.114219 | 38,872,314 | C | T | 2339 |
| rs17756489 | 9.55E−03 | 0.029719 | 0.440908 | 39,519,947 | C | T | 2340 |
| rs272576 | 9.65E−03 | −0.058899 | 0.929157 | 38,233,183 | C | T | 2341 |
| chr3: 39691686 | 9.76E−03 | −0.043787 | 0.650087 | 39,691,686 | A | C | 2342 |
| rs9871799 | 9.85E−03 | −0.030632 | 0.663931 | 39,459,324 | A | G | 2343 |
| chr3: 38559849 | 9.95E−03 | 0.084372 | 0.940349 | 38,559,849 | A | G | 2344 |
| rs528364 | 9.98E−03 | 0.030567 | 0.336385 | 39,461,253 | A | G | 2345 |

TABLE 19
Association results for QRS interval in a 2
Mb region flanking rs6795970 on chromosome 3.

| | | | | | effect | other | SEQ ID |
|---|---|---|---|---|---|---|---|
| marker | p-value | effect | freq | position | allele | allele | NO: |
| rs6781009 | 2.32E−08 | 0.07282 | 0.236685 | 38,560,438 | C | T | 2346 |
| rs6762565 | 2.33E−08 | −0.074246 | 0.771542 | 38,557,195 | C | T | 2347 |
| rs10865879 | 2.39E−08 | −0.074258 | 0.771705 | 38,552,366 | A | C | 2348 |
| rs55880069 | 2.77E−08 | 0.074014 | 0.227407 | 38,558,045 | C | T | 2349 |
| rs55872442 | 3.50E−08 | 0.073641 | 0.226204 | 38,556,439 | A | G | 2350 |
| rs1473805 | 4.57E−08 | −0.07314 | 0.775038 | 38,552,683 | A | G | 2351 |
| rs9880327 | 4.78E−08 | −0.068249 | 0.265034 | 38,657,897 | A | G | 2352 |
| rs11710498 | 4.90E−08 | 0.072963 | 0.233457 | 38,551,669 | C | T | 2353 |
| rs9856387 | 5.20E−08 | 0.06822 | 0.735989 | 38,662,230 | C | T | 2354 |
| rs41315485 | 5.54E−08 | −0.073575 | 0.767815 | 38,565,279 | A | G | 2355 |
| rs35231307 | 6.10E−08 | −0.072141 | 0.776282 | 38,564,412 | C | T | 2356 |
| rs1473804 | 6.46E−08 | −0.072321 | 0.776438 | 38,551,394 | C | T | 2357 |
| rs11129795 | 6.56E−08 | 0.071955 | 0.223435 | 38,564,167 | A | G | 2358 |
| rs12497866 | 6.60E−08 | −0.072279 | 0.776514 | 38,550,869 | C | T | 2359 |
| rs62239356 | 6.63E−08 | 0.072007 | 0.223882 | 38,562,097 | C | T | 2360 |
| rs56283404 | 6.77E−08 | 0.071957 | 0.223353 | 38,561,710 | A | C | 2361 |
| rs12490047 | 6.80E−08 | −0.071957 | 0.776656 | 38,561,419 | C | G | 2362 |
| rs6771881 | 7.21E−08 | −0.072001 | 0.776836 | 38,553,268 | A | G | 2363 |
| chr3: 38643196 | 1.38E−07 | −0.078518 | 0.202742 | 38,643,196 | A | G | 2364 |
| rs6599233 | 1.63E−07 | 0.06473 | 0.721856 | 38,687,884 | C | T | 2365 |
| rs13091192 | 2.23E−07 | 0.057884 | 0.427403 | 38,709,756 | A | G | 2366 |
| chr3: 38357854 | 2.41E−07 | −0.134945 | 0.132454 | 38,357,854 | A | T | 2367 |
| rs6599240 | 2.52E−07 | 0.056796 | 0.435855 | 38,713,721 | A | G | 1 |
| rs11129800 | 2.59E−07 | −0.056854 | 0.563857 | 38,719,374 | C | T | 2 |
| rs7375123 | 3.40E−07 | −0.077633 | 0.1914 | 38,645,102 | A | G | 2368 |
| rs6782237 | 4.57E−07 | 0.062228 | 0.720125 | 38,671,557 | C | G | 2369 |
| rs4131768 | 4.65E−07 | 0.062186 | 0.720193 | 38,670,178 | A | G | 2370 |
| rs6599232 | 5.01E−07 | −0.062159 | 0.280398 | 38,674,764 | C | T | 2371 |
| rs9851724 | 5.50E−07 | 0.062781 | 0.718205 | 38,694,939 | T | C | 2372 |
| rs7624535 | 8.88E−07 | −0.075192 | 0.185688 | 38,640,206 | G | T | 2373 |
| rs11129801 | 9.71E−07 | −0.05894 | 0.295848 | 38,725,379 | A | G | 3 |
| rs9832895 | 1.11E−06 | 0.061256 | 0.563513 | 38,636,537 | C | T | 2374 |
| rs12631535 | 1.46E−06 | 0.066438 | 0.78803 | 38,706,121 | T | C | 2375 |
| rs6599234 | 1.91E−06 | −0.057585 | 0.29089 | 38,690,304 | A | T | 2376 |
| rs62243862 | 2.02E−06 | −0.065433 | 0.201294 | 38,716,553 | C | T | 2377 |
| rs6599210 | 2.57E−06 | 0.073235 | 0.140932 | 38,533,214 | A | G | 2378 |
| rs9860525 | 2.63E−06 | 0.073236 | 0.141056 | 38,547,009 | A | T | 2379 |
| rs7430391 | 2.77E−06 | −0.057354 | 0.290387 | 38,698,971 | A | G | 2380 |
| rs7633988 | 2.96E−06 | 0.057118 | 0.709848 | 38,698,221 | A | T | 2381 |
| rs62242859 | 3.04E−06 | 0.064437 | 0.800065 | 38,712,327 | C | T | 2382 |
| rs6779704 | 3.08E−06 | −0.072606 | 0.857669 | 38,515,268 | A | T | 2383 |
| rs7430191 | 3.15E−06 | 0.069543 | 0.823635 | 38,629,028 | C | T | 2384 |
| rs9851710 | 3.21E−06 | 0.056223 | 0.709099 | 38,694,905 | A | C | 2385 |
| rs12639182 | 3.55E−06 | 0.06402 | 0.800504 | 38,711,234 | C | T | 2386 |
| rs6599230 | 4.35E−06 | 0.07161 | 0.836514 | 38,649,716 | C | T | 2387 |
| rs13097780 | 4.46E−06 | 0.068364 | 0.832343 | 38,658,025 | C | G | 2388 |
| rs3796387 | 4.98E−06 | 0.068856 | 0.171953 | 38,554,211 | A | G | 2389 |
| rs6783110 | 5.00E−06 | 0.052424 | 0.359181 | 38,727,939 | A | G | 2390 |
| rs11924846 | 5.02E−06 | 0.052378 | 0.359263 | 38,731,570 | C | T | 5 |
| rs11710077 | 5.19E−06 | 0.069266 | 0.824592 | 38,632,903 | A | T | 2391 |
| rs4076737 | 5.80E−06 | 0.051848 | 0.359175 | 38,739,786 | G | T | 10 |
| rs6795970 | 6.45E−06 | 0.051815 | 0.36095 | 38,741,679 | A | G | 13 |
| rs6773331 | 7.45E−06 | −0.185657 | 0.02334 | 38,659,401 | A | T | 2392 |
| rs7373492 | 7.89E−06 | 0.185316 | 0.976569 | 38,662,373 | C | T | 2393 |
| rs6810361 | 8.12E−06 | 0.069814 | 0.164832 | 38,549,972 | C | T | 2394 |
| rs7638275 | 8.74E−06 | −0.18721 | 0.022746 | 38,640,827 | A | G | 2395 |
| chr3: 38512241 | 8.93E−06 | 0.129077 | 0.046847 | 38,512,241 | A | G | 2396 |
| rs12490478 | 1.20E−05 | −0.053893 | 0.346668 | 38,792,703 | G | T | 2397 |
| rs35036319 | 1.52E−05 | −0.059548 | 0.216467 | 38,702,330 | A | G | 2398 |
| rs7373065 | 1.56E−05 | 0.178357 | 0.975596 | 38,685,319 | C | T | 2399 |
| rs9878604 | 2.38E−05 | 0.051985 | 0.669519 | 38,801,665 | C | T | 2400 |
| rs11926158 | 2.48E−05 | −0.052169 | 0.333793 | 38,798,319 | C | G | 2401 |
| rs7433306 | 2.50E−05 | 0.048195 | 0.362944 | 38,745,643 | C | G | 15 |
| rs6804918 | 2.58E−05 | −0.052851 | 0.714411 | 38,573,960 | A | G | 2402 |
| rs6800541 | 2.60E−05 | 0.047776 | 0.363841 | 38,749,836 | C | T | 18 |
| rs9990137 | 2.76E−05 | 0.048209 | 0.652707 | 38,734,469 | A | G | 6 |
| rs7645178 | 2.82E−05 | −0.052193 | 0.712714 | 38,572,562 | A | G | 2403 |
| rs6805187 | 2.82E−05 | −0.048153 | 0.347023 | 38,735,510 | A | C | 7 |
| rs9820042 | 2.85E−05 | 0.047577 | 0.363935 | 38,754,120 | C | T | 2404 |
| rs9844378 | 2.91E−05 | −0.05401 | 0.262317 | 38,675,875 | A | G | 2405 |
| rs12053903 | 3.04E−05 | 0.05118 | 0.278414 | 38,568,397 | C | T | 2406 |
| rs7373779 | 3.09E−05 | 0.051192 | 0.278326 | 38,568,947 | C | T | 2407 |
| rs6599250 | 3.18E−05 | −0.047314 | 0.635957 | 38,759,033 | C | T | 20 |
| rs9843500 | 3.33E−05 | 0.04971 | 0.303238 | 38,563,099 | C | G | 2408 |
| rs4073796 | 3.33E−05 | 0.049502 | 0.304242 | 38,565,853 | A | G | 2409 |
| rs4073797 | 3.34E−05 | −0.049502 | 0.695758 | 38,565,854 | A | T | 2410 |
| rs9844577 | 3.37E−05 | −0.04722 | 0.637157 | 38,763,732 | A | C | 2411 |
| rs6793245 | 3.49E−05 | 0.052018 | 0.276953 | 38,574,041 | A | G | 2412 |
| rs7429945 | 3.50E−05 | 0.048921 | 0.310687 | 38,566,693 | C | T | 2413 |
| rs6599254 | 3.61E−05 | 0.047051 | 0.36327 | 38,770,559 | A | G | 23 |
| rs1805126 | 3.61E−05 | −0.048802 | 0.689248 | 38,567,410 | A | G | 2414 |
| rs6771157 | 5.11E−05 | −0.051959 | 0.241736 | 38,738,867 | C | G | 9 |
| rs12632942 | 5.11E−05 | 0.051913 | 0.758469 | 38,740,002 | A | G | 11 |
| rs61487238 | 5.13E−05 | 0.051941 | 0.758316 | 38,739,075 | C | T | 2415 |
| rs62242434 | 5.15E−05 | 0.052048 | 0.757732 | 38,734,533 | C | T | 2416 |
| rs62242435 | 5.29E−05 | −0.051952 | 0.242103 | 38,734,621 | A | G | 2417 |
| rs62242430 | 5.30E−05 | −0.051976 | 0.242208 | 38,729,180 | C | T | 2418 |
| rs61014925 | 5.31E−05 | 0.051976 | 0.757898 | 38,730,904 | A | G | 2419 |
| rs6781740 | 5.32E−05 | 0.051965 | 0.757796 | 38,728,981 | C | T | 2420 |
| rs11710006 | 5.33E−05 | 0.051972 | 0.757762 | 38,727,794 | A | T | 4 |
| rs7428779 | 6.04E−05 | −0.056462 | 0.189218 | 38,621,427 | T | C | 2421 |
| rs41312433 | 6.44E−05 | 0.056236 | 0.810742 | 38,622,646 | G | T | 2422 |
| rs9818148 | 6.49E−05 | −0.055328 | 0.275009 | 38,643,260 | G | T | 2423 |
| rs58454174 | 7.38E−05 | 0.055582 | 0.803661 | 38,590,537 | C | T | 2424 |
| rs11711602 | 7.39E−05 | 0.055258 | 0.809355 | 38,622,051 | C | T | 2425 |
| rs6801957 | 7.65E−05 | −0.045001 | 0.635242 | 38,742,319 | C | T | 14 |
| rs9833775 | 7.82E−05 | 0.054825 | 0.805395 | 38,616,088 | C | T | 2426 |
| rs10212202 | 8.28E−05 | −0.054695 | 0.194835 | 38,613,394 | C | G | 2427 |
| rs11721012 | 8.60E−05 | −0.054582 | 0.194968 | 38,610,878 | A | G | 2428 |
| rs9311194 | 8.70E−05 | −0.054544 | 0.195007 | 38,610,076 | A | G | 2429 |
| rs7355944 | 8.73E−05 | −0.05483 | 0.194552 | 38,601,197 | A | G | 2430 |
| rs9311192 | 8.77E−05 | 0.054518 | 0.804968 | 38,609,615 | C | T | 2431 |
| rs9311191 | 9.23E−05 | −0.054336 | 0.195189 | 38,608,114 | C | T | 2432 |
| rs10154914 | 9.36E−05 | −0.05431 | 0.195228 | 38,607,634 | A | T | 2433 |
| rs12636153 | 9.41E−05 | 0.050819 | 0.760858 | 38,744,301 | A | C | 2434 |
| rs55981643 | 9.62E−05 | −0.054255 | 0.195307 | 38,606,959 | A | G | 2435 |
| rs11711941 | 1.03E−04 | −0.044279 | 0.607063 | 38,753,544 | C | T | 2436 |
| rs6790396 | 1.07E−04 | 0.044083 | 0.369591 | 38,746,929 | C | G | 17 |
| rs10428132 | 1.18E−04 | −0.043583 | 0.629702 | 38,752,558 | G | T | 2437 |
| rs62242438 | 1.19E−04 | −0.049359 | 0.242986 | 38,741,352 | C | T | 2438 |
| rs6599220 | 1.20E−04 | 0.168846 | 0.981567 | 38,612,998 | C | T | 2439 |
| rs62245110 | 1.22E−04 | −0.053542 | 0.195859 | 38,605,315 | A | C | 2440 |
| chr3: 38397851 | 1.25E−04 | −0.126088 | 0.958191 | 38,397,851 | C | T | 2441 |
| chr3: 38519917 | 1.31E−04 | 0.152069 | 0.02794 | 38,519,917 | A | C | 2442 |
| rs7433723 | 1.32E−04 | −0.043369 | 0.630913 | 38,759,961 | A | G | 2443 |
| rs6599257 | 1.32E−04 | −0.048773 | 0.724409 | 38,779,592 | T | C | 33 |
| rs6599255 | 1.40E−04 | 0.043248 | 0.3701 | 38,771,419 | A | C | 24 |
| rs62241188 | 1.41E−04 | −0.065165 | 0.841749 | 38,578,073 | C | G | 2444 |
| rs6768664 | 1.42E−04 | 0.043023 | 0.521816 | 38,659,470 | A | C | 2445 |
| rs4414778 | 1.50E−04 | −0.047113 | 0.269836 | 38,787,169 | C | T | 36 |
| rs7430451 | 1.59E−04 | 0.043488 | 0.660228 | 38,770,499 | C | G | 22 |
| chr3: 38717393 | 1.66E−04 | 0.084214 | 0.091492 | 38,717,393 | A | G | 2446 |
| rs62244105 | 1.67E−04 | 0.048029 | 0.741276 | 38,789,587 | A | G | 2447 |
| rs3922843 | 1.68E−04 | −0.048988 | 0.251481 | 38,599,347 | A | G | 2448 |
| rs11129807 | 1.75E−04 | −0.047784 | 0.253152 | 38,782,421 | A | T | 2449 |
| chr3: 38782569 | 1.75E−04 | 0.047793 | 0.746893 | 38,782,569 | A | G | 2450 |
| chr3: 38780863 | 1.76E−04 | −0.047752 | 0.253144 | 38,780,863 | A | G | 2451 |
| rs62244077 | 1.76E−04 | 0.047756 | 0.746846 | 38,781,392 | A | G | 2452 |
| rs12635859 | 1.77E−04 | 0.047777 | 0.746804 | 38,783,890 | A | G | 2453 |
| rs12635869 | 1.77E−04 | 0.047778 | 0.746802 | 38,783,971 | A | G | 2454 |
| rs62244078 | 1.77E−04 | −0.047787 | 0.253202 | 38,784,646 | C | T | 2455 |
| rs62244075 | 1.78E−04 | −0.047627 | 0.25241 | 38,778,938 | C | G | 2456 |
| rs62244074 | 1.78E−04 | −0.047614 | 0.252353 | 38,778,904 | A | G | 2457 |
| rs62244080 | 1.78E−04 | −0.047795 | 0.253282 | 38,786,821 | A | G | 2458 |
| rs7432804 | 1.80E−04 | 0.04624 | 0.732109 | 38,778,513 | A | G | 30 |
| rs62244079 | 1.80E−04 | −0.047786 | 0.253159 | 38,786,782 | C | T | 2459 |
| rs62244081 | 1.82E−04 | −0.047856 | 0.253389 | 38,788,013 | C | T | 2460 |
| rs7651106 | 1.82E−04 | −0.045647 | 0.720684 | 38,779,345 | C | T | 32 |
| rs62244103 | 1.85E−04 | −0.047929 | 0.253575 | 38,788,933 | C | G | 2461 |
| rs62244104 | 1.86E−04 | 0.046301 | 0.343389 | 38,789,222 | A | C | 2462 |
| rs13098827 | 1.91E−04 | 0.044125 | 0.365009 | 38,735,947 | A | G | 2463 |
| rs12497173 | 1.92E−04 | 0.045472 | 0.279043 | 38,780,394 | G | T | 2464 |
| rs7610489 | 1.93E−04 | 0.045474 | 0.27906 | 38,781,482 | A | G | 34 |
| rs59669930 | 1.94E−04 | 0.045474 | 0.27906 | 38,781,515 | C | T | 2465 |
| rs7641844 | 1.96E−04 | 0.046709 | 0.736415 | 38,777,255 | A | G | 29 |
| rs4417808 | 2.00E−04 | 0.045475 | 0.279116 | 38,785,241 | A | G | 2466 |
| rs56040630 | 2.00E−04 | −0.045473 | 0.720867 | 38,785,529 | C | T | 2467 |
| rs7430477 | 2.09E−04 | 0.040587 | 0.52152 | 38,740,494 | C | T | 12 |
| rs10212338 | 2.12E−04 | 0.045451 | 0.279278 | 38,787,654 | A | G | 37 |
| rs6599256 | 2.18E−04 | 0.046811 | 0.739804 | 38,776,229 | G | T | 28 |
| rs6798015 | 2.28E−04 | 0.043863 | 0.323672 | 38,773,840 | C | T | 26 |
| rs6763876 | 2.35E−04 | −0.046762 | 0.258382 | 38,775,751 | C | T | 27 |
| rs11720166 | 2.36E−04 | −0.049316 | 0.739489 | 38,574,816 | C | G | 2468 |
| rs6599251 | 2.40E−04 | 0.041407 | 0.383888 | 38,760,813 | G | T | 21 |
| chr3: 38640584 | 2.52E−04 | 0.079704 | 0.908864 | 38,640,584 | A | G | 2469 |
| chr3: 38716846 | 2.57E−04 | 0.115032 | 0.959158 | 38,716,846 | C | T | 2470 |
| rs6799868 | 2.60E−04 | 0.048028 | 0.258119 | 38,576,560 | C | T | 2471 |
| chr3: 38762560 | 2.63E−04 | 0.053246 | 0.659245 | 38,762,560 | A | G | 2472 |
| rs2169552 | 2.63E−04 | 0.09501 | 0.051399 | 38,412,972 | A | G | 2473 |
| rs7374165 | 2.73E−04 | 0.042926 | 0.690326 | 38,701,689 | A | C | 2474 |
| rs6777775 | 2.83E−04 | −0.041731 | 0.581722 | 38,796,125 | A | G | 2475 |
| chr3: 38640691 | 3.16E−04 | −0.0806 | 0.908585 | 38,640,691 | C | T | 2476 |
| rs59858965 | 3.19E−04 | 0.047182 | 0.76677 | 38,742,965 | A | C | 2477 |
| rs59468016 | 3.28E−04 | −0.047116 | 0.233104 | 38,743,251 | A | G | 2478 |
| rs7615140 | 3.30E−04 | −0.041491 | 0.333662 | 38,757,030 | C | T | 19 |
| rs57326399 | 3.30E−04 | −0.047102 | 0.233081 | 38,743,304 | C | T | 2479 |
| rs12638572 | 3.67E−04 | 0.041105 | 0.664935 | 38,762,801 | A | G | 2480 |
| rs13061927 | 3.87E−04 | −0.038967 | 0.595643 | 38,682,356 | G | T | 2481 |
| chr3: 38658246 | 4.00E−04 | −0.077337 | 0.910479 | 38,658,246 | A | T | 2482 |
| rs6784303 | 4.16E−04 | 0.040703 | 0.665051 | 38,769,919 | C | T | 2483 |
| rs6599253 | 4.16E−04 | 0.040695 | 0.665049 | 38,769,364 | A | C | 2484 |
| rs4420805 | 4.21E−04 | −0.044695 | 0.720896 | 38,789,237 | A | C | 2485 |
| rs62244070 | 4.23E−04 | 0.046221 | 0.764885 | 38,773,175 | C | T | 2486 |
| rs12630795 | 4.52E−04 | 0.045979 | 0.765737 | 38,771,989 | A | G | 25 |
| chr3: 38422797 | 4.68E−04 | 0.089849 | 0.053363 | 38,422,797 | C | T | 2487 |
| rs7431305 | 4.68E−04 | 0.046335 | 0.726542 | 38,639,168 | G | T | 2488 |
| rs6599231 | 4.75E−04 | 0.039262 | 0.51977 | 38,659,745 | C | T | 2489 |
| rs34786326 | 4.77E−04 | 0.045914 | 0.770275 | 38,744,872 | C | T | 2490 |
| rs6809970 | 5.20E−04 | 0.093474 | 0.04791 | 38,416,547 | A | C | 2491 |
| rs9875610 | 5.36E−04 | 0.040836 | 0.398496 | 38,805,121 | A | G | 2492 |
| rs41312391 | 5.36E−04 | −0.05853 | 0.849766 | 38,573,610 | C | T | 2493 |
| rs7611456 | 5.59E−04 | 0.043151 | 0.280545 | 38,788,469 | C | T | 2494 |
| rs7372839 | 5.61E−04 | −0.037769 | 0.464923 | 38,660,651 | A | C | 2495 |
| rs9861852 | 5.85E−04 | −0.092919 | 0.952565 | 38,425,837 | C | T | 2496 |
| rs7645704 | 6.04E−04 | −0.091577 | 0.947447 | 38,472,085 | A | C | 2497 |
| rs62242444 | 6.08E−04 | 0.044772 | 0.770208 | 38,752,237 | C | T | 2498 |
| rs6806209 | 6.23E−04 | 0.092588 | 0.047183 | 38,432,787 | C | T | 2499 |
| rs62242448 | 6.28E−04 | −0.044649 | 0.229753 | 38,755,623 | A | C | 2500 |
| rs60554541 | 6.40E−04 | 0.04458 | 0.770269 | 38,757,476 | A | G | 2501 |
| rs9879824 | 6.61E−04 | −0.092344 | 0.953072 | 38,439,692 | C | T | 2502 |
| rs60969309 | 6.77E−04 | −0.044368 | 0.229666 | 38,763,008 | C | T | 2503 |
| rs7617919 | 6.78E−04 | −0.044371 | 0.229628 | 38,768,993 | A | G | 2504 |
| rs6599252 | 6.80E−04 | −0.044354 | 0.229653 | 38,764,695 | A | T | 2505 |
| rs12634001 | 6.80E−04 | −0.044349 | 0.229659 | 38,763,702 | A | G | 2506 |
| rs7651170 | 6.95E−04 | −0.092032 | 0.953262 | 38,449,113 | A | G | 2507 |
| rs62241189 | 6.97E−04 | −0.06653 | 0.155663 | 38,581,750 | A | G | 2508 |
| chr3: 38450284 | 7.02E−04 | 0.09193 | 0.04673 | 38,450,284 | A | G | 2509 |
| rs6785849 | 7.22E−04 | 0.091768 | 0.046644 | 38,461,700 | G | T | 2510 |
| chr3: 38764954 | 7.24E−04 | −0.107947 | 0.044322 | 38,764,954 | A | G | 2511 |
| chr3: 38467860 | 7.35E−04 | −0.091661 | 0.953431 | 38,467,860 | A | G | 2512 |
| rs9871059 | 7.35E−04 | −0.091661 | 0.953431 | 38,467,870 | C | T | 2513 |
| rs9809948 | 7.51E−04 | −0.091527 | 0.953524 | 38,475,651 | C | G | 2514 |
| rs9812912 | 7.58E−04 | 0.065786 | 0.845333 | 38,582,233 | C | T | 2515 |
| rs57475654 | 7.59E−04 | −0.091466 | 0.953561 | 38,479,392 | A | G | 2516 |
| chr3: 38601173 | 7.87E−04 | −0.082412 | 0.079393 | 38,601,173 | A | G | 2517 |
| rs6772948 | 7.92E−04 | −0.049851 | 0.831966 | 38,590,320 | C | T | 2518 |
| rs9311197 | 7.93E−04 | 0.03688 | 0.459247 | 38,751,607 | A | G | 2519 |
| rs11711260 | 7.98E−04 | −0.061599 | 0.902742 | 38,546,293 | C | T | 2520 |
| rs13062680 | 7.99E−04 | −0.03732 | 0.593383 | 38,699,853 | C | T | 2521 |
| rs11717146 | 8.00E−04 | 0.061511 | 0.097231 | 38,531,806 | A | G | 2522 |
| rs41313243 | 8.19E−04 | −0.07126 | 0.902648 | 38,651,584 | A | T | 2523 |
| rs7373102 | 8.23E−04 | 0.03751 | 0.532002 | 38,655,632 | C | T | 2524 |
| rs12491987 | 8.32E−04 | 0.064962 | 0.867744 | 38,624,048 | C | T | 2525 |
| chr3: 38406049 | 8.39E−04 | −0.091666 | 0.938918 | 38,406,049 | C | T | 2526 |
| rs9809798 | 8.42E−04 | 0.036669 | 0.459673 | 38,748,809 | A | C | 2527 |
| rs7428167 | 8.98E−04 | −0.036472 | 0.540286 | 38,753,195 | C | T | 2528 |
| rs62244110 | 9.04E−04 | −0.037903 | 0.619781 | 38,807,434 | G | T | 2529 |
| chr3: 38583447 | 9.09E−04 | −0.155733 | 0.967401 | 38,583,447 | C | T | 2530 |
| rs73060568 | 1.00E−03 | 0.219707 | 0.012049 | 39,646,839 | A | G | 2531 |
| chr3: 39404899 | 1.01E−03 | −0.158546 | 0.022635 | 39,404,899 | C | G | 2532 |
| chr3: 38343423 | 1.02E−03 | −0.194951 | 0.023826 | 38,343,423 | C | G | 2533 |
| rs3934936 | 1.03E−03 | −0.039986 | 0.649493 | 38,639,313 | C | T | 2534 |
| rs73065166 | 1.05E−03 | 0.220018 | 0.012078 | 39,698,404 | C | T | 2535 |
| rs62244111 | 1.08E−03 | −0.036958 | 0.615751 | 38,807,690 | C | G | 2536 |
| rs9815891 | 1.08E−03 | −0.03695 | 0.615745 | 38,808,001 | C | T | 2537 |
| rs12489820 | 1.09E−03 | −0.059904 | 0.90126 | 38,455,975 | C | T | 2538 |
| rs62244112 | 1.10E−03 | −0.036862 | 0.615677 | 38,812,037 | A | G | 2539 |
| rs9828912 | 1.11E−03 | 0.03686 | 0.38433 | 38,812,013 | A | T | 2540 |
| rs62244113 | 1.11E−03 | 0.036844 | 0.384361 | 38,812,742 | A | G | 2541 |
| chr3: 38766563 | 1.12E−03 | −0.34684 | 0.988562 | 38,766,563 | C | T | 2542 |
| rs6599261 | 1.12E−03 | 0.036838 | 0.384143 | 38,809,846 | A | T | 2543 |
| rs12636576 | 1.12E−03 | −0.036797 | 0.615591 | 38,814,845 | A | G | 2544 |
| rs4622847 | 1.20E−03 | −0.040749 | 0.291505 | 38,799,347 | A | G | 2545 |
| rs59478900 | 1.20E−03 | 0.036547 | 0.384637 | 38,828,607 | A | G | 2546 |
| rs62244119 | 1.21E−03 | 0.036901 | 0.385001 | 38,834,862 | A | C | 2547 |
| rs62244117 | 1.24E−03 | 0.036475 | 0.384486 | 38,827,497 | C | T | 2548 |
| rs28619020 | 1.24E−03 | −0.088074 | 0.955272 | 38,496,005 | C | T | 2549 |
| rs6797133 | 1.29E−03 | −0.037255 | 0.371291 | 38,631,037 | A | G | 2550 |
| chr3: 38109696 | 1.30E−03 | 0.12399 | 0.979851 | 38,109,696 | C | T | 2551 |
| rs73070981 | 1.33E−03 | 0.05551 | 0.121417 | 38,606,842 | C | T | 2552 |
| rs11717818 | 1.33E−03 | −0.035258 | 0.511907 | 38,697,700 | C | T | 2553 |
| rs11708996 | 1.40E−03 | 0.05528 | 0.12072 | 38,608,927 | C | G | 2554 |
| rs9836531 | 1.40E−03 | 0.062814 | 0.893756 | 38,804,578 | A | G | 2555 |
| chr3: 38400812 | 1.45E−03 | −0.079238 | 0.935556 | 38,400,812 | C | T | 2556 |
| rs9874436 | 1.47E−03 | 0.034842 | 0.474006 | 38,750,328 | C | G | 2557 |
| rs6771945 | 1.47E−03 | 0.079011 | 0.064664 | 38,397,911 | A | G | 2558 |
| chr3: 38400750 | 1.52E−03 | 0.078704 | 0.064467 | 38,400,750 | A | G | 2559 |
| rs73056438 | 1.52E−03 | −0.0631 | 0.897729 | 38,646,478 | C | T | 2560 |
| rs9841329 | 1.54E−03 | 0.034855 | 0.435636 | 38,662,807 | A | G | 2561 |
| rs6771940 | 1.55E−03 | 0.078624 | 0.064515 | 38,397,901 | A | G | 2562 |
| rs17037638 | 1.57E−03 | −0.078499 | 0.935612 | 38,400,455 | C | T | 2563 |
| rs7620883 | 1.59E−03 | 0.041757 | 0.221489 | 38,589,484 | A | G | 2564 |
| chr3: 38397728 | 1.62E−03 | −0.078355 | 0.935547 | 38,397,728 | A | G | 2565 |
| rs59856101 | 1.80E−03 | 0.041184 | 0.246351 | 38,794,198 | A | C | 2566 |
| chr3: 38225480 | 1.81E−03 | 0.082682 | 0.068067 | 38,225,480 | C | T | 2567 |
| rs73064540 | 1.89E−03 | 0.041118 | 0.246011 | 38,796,286 | A | T | 2568 |
| chr3: 38712931 | 1.97E−03 | −0.296998 | 0.010394 | 38,712,931 | A | G | 2569 |
| rs55787218 | 2.11E−03 | −0.092149 | 0.958282 | 38,558,267 | C | T | 2570 |
| chr3: 37936400 | 2.19E−03 | −0.125399 | 0.972834 | 37,936,400 | C | G | 2571 |
| chr3: 38638409 | 2.23E−03 | −0.067678 | 0.89742 | 38,638,409 | C | G | 2572 |
| chr3: 39059475 | 2.26E−03 | 0.046083 | 0.302671 | 39,059,475 | A | G | 2573 |
| rs7638910 | 2.40E−03 | −0.03323 | 0.495296 | 38,695,720 | A | C | 2574 |
| chr3: 38801183 | 2.41E−03 | −0.040607 | 0.753603 | 38,801,183 | A | G | 2575 |
| rs169044 | 2.42E−03 | −0.036569 | 0.566791 | 38,394,919 | C | T | 2576 |
| rs13096318 | 2.47E−03 | 0.046941 | 0.770644 | 39,400,761 | C | T | 2577 |
| chr3: 38640731 | 2.54E−03 | 0.042332 | 0.355996 | 38,640,731 | C | T | 2578 |
| chr3: 38639721 | 2.64E−03 | −0.040919 | 0.744223 | 38,639,721 | A | G | 2579 |
| chr3: 37880557 | 2.71E−03 | −0.180411 | 0.989494 | 37,880,557 | C | T | 2580 |
| chr3: 39650476 | 2.76E−03 | −0.302381 | 0.994699 | 39,650,476 | A | C | 2581 |
| rs9844644 | 2.85E−03 | 0.039528 | 0.764047 | 38,814,797 | C | T | 2582 |
| rs9861242 | 2.87E−03 | 0.039945 | 0.218534 | 38,584,338 | A | G | 2583 |
| rs11705730 | 3.02E−03 | 0.037363 | 0.61091 | 38,789,238 | A | C | 2584 |
| chr3: 38598253 | 3.20E−03 | 0.180695 | 0.977627 | 38,598,253 | C | T | 2585 |
| rs7374891 | 3.39E−03 | 0.035921 | 0.562929 | 38,643,509 | A | T | 2586 |
| rs6777141 | 3.41E−03 | −0.107703 | 0.02983 | 38,075,320 | A | G | 2587 |
| rs6795580 | 3.43E−03 | −0.035875 | 0.437472 | 38,642,577 | C | G | 2588 |
| rs7638909 | 3.49E−03 | 0.038794 | 0.251338 | 38,569,977 | G | T | 2589 |
| rs1805124 | 3.54E−03 | −0.036125 | 0.276397 | 38,620,424 | C | T | 2590 |
| rs73058909 | 3.64E−03 | −0.070689 | 0.055706 | 37,923,577 | C | T | 2591 |
| chr3: 38642089 | 3.64E−03 | −0.336874 | 0.004018 | 38,642,089 | A | G | 2592 |
| rs73058914 | 3.66E−03 | −0.070567 | 0.055785 | 37,924,492 | A | G | 2593 |
| rs6801131 | 3.69E−03 | 0.19963 | 0.009584 | 39,371,560 | G | T | 2594 |
| chr3: 38062226 | 3.72E−03 | 0.386993 | 0.99311 | 38,062,226 | C | T | 2595 |
| rs9861030 | 3.84E−03 | −0.093246 | 0.028889 | 38,032,610 | C | T | 2596 |
| chr3: 38574711 | 3.99E−03 | −0.087102 | 0.957374 | 38,574,711 | C | T | 2597 |
| rs7374540 | 4.01E−03 | −0.032484 | 0.625127 | 38,609,146 | A | C | 2598 |
| rs4016647 | 4.04E−03 | 0.190626 | 0.009351 | 39,369,355 | G | T | 2599 |
| rs56808891 | 4.05E−03 | 0.076627 | 0.064269 | 38,641,225 | A | G | 2600 |
| rs7430439 | 4.07E−03 | −0.032744 | 0.624811 | 38,778,643 | A | G | 31 |
| rs13083907 | 4.07E−03 | −0.188706 | 0.991285 | 37,765,116 | C | T | 2601 |
| rs4016648 | 4.09E−03 | −0.191103 | 0.990631 | 39,369,596 | A | T | 2602 |
| chr3: 38753590 | 4.13E−03 | −0.152112 | 0.984001 | 38,753,590 | C | T | 2603 |
| rs7633974 | 4.14E−03 | −0.03997 | 0.315135 | 38,640,927 | C | G | 2604 |
| chr3: 39387876 | 4.15E−03 | −0.259866 | 0.987019 | 39,387,876 | C | T | 2605 |
| chr3: 39566928 | 4.19E−03 | −0.213987 | 0.989968 | 39,566,928 | C | T | 2606 |
| rs73070977 | 4.19E−03 | 0.050071 | 0.111239 | 38,606,435 | G | T | 2607 |
| rs1909555 | 4.23E−03 | 0.19623 | 0.009339 | 39,357,200 | A | G | 2608 |
| rs41312411 | 4.29E−03 | −0.050144 | 0.888794 | 38,596,241 | C | G | 2609 |
| rs2649755 | 4.34E−03 | −0.198807 | 0.990663 | 39,351,973 | C | T | 2610 |
| rs4016649 | 4.36E−03 | 0.201489 | 0.009326 | 39,369,954 | A | G | 2611 |
| rs41312045 | 4.64E−03 | 0.130436 | 0.024856 | 38,645,025 | C | G | 2612 |
| rs11927181 | 4.70E−03 | 0.103949 | 0.977238 | 38,043,540 | G | T | 2613 |
| chr3: 38721547 | 4.79E−03 | −0.150905 | 0.984657 | 38,721,547 | C | T | 2614 |
| chr3: 38575105 | 4.91E−03 | 0.078055 | 0.052822 | 38,575,105 | A | G | 2615 |
| rs73825594 | 4.91E−03 | 0.079814 | 0.052949 | 38,577,610 | A | G | 2616 |
| rs56990533 | 4.91E−03 | −0.077544 | 0.947176 | 38,569,611 | A | G | 2617 |
| rs45505695 | 4.93E−03 | 0.077853 | 0.052743 | 38,573,403 | A | G | 2618 |
| chr3: 39385846 | 4.94E−03 | 0.158017 | 0.012201 | 39,385,846 | C | T | 2619 |
| rs169046 | 4.95E−03 | −0.033474 | 0.431862 | 38,392,371 | C | T | 2620 |
| chr3: 38598527 | 4.95E−03 | −0.186028 | 0.009649 | 38,598,527 | C | G | 2621 |
| rs73064557 | 4.96E−03 | 0.050584 | 0.861009 | 38,804,641 | C | T | 2622 |
| chr3: 38048625 | 5.02E−03 | 0.398091 | 0.993517 | 38,048,625 | G | T | 2623 |
| rs9818242 | 5.06E−03 | −0.046084 | 0.139186 | 37,957,695 | A | G | 2624 |
| rs169045 | 5.09E−03 | −0.033083 | 0.434819 | 38,394,586 | C | T | 2625 |
| chr3: 38678925 | 5.12E−03 | 0.146795 | 0.018207 | 38,678,925 | A | G | 2626 |
| rs10212167 | 5.14E−03 | −0.04336 | 0.149071 | 37,954,063 | C | T | 2627 |
| rs9311193 | 5.16E−03 | −0.033036 | 0.318703 | 38,609,663 | C | T | 2628 |
| rs7627207 | 5.17E−03 | −0.04335 | 0.14903 | 37,954,562 | A | T | 2629 |
| rs6806563 | 5.18E−03 | −0.217751 | 0.990708 | 39,375,945 | C | T | 2630 |
| rs196378 | 5.21E−03 | −0.033087 | 0.434786 | 38,392,349 | C | G | 2631 |
| chr3: 38348396 | 5.21E−03 | 0.064191 | 0.146456 | 38,348,396 | A | G | 2632 |
| rs3749386 | 5.22E−03 | 0.035148 | 0.367493 | 38,471,069 | C | T | 2633 |
| rs4131778 | 5.38E−03 | −0.034034 | 0.721166 | 38,587,230 | A | T | 2634 |
| rs41315507 | 5.64E−03 | −0.076618 | 0.943966 | 38,572,871 | C | T | 2635 |
| rs62242769 | 5.65E−03 | −0.034334 | 0.262457 | 38,619,266 | A | G | 2636 |
| rs7430438 | 5.68E−03 | −0.031416 | 0.608505 | 38,778,622 | A | G | 2637 |
| rs11714074 | 5.72E−03 | 0.034293 | 0.736918 | 38,618,789 | C | T | 2638 |
| chr3: 39446258 | 5.82E−03 | 0.166981 | 0.011217 | 39,446,258 | C | G | 2639 |
| chr3: 39103060 | 5.94E−03 | 0.120579 | 0.977873 | 39,103,060 | A | G | 2640 |
| chr3: 39249243 | 5.97E−03 | 0.117856 | 0.977845 | 39,249,243 | A | G | 2641 |
| chr3: 39231916 | 6.02E−03 | −0.118132 | 0.02171 | 39,231,916 | C | G | 2642 |
| rs7373076 | 6.04E−03 | 0.037363 | 0.260695 | 38,646,140 | A | G | 2643 |
| chr3: 38822386 | 6.07E−03 | 0.093931 | 0.968499 | 38,822,386 | A | G | 2644 |
| chr3: 38817911 | 6.16E−03 | 0.093725 | 0.968449 | 38,817,911 | C | T | 2645 |
| rs4130467 | 6.19E−03 | 0.033289 | 0.279216 | 38,586,708 | C | T | 2646 |
| chr3: 39595718 | 6.28E−03 | −0.189551 | 0.982702 | 39,595,718 | C | T | 2647 |
| rs13087327 | 6.29E−03 | 0.034945 | 0.267036 | 38,673,893 | A | G | 2648 |
| chr3: 38792243 | 6.29E−03 | −0.085756 | 0.038754 | 38,792,243 | C | T | 2649 |
| rs73054549 | 6.31E−03 | −0.040782 | 0.843494 | 38,631,999 | A | G | 2650 |
| chr3: 38777576 | 6.35E−03 | 0.083656 | 0.959603 | 38,777,576 | G | T | 2651 |
| rs3136660 | 6.35E−03 | −0.229526 | 0.990736 | 39,375,175 | C | G | 2652 |
| chr3: 38791169 | 6.37E−03 | −0.085566 | 0.038776 | 38,791,169 | A | G | 2653 |
| rs41312945 | 6.39E−03 | 0.040691 | 0.156833 | 38,631,849 | A | G | 2654 |
| chr3: 38099684 | 6.48E−03 | −0.070029 | 0.053637 | 38,099,684 | A | G | 2655 |
| chr3: 39454605 | 6.51E−03 | −0.160224 | 0.017293 | 39,454,605 | C | T | 2656 |
| rs3924120 | 6.52E−03 | −0.034552 | 0.746142 | 38,611,159 | A | G | 2657 |
| chr3: 38580815 | 6.55E−03 | −0.168559 | 0.975393 | 38,580,815 | C | T | 2658 |
| chr3: 37778137 | 6.57E−03 | 0.168597 | 0.013076 | 37,778,137 | C | T | 2659 |
| chr3: 38725868 | 6.67E−03 | −0.081703 | 0.039683 | 38,725,868 | C | T | 2660 |
| chr3: 39000860 | 6.70E−03 | 0.118765 | 0.977603 | 39,000,860 | A | G | 2661 |
| rs9824157 | 6.83E−03 | 0.034333 | 0.254027 | 38,608,694 | C | T | 2662 |
| chr3: 38699887 | 6.93E−03 | −0.630395 | 0.996079 | 38,699,887 | C | T | 2663 |
| chr3: 38783400 | 7.01E−03 | 0.084031 | 0.961109 | 38,783,400 | C | T | 2664 |
| chr3: 38979166 | 7.02E−03 | 0.118974 | 0.978204 | 38,979,166 | A | G | 2665 |
| chr3: 37810181 | 7.08E−03 | 0.16429 | 0.008763 | 37,810,181 | C | G | 2666 |
| rs28707243 | 7.22E−03 | −0.043819 | 0.68822 | 38,428,495 | C | T | 2667 |
| chr3: 38825716 | 7.23E−03 | −0.143291 | 0.986259 | 38,825,716 | G | T | 2668 |
| rs4676595 | 7.23E−03 | −0.038325 | 0.272701 | 38,805,897 | T | C | 2669 |
| rs7432787 | 7.26E−03 | 0.032217 | 0.459274 | 38,778,334 | A | T | 2670 |
| rs73054554 | 7.29E−03 | −0.040867 | 0.845257 | 38,633,061 | A | T | 2671 |
| rs13095741 | 7.41E−03 | −0.162981 | 0.986469 | 37,759,529 | G | T | 2672 |
| rs6599219 | 7.42E−03 | −0.034015 | 0.749036 | 38,612,714 | A | G | 2673 |
| chr3: 37800138 | 7.46E−03 | 0.168254 | 0.008244 | 37,800,138 | C | T | 2674 |
| chr3: 38770376 | 7.50E−03 | −0.164004 | 0.014192 | 38,770,376 | A | G | 2675 |
| chr3: 38800367 | 7.62E−03 | −0.165667 | 0.013685 | 38,800,367 | C | T | 2676 |
| rs7630012 | 7.63E−03 | −0.030131 | 0.574903 | 38,527,254 | A | G | 2677 |
| rs7373862 | 7.80E−03 | −0.03378 | 0.748851 | 38,609,347 | A | G | 2678 |
| rs41315501 | 7.85E−03 | 0.086808 | 0.044081 | 38,572,062 | A | G | 2679 |
| chr3: 39532775 | 7.86E−03 | −0.187174 | 0.012477 | 39,532,775 | A | G | 2680 |
| rs73825422 | 7.90E−03 | 0.162604 | 0.008449 | 37,804,876 | C | T | 2681 |
| chr3: 37819864 | 7.98E−03 | −0.157731 | 0.99093 | 37,819,864 | A | C | 2682 |
| rs7622209 | 8.03E−03 | 0.043822 | 0.851599 | 37,957,112 | C | T | 2683 |
| rs3845952 | 8.14E−03 | −0.095967 | 0.032033 | 38,060,814 | A | G | 2684 |
| rs3935184 | 8.21E−03 | 0.03311 | 0.260777 | 38,613,206 | C | G | 2685 |
| chr3: 38468585 | 8.25E−03 | 0.059362 | 0.876416 | 38,468,585 | C | T | 2686 |
| chr3: 38244641 | 8.33E−03 | 0.076019 | 0.03839 | 38,244,641 | A | C | 2687 |
| chr3: 38220480 | 8.34E−03 | −0.076011 | 0.961613 | 38,220,480 | C | T | 2688 |
| rs41312405 | 8.38E−03 | −0.163132 | 0.983185 | 38,593,697 | C | T | 2689 |
| chr3: 38104326 | 8.59E−03 | −0.090104 | 0.028302 | 38,104,326 | A | G | 2690 |
| rs13073053 | 8.70E−03 | 0.157589 | 0.014588 | 37,743,906 | A | T | 2691 |
| chr3: 38509296 | 8.73E−03 | −0.097091 | 0.972927 | 38,509,296 | C | T | 2692 |
| chr3: 39455456 | 8.73E−03 | −0.083959 | 0.048855 | 39,455,456 | A | C | 2693 |
| rs7432727 | 8.74E−03 | 0.029325 | 0.576485 | 38,774,400 | C | T | 2694 |
| chr3: 38244833 | 8.95E−03 | 0.130779 | 0.986126 | 38,244,833 | A | G | 2695 |
| chr3: 38809429 | 9.00E−03 | −0.249112 | 0.003695 | 38,809,429 | C | T | 2696 |
| chr3: 38816737 | 9.02E−03 | −0.248187 | 0.003704 | 38,816,737 | C | T | 2697 |
| chr3: 38823047 | 9.06E−03 | −0.247522 | 0.003714 | 38,823,047 | C | G | 2698 |
| rs73825421 | 9.08E−03 | 0.160725 | 0.008248 | 37,801,321 | A | G | 2699 |
| chr3: 38080696 | 9.08E−03 | 0.080164 | 0.966703 | 38,080,696 | G | T | 2700 |
| chr3: 38827605 | 9.08E−03 | 0.247875 | 0.996288 | 38,827,605 | A | C | 2701 |
| chr3: 38823572 | 9.10E−03 | −0.248426 | 0.003711 | 38,823,572 | A | G | 2702 |
| rs4676617 | 9.11E−03 | 0.02886 | 0.569967 | 39,255,386 | A | C | 2703 |
| chr3: 38595202 | 9.19E−03 | −0.161195 | 0.983701 | 38,595,202 | C | T | 2704 |
| rs13064555 | 9.21E−03 | 0.029079 | 0.512759 | 39,267,390 | A | C | 2705 |
| chr3: 38300747 | 9.61E−03 | −0.074847 | 0.961938 | 38,300,747 | A | G | 2706 |
| chr3: 39120671 | 9.67E−03 | −0.426208 | 0.004981 | 39,120,671 | A | G | 2707 |
| rs12635900 | 9.68E−03 | −0.088637 | 0.975736 | 38,537,396 | A | G | 2708 |
| rs17037819 | 9.68E−03 | −0.088637 | 0.975736 | 38,537,052 | C | T | 2709 |
| rs17037814 | 9.68E−03 | −0.088637 | 0.975737 | 38,536,311 | C | T | 2710 |
| rs17037809 | 9.68E−03 | 0.088637 | 0.024262 | 38,536,083 | A | G | 2711 |
| rs56858708 | 9.70E−03 | 0.088632 | 0.024261 | 38,538,253 | C | T | 2712 |
| rs62239347 | 9.71E−03 | −0.088626 | 0.975742 | 38,539,290 | C | T | 2713 |
| rs62239348 | 9.78E−03 | −0.088605 | 0.975752 | 38,542,729 | A | T | 2714 |
| rs2298422 | 9.78E−03 | −0.091273 | 0.977038 | 38,519,920 | C | T | 2715 |
| rs62239349 | 9.78E−03 | 0.088603 | 0.024247 | 38,543,085 | A | G | 2716 |
| rs62241771 | 9.79E−03 | 0.091276 | 0.022953 | 38,519,423 | C | G | 2717 |
| rs12631864 | 9.82E−03 | 0.088592 | 0.024242 | 38,544,768 | A | G | 2718 |
| rs12638090 | 9.83E−03 | −0.091289 | 0.977106 | 38,516,165 | A | C | 2719 |
| chr3: 37800547 | 9.84E−03 | −0.16015 | 0.991253 | 37,800,547 | A | G | 2720 |
| rs73067109 | 9.85E−03 | −0.08858 | 0.975763 | 38,546,616 | C | T | 2721 |
| chr3: 38525442 | 9.86E−03 | −0.088825 | 0.975861 | 38,525,442 | A | G | 2722 |
| rs2649756 | 9.87E−03 | −0.240937 | 0.990777 | 39,351,489 | C | T | 2723 |
| rs9845438 | 9.88E−03 | −0.03821 | 0.801105 | 38,575,460 | A | C | 2724 |
| rs59854949 | 9.90E−03 | 0.088638 | 0.02419 | 38,533,442 | A | G | 2725 |

Shown is marker identity, p-value of the association, magnitude of effect (in units of fractions of standard deviations), frequency of effect allele, position in NCBI Build 36, identity of effect allele, identity of other allele, SEQ ID for the marker. It should be noted that a positive value for an effect represents a predicted increase in the interval by the effect allele, while a negative value for the effect represents a decrease (i.e. the allele correlated with the negative effect is predictive of a decreased value of the measure).

TABLE 20
Association results for Pacemaker Placement in a
2 Mb region flanking rs6795970 on chromosome 3.

| | | | | | | effect | other | SEQ ID |
|---|---|---|---|---|---|---|---|---|
| marker | p-value | OR | freq aff | freq ctl | position | allele | allele | NO: |
| rs28501975 | 5.02E−04 | 0.758009 | 0.784217 | 0.811755 | 39,003,800 | A | T | 1364 |
| rs62244116 | 5.14E−04 | 0.676534 | 0.909373 | 0.928362 | 38,820,551 | A | C | 1365 |
| rs2239621 | 1.09E−03 | 1.211487 | 0.284918 | 0.249552 | 38,148,737 | T | C | 1366 |
| rs62244210 | 1.21E−03 | 0.71302 | 0.904603 | 0.923717 | 39,275,690 | A | G | 1367 |
| rs630934 | 1.39E−03 | 1.177481 | 0.50036 | 0.460187 | 39,464,623 | T | G | 1368 |
| chr3: 39383527 | 1.39E−03 | 109.219528 | 0.998154 | 0.995679 | 39,383,527 | C | G | 1369 |
| chr3: 38742425 | 1.41E−03 | 0.683169 | 0.923832 | 0.940342 | 38,742,425 | A | G | 1370 |
| rs28810495 | 1.48E−03 | 0.837803 | 0.678286 | 0.714649 | 39,485,659 | A | G | 1371 |
| chr3: 39333429 | 1.61E−03 | 0.012679 | 0.001835 | 0.004332 | 39,333,429 | C | T | 1372 |
| chr3: 39628515 | 1.62E−03 | 0.437758 | 0.980605 | 0.987523 | 39,628,515 | C | T | 1373 |
| chr3: 39620185 | 1.71E−03 | 2.277005 | 0.019384 | 0.012475 | 39,620,185 | A | G | 1374 |
| rs7434229 | 1.88E−03 | 1.239965 | 0.839246 | 0.809734 | 38,945,625 | C | T | 1375 |
| chr3: 38766563 | 1.92E−03 | 0.255389 | 0.984393 | 0.988656 | 38,766,563 | C | T | 1376 |
| rs1278142 | 1.95E−03 | 1.170341 | 0.502359 | 0.463197 | 39,458,156 | C | T | 1377 |
| chr3: 39580601 | 1.96E−03 | 0.440326 | 0.980702 | 0.987475 | 39,580,601 | A | C | 1378 |
| rs2370969 | 1.98E−03 | 1.18672 | 0.582051 | 0.546054 | 39,511,151 | C | T | 1379 |
| rs13067227 | 2.00E−03 | 1.268851 | 0.799701 | 0.773381 | 39,485,556 | C | T | 1380 |
| chr3: 39059469 | 2.01E−03 | 1.269706 | 0.856504 | 0.830225 | 39,059,469 | A | G | 1381 |
| rs478514 | 2.05E−03 | 0.855401 | 0.499843 | 0.53889 | 39,475,377 | C | T | 1382 |
| rs11708828 | 2.08E−03 | 1.168689 | 0.50035 | 0.461337 | 39,474,653 | C | T | 1383 |
| rs694611 | 2.10E−03 | 0.855756 | 0.499485 | 0.538453 | 39,473,151 | C | G | 1384 |
| rs4676496 | 2.10E−03 | 1.168551 | 0.50053 | 0.461567 | 39,473,009 | A | G | 1385 |
| rs645567 | 2.10E−03 | 0.85577 | 0.499525 | 0.538504 | 39,473,515 | A | G | 1386 |
| rs615479 | 2.13E−03 | 0.855581 | 0.497037 | 0.535862 | 39,461,604 | C | G | 1387 |
| rs68181851 | 2.19E−03 | 1.175121 | 0.572661 | 0.535074 | 37,806,100 | A | C | 1388 |
| rs6765697 | 2.21E−03 | 1.167603 | 0.501108 | 0.462301 | 39,468,243 | C | T | 1389 |
| rs34403692 | 2.22E−03 | 0.856519 | 0.498865 | 0.537664 | 39,468,064 | A | G | 1390 |
| rs9852186 | 2.22E−03 | 1.167447 | 0.501214 | 0.462435 | 39,467,598 | A | C | 1391 |
| chr3: 39307244 | 2.23E−03 | 0.018392 | 0.002035 | 0.004505 | 39,307,244 | A | G | 1392 |
| rs6772037 | 2.25E−03 | 1.167223 | 0.501339 | 0.462595 | 39,466,933 | A | G | 1393 |
| rs11129835 | 2.25E−03 | 0.856792 | 0.498068 | 0.536813 | 39,463,456 | C | T | 1394 |
| rs6774232 | 2.30E−03 | 1.166673 | 0.501546 | 0.462858 | 39,465,877 | C | G | 1395 |
| rs2370964 | 2.34E−03 | 1.166273 | 0.50171 | 0.463066 | 39,465,065 | C | T | 1396 |
| rs2887901 | 2.34E−03 | 0.85743 | 0.498297 | 0.536943 | 39,465,100 | A | G | 1397 |
| rs533577 | 2.37E−03 | 0.85764 | 0.498207 | 0.536828 | 39,464,655 | C | T | 1398 |
| rs9820623 | 2.47E−03 | 1.166828 | 0.50486 | 0.466754 | 39,468,862 | G | T | 1399 |
| chr3: 39725781 | 2.54E−03 | 2.089805 | 0.019651 | 0.0124 | 39,725,781 | C | T | 1400 |
| rs62243409 | 2.64E−03 | 1.342543 | 0.10326 | 0.084199 | 39,519,641 | C | T | 1401 |
| rs34457487 | 2.80E−03 | 0.85507 | 0.531806 | 0.568339 | 39,485,309 | A | G | 1402 |
| rs12636153 | 2.92E−03 | 1.202702 | 0.791481 | 0.760172 | 38,744,301 | A | C | 1403 |
| chr3: 39481484 | 3.09E−03 | 0.022599 | 0.002863 | 0.005187 | 39,481,484 | A | G | 1404 |
| rs62243433 | 3.11E−03 | 0.744922 | 0.899236 | 0.917674 | 39,570,468 | C | T | 1405 |
| rs62244135 | 3.14E−03 | 1.365478 | 0.098877 | 0.081522 | 38,864,832 | C | T | 1406 |
| chr3: 38515142 | 3.17E−03 | 0.038445 | 0.006958 | 0.009091 | 38,515,142 | A | G | 1407 |
| rs13081054 | 3.19E−03 | 0.857821 | 0.450985 | 0.487457 | 39,447,451 | A | G | 1408 |
| rs13067055 | 3.30E−03 | 1.176356 | 0.339664 | 0.305836 | 39,485,521 | A | G | 1409 |
| rs2233204 | 3.35E−03 | 0.831243 | 0.357798 | 0.387931 | 39,529,790 | T | C | 1410 |
| rs28362641 | 3.49E−03 | 1.22686 | 0.84275 | 0.815361 | 39,124,578 | C | T | 1411 |
| chr3: 38895721 | 3.62E−03 | 0.799103 | 0.758659 | 0.78256 | 38,895,721 | A | C | 1412 |
| rs674243 | 3.91E−03 | 0.86087 | 0.461902 | 0.49759 | 39,453,699 | C | T | 1413 |
| rs28615971 | 3.94E−03 | 1.175123 | 0.453796 | 0.420816 | 39,454,994 | C | G | 1414 |
| rs4955408 | 3.96E−03 | 1.171853 | 0.325634 | 0.292177 | 38,227,410 | A | G | 1415 |
| chr3: 39636519 | 3.96E−03 | 0.551702 | 0.028023 | 0.03769 | 39,636,519 | C | T | 1416 |
| rs4622847 | 4.00E−03 | 0.842279 | 0.260874 | 0.292191 | 38,799,347 | A | G | 1417 |
| rs7433733 | 4.15E−03 | 0.817746 | 0.167803 | 0.194591 | 38,945,628 | A | G | 1418 |
| chr3: 38801183 | 4.18E−03 | 0.840125 | 0.724404 | 0.754256 | 38,801,183 | A | G | 1419 |
| rs12638676 | 4.31E−03 | 1.159461 | 0.521705 | 0.486409 | 39,477,729 | A | G | 1420 |
| rs73064540 | 4.47E−03 | 1.186521 | 0.275273 | 0.245355 | 38,796,286 | A | T | 1421 |
| rs12495623 | 4.47E−03 | 0.863222 | 0.462036 | 0.497316 | 39,454,885 | G | T | 1422 |
| rs2507948 | 4.52E−03 | 0.862397 | 0.416218 | 0.451517 | 37,807,176 | A | G | 1423 |
| rs59856101 | 4.59E−03 | 1.185264 | 0.275653 | 0.245695 | 38,794,198 | A | C | 1424 |
| rs1513219 | 4.60E−03 | 0.852954 | 0.694411 | 0.72649 | 39,508,763 | C | T | 1425 |
| rs545397 | 4.62E−03 | 0.853093 | 0.694258 | 0.726361 | 39,505,087 | C | T | 1426 |
| rs616147 | 4.63E−03 | 1.172133 | 0.305813 | 0.273703 | 39,509,485 | A | G | 1427 |
| rs1009966 | 4.63E−03 | 1.157587 | 0.538142 | 0.502899 | 39,448,595 | A | G | 1428 |
| rs11129833 | 4.67E−03 | 1.157403 | 0.538182 | 0.502958 | 39,451,385 | G | T | 1429 |
| rs11706071 | 4.71E−03 | 0.863383 | 0.486405 | 0.521263 | 39,467,250 | A | C | 1430 |
| rs1708104 | 4.76E−03 | 0.852722 | 0.696181 | 0.728 | 39,509,746 | C | T | 1431 |
| rs1768208 | 4.76E−03 | 0.855002 | 0.694546 | 0.726881 | 39,498,007 | C | T | 1432 |
| rs62244161 | 4.78E−03 | 0.740111 | 0.907426 | 0.923811 | 39,175,081 | C | T | 1433 |
| chr3: 38717393 | 4.82E−03 | 1.319818 | 0.108924 | 0.091101 | 38,717,393 | A | G | 1434 |
| rs34897882 | 4.83E−03 | 1.156535 | 0.538542 | 0.50349 | 39,473,313 | C | T | 1435 |
| rs59858965 | 4.85E−03 | 1.192378 | 0.795546 | 0.766126 | 38,742,965 | A | C | 1436 |
| rs2853709 | 4.85E−03 | 1.193126 | 0.671223 | 0.642136 | 39,297,829 | C | T | 1437 |
| rs7638977 | 4.86E−03 | 1.156463 | 0.536172 | 0.50115 | 39,448,105 | A | T | 1438 |
| rs59468016 | 4.93E−03 | 0.83887 | 0.204425 | 0.233746 | 38,743,251 | A | G | 1439 |
| rs1768190 | 4.94E−03 | 0.855271 | 0.693726 | 0.725856 | 39,484,444 | C | T | 1440 |
| chr3: 39419719 | 4.95E−03 | 0.785786 | 0.174864 | 0.196376 | 39,419,719 | A | C | 1441 |
| rs57326399 | 4.98E−03 | 0.839015 | 0.20442 | 0.233723 | 38,743,304 | C | T | 1442 |
| rs12495185 | 5.01E−03 | 0.865172 | 0.463693 | 0.498656 | 39,447,794 | A | G | 1443 |
| rs28362640 | 5.06E−03 | 0.820326 | 0.151199 | 0.17724 | 39,124,871 | C | G | 1444 |
| rs11129834 | 5.16E−03 | 1.155047 | 0.53972 | 0.504857 | 39,463,350 | A | G | 1445 |
| rs879444 | 5.23E−03 | 1.155695 | 0.538959 | 0.504325 | 39,454,482 | C | T | 1446 |
| rs784504 | 5.25E−03 | 1.250931 | 0.77194 | 0.749283 | 39,170,264 | C | G | 1447 |
| rs35310432 | 5.32E−03 | 1.154438 | 0.535008 | 0.500236 | 39,449,962 | A | G | 1448 |
| rs6770797 | 5.34E−03 | 0.866379 | 0.460599 | 0.495323 | 39,464,881 | A | C | 1449 |
| rs6770798 | 5.34E−03 | 1.154228 | 0.539401 | 0.504677 | 39,464,883 | C | T | 1450 |
| rs11711484 | 5.46E−03 | 0.865553 | 0.469903 | 0.504277 | 39,251,905 | C | T | 1451 |
| rs11129832 | 5.48E−03 | 1.153751 | 0.534306 | 0.499636 | 39,447,529 | C | T | 1452 |
| rs13059755 | 5.48E−03 | 1.154013 | 0.536713 | 0.502161 | 39,459,515 | C | T | 1453 |
| rs2685080 | 5.54E−03 | 1.207319 | 0.186069 | 0.160236 | 37,807,390 | C | T | 1454 |
| chr3: 39475164 | 5.54E−03 | 0.867 | 0.465296 | 0.499902 | 39,475,164 | C | T | 1455 |
| rs11705945 | 5.58E−03 | 1.153651 | 0.536358 | 0.501863 | 39,459,727 | A | G | 1456 |
| rs34029841 | 5.59E−03 | 0.866961 | 0.463788 | 0.498354 | 39,460,244 | C | G | 1457 |
| rs34629457 | 5.62E−03 | 1.153239 | 0.53709 | 0.502572 | 39,466,571 | C | T | 1458 |
| rs12494100 | 5.65E−03 | 0.867271 | 0.465677 | 0.500222 | 39,456,839 | A | G | 1459 |
| rs2685108 | 5.73E−03 | 0.865994 | 0.410317 | 0.444678 | 37,804,178 | A | T | 1460 |
| rs11709075 | 5.78E−03 | 0.867743 | 0.466225 | 0.500703 | 39,475,249 | A | G | 1461 |
| rs11709044 | 5.78E−03 | 0.867767 | 0.466242 | 0.500715 | 39,475,204 | A | G | 1462 |
| chr3: 39299837 | 5.80E−03 | 3.75544 | 0.006409 | 0.003293 | 39,299,837 | C | T | 1463 |
| rs7635472 | 5.85E−03 | 0.86268 | 0.486717 | 0.519656 | 38,839,391 | C | T | 1464 |
| rs34786326 | 5.86E−03 | 1.188305 | 0.798347 | 0.769646 | 38,744,872 | C | T | 1465 |
| rs57345776 | 5.94E−03 | 1.161993 | 0.475101 | 0.442742 | 39,285,273 | C | T | 1466 |
| rs9875428 | 5.99E−03 | 1.153642 | 0.590449 | 0.556231 | 37,804,795 | G | T | 1467 |
| chr3: 39605077 | 6.06E−03 | 0.584316 | 0.030771 | 0.040466 | 39,605,077 | C | T | 1468 |
| rs56055643 | 6.25E−03 | 0.866782 | 0.448097 | 0.481755 | 39,447,633 | A | G | 1469 |
| chr3: 38218161 | 6.28E−03 | 1.372684 | 0.925825 | 0.909997 | 38,218,161 | C | T | 1470 |
| rs11919723 | 6.31E−03 | 1.196594 | 0.702611 | 0.675651 | 38,846,846 | C | T | 1471 |
| chr3: 39551395 | 6.38E−03 | 1.262307 | 0.21739 | 0.197127 | 39,551,395 | C | T | 1472 |
| rs4525803 | 6.44E−03 | 1.150037 | 0.5348 | 0.500686 | 39,465,626 | G | T | 1473 |
| rs2844399 | 6.45E−03 | 0.868295 | 0.407435 | 0.441449 | 37,806,039 | A | G | 1474 |
| rs36020975 | 6.55E−03 | 0.869873 | 0.466298 | 0.500324 | 39,466,484 | A | G | 1475 |
| rs3914283 | 6.61E−03 | 0.870034 | 0.466347 | 0.500348 | 39,466,288 | C | T | 1476 |
| rs62242335 | 6.67E−03 | 0.74884 | 0.908245 | 0.924044 | 39,153,249 | C | T | 1477 |
| chr3: 39253174 | 6.80E−03 | 1.235129 | 0.831869 | 0.80919 | 39,253,174 | C | T | 1478 |
| rs9874436 | 6.94E−03 | 1.146546 | 0.507488 | 0.473256 | 38,750,328 | C | G | 1479 |
| chr3: 38350073 | 6.96E−03 | 0.144084 | 0.010385 | 0.013048 | 38,350,073 | A | C | 1480 |
| chr3: 38249507 | 7.04E−03 | 0.731922 | 0.074308 | 0.089932 | 38,249,507 | A | G | 1481 |
| rs13073538 | 7.30E−03 | 1.150118 | 0.544753 | 0.511691 | 39,449,416 | A | G | 1482 |
| rs7651106 | 7.31E−03 | 0.862035 | 0.690583 | 0.721358 | 38,779,345 | C | T | 32 |
| rs62244166 | 7.33E−03 | 1.333524 | 0.088571 | 0.07304 | 39,203,680 | G | T | 1483 |
| rs631312 | 7.33E−03 | 0.860145 | 0.700744 | 0.731099 | 39,483,972 | A | G | 1484 |
| rs6599257 | 7.47E−03 | 1.159476 | 0.309123 | 0.278357 | 38,779,592 | C | T | 33 |
| rs4679028 | 7.47E−03 | 1.164386 | 0.28287 | 0.252867 | 38,302,296 | A | G | 1485 |
| rs56040630 | 7.52E−03 | 0.86214 | 0.690921 | 0.721537 | 38,785,529 | C | T | 1486 |
| rs12497173 | 7.52E−03 | 1.159353 | 0.309116 | 0.27837 | 38,780,394 | G | T | 1487 |
| rs4417808 | 7.52E−03 | 1.159834 | 0.30907 | 0.278445 | 38,785,241 | A | G | 1488 |
| rs11927034 | 7.55E−03 | 0.864306 | 0.383319 | 0.414973 | 39,569,436 | C | G | 1489 |
| rs9311197 | 7.58E−03 | 1.145221 | 0.492242 | 0.458508 | 38,751,607 | A | G | 1490 |
| rs7610489 | 7.59E−03 | 1.159292 | 0.309106 | 0.278387 | 38,781,482 | A | G | 34 |
| rs59669930 | 7.60E−03 | 1.159294 | 0.309106 | 0.278388 | 38,781,515 | C | T | 1491 |
| rs10212338 | 7.67E−03 | 1.16007 | 0.309065 | 0.278611 | 38,787,654 | A | G | 37 |
| chr3: 38311897 | 7.72E−03 | 0.756957 | 0.909754 | 0.925772 | 38,311,897 | C | T | 1492 |
| rs9809798 | 7.72E−03 | 1.144791 | 0.492611 | 0.458935 | 38,748,809 | A | C | 1493 |
| rs6777775 | 7.77E−03 | 0.868157 | 0.550334 | 0.582424 | 38,796,125 | A | G | 1494 |
| rs13095741 | 7.85E−03 | 0.532327 | 0.979944 | 0.986614 | 37,759,529 | G | T | 1495 |
| chr3: 38784696 | 7.91E−03 | 0.610406 | 0.019851 | 0.029748 | 38,784,696 | C | T | 1496 |
| rs2685111 | 7.93E−03 | 0.837856 | 0.812896 | 0.838119 | 37,801,178 | A | G | 1497 |
| rs7428167 | 8.04E−03 | 0.874184 | 0.507512 | 0.54102 | 38,753,195 | C | T | 1498 |
| rs17737239 | 8.10E−03 | 0.781659 | 0.885235 | 0.903005 | 39,334,643 | C | T | 1499 |
| chr3: 37778137 | 8.19E−03 | 1.887906 | 0.019402 | 0.012935 | 37,778,137 | C | T | 1500 |
| rs871144 | 8.20E−03 | 1.166098 | 0.701942 | 0.672415 | 39,297,470 | C | T | 1501 |
| rs62242444 | 8.28E−03 | 1.178226 | 0.797308 | 0.769601 | 38,752,237 | C | T | 1502 |
| rs1464047 | 8.30E−03 | 1.147022 | 0.524285 | 0.491616 | 39,501,878 | C | T | 1503 |
| rs62242448 | 8.46E−03 | 0.849142 | 0.202727 | 0.230358 | 38,755,623 | A | C | 1504 |
| rs938183 | 8.47E−03 | 0.869736 | 0.472896 | 0.504767 | 39,455,298 | A | G | 1505 |
| rs11926158 | 8.52E−03 | 0.858034 | 0.305224 | 0.334433 | 38,798,319 | C | G | 1506 |
| rs60554541 | 8.57E−03 | 1.177315 | 0.797254 | 0.769665 | 38,757,476 | A | G | 1507 |
| chr3: 39731526 | 8.61E−03 | 2.161184 | 0.989523 | 0.983045 | 39,731,526 | A | G | 1508 |
| rs2844397 | 8.64E−03 | 1.18509 | 0.214625 | 0.188864 | 37,801,726 | A | G | 1509 |
| rs13079368 | 8.78E−03 | 1.144229 | 0.519979 | 0.487262 | 39,487,555 | C | T | 1510 |
| rs11915103 | 8.79E−03 | 0.783485 | 0.884493 | 0.902057 | 39,326,735 | C | T | 1511 |
| rs7617919 | 8.90E−03 | 0.850085 | 0.202759 | 0.230229 | 38,768,993 | A | G | 1512 |
| rs60969309 | 8.91E−03 | 0.850127 | 0.202802 | 0.230268 | 38,763,008 | C | T | 1513 |
| rs6599252 | 8.92E−03 | 0.850168 | 0.202797 | 0.230255 | 38,764,695 | A | T | 1514 |
| rs12634001 | 8.94E−03 | 0.850219 | 0.202805 | 0.230261 | 38,763,702 | A | G | 1515 |
| rs7610619 | 9.25E−03 | 1.37702 | 0.956126 | 0.941878 | 39,161,204 | C | G | 1516 |
| chr3: 39582012 | 9.43E−03 | 2.104265 | 0.987645 | 0.98116 | 39,582,012 | C | T | 1517 |
| rs7621922 | 9.47E−03 | 1.37551 | 0.956094 | 0.941882 | 39,161,154 | A | C | 1518 |
| rs7625290 | 9.49E−03 | 1.38246 | 0.057364 | 0.044456 | 38,153,294 | G | T | 1519 |
| rs194707 | 9.50E−03 | 0.850156 | 0.708215 | 0.734736 | 38,330,719 | A | G | 1520 |
| rs28863770 | 9.70E−03 | 2.099834 | 0.987677 | 0.981214 | 39,597,342 | G | T | 1521 |
| rs34743428 | 9.73E−03 | 0.862405 | 0.622513 | 0.651498 | 39,573,199 | A | T | 1522 |
| rs2229528 | 9.87E−03 | 1.346881 | 0.920444 | 0.90548 | 38,142,099 | A | G | 1523 |
| chr3: 39282836 | 9.92E−03 | 1.9354 | 0.016904 | 0.010925 | 39,282,836 | C | T | 1524 |
| chr3: 39598938 | 9.93E−03 | 4.304103 | 0.992819 | 0.98961 | 39,598,938 | C | T | 1525 |
| rs13073053 | 9.97E−03 | 1.838771 | 0.020947 | 0.014446 | 37,743,906 | A | T | 1526 |

Shown is marker identity, p-value of the association, odds ratio (OR), frequency of effect allele in affecteds and controls, position in NCBI Build 36, identity of effect allele, identity of other allele, and Seq ID for the marker. It should be noted that when reported OR values are larger than unity, the effect allele is the at-risk allele, while, when reported OR values are less than unity, the effect allele is the protective allele, and the other allele is the at-risk allele. The OR value for the at-risk allele is in those cases equal to 1/OR for the protective (effect) allele.

TABLE 21
Association results for Heart Rate in a 2 Mb
region flanking rs365990 on chromosome 14.

| | | | | | effect | other | SEQ ID |
|---|---|---|---|---|---|---|---|
| marker | p-value | effect | freq | position | allele | allele | NO: |
| rs412768 | 9.26E−08 | −0.060282 | 0.711016 | 22,936,553 | T | C | 300 |
| rs445754 | 3.76E−07 | −0.061402 | 0.776285 | 22,933,642 | G | T | 297 |
| rs7155512 | 8.29E−07 | 0.092466 | 0.903438 | 22,838,709 | A | G | 1195 |
| rs56230144 | 9.23E−07 | 0.204067 | 0.9568 | 23,005,073 | C | T | 1196 |
| rs365990 | 1.32E−06 | −0.051564 | 0.661001 | 22,931,651 | A | G | 296 |
| rs422068 | 2.46E−06 | 0.051142 | 0.321833 | 22,934,644 | C | T | 1197 |
| rs452036 | 2.77E−06 | 0.049966 | 0.332923 | 22,935,725 | A | G | 299 |
| rs10438012 | 3.79E−06 | 0.095621 | 0.927681 | 22,861,062 | G | T | 1198 |
| rs403739 | 4.05E−06 | 0.050182 | 0.31069 | 22,938,616 | C | T | 1199 |
| rs7161630 | 4.14E−06 | −0.083217 | 0.107745 | 22,839,004 | C | T | 1200 |
| rs6573092 | 4.70E−06 | 0.101004 | 0.936831 | 22,842,180 | A | G | 1201 |
| chr14: 22936019 | 7.95E−06 | −0.280387 | 0.018172 | 22,936,019 | A | G | 1202 |
| rs432256 | 9.16E−06 | 0.052363 | 0.239976 | 22,940,592 | A | G | 1203 |
| rs403720 | 1.00E−05 | −0.052126 | 0.75906 | 22,938,868 | C | T | 1204 |
| rs439735 | 1.06E−05 | 0.051958 | 0.241396 | 22,938,125 | A | G | 301 |
| rs388914 | 1.14E−05 | 0.052184 | 0.235557 | 22,942,932 | A | G | 302 |
| rs2277474 | 1.29E−05 | −0.052338 | 0.762711 | 22,944,363 | C | T | 304 |
| rs1950252 | 1.48E−05 | −0.089798 | 0.063048 | 22,848,538 | A | G | 1205 |
| rs9323298 | 1.50E−05 | −0.089783 | 0.063031 | 22,852,609 | A | G | 1206 |
| rs10146155 | 1.51E−05 | −0.089785 | 0.063027 | 22,856,475 | A | G | 1207 |
| rs10134354 | 1.51E−05 | 0.089785 | 0.936973 | 22,856,476 | A | C | 1208 |
| rs7142474 | 1.54E−05 | 0.090012 | 0.937191 | 22,855,445 | A | G | 1209 |
| rs7161120 | 1.60E−05 | 0.089766 | 0.936997 | 22,867,041 | C | G | 1210 |
| rs1955559 | 1.66E−05 | −0.090756 | 0.062002 | 22,857,807 | A | G | 1211 |
| rs8020117 | 2.34E−05 | 0.084785 | 0.92704 | 22,861,859 | C | T | 1212 |
| chr14: 22962537 | 2.95E−05 | −0.598248 | 0.009444 | 22,962,537 | C | G | 1213 |
| rs376439 | 3.07E−05 | −0.044229 | 0.649176 | 22,938,869 | A | G | 1214 |
| rs440466 | 3.82E−05 | 0.044032 | 0.350475 | 22,943,957 | C | T | 303 |
| rs12147570 | 6.50E−05 | 0.058293 | 0.153294 | 22,951,760 | T | G | 306 |
| chr14: 22984943 | 7.89E−05 | 0.313082 | 0.987441 | 22,984,943 | A | C | 1215 |
| rs178640 | 9.52E−05 | −0.048306 | 0.55051 | 22,925,409 | A | G | 1216 |
| rs28730774 | 1.06E−04 | −0.179402 | 0.026907 | 22,936,280 | A | G | 1217 |
| rs2231801 | 1.22E−04 | 0.119449 | 0.966129 | 22,899,330 | C | T | 1218 |
| chr14: 22924192 | 1.48E−04 | −0.045942 | 0.52553 | 22,924,192 | A | T | 1219 |
| rs74037001 | 1.65E−04 | −0.062767 | 0.888893 | 22,917,034 | A | G | 1220 |
| rs178636 | 1.66E−04 | −0.045747 | 0.514228 | 22,921,714 | A | G | 1221 |
| rs73602471 | 1.67E−04 | −0.091203 | 0.051562 | 22,833,285 | A | G | 1222 |
| rs7145023 | 1.78E−04 | −0.051281 | 0.842778 | 22,952,429 | C | T | 1223 |
| rs3729833 | 1.78E−04 | −0.051281 | 0.842776 | 22,953,024 | C | T | 310 |
| rs3729825 | 1.78E−04 | −0.051284 | 0.842746 | 22,956,104 | C | T | 315 |
| rs735711 | 1.82E−04 | −0.051467 | 0.841435 | 22,968,867 | C | T | 1224 |
| rs12147533 | 1.86E−04 | −0.051175 | 0.842005 | 22,960,531 | A | G | 319 |
| rs396024 | 1.92E−04 | −0.064077 | 0.903422 | 22,925,318 | C | G | 1225 |
| rs45520434 | 1.93E−04 | −0.064042 | 0.90349 | 22,925,266 | C | T | 1226 |
| rs382872 | 1.94E−04 | 0.06403 | 0.096503 | 22,925,160 | A | G | 1227 |
| rs8004990 | 1.94E−04 | 0.063991 | 0.096523 | 22,923,995 | A | G | 1228 |
| rs10149621 | 1.95E−04 | −0.184387 | 0.049272 | 23,049,920 | G | T | 1229 |
| rs11624298 | 1.96E−04 | 0.063889 | 0.096403 | 22,922,992 | A | G | 1230 |
| rs453361 | 1.97E−04 | −0.063811 | 0.903618 | 22,921,240 | C | T | 1231 |
| rs433673 | 1.98E−04 | −0.063787 | 0.903624 | 22,920,742 | A | G | 1232 |
| chr14: 22919821 | 2.00E−04 | 0.063845 | 0.096264 | 22,919,821 | A | G | 1233 |
| chr14: 22919370 | 2.04E−04 | −0.063925 | 0.903902 | 22,919,370 | C | T | 1234 |
| chr14: 22919323 | 2.05E−04 | −0.063933 | 0.903919 | 22,919,323 | C | T | 1235 |
| rs451794 | 2.16E−04 | −0.063726 | 0.879057 | 22,928,072 | C | T | 1236 |
| chr14: 22954770 | 2.20E−04 | −0.595327 | 0.00779 | 22,954,770 | C | G | 1237 |
| rs45553533 | 2.62E−04 | 0.171455 | 0.974094 | 22,952,736 | C | T | 1238 |
| rs28535772 | 2.73E−04 | 0.049575 | 0.377413 | 22,936,984 | C | T | 1239 |
| chr14: 22673837 | 2.74E−04 | −0.138528 | 0.028992 | 22,673,837 | A | C | 1240 |
| rs2331979 | 2.86E−04 | −0.04045 | 0.682866 | 22,952,695 | A | G | 309 |
| chr14: 22966495 | 3.12E−04 | 0.591667 | 0.992522 | 22,966,495 | C | T | 1241 |
| rs178638 | 3.50E−04 | −0.043533 | 0.522228 | 22,924,164 | A | G | 1242 |
| chr14: 22936498 | 4.15E−04 | 0.591641 | 0.992777 | 22,936,498 | C | T | 1243 |
| rs723840 | 4.76E−04 | −0.042762 | 0.544287 | 22,918,151 | C | T | 1244 |
| chr14: 22412337 | 4.84E−04 | −0.115223 | 0.031937 | 22,412,337 | A | G | 1245 |
| chr14: 22715214 | 4.94E−04 | −0.117037 | 0.037954 | 22,715,214 | A | T | 1246 |
| chr14: 22397976 | 5.34E−04 | −0.111712 | 0.033185 | 22,397,976 | C | T | 1247 |
| rs4981468 | 5.37E−04 | −0.086043 | 0.041861 | 22,867,359 | A | G | 1248 |
| rs1535094 | 5.41E−04 | −0.086434 | 0.041667 | 22,849,474 | C | G | 1249 |
| rs10438119 | 5.87E−04 | 0.109608 | 0.965714 | 22,395,924 | C | T | 1250 |
| rs2754163 | 6.00E−04 | 0.039826 | 0.26027 | 22,967,347 | C | T | 322 |
| rs743567 | 6.32E−04 | −0.039097 | 0.696112 | 22,960,822 | A | C | 320 |
| rs7157716 | 6.45E−04 | −0.039578 | 0.740816 | 22,962,728 | A | G | 321 |
| chr14: 22936987 | 6.50E−04 | 0.051127 | 0.218558 | 22,936,987 | C | T | 1251 |
| chr14: 23430078 | 6.91E−04 | −0.170432 | 0.970111 | 23,430,078 | A | G | 1252 |
| chr14: 23578000 | 6.97E−04 | −0.206665 | 0.97524 | 23,578,000 | G | T | 1253 |
| chr14: 22742893 | 6.98E−04 | 0.084349 | 0.945903 | 22,742,893 | C | T | 1254 |
| rs11621983 | 7.49E−04 | −0.081523 | 0.059298 | 21,965,326 | A | G | 1255 |
| chr14: 23824127 | 7.54E−04 | −0.232008 | 0.993245 | 23,824,127 | A | C | 1256 |
| rs8019322 | 7.88E−04 | 0.03892 | 0.258234 | 22,962,421 | C | T | 1257 |
| chr14: 23798859 | 8.12E−04 | 0.256819 | 0.007195 | 23,798,859 | C | T | 1258 |
| rs45508899 | 8.22E−04 | −0.058559 | 0.876563 | 22,862,256 | A | C | 1259 |
| rs11849140 | 8.48E−04 | 0.163294 | 0.967882 | 23,028,156 | A | C | 1260 |
| rs7140721 | 9.19E−04 | 0.037915 | 0.296376 | 22,955,534 | A | G | 312 |
| chr14: 23515571 | 1.02E−03 | −0.204913 | 0.97704 | 23,515,571 | C | T | 1261 |
| chr14: 22386328 | 1.05E−03 | −0.110207 | 0.033748 | 22,386,328 | A | G | 1262 |
| rs765020 | 1.08E−03 | −0.035969 | 0.683431 | 22,953,579 | A | G | 1263 |
| rs8022522 | 1.25E−03 | 0.042912 | 0.344311 | 22,927,191 | A | G | 295 |
| rs7143356 | 1.35E−03 | 0.035248 | 0.31552 | 22,950,923 | C | T | 305 |
| chr14: 22830264 | 1.35E−03 | 0.080223 | 0.94409 | 22,830,264 | A | G | 1264 |
| rs61731179 | 1.37E−03 | −0.098117 | 0.055699 | 22,939,833 | A | G | 1265 |
| rs59799414 | 1.38E−03 | 0.035152 | 0.315779 | 22,955,837 | A | G | 1266 |
| rs2284651 | 1.38E−03 | 0.035172 | 0.31532 | 22,951,984 | C | T | 307 |
| rs3729829 | 1.38E−03 | 0.035168 | 0.315414 | 22,955,727 | A | G | 313 |
| rs7149517 | 1.39E−03 | 0.035159 | 0.315279 | 22,952,026 | G | T | 308 |
| rs765021 | 1.42E−03 | 0.035107 | 0.315106 | 22,953,439 | A | G | 311 |
| rs7159367 | 1.52E−03 | 0.034811 | 0.316165 | 22,957,485 | C | T | 316 |
| rs7145543 | 1.53E−03 | −0.034943 | 0.685438 | 22,952,400 | A | G | 1267 |
| rs1055061 | 1.55E−03 | 0.067996 | 0.944532 | 22,814,772 | C | T | 1268 |
| rs12894524 | 1.56E−03 | −0.034694 | 0.68378 | 22,957,880 | G | T | 317 |
| rs2277475 | 1.60E−03 | −0.035099 | 0.687998 | 22,958,505 | A | T | 318 |
| chr14: 23515358 | 1.65E−03 | 0.186672 | 0.025027 | 23,515,358 | A | C | 1269 |
| rs178762 | 1.78E−03 | −0.144588 | 0.019868 | 22,775,560 | C | T | 1270 |
| rs28631169 | 2.11E−03 | −0.038754 | 0.800716 | 22,958,023 | C | T | 1271 |
| rs55752686 | 2.14E−03 | −0.0786 | 0.053148 | 22,101,197 | C | T | 1272 |
| chr14: 22875956 | 2.42E−03 | −0.20096 | 0.015885 | 22,875,956 | A | G | 1273 |
| rs28564768 | 2.65E−03 | 0.061555 | 0.925538 | 21,942,608 | A | G | 1274 |
| chr14: 22822490 | 2.76E−03 | 0.072671 | 0.952063 | 22,822,490 | C | G | 1275 |
| rs434273 | 2.84E−03 | 0.038831 | 0.740022 | 22,942,506 | C | T | 1276 |
| chr14: 23469758 | 3.38E−03 | 0.155708 | 0.027016 | 23,469,758 | A | G | 1277 |
| chr14: 22821045 | 3.39E−03 | −0.071615 | 0.047713 | 22,821,045 | A | G | 1278 |
| chr14: 23415421 | 3.41E−03 | 0.168099 | 0.021891 | 23,415,421 | A | G | 1279 |
| chr14: 23927040 | 3.48E−03 | −0.238325 | 0.007866 | 23,927,040 | A | G | 1280 |
| chr14: 21945342 | 3.49E−03 | −0.057506 | 0.077776 | 21,945,342 | A | G | 1281 |
| rs72686239 | 3.50E−03 | −0.047909 | 0.853155 | 22,990,121 | A | G | 1282 |
| rs8003073 | 3.52E−03 | −0.064533 | 0.938924 | 22,911,233 | C | T | 1283 |
| rs11626025 | 3.53E−03 | 0.057762 | 0.922399 | 21,961,632 | G | T | 1284 |
| rs178641 | 3.64E−03 | 0.186101 | 0.978764 | 22,926,238 | C | T | 1285 |
| chr14: 23901245 | 3.67E−03 | −0.23634 | 0.007623 | 23,901,245 | A | G | 1286 |
| rs35775107 | 3.68E−03 | 0.347709 | 0.99338 | 22,771,085 | C | T | 1287 |
| chr14: 22820813 | 3.74E−03 | −0.069795 | 0.048153 | 22,820,813 | A | G | 1288 |
| chr14: 22694028 | 3.90E−03 | 0.088602 | 0.071583 | 22,694,028 | A | C | 1289 |
| rs9743907 | 4.12E−03 | −0.065778 | 0.048938 | 22,482,506 | A | G | 1290 |
| chr14: 22480427 | 4.15E−03 | −0.06573 | 0.048968 | 22,480,427 | A | G | 1291 |
| rs9285579 | 4.18E−03 | −0.065668 | 0.049005 | 22,477,575 | A | G | 1292 |
| rs10162421 | 4.19E−03 | 0.065659 | 0.95099 | 22,477,346 | A | G | 1293 |
| rs11849991 | 4.20E−03 | 0.06564 | 0.950979 | 22,476,293 | C | G | 1294 |
| chr14: 22476144 | 4.20E−03 | −0.065633 | 0.049025 | 22,476,144 | A | G | 1295 |
| rs59951568 | 4.25E−03 | 0.065564 | 0.950946 | 22,474,711 | G | T | 1296 |
| rs60947314 | 4.26E−03 | 0.06555 | 0.95094 | 22,474,634 | C | G | 1297 |
| rs10130976 | 4.27E−03 | −0.065543 | 0.049062 | 22,474,546 | A | T | 1298 |
| rs7159893 | 4.30E−03 | 0.065494 | 0.950918 | 22,474,163 | A | G | 1299 |
| chr14: 22502247 | 4.31E−03 | −0.067143 | 0.047766 | 22,502,247 | A | G | 1300 |
| chr14: 22470501 | 4.44E−03 | 0.065303 | 0.950842 | 22,470,501 | A | G | 1301 |
| rs60613561 | 4.45E−03 | −0.065287 | 0.049166 | 22,470,431 | A | C | 1302 |
| rs45536732 | 4.52E−03 | −0.065227 | 0.049194 | 22,459,636 | C | G | 1303 |
| rs73594486 | 4.54E−03 | 0.065196 | 0.950798 | 22,462,719 | C | T | 1304 |
| chr14: 22506209 | 4.55E−03 | −0.06741 | 0.047332 | 22,506,209 | A | C | 1305 |
| rs8011055 | 4.56E−03 | 0.065142 | 0.950777 | 22,451,358 | C | T | 1306 |
| rs73590530 | 4.56E−03 | 0.06514 | 0.950774 | 22,448,211 | C | T | 1307 |
| rs8021269 | 4.56E−03 | −0.065139 | 0.049223 | 22,453,485 | A | G | 1308 |
| rs73590540 | 4.56E−03 | 0.065139 | 0.950776 | 22,453,171 | C | T | 1309 |
| rs11557895 | 4.56E−03 | −0.065138 | 0.049222 | 22,458,071 | A | G | 1310 |
| chr14: 22452324 | 4.57E−03 | −0.065128 | 0.04923 | 22,452,324 | A | G | 1311 |
| chr14: 23785612 | 4.63E−03 | 0.043331 | 0.26805 | 23,785,612 | A | T | 1312 |
| rs17128397 | 4.64E−03 | 0.069004 | 0.952653 | 22,819,722 | A | C | 1313 |
| rs2005133 | 4.64E−03 | 0.067447 | 0.952719 | 22,507,569 | C | T | 1314 |
| chr14: 23018492 | 4.71E−03 | −0.606902 | 0.006103 | 23,018,492 | A | G | 1315 |
| chr14: 22561348 | 4.73E−03 | 0.071283 | 0.954209 | 22,561,348 | G | T | 1316 |
| rs17198715 | 4.89E−03 | 0.05266 | 0.918097 | 21,951,337 | A | C | 1317 |
| rs2231806 | 5.03E−03 | 0.055331 | 0.093569 | 22,898,733 | A | G | 1318 |
| rs10147083 | 5.11E−03 | 0.052484 | 0.918271 | 21,943,219 | A | C | 1319 |
| rs28538737 | 5.14E−03 | −0.052423 | 0.081739 | 21,942,713 | A | G | 1320 |
| rs28733600 | 5.14E−03 | 0.052459 | 0.917972 | 21,943,910 | A | T | 1321 |
| rs1242631 | 5.24E−03 | 0.064561 | 0.950507 | 22,441,620 | C | T | 1322 |
| rs28687681 | 5.29E−03 | −0.052214 | 0.082444 | 21,943,756 | C | G | 1323 |
| rs8014568 | 5.34E−03 | 0.031318 | 0.355439 | 22,913,203 | T | C | 1324 |
| chr14: 23705402 | 5.66E−03 | −0.063006 | 0.245717 | 23,705,402 | G | T | 1325 |
| rs941722 | 5.73E−03 | −0.067125 | 0.046105 | 22,517,978 | A | C | 1326 |
| rs56044156 | 5.96E−03 | 0.233206 | 0.012005 | 22,851,539 | A | G | 1327 |
| chr14: 22136882 | 6.15E−03 | 0.054308 | 0.908278 | 22,136,882 | A | G | 1328 |
| chr14: 23008616 | 6.26E−03 | 0.399831 | 0.993578 | 23,008,616 | C | G | 1329 |
| rs4048584 | 6.33E−03 | 0.053325 | 0.185322 | 23,509,428 | A | G | 1330 |
| rs2118499 | 6.48E−03 | 0.039023 | 0.829657 | 23,235,512 | T | C | 1331 |
| chr14: 22886092 | 6.56E−03 | 0.04428 | 0.354469 | 22,886,092 | G | T | 1332 |
| chr14: 22433946 | 6.57E−03 | −0.064167 | 0.049328 | 22,433,946 | A | G | 1333 |
| chr14: 22719959 | 6.80E−03 | 0.14378 | 0.989148 | 22,719,959 | C | G | 1334 |
| chr14: 22534064 | 6.85E−03 | 0.189526 | 0.991378 | 22,534,064 | C | G | 1335 |
| chr14: 23059444 | 6.90E−03 | 0.051019 | 0.495739 | 23,059,444 | A | G | 1336 |
| rs10149449 | 6.98E−03 | −0.057097 | 0.930073 | 22,910,351 | A | G | 1337 |
| rs1997903 | 7.07E−03 | −0.079799 | 0.039086 | 22,839,197 | A | G | 1338 |
| rs2332155 | 7.39E−03 | 0.189725 | 0.990531 | 23,534,789 | A | G | 1339 |
| chr14: 22749845 | 7.58E−03 | −0.039311 | 0.390277 | 22,749,845 | A | G | 1340 |
| rs10148260 | 7.74E−03 | 0.031658 | 0.761237 | 21,996,162 | A | C | 1341 |
| chr14: 23003012 | 7.94E−03 | 0.061159 | 0.121219 | 23,003,012 | C | T | 1342 |
| chr14: 23655278 | 8.12E−03 | 0.201716 | 0.992658 | 23,655,278 | G | T | 1343 |
| chr14: 23667214 | 8.12E−03 | 0.407006 | 0.996219 | 23,667,214 | A | G | 1344 |
| rs4982751 | 8.15E−03 | −0.031185 | 0.401469 | 22,875,004 | C | T | 1345 |
| chr14: 22819803 | 8.16E−03 | −0.094432 | 0.086725 | 22,819,803 | A | T | 1346 |
| chr14: 23510084 | 8.43E−03 | 0.183548 | 0.990232 | 23,510,084 | A | G | 1347 |
| rs1805061 | 8.58E−03 | 0.110598 | 0.867628 | 22,317,952 | A | G | 1348 |
| rs17794083 | 8.63E−03 | −0.035902 | 0.173995 | 21,996,759 | A | C | 1349 |
| rs221697 | 8.92E−03 | 0.05995 | 0.950283 | 23,209,497 | A | G | 1350 |
| rs221698 | 8.92E−03 | −0.059982 | 0.049691 | 23,207,946 | A | G | 1351 |
| rs221700 | 8.92E−03 | −0.060032 | 0.049652 | 23,205,823 | A | G | 1352 |
| rs221701 | 8.92E−03 | −0.060055 | 0.049633 | 23,205,675 | G | T | 1353 |
| rs221694 | 8.97E−03 | −0.060481 | 0.049278 | 23,216,565 | A | G | 1354 |
| rs221703 | 9.02E−03 | −0.061722 | 0.048304 | 23,204,385 | A | T | 1355 |
| rs221691 | 9.06E−03 | 0.060422 | 0.950718 | 23,220,495 | G | T | 1356 |
| rs221690 | 9.07E−03 | −0.060412 | 0.049283 | 23,220,862 | A | T | 1357 |
| chr14: 23493423 | 9.09E−03 | −0.18018 | 0.010131 | 23,493,423 | C | T | 1358 |
| rs2577695 | 9.11E−03 | 0.060381 | 0.950714 | 23,221,610 | A | G | 1359 |
| rs8022177 | 9.35E−03 | 0.064516 | 0.955576 | 22,543,546 | C | T | 1360 |
| rs221689 | 9.42E−03 | 0.060167 | 0.950699 | 23,223,883 | C | T | 1361 |
| chr14: 22543249 | 9.46E−03 | −0.064417 | 0.04457 | 22,543,249 | G | T | 1362 |
| rs1956955 | 9.53E−03 | −0.042886 | 0.8537 | 22,997,768 | A | T | 1363 |

Shown is marker identity, p-value of the association, magnitude of effect (in units of fractions of standard deviations), frequency of effect allele, position in NCBI Build 36, identity of effect allele, identity of other allele, SEQ ID for the marker. It should be noted that a positive value for an effect represents an increase in heart rate conferred by the effect allele, while a negative value for the effect represents a decrease (i.e. the allele correlated with the negative effect is predictive of a decreased heart rate).

TABLE 22
Association results for PR interval in a 2 Mb
region flanking rs7660702 on chromosome 4.

| | | | | | eff | other | Seq ID |
|---|---|---|---|---|---|---|---|
| marker | p-value | effect | freq | position | all | all | NO: |
| rs13105921 | 7.32971E−09 | 0.073838 | 0.707288 | 86,918,750 | A | C | 106 |
| rs13137008 | 2.3531E−08 | −0.071958 | 0.270268 | 86,893,223 | G | T | 88 |
| rs17010812 | 2.80668E−08 | 0.071513 | 0.726925 | 86,907,441 | C | T | 323 |
| rs17010816 | 2.80746E−08 | −0.071521 | 0.273056 | 86,908,395 | A | C | 324 |
| rs7660252 | 2.81432E−08 | −0.071589 | 0.272898 | 86,909,580 | A | G | 325 |
| rs34422670 | 2.82073E−08 | 0.071643 | 0.727249 | 86,910,056 | A | G | 326 |
| rs2869384 | 2.82589E−08 | 0.071495 | 0.726849 | 86,905,788 | C | T | 327 |
| rs17010847 | 2.85086E−08 | −0.071869 | 0.272174 | 86,914,002 | A | G | 328 |
| rs34949750 | 2.88148E−08 | −0.071125 | 0.274281 | 86,903,197 | A | G | 329 |
| rs11736641 | 2.88333E−08 | 0.071117 | 0.725697 | 86,902,753 | G | T | 94 |
| rs13111662 | 2.88589E−08 | −0.071107 | 0.274334 | 86,902,145 | C | T | 92 |
| rs13111293 | 2.88654E−08 | −0.071104 | 0.274341 | 86,901,992 | C | T | 330 |
| rs13112030 | 3.44218E−08 | 0.072761 | 0.742552 | 86,893,164 | A | T | 331 |
| rs11724267 | 3.50834E−08 | 0.071154 | 0.735301 | 86,896,100 | A | G | 332 |
| rs17010755 | 3.70241E−08 | 0.070955 | 0.735277 | 86,891,118 | C | G | 333 |
| rs13113783 | 3.74657E−08 | −0.070914 | 0.264759 | 86,889,899 | C | T | 334 |
| rs10012090 | 3.74818E−08 | −0.070923 | 0.267915 | 86,857,950 | A | G | 63 |
| rs7660702 | 3.77122E−08 | 0.070732 | 0.734626 | 86,870,488 | T | C | 72 |
| rs28490560 | 3.86083E−08 | −0.070817 | 0.264827 | 86,887,076 | C | T | 335 |
| rs10516755 | 3.95077E−08 | 0.070744 | 0.735131 | 86,885,065 | C | T | 83 |
| rs2601855 | 4.00195E−08 | −0.070704 | 0.264892 | 86,883,964 | A | T | 80 |
| rs343849 | 4.09287E−08 | −0.070632 | 0.26493 | 86,882,079 | A | T | 78 |
| rs13150566 | 4.09565E−08 | 0.07052 | 0.695445 | 86,887,697 | A | G | 336 |
| rs7692808 | 4.15085E−08 | −0.072436 | 0.256663 | 86,860,173 | A | G | 65 |
| rs35361328 | 4.17298E−08 | 0.070539 | 0.734853 | 86,858,670 | A | C | 337 |
| rs2198328 | 4.20516E−08 | 0.07054 | 0.735018 | 86,879,268 | A | C | 338 |
| rs994285 | 4.22739E−08 | 0.070518 | 0.735002 | 86,878,138 | G | T | 76 |
| rs7658797 | 4.24369E−08 | −0.070481 | 0.26511 | 86,861,738 | A | C | 66 |
| rs2624021 | 4.26365E−08 | −0.070481 | 0.265022 | 86,876,444 | A | T | 339 |
| rs11937419 | 4.31269E−08 | 0.070432 | 0.734949 | 86,874,379 | C | T | 340 |
| rs6813799 | 4.3233E−08 | −0.070421 | 0.265057 | 86,873,960 | A | G | 341 |
| rs4507354 | 4.32708E−08 | 0.070417 | 0.734941 | 86,873,813 | C | G | 342 |
| rs4302455 | 4.33438E−08 | −0.07041 | 0.265063 | 86,873,532 | C | G | 343 |
| rs343860 | 4.33489E−08 | 0.070406 | 0.734931 | 86,863,360 | C | T | 68 |
| rs2869380 | 4.3352E−08 | −0.070409 | 0.265064 | 86,873,501 | A | G | 344 |
| rs2601861 | 4.33838E−08 | −0.07041 | 0.265069 | 86,873,687 | C | T | 345 |
| rs2869379 | 4.33953E−08 | −0.070405 | 0.265066 | 86,873,336 | G | T | 346 |
| rs343856 | 4.36698E−08 | 0.07038 | 0.734954 | 86,866,681 | G | T | 347 |
| rs1482093 | 4.36954E−08 | 0.070376 | 0.734925 | 86,872,160 | C | T | 348 |
| rs13127274 | 4.36961E−08 | −0.07038 | 0.265069 | 86,867,032 | A | G | 349 |
| rs13102580 | 4.3712E−08 | −0.070376 | 0.265046 | 86,867,122 | G | T | 350 |
| rs2869377 | 4.37492E−08 | −0.070372 | 0.26507 | 86,871,884 | A | G | 351 |
| rs2062097 | 4.38607E−08 | −0.070364 | 0.26506 | 86,871,330 | A | G | 352 |
| rs2062098 | 4.38726E−08 | 0.070363 | 0.734941 | 86,871,272 | C | T | 73 |
| rs1482094 | 4.38972E−08 | 0.070362 | 0.734955 | 86,869,043 | C | T | 70 |
| rs7677114 | 4.39885E−08 | 0.070358 | 0.734916 | 86,870,104 | A | T | 353 |
| rs2601860 | 2.50335E−07 | −0.069567 | 0.227477 | 86,876,210 | A | G | 354 |
| rs41527748 | 2.66346E−07 | −0.069368 | 0.227498 | 86,867,769 | A | G | 355 |
| rs67115123 | 1.63866E−06 | −0.068387 | 0.25417 | 86,871,206 | C | G | 356 |
| rs6849659 | 8.94649E−06 | 0.066383 | 0.813789 | 86,829,480 | A | G | 39 |
| rs34587524 | 1.06659E−05 | 0.065673 | 0.809343 | 86,829,889 | G | T | 357 |
| rs7655100 | 1.29412E−05 | 0.05296 | 0.676096 | 86,839,892 | A | T | 45 |
| rs900205 | 1.30707E−05 | −0.052842 | 0.323961 | 86,853,373 | A | G | 358 |
| rs1482085 | 1.30772E−05 | −0.052833 | 0.323957 | 86,849,536 | A | G | 55 |
| rs28505541 | 1.31964E−05 | 0.052821 | 0.675899 | 86,852,811 | C | T | 359 |
| rs28897130 | 1.32575E−05 | −0.07018 | 0.168685 | 86,828,022 | C | T | 360 |
| chr4: 86830387 | 1.42738E−05 | −0.053453 | 0.322626 | 86,830,387 | G | T | 361 |
| rs343854 | 2.82231E−05 | −0.052635 | 0.318988 | 86,873,178 | A | G | 362 |
| rs6531851 | 2.91995E−05 | 0.062505 | 0.809411 | 86,830,922 | A | G | 363 |
| rs11732231 | 4.06784E−05 | 0.05228 | 0.663288 | 86,902,584 | C | G | 93 |
| rs900203 | 4.51054E−05 | 0.051237 | 0.708374 | 86,853,445 | A | T | 364 |
| rs12510813 | 4.83536E−05 | 0.061137 | 0.82462 | 86,892,745 | A | G | 86 |
| rs13135703 | 4.84749E−05 | −0.061128 | 0.175363 | 86,892,681 | A | G | 365 |
| rs13110485 | 4.87995E−05 | −0.061102 | 0.175318 | 86,892,511 | C | T | 366 |
| rs34322771 | 5.04952E−05 | 0.060971 | 0.824907 | 86,891,649 | C | T | 367 |
| rs13121597 | 5.19506E−05 | −0.060859 | 0.174907 | 86,890,942 | A | G | 368 |
| rs9998523 | 5.32615E−05 | 0.06076 | 0.825253 | 86,890,329 | A | C | 369 |
| rs35341700 | 5.74296E−05 | 0.065258 | 0.845169 | 86,876,318 | A | G | 370 |
| rs35577842 | 6.16619E−05 | −0.064978 | 0.154733 | 86,870,664 | A | G | 371 |
| rs55982788 | 7.53301E−05 | 0.068171 | 0.868999 | 86,934,957 | C | G | 372 |
| rs13127367 | 8.31192E−05 | 0.063936 | 0.846892 | 86,867,257 | C | G | 373 |
| rs17010851 | 8.48907E−05 | 0.063606 | 0.853142 | 86,914,746 | A | T | 103 |
| rs4693736 | 8.50581E−05 | −0.063615 | 0.146993 | 86,915,344 | A | G | 105 |
| rs17010857 | 8.50702E−05 | 0.063603 | 0.853071 | 86,915,002 | G | T | 104 |
| chr4: 86910872 | 8.71709E−05 | 0.063353 | 0.853157 | 86,910,872 | A | G | 374 |
| rs11945319 | 8.74164E−05 | −0.063325 | 0.146833 | 86,910,619 | A | G | 100 |
| rs12498959 | 8.80655E−05 | −0.063246 | 0.146785 | 86,910,076 | A | T | 375 |
| chr4: 86909574 | 8.88077E−05 | −0.063162 | 0.146766 | 86,909,574 | A | G | 376 |
| rs1966862 | 8.98007E−05 | 0.063051 | 0.853259 | 86,907,085 | A | G | 97 |
| rs12508919 | 9.00482E−05 | −0.063094 | 0.146605 | 86,905,200 | C | T | 377 |
| rs11097071 | 9.09805E−05 | 0.063221 | 0.853831 | 86,904,360 | A | T | 95 |
| rs17010863 | 9.24387E−05 | −0.064429 | 0.143781 | 86,917,329 | A | G | 378 |
| rs17010925 | 9.92671E−05 | −0.066805 | 0.131647 | 86,932,148 | G | T | 118 |
| rs28491994 | 0.000106357 | −0.066578 | 0.131986 | 86,936,477 | C | T | 379 |
| rs12507198 | 0.000132982 | 0.063111 | 0.859816 | 86,901,712 | G | T | 91 |
| rs13106553 | 0.000153797 | −0.062586 | 0.139605 | 86,888,786 | A | G | 85 |
| rs4693735 | 0.000155414 | −0.062547 | 0.138754 | 86,896,131 | C | T | 90 |
| rs4693127 | 0.000156325 | −0.062524 | 0.138704 | 86,895,937 | A | G | 380 |
| chr4: 86917409 | 0.000183645 | 0.063505 | 0.861236 | 86,917,409 | C | T | 381 |
| rs4693734 | 0.000214819 | −0.061178 | 0.136727 | 86,883,707 | A | T | 382 |
| rs2004613 | 0.000215469 | −0.061167 | 0.136721 | 86,883,411 | C | T | 383 |
| rs3889735 | 0.000216691 | −0.061145 | 0.13671 | 86,882,860 | C | T | 79 |
| rs13139248 | 0.000220221 | 0.061083 | 0.863321 | 86,881,304 | C | G | 384 |
| rs13137575 | 0.000222186 | 0.061048 | 0.863338 | 86,880,459 | C | T | 385 |
| rs35472747 | 0.000223797 | 0.061019 | 0.863352 | 86,879,770 | C | T | 386 |
| rs12510148 | 0.000224423 | −0.061008 | 0.136642 | 86,879,503 | A | G | 387 |
| rs13136153 | 0.000231323 | −0.060885 | 0.136583 | 86,876,657 | C | T | 388 |
| rs12505724 | 0.00023429 | −0.060832 | 0.136558 | 86,875,484 | C | G | 389 |
| rs6813860 | 0.000237749 | 0.06077 | 0.863469 | 86,874,152 | C | G | 75 |
| rs58146546 | 0.000249228 | 0.060567 | 0.863545 | 86,870,232 | A | T | 390 |
| rs34820504 | 0.000249231 | −0.060567 | 0.136455 | 86,870,223 | A | G | 391 |
| rs7676486 | 0.00024946 | 0.060564 | 0.863546 | 86,869,655 | C | T | 71 |
| rs13108523 | 0.000250355 | −0.060554 | 0.136452 | 86,867,448 | C | T | 69 |
| rs13102863 | 0.000250421 | −0.060553 | 0.136451 | 86,867,285 | C | T | 392 |
| rs1014642 | 0.000251049 | −0.060545 | 0.13645 | 86,865,746 | A | T | 393 |
| rs12506038 | 0.000256847 | −0.060474 | 0.136434 | 86,861,445 | C | T | 394 |
| rs34056429 | 0.000259003 | 0.060447 | 0.863572 | 86,859,867 | C | T | 395 |
| rs71599423 | 0.000260222 | −0.060431 | 0.136425 | 86,858,180 | A | G | 396 |
| rs2101134 | 0.000355029 | 0.043757 | 0.647354 | 86,831,373 | A | G | 40 |
| rs11729287 | 0.000397963 | 0.053635 | 0.828631 | 86,945,849 | C | T | 397 |
| rs12507272 | 0.000406317 | −0.054782 | 0.181383 | 86,892,912 | C | T | 87 |
| chr4: 86710577 | 0.000447393 | 0.057989 | 0.411596 | 86,710,577 | C | T | 398 |
| rs7675429 | 0.000459244 | −0.052048 | 0.179097 | 86,940,109 | A | C | 120 |
| rs7689056 | 0.000473781 | −0.0519 | 0.179048 | 86,942,197 | A | G | 121 |
| rs7693640 | 0.000475265 | 0.051885 | 0.820957 | 86,942,405 | C | T | 122 |
| rs12511071 | 0.000505434 | −0.082081 | 0.064358 | 87,427,733 | A | C | 399 |
| rs1116117 | 0.000507908 | −0.043143 | 0.354438 | 86,829,025 | A | T | 400 |
| rs343852 | 0.000552915 | −0.066724 | 0.106658 | 86,880,423 | G | T | 401 |
| rs17010697 | 0.000557081 | 0.05824 | 0.871186 | 86,862,179 | A | C | 67 |
| chr4: 86886221 | 0.000559403 | 0.062422 | 0.724383 | 86,886,221 | A | T | 402 |
| rs343848 | 0.000569142 | −0.060241 | 0.123554 | 86,882,270 | A | C | 403 |
| rs41477846 | 0.000570896 | 0.058131 | 0.870967 | 86,863,154 | A | G | 404 |
| rs6531862 | 0.000572009 | 0.05809 | 0.871404 | 86,865,589 | C | T | 405 |
| rs72656252 | 0.000573163 | −0.058099 | 0.128865 | 86,865,028 | C | T | 406 |
| rs1482092 | 0.000592754 | −0.057921 | 0.128586 | 86,872,282 | A | C | 407 |
| rs2624023 | 0.000606069 | 0.057878 | 0.871532 | 86,875,748 | C | T | 408 |
| chr4: 86299715 | 0.000609435 | −0.591028 | 0.005277 | 86,299,715 | A | G | 409 |
| rs1020584 | 0.000625765 | 0.057959 | 0.872008 | 86,888,074 | A | G | 84 |
| rs13128115 | 0.000643615 | 0.05698 | 0.861234 | 86,852,508 | A | T | 60 |
| rs13112493 | 0.000644845 | 0.058604 | 0.873892 | 86,895,103 | A | G | 89 |
| rs72656285 | 0.000647566 | −0.058646 | 0.12595 | 86,895,620 | A | G | 410 |
| rs56989679 | 0.000650887 | −0.057798 | 0.134091 | 86,901,973 | C | T | 411 |
| rs6819529 | 0.000659015 | −0.05879 | 0.125363 | 86,897,550 | A | C | 412 |
| chr4: 86168511 | 0.000677591 | 0.584913 | 0.994936 | 86,168,511 | A | G | 413 |
| chr4: 86840597 | 0.000696875 | 0.052657 | 0.743203 | 86,840,597 | A | G | 414 |
| rs72656294 | 0.000707487 | −0.05788 | 0.132396 | 86,903,665 | G | T | 415 |
| chr4: 85904875 | 0.000747281 | 0.573762 | 0.99509 | 85,904,875 | A | G | 416 |
| rs17010599 | 0.000898739 | −0.065803 | 0.091057 | 86,838,704 | G | T | 44 |
| rs12646641 | 0.000901366 | −0.065591 | 0.091246 | 86,847,010 | A | G | 417 |
| rs11731040 | 0.000902874 | 0.06572 | 0.908744 | 86,841,367 | C | T | 50 |
| rs1811576 | 0.000914764 | −0.071004 | 0.089246 | 86,825,759 | A | G | 418 |
| rs4585295 | 0.000930373 | −0.054204 | 0.143526 | 86,844,610 | C | T | 419 |
| chr4: 86878564 | 0.000939511 | −0.061557 | 0.118297 | 86,878,564 | C | T | 420 |
| chr4: 86843230 | 0.000965887 | −0.065372 | 0.093553 | 86,843,230 | A | G | 421 |
| rs12648692 | 0.00101017 | 0.065151 | 0.905396 | 86,837,105 | A | G | 42 |
| rs7674888 | 0.00110772 | −0.056608 | 0.126541 | 86,904,770 | A | G | 96 |
| rs11097072 | 0.00112591 | −0.056535 | 0.126374 | 86,906,707 | A | T | 422 |
| rs12054628 | 0.00113006 | −0.056526 | 0.126326 | 86,907,793 | A | T | 98 |
| rs11097073 | 0.00113346 | −0.056555 | 0.126231 | 86,909,260 | C | T | 423 |
| rs6831420 | 0.00115842 | −0.056757 | 0.125566 | 86,911,689 | A | G | 101 |
| rs7680588 | 0.00116462 | 0.056804 | 0.874593 | 86,912,777 | C | T | 102 |
| rs11097074 | 0.00117245 | −0.056861 | 0.125211 | 86,915,644 | C | T | 424 |
| chr4: 86505218 | 0.00117643 | −0.593205 | 0.005878 | 86,505,218 | A | T | 425 |
| chr4: 86978387 | 0.00125428 | −0.060538 | 0.214821 | 86,978,387 | G | T | 426 |
| rs12650494 | 0.0012805 | 0.063461 | 0.90868 | 86,856,334 | C | T | 62 |
| rs1031987 | 0.0013415 | 0.072707 | 0.925265 | 86,858,565 | C | T | 427 |
| rs17011540 | 0.00135173 | −0.057466 | 0.109479 | 87,369,493 | G | T | 428 |
| rs1482091 | 0.00138704 | −0.063065 | 0.090711 | 86,872,385 | T | C | 74 |
| rs7434773 | 0.00141055 | −0.051717 | 0.145763 | 86,836,580 | A | G | 429 |
| rs4413396 | 0.00143172 | −0.050768 | 0.148201 | 86,851,795 | A | G | 57 |
| rs11735639 | 0.00143175 | −0.050769 | 0.148199 | 86,850,942 | G | T | 56 |
| rs36095037 | 0.0014318 | 0.050771 | 0.851806 | 86,849,510 | C | T | 430 |
| rs1871864 | 0.00143184 | 0.050773 | 0.85181 | 86,848,126 | A | T | 53 |
| rs1482084 | 0.00143186 | 0.050774 | 0.851811 | 86,847,677 | A | G | 431 |
| rs4488930 | 0.00143195 | 0.050778 | 0.851819 | 86,845,208 | C | T | 432 |
| rs13146939 | 0.00143197 | 0.050767 | 0.851799 | 86,852,284 | A | G | 58 |
| rs17010632 | 0.001432 | 0.05078 | 0.851824 | 86,844,920 | A | G | 52 |
| rs13152150 | 0.00143224 | 0.050766 | 0.851799 | 86,852,331 | A | G | 59 |
| rs12513384 | 0.00143278 | 0.050814 | 0.85189 | 86,843,716 | C | T | 433 |
| rs931195 | 0.00143381 | 0.050856 | 0.851976 | 86,843,465 | A | G | 51 |
| rs876107 | 0.0014339 | −0.05086 | 0.148017 | 86,842,332 | A | G | 434 |
| rs1110777 | 0.0014339 | 0.05086 | 0.851982 | 86,842,397 | A | G | 435 |
| rs7439720 | 0.00143414 | 0.050867 | 0.851998 | 86,840,878 | A | G | 48 |
| rs10033273 | 0.00143561 | −0.050904 | 0.14792 | 86,840,097 | A | C | 47 |
| rs7677064 | 0.00143566 | −0.050905 | 0.147917 | 86,839,908 | C | T | 46 |
| rs6531854 | 0.00143586 | −0.05091 | 0.147906 | 86,838,681 | C | T | 436 |
| rs6830971 | 0.00143756 | 0.050511 | 0.779208 | 86,823,896 | A | G | 437 |
| rs6531897 | 0.00143779 | 0.059874 | 0.159415 | 87,251,718 | A | G | 438 |
| rs10007242 | 0.00144145 | 0.05104 | 0.852386 | 86,835,195 | A | T | 439 |
| rs10017047 | 0.0014436 | −0.051096 | 0.147493 | 86,834,415 | A | G | 41 |
| rs2101135 | 0.00145472 | −0.051305 | 0.146997 | 86,831,412 | A | T | 440 |
| rs6531852 | 0.00145669 | 0.051038 | 0.852429 | 86,834,645 | A | C | 441 |
| chr4: 86860735 | 0.00149194 | −0.062481 | 0.091104 | 86,860,735 | C | T | 442 |
| rs6845071 | 0.00151325 | −0.05208 | 0.145045 | 86,829,316 | A | G | 443 |
| chr4: 86925126 | 0.00153117 | 0.055799 | 0.740299 | 86,925,126 | A | C | 444 |
| rs6842671 | 0.0015338 | 0.072184 | 0.070328 | 87,473,202 | C | G | 445 |
| chr4: 87485450 | 0.00154596 | 0.072132 | 0.070325 | 87,485,450 | C | G | 446 |
| rs28791572 | 0.00162583 | −0.056809 | 0.106656 | 87,378,508 | G | T | 447 |
| rs28870188 | 0.00163999 | −0.056788 | 0.106738 | 87,309,151 | A | C | 448 |
| rs7697134 | 0.00165162 | 0.061822 | 0.908957 | 86,866,099 | A | C | 449 |
| chr4: 86867589 | 0.00166677 | −0.061763 | 0.091041 | 86,867,589 | A | G | 450 |
| rs1482095 | 0.00168212 | −0.061706 | 0.09108 | 86,868,956 | A | G | 451 |
| rs6853196 | 0.00168258 | −0.061707 | 0.091109 | 86,868,909 | C | T | 452 |
| rs12647014 | 0.00169927 | 0.062248 | 0.90982 | 86,889,710 | A | G | 453 |
| rs4455406 | 0.0016995 | −0.062045 | 0.09048 | 86,887,976 | A | T | 454 |
| rs7682971 | 0.00169998 | −0.061936 | 0.090623 | 86,884,320 | C | T | 82 |
| rs11722392 | 0.00170011 | 0.061912 | 0.909346 | 86,883,521 | C | T | 455 |
| rs7661884 | 0.00170065 | 0.061816 | 0.90922 | 86,880,315 | A | C | 456 |
| rs57065351 | 0.00170169 | 0.062561 | 0.910315 | 86,891,407 | A | C | 457 |
| rs7685320 | 0.00170172 | −0.061793 | 0.090806 | 86,879,524 | C | T | 458 |
| rs6531863 | 0.00170313 | −0.061767 | 0.090836 | 86,878,621 | C | T | 459 |
| chr4: 86876836 | 0.00170595 | 0.061714 | 0.909106 | 86,876,836 | A | G | 460 |
| rs6839040 | 0.00170736 | 0.061688 | 0.909077 | 86,875,944 | A | G | 461 |
| rs12644984 | 0.0017085 | −0.061667 | 0.090946 | 86,875,230 | C | G | 462 |
| rs13107701 | 0.00171491 | 0.052136 | 0.861651 | 86,947,171 | C | T | 463 |
| rs36104548 | 0.00175186 | −0.051592 | 0.140881 | 86,948,615 | G | T | 464 |
| chr4: 86899196 | 0.00176532 | −0.063727 | 0.087382 | 86,899,196 | C | T | 465 |
| rs7675688 | 0.00176739 | 0.063744 | 0.912662 | 86,899,342 | C | G | 466 |
| chr4: 86899554 | 0.00177047 | 0.063769 | 0.912725 | 86,899,554 | A | T | 467 |
| rs11733669 | 0.00177193 | −0.052086 | 0.136291 | 86,945,753 | C | T | 468 |
| rs7686507 | 0.00182477 | 0.092137 | 0.958845 | 86,859,200 | C | T | 469 |
| rs35201853 | 0.00183721 | −0.091845 | 0.041284 | 86,857,328 | A | C | 470 |
| chr4: 87619908 | 0.00187452 | 0.201429 | 0.008297 | 87,619,908 | C | T | 471 |
| rs1871865 | 0.00191246 | −0.049085 | 0.156481 | 86,848,557 | C | G | 54 |
| rs2167718 | 0.00191296 | −0.050741 | 0.142897 | 86,951,747 | G | T | 472 |
| chr4: 86895034 | 0.00201238 | −0.318629 | 0.991829 | 86,895,034 | A | C | 473 |
| chr4: 87608123 | 0.00202509 | −0.20084 | 0.991806 | 87,608,123 | C | T | 474 |
| rs189397 | 0.00202711 | 0.035679 | 0.412464 | 87,069,007 | C | T | 475 |
| rs7699404 | 0.0021147 | 0.034438 | 0.458615 | 86,939,862 | T | C | 476 |
| rs60426074 | 0.00212887 | 0.075455 | 0.93114 | 86,830,412 | C | T | 477 |
| rs6815441 | 0.00217148 | −0.051698 | 0.13714 | 86,949,538 | T | C | 478 |
| chr4: 87629257 | 0.00238127 | 0.066462 | 0.074589 | 87,629,257 | A | C | 479 |
| rs34868396 | 0.00238845 | 0.09639 | 0.959179 | 86,830,386 | C | T | 480 |
| chr4: 87707600 | 0.00244825 | 0.066201 | 0.074681 | 87,707,600 | C | T | 481 |
| chr4: 87574965 | 0.00253969 | 0.091751 | 0.038228 | 87,574,965 | C | G | 482 |
| chr4: 87772826 | 0.00265726 | −0.065552 | 0.924794 | 87,772,826 | C | T | 483 |
| rs7666211 | 0.00302491 | −0.047735 | 0.155106 | 86,846,755 | C | T | 484 |
| rs13123125 | 0.00313846 | 0.060151 | 0.910974 | 86,908,641 | C | T | 485 |
| rs17010839 | 0.00317337 | −0.060349 | 0.088654 | 86,910,281 | G | T | 99 |
| rs73834264 | 0.00326877 | 0.191644 | 0.00822 | 87,434,185 | C | T | 486 |
| rs62305526 | 0.0035209 | 0.06086 | 0.134415 | 87,275,021 | A | G | 487 |
| rs4693126 | 0.00359942 | −0.057115 | 0.111692 | 86,895,823 | G | T | 488 |
| rs6855390 | 0.00371043 | 0.067536 | 0.917963 | 86,873,236 | A | C | 489 |
| chr4: 87413305 | 0.00397503 | 0.187463 | 0.008 | 87,413,305 | A | G | 490 |
| chr4: 87407757 | 0.00424805 | 0.186387 | 0.007973 | 87,407,757 | A | C | 491 |
| chr4: 87485357 | 0.00440884 | 0.19031 | 0.007921 | 87,485,357 | A | G | 492 |
| rs7439226 | 0.00482897 | −0.113617 | 0.021597 | 86,526,830 | A | G | 493 |
| chr4: 86789212 | 0.00486636 | 0.101734 | 0.949574 | 86,789,212 | G | T | 494 |
| rs12499867 | 0.00492277 | 0.036739 | 0.687759 | 86,919,587 | C | T | 495 |
| chr4: 86852629 | 0.00495728 | −0.167604 | 0.013774 | 86,852,629 | A | C | 496 |
| rs6531908 | 0.00514753 | −0.034536 | 0.340328 | 87,395,339 | G | T | 497 |
| chr4: 86280463 | 0.00531648 | 0.423296 | 0.993146 | 86,280,463 | C | G | 498 |
| rs11726269 | 0.00535877 | −0.080875 | 0.949985 | 87,452,008 | A | G | 499 |
| chr4: 86641656 | 0.00550462 | 0.072723 | 0.073355 | 86,641,656 | G | T | 500 |
| chr4: 86467844 | 0.00602276 | 0.106597 | 0.977124 | 86,467,844 | C | T | 501 |
| chr4: 86793655 | 0.00628914 | 0.061555 | 0.882233 | 86,793,655 | A | T | 502 |
| rs13147689 | 0.00633548 | 0.040186 | 0.806705 | 86,839,564 | C | T | 503 |
| rs72656271 | 0.00639039 | −0.063427 | 0.892981 | 86,882,960 | A | T | 504 |
| chr4: 87114168 | 0.00639719 | 0.133279 | 0.978079 | 87,114,168 | C | T | 505 |
| rs13110928 | 0.00641402 | 0.046265 | 0.840253 | 86,864,521 | A | G | 506 |
| rs900204 | 0.00649366 | −0.04013 | 0.196321 | 86,841,290 | A | G | 49 |
| chr4: 86779965 | 0.00655558 | −0.103013 | 0.963003 | 86,779,965 | C | T | 507 |
| chr4: 86925138 | 0.00670509 | −0.152861 | 0.956976 | 86,925,138 | C | G | 508 |
| chr4: 86610824 | 0.00681298 | −0.124018 | 0.980054 | 86,610,824 | C | T | 509 |
| rs13125320 | 0.00681693 | 0.031897 | 0.452012 | 86,917,411 | C | T | 510 |
| rs2126021 | 0.00707073 | −0.092261 | 0.969344 | 87,339,787 | A | G | 511 |
| rs1460762 | 0.00714074 | 0.092287 | 0.030753 | 87,316,997 | A | G | 512 |
| chr4: 87563777 | 0.00720514 | 0.127254 | 0.01506 | 87,563,777 | A | G | 513 |
| rs343847 | 0.00751356 | 0.030177 | 0.424237 | 86,882,283 | A | C | 514 |
| chr4: 87348930 | 0.00762078 | −0.074873 | 0.042397 | 87,348,930 | A | G | 515 |
| chr4: 87309739 | 0.00767551 | 0.074872 | 0.957633 | 87,309,739 | C | T | 516 |
| chr4: 87035189 | 0.00769285 | 0.049587 | 0.820573 | 87,035,189 | A | C | 517 |
| chr4: 87559726 | 0.00771327 | −0.12585 | 0.984077 | 87,559,726 | C | T | 518 |
| rs7696763 | 0.00790151 | 0.045816 | 0.123989 | 87,048,553 | A | G | 519 |
| chr4: 87609737 | 0.00800871 | 0.181388 | 0.007542 | 87,609,737 | C | T | 520 |
| chr4: 87287406 | 0.00801144 | −0.074855 | 0.04769 | 87,287,406 | C | G | 521 |
| chr4: 87367662 | 0.00818169 | −0.059243 | 0.079545 | 87,367,662 | A | T | 522 |
| rs6827405 | 0.00818691 | −0.034783 | 0.235024 | 87,308,668 | C | T | 523 |
| rs67712948 | 0.00834173 | −0.034246 | 0.262558 | 87,345,843 | A | C | 524 |
| rs17011564 | 0.00848628 | −0.034238 | 0.241401 | 87,372,467 | C | G | 525 |
| rs17400759 | 0.0086636 | −0.033168 | 0.721823 | 86,979,041 | C | G | 526 |
| chr4: 87749368 | 0.00869834 | 0.179658 | 0.007414 | 87,749,368 | A | G | 527 |
| chr4: 87661852 | 0.00872399 | 0.179605 | 0.007416 | 87,661,852 | C | T | 528 |
| chr4: 86735324 | 0.00918611 | 0.147986 | 0.979127 | 86,735,324 | A | G | 529 |
| rs28896887 | 0.00923726 | 0.04068 | 0.84515 | 87,341,205 | A | G | 530 |
| rs66544335 | 0.00943407 | −0.033713 | 0.265842 | 87,345,890 | G | T | 531 |
| chr4: 87284496 | 0.00944825 | −0.093322 | 0.968736 | 87,284,496 | A | G | 532 |
| chr4: 87075373 | 0.00960776 | −0.131898 | 0.019991 | 87,075,373 | A | G | 533 |
| chr4: 87230422 | 0.00972013 | −0.233391 | 0.007177 | 87,230,422 | A | G | 534 |
| chr4: 87229705 | 0.00980706 | −0.22976 | 0.00726 | 87,229,705 | A | T | 535 |

Shown is marker identity, p-value of the association, magnitude of effect (in units of fractions of standard deviations), frequency of effect allele, position in NCBI Build 36, identity of effect allele, identity of other allele, SEQ ID for the marker. It should be noted that a positive value for an effect represents predicted increase in the interval conferred by the effect allele, while a negative value for the effect represents a decrease (i.e. the allele correlated with the negative effect is predictive of a decreased value of the measure).

TABLE 23
Association results for QRS interval in a 2
Mb region flanking rs1321311 on chromosome 6.

| | | | | | effect | other | SEQ ID |
|---|---|---|---|---|---|---|---|
| marker | P-value | effect | freq | position | allele | allele | NO: |
| rs4151702 | 3.45E−08 | 0.087491 | 0.157695 | 36,753,966 | C | G | 808 |
| rs730506 | 3.48E−08 | 0.08747 | 0.157761 | 36,753,946 | C | G | 809 |
| rs12199346 | 3.63E−08 | 0.07448 | 0.217378 | 36,749,524 | A | C | 810 |
| rs3176326 | 5.61E−08 | 0.086528 | 0.158602 | 36,755,267 | A | G | 811 |
| rs66761782 | 7.74E−08 | 0.072326 | 0.211639 | 36,744,058 | C | T | 812 |
| rs7742159 | 8.46E−08 | −0.072401 | 0.792124 | 36,727,430 | A | C | 813 |
| rs9470358 | 8.48E−08 | 0.072374 | 0.207876 | 36,728,480 | A | G | 814 |
| rs9918353 | 8.49E−08 | −0.072286 | 0.792087 | 36,730,655 | A | C | 815 |
| rs1321311 | 8.51E−08 | 0.072246 | 0.207922 | 36,730,878 | A | C | 127 |
| rs1321310 | 8.74E−08 | 0.071815 | 0.21096 | 36,731,102 | C | T | 128 |
| rs9470361 | 8.75E−08 | 0.071814 | 0.210961 | 36,731,357 | A | G | 130 |
| rs9462210 | 8.75E−08 | 0.071816 | 0.210998 | 36,736,931 | A | G | 141 |
| rs9462207 | 8.84E−08 | 0.071785 | 0.211002 | 36,735,577 | C | T | 137 |
| rs7756236 | 1.14E−07 | −0.071754 | 0.786852 | 36,735,031 | A | G | 136 |
| rs3176342 | 1.47E−07 | −0.088181 | 0.858994 | 36,757,571 | A | G | 816 |
| rs4331968 | 2.04E−07 | −0.071515 | 0.796717 | 36,731,221 | A | T | 129 |
| rs1321313 | 2.68E−07 | −0.069028 | 0.78164 | 36,726,799 | C | T | 125 |
| rs4711457 | 3.72E−07 | −0.060591 | 0.68523 | 36,741,138 | C | T | 146 |
| rs4711456 | 4.01E−07 | 0.060599 | 0.314693 | 36,740,460 | C | T | 817 |
| rs56100429 | 4.39E−07 | −0.071194 | 0.728779 | 36,724,578 | C | T | 818 |
| rs7774130 | 4.78E−07 | −0.069935 | 0.799231 | 36,731,734 | C | T | 819 |
| rs3176323 | 5.20E−07 | 0.064494 | 0.311257 | 36,754,827 | C | T | 820 |
| rs9470366 | 5.49E−07 | 0.069445 | 0.198528 | 36,733,540 | A | G | 133 |
| rs4713999 | 6.70E−07 | 0.059673 | 0.311916 | 36,741,047 | A | G | 145 |
| rs10807170 | 6.80E−07 | 0.059214 | 0.310456 | 36,737,422 | C | T | 142 |
| rs4713996 | 6.82E−07 | 0.059205 | 0.310473 | 36,737,692 | C | T | 143 |
| rs9394368 | 6.88E−07 | 0.059184 | 0.310498 | 36,738,503 | C | G | 144 |
| rs6930083 | 6.90E−07 | 0.059216 | 0.310657 | 36,742,134 | A | G | 147 |
| chr6: 36743712 | 1.14E−06 | 0.081981 | 0.159842 | 36,743,712 | G | T | 821 |
| rs6937605 | 1.51E−06 | −0.07766 | 0.849948 | 36,767,910 | C | T | 156 |
| rs12207916 | 4.76E−06 | 0.060537 | 0.262669 | 36,725,630 | C | T | 124 |
| rs733590 | 7.31E−06 | 0.053502 | 0.347639 | 36,753,181 | C | T | 150 |
| rs7767246 | 9.13E−06 | −0.067918 | 0.836796 | 36,767,193 | C | G | 155 |
| rs12191972 | 9.47E−06 | 0.068054 | 0.161084 | 36,766,720 | C | T | 154 |
| rs4714001 | 1.17E−05 | −0.05075 | 0.644104 | 36,746,153 | A | G | 148 |
| rs13196885 | 1.78E−05 | 0.047786 | 0.429447 | 36,740,666 | C | T | 822 |
| rs12207548 | 1.92E−05 | 0.066279 | 0.195542 | 36,764,234 | T | C | 153 |
| rs60598739 | 2.51E−05 | −0.057103 | 0.445117 | 37,272,411 | C | T | 823 |
| rs6457933 | 2.79E−05 | 0.047405 | 0.395995 | 36,726,246 | C | T | 824 |
| rs3176320 | 3.39E−05 | −0.050655 | 0.648488 | 36,754,766 | A | G | 825 |
| rs7740181 | 3.47E−05 | −0.048002 | 0.618452 | 36,732,089 | C | T | 826 |
| rs9368950 | 3.48E−05 | 0.045788 | 0.512641 | 36,735,850 | T | C | 138 |
| rs2395655 | 3.80E−05 | −0.048476 | 0.618345 | 36,753,674 | A | G | 151 |
| rs4135240 | 4.25E−05 | 0.049908 | 0.362913 | 36,755,658 | C | T | 827 |
| rs9470367 | 4.95E−05 | −0.044679 | 0.489068 | 36,734,910 | C | G | 135 |
| rs4713994 | 5.00E−05 | −0.04475 | 0.48902 | 36,729,511 | C | T | 126 |
| rs9462209 | 6.04E−05 | −0.044909 | 0.595105 | 36,736,020 | T | G | 140 |
| rs9470363 | 6.06E−05 | 0.04492 | 0.404642 | 36,732,691 | A | G | 828 |
| rs9462208 | 6.11E−05 | 0.044873 | 0.404858 | 36,735,868 | C | T | 139 |
| rs6936993 | 6.11E−05 | 0.044873 | 0.404858 | 36,734,300 | C | T | 134 |
| rs6936598 | 6.11E−05 | 0.044873 | 0.404858 | 36,734,074 | C | T | 829 |
| rs11969445 | 6.11E−05 | 0.044873 | 0.404858 | 36,733,360 | C | T | 132 |
| rs6930671 | 6.11E−05 | 0.044873 | 0.404858 | 36,733,250 | C | T | 131 |
| rs6907793 | 6.11E−05 | −0.044873 | 0.595142 | 36,733,184 | A | G | 830 |
| rs6907437 | 6.11E−05 | −0.044873 | 0.595142 | 36,732,999 | A | G | 831 |
| rs9470362 | 6.11E−05 | 0.044872 | 0.404857 | 36,732,627 | C | T | 832 |
| rs4568449 | 6.18E−05 | −0.044886 | 0.595113 | 36,729,121 | A | G | 833 |
| rs3176334 | 6.73E−05 | 0.048552 | 0.381061 | 36,756,342 | C | T | 834 |
| rs7753169 | 7.47E−05 | −0.052258 | 0.648381 | 36,722,304 | A | C | 835 |
| rs626516 | 8.04E−05 | −0.056717 | 0.791023 | 36,573,358 | A | G | 836 |
| rs3176336 | 9.26E−05 | −0.046128 | 0.602036 | 36,756,794 | A | T | 837 |
| rs9380584 | 1.17E−04 | 0.044982 | 0.49057 | 36,724,676 | C | T | 838 |
| rs1321309 | 1.39E−04 | −0.042256 | 0.524934 | 36,746,614 | A | G | 149 |
| rs3829964 | 1.52E−04 | 0.042475 | 0.467194 | 36,752,476 | C | T | 839 |
| rs762624 | 1.52E−04 | −0.054999 | 0.794736 | 36,753,566 | A | C | 840 |
| chr6: 36699107 | 1.78E−04 | 0.111815 | 0.044907 | 36,699,107 | A | T | 841 |
| chr6: 36699106 | 1.80E−04 | 0.111766 | 0.044942 | 36,699,106 | A | C | 842 |
| rs6457931 | 1.89E−04 | 0.045567 | 0.382831 | 36,721,790 | G | T | 123 |
| chr6: 36729910 | 2.11E−04 | 0.214064 | 0.015748 | 36,729,910 | A | C | 843 |
| chr6: 36605513 | 2.12E−04 | −0.144656 | 0.027308 | 36,605,513 | C | T | 844 |
| chr6: 36554548 | 2.13E−04 | 0.144603 | 0.97272 | 36,554,548 | C | G | 845 |
| rs56361858 | 2.14E−04 | −0.144609 | 0.027269 | 36,555,040 | C | G | 846 |
| chr6: 36570540 | 2.14E−04 | −0.144651 | 0.02725 | 36,570,540 | C | T | 847 |
| chr6: 36601182 | 2.14E−04 | −0.144272 | 0.027374 | 36,601,182 | C | T | 848 |
| rs3176352 | 2.17E−04 | −0.050625 | 0.78123 | 36,760,317 | C | G | 152 |
| chr6: 36559248 | 2.19E−04 | −0.143949 | 0.027685 | 36,559,248 | A | C | 849 |
| rs611831 | 2.23E−04 | 0.110271 | 0.044447 | 36,673,286 | C | G | 850 |
| rs9380583 | 2.24E−04 | −0.044064 | 0.427639 | 36,724,619 | A | G | 851 |
| rs646574 | 2.26E−04 | −0.110021 | 0.9557 | 36,684,612 | C | T | 852 |
| rs9470439 | 2.54E−04 | 0.061285 | 0.813713 | 37,010,617 | C | T | 853 |
| rs4713992 | 2.61E−04 | −0.052038 | 0.624632 | 36,720,183 | A | G | 854 |
| rs11754995 | 3.78E−04 | −0.048344 | 0.383014 | 36,720,301 | A | G | 855 |
| rs12193641 | 4.01E−04 | −0.072645 | 0.098879 | 36,600,748 | A | G | 856 |
| rs1321308 | 4.67E−04 | 0.039336 | 0.490618 | 36,746,669 | A | C | 857 |
| chr6: 36688262 | 4.88E−04 | 0.13279 | 0.032901 | 36,688,262 | A | T | 858 |
| chr6: 36704523 | 5.06E−04 | −0.132482 | 0.967394 | 36,704,523 | C | G | 859 |
| rs72853335 | 5.85E−04 | 0.102879 | 0.045526 | 36,738,987 | A | G | 860 |
| rs10947663 | 6.77E−04 | −0.038716 | 0.570174 | 37,272,580 | A | C | 861 |
| chr6: 36799586 | 7.54E−04 | 0.092462 | 0.050005 | 36,799,586 | C | T | 862 |
| rs3176337 | 9.18E−04 | −0.038083 | 0.458869 | 36,756,898 | A | C | 863 |
| rs7773453 | 1.07E−03 | −0.047549 | 0.806031 | 36,349,747 | G | T | 864 |
| rs73412244 | 1.09E−03 | 0.10759 | 0.044114 | 36,757,272 | C | T | 865 |
| rs12202603 | 1.13E−03 | 0.041699 | 0.236491 | 36,351,472 | G | T | 866 |
| rs9368919 | 1.13E−03 | −0.04182 | 0.76359 | 36,350,268 | A | T | 867 |
| rs12201879 | 1.13E−03 | 0.041887 | 0.236363 | 36,349,589 | A | G | 868 |
| rs35968576 | 1.16E−03 | 0.041627 | 0.236357 | 36,351,618 | C | T | 869 |
| chr6: 37114553 | 1.21E−03 | −0.118402 | 0.027449 | 37,114,553 | A | G | 870 |
| chr6: 37114847 | 1.22E−03 | −0.118631 | 0.026936 | 37,114,847 | A | C | 871 |
| chr6: 36631721 | 1.30E−03 | −0.112457 | 0.040497 | 36,631,721 | A | C | 872 |
| rs12191389 | 1.30E−03 | −0.07331 | 0.082452 | 36,535,800 | A | T | 873 |
| rs62406593 | 1.33E−03 | −0.039736 | 0.714392 | 36,776,141 | C | T | 874 |
| rs12200049 | 1.38E−03 | −0.040447 | 0.695212 | 36,348,819 | C | T | 875 |
| rs58708371 | 1.40E−03 | 0.039528 | 0.28537 | 36,774,363 | A | C | 876 |
| chr6: 36559632 | 1.43E−03 | 0.175604 | 0.981526 | 36,559,632 | C | T | 877 |
| rs1541316 | 1.54E−03 | −0.040693 | 0.764876 | 36,352,961 | G | T | 878 |
| rs72851235 | 1.57E−03 | −0.069714 | 0.0862 | 36,545,075 | A | G | 879 |
| chr6: 36127016 | 1.58E−03 | 0.048539 | 0.742109 | 36,127,016 | A | C | 880 |
| rs736348 | 1.67E−03 | 0.05137 | 0.18588 | 36,859,223 | A | G | 881 |
| rs3734341 | 1.68E−03 | 0.113441 | 0.9685 | 37,083,209 | A | G | 882 |
| rs9368920 | 1.69E−03 | −0.039126 | 0.735616 | 36,350,295 | C | T | 883 |
| rs56285849 | 2.05E−03 | 0.068598 | 0.07624 | 36,812,271 | C | T | 884 |
| chr6: 36184608 | 2.10E−03 | −0.04496 | 0.30025 | 36,184,608 | G | T | 885 |
| rs8180685 | 2.33E−03 | −0.039333 | 0.765769 | 36,355,081 | C | T | 886 |
| rs6923899 | 2.43E−03 | 0.03853 | 0.268786 | 36,778,169 | A | G | 887 |
| rs743851 | 2.45E−03 | −0.038917 | 0.73929 | 36,341,442 | A | G | 888 |
| rs60143683 | 2.50E−03 | −0.039028 | 0.739173 | 36,339,365 | A | G | 889 |
| rs56146544 | 2.57E−03 | −0.041921 | 0.799416 | 36,519,042 | G | T | 890 |
| chr6: 36757163 | 2.79E−03 | −0.044148 | 0.60248 | 36,757,163 | C | T | 891 |
| chr6: 36793359 | 2.79E−03 | 0.05065 | 0.785176 | 36,793,359 | A | G | 892 |
| chr6: 36625084 | 3.08E−03 | −0.059915 | 0.817492 | 36,625,084 | C | T | 893 |
| chr6: 37183956 | 3.11E−03 | −0.0819 | 0.052541 | 37,183,956 | A | C | 894 |
| rs12201679 | 3.23E−03 | −0.045145 | 0.835569 | 36,856,018 | C | T | 895 |
| chr6: 36845486 | 3.27E−03 | −0.104159 | 0.966449 | 36,845,486 | G | T | 896 |
| chr6: 36045139 | 3.28E−03 | −0.235991 | 0.00745 | 36,045,139 | A | G | 897 |
| rs56317508 | 3.38E−03 | 0.034942 | 0.349792 | 36,351,806 | C | T | 898 |
| rs6457942 | 3.43E−03 | −0.033672 | 0.623037 | 36,775,141 | C | T | 899 |
| rs13199306 | 3.64E−03 | −0.04008 | 0.234483 | 37,010,286 | C | T | 900 |
| rs6457940 | 3.64E−03 | 0.035019 | 0.320265 | 36,771,335 | A | C | 901 |
| rs12215712 | 3.67E−03 | 0.053327 | 0.885248 | 36,660,855 | A | T | 902 |
| rs12201085 | 3.72E−03 | −0.065675 | 0.081995 | 36,509,359 | A | G | 903 |
| chr6: 36699733 | 3.84E−03 | −0.052823 | 0.115238 | 36,699,733 | A | C | 904 |
| rs6912891 | 3.90E−03 | 0.032656 | 0.608639 | 36,959,742 | G | T | 905 |
| chr6: 36958508 | 3.91E−03 | −0.035802 | 0.362934 | 36,958,508 | A | G | 906 |
| rs1738455 | 3.92E−03 | 0.035559 | 0.61412 | 37,711,080 | C | T | 907 |
| rs9357229 | 3.98E−03 | 0.032568 | 0.608444 | 36,962,671 | A | G | 908 |
| rs665793 | 4.13E−03 | −0.046889 | 0.856232 | 36,491,935 | A | G | 909 |
| rs62408365 | 4.19E−03 | −0.061763 | 0.076281 | 37,197,366 | A | G | 910 |
| chr6: 36575432 | 4.20E−03 | 0.126755 | 0.023603 | 36,575,432 | C | T | 911 |
| chr6: 37129521 | 4.23E−03 | −0.042587 | 0.310816 | 37,129,521 | G | T | 912 |
| rs10947594 | 4.27E−03 | 0.036543 | 0.280193 | 36,338,556 | A | G | 913 |
| chr6: 36236713 | 4.38E−03 | −0.216018 | 0.008927 | 36,236,713 | C | T | 914 |
| rs6930941 | 4.41E−03 | 0.034145 | 0.336031 | 36,770,679 | C | T | 915 |
| rs11963261 | 4.42E−03 | −0.035361 | 0.281079 | 36,968,944 | C | T | 916 |
| rs12213912 | 4.42E−03 | 0.03534 | 0.719237 | 36,968,070 | C | T | 917 |
| rs7772573 | 4.49E−03 | 0.033905 | 0.313511 | 36,349,212 | A | G | 918 |
| rs1023037 | 4.50E−03 | −0.042185 | 0.161484 | 37,023,932 | A | G | 919 |
| rs2177845 | 4.55E−03 | 0.042156 | 0.838328 | 37,023,072 | C | T | 920 |
| rs1100858 | 4.60E−03 | −0.041493 | 0.177413 | 36,162,372 | A | G | 921 |
| chr6: 36184598 | 4.67E−03 | 0.039168 | 0.411032 | 36,184,598 | A | C | 922 |
| rs236448 | 4.77E−03 | 0.033177 | 0.639691 | 36,811,073 | A | C | 923 |
| rs6903663 | 4.78E−03 | −0.034993 | 0.684929 | 36,338,642 | A | C | 924 |
| rs851025 | 4.81E−03 | 0.044992 | 0.849725 | 36,107,217 | A | G | 925 |
| rs10807171 | 5.11E−03 | −0.033863 | 0.68829 | 36,770,878 | A | G | 926 |
| rs6457938 | 5.14E−03 | −0.033986 | 0.429634 | 36,768,431 | A | G | 927 |
| chr6: 36655330 | 5.17E−03 | 0.050812 | 0.21913 | 36,655,330 | A | G | 928 |
| chr6: 36513839 | 5.19E−03 | 0.043731 | 0.146784 | 36,513,839 | C | T | 929 |
| chr6: 36727927 | 5.33E−03 | −0.090455 | 0.939679 | 36,727,927 | C | T | 930 |
| rs1547421 | 5.34E−03 | −0.033132 | 0.671274 | 36,354,548 | A | T | 931 |
| rs4711471 | 5.38E−03 | 0.031352 | 0.45574 | 36,921,233 | C | T | 932 |
| chr6: 36665767 | 5.45E−03 | −0.125587 | 0.01862 | 36,665,767 | A | G | 933 |
| chr6: 37148839 | 5.49E−03 | 0.053277 | 0.849092 | 37,148,839 | C | T | 934 |
| chr6: 36696279 | 5.51E−03 | 0.326213 | 0.004339 | 36,696,279 | A | G | 935 |
| chr6: 36729139 | 5.54E−03 | −0.149407 | 0.985856 | 36,729,139 | A | G | 936 |
| rs3778022 | 5.59E−03 | 0.043761 | 0.862796 | 37,056,224 | C | T | 937 |
| rs10947595 | 5.62E−03 | −0.033683 | 0.689545 | 36,353,239 | T | C | 938 |
| chr6: 35837314 | 5.67E−03 | 0.044841 | 0.276259 | 35,837,314 | A | G | 939 |
| rs11550973 | 5.73E−03 | −0.14267 | 0.977484 | 36,762,942 | A | G | 940 |
| rs9462167 | 5.79E−03 | 0.033062 | 0.315519 | 36,348,089 | A | G | 941 |
| chr6: 37267550 | 5.79E−03 | −0.030893 | 0.516379 | 37,267,550 | A | T | 942 |
| rs2234066 | 5.92E−03 | −0.146617 | 0.983951 | 36,463,821 | G | T | 943 |
| rs6457939 | 5.96E−03 | 0.033077 | 0.311341 | 36,771,161 | C | T | 944 |
| chr6: 36756095 | 5.99E−03 | 0.057117 | 0.135769 | 36,756,095 | C | G | 945 |
| rs236458 | 6.06E−03 | −0.03232 | 0.383448 | 36,808,986 | A | G | 946 |
| rs6906101 | 6.32E−03 | −0.031109 | 0.603973 | 36,775,588 | A | G | 947 |
| rs236453 | 6.43E−03 | 0.031054 | 0.513853 | 36,810,252 | A | G | 948 |
| rs1753283 | 6.47E−03 | 0.21229 | 0.010519 | 37,063,236 | A | G | 949 |
| rs649804 | 6.62E−03 | −0.092315 | 0.964525 | 36,631,688 | A | T | 950 |
| rs1776447 | 6.64E−03 | −0.044111 | 0.131582 | 37,710,438 | T | C | 951 |
| chr6: 36578099 | 6.71E−03 | 0.306813 | 0.004806 | 36,578,099 | A | G | 952 |
| rs86702 | 6.75E−03 | −0.031345 | 0.381934 | 36,808,415 | T | C | 953 |
| chr6: 37135761 | 6.94E−03 | 0.082018 | 0.955277 | 37,135,761 | C | T | 954 |
| rs707992 | 7.06E−03 | −0.079507 | 0.039009 | 36,087,660 | A | G | 955 |
| chr6: 37019268 | 7.07E−03 | −0.041274 | 0.30547 | 37,019,268 | A | T | 956 |
| rs9357230 | 7.16E−03 | −0.030089 | 0.408957 | 36,963,360 | C | G | 957 |
| rs12664239 | 7.17E−03 | −0.030085 | 0.408964 | 36,960,498 | A | G | 958 |
| rs2395658 | 7.19E−03 | 0.030345 | 0.403628 | 36,774,728 | G | T | 959 |
| rs2395659 | 7.19E−03 | 0.030345 | 0.403628 | 36,774,729 | G | T | 960 |
| rs6457941 | 7.20E−03 | 0.030339 | 0.403672 | 36,775,043 | A | G | 961 |
| rs10807172 | 7.20E−03 | 0.030332 | 0.40373 | 36,775,861 | C | G | 962 |
| rs6905861 | 7.20E−03 | 0.030334 | 0.4037 | 36,775,245 | A | G | 963 |
| rs6928344 | 7.20E−03 | 0.030332 | 0.403713 | 36,775,343 | C | T | 964 |
| rs9470530 | 7.27E−03 | 0.082785 | 0.946616 | 37,271,954 | A | T | 965 |
| rs2293389 | 7.27E−03 | −0.042377 | 0.138305 | 37,053,260 | A | C | 966 |
| rs2788072 | 7.37E−03 | 0.029377 | 0.477653 | 37,274,753 | A | T | 967 |
| rs3176344 | 7.54E−03 | 0.077556 | 0.049128 | 36,758,525 | A | G | 968 |
| rs1015330 | 7.55E−03 | 0.035757 | 0.773277 | 36,894,050 | C | T | 969 |
| rs236451 | 7.56E−03 | −0.032443 | 0.344033 | 36,810,357 | A | G | 970 |
| rs2776790 | 7.68E−03 | −0.030836 | 0.536618 | 37,265,725 | C | T | 971 |
| rs6910832 | 7.76E−03 | −0.029252 | 0.524045 | 36,964,717 | T | C | 972 |
| rs2273109 | 7.81E−03 | 0.031703 | 0.69978 | 37,728,107 | A | G | 973 |
| rs588496 | 7.81E−03 | −0.078786 | 0.95924 | 36,584,025 | A | G | 974 |
| rs86703 | 7.83E−03 | 0.030351 | 0.618443 | 36,808,446 | C | T | 975 |
| rs3846873 | 7.83E−03 | 0.031694 | 0.699789 | 37,727,578 | A | G | 976 |
| rs236468 | 7.86E−03 | −0.030723 | 0.389956 | 36,804,882 | A | G | 977 |
| rs2842632 | 7.90E−03 | −0.029052 | 0.527457 | 37,273,796 | C | G | 978 |
| rs4714020 | 8.02E−03 | 0.029791 | 0.592779 | 36,956,027 | C | T | 979 |
| rs1830578 | 8.06E−03 | −0.04097 | 0.817923 | 36,489,180 | A | T | 980 |
| rs17624895 | 8.22E−03 | 0.041403 | 0.860362 | 37,056,013 | A | T | 981 |
| chr6: 37717751 | 8.24E−03 | 0.101227 | 0.032555 | 37,717,751 | C | T | 982 |
| rs62408382 | 8.28E−03 | −0.056439 | 0.093242 | 37,217,820 | A | G | 983 |
| rs3798478 | 8.44E−03 | 0.041266 | 0.860332 | 37,054,926 | C | T | 984 |
| rs72846864 | 8.58E−03 | 0.032493 | 0.700764 | 37,020,083 | A | G | 985 |
| rs753634 | 8.60E−03 | 0.041193 | 0.859747 | 37,042,672 | A | T | 986 |
| chr6: 36813797 | 8.68E−03 | 0.083737 | 0.040499 | 36,813,797 | A | G | 987 |
| rs11752000 | 8.69E−03 | −0.028678 | 0.519665 | 37,268,360 | G | T | 988 |
| rs2776791 | 8.77E−03 | 0.028497 | 0.474556 | 37,268,767 | A | G | 989 |
| rs900002 | 8.80E−03 | −0.028475 | 0.525472 | 37,269,369 | C | T | 990 |
| rs1757004 | 8.81E−03 | −0.028477 | 0.525475 | 37,269,101 | A | C | 991 |
| rs2842631 | 8.82E−03 | −0.028576 | 0.525194 | 37,273,687 | A | G | 992 |
| rs2788073 | 8.82E−03 | 0.028842 | 0.479678 | 37,274,867 | A | G | 993 |
| rs62408392 | 8.86E−03 | −0.068862 | 0.069925 | 37,253,444 | A | G | 994 |
| rs2776792 | 8.89E−03 | 0.028454 | 0.474483 | 37,270,263 | C | G | 995 |
| rs2842625 | 8.92E−03 | −0.028448 | 0.525531 | 37,270,534 | A | G | 996 |
| rs2293388 | 8.95E−03 | −0.04095 | 0.139722 | 37,053,040 | C | G | 997 |
| rs9394421 | 9.03E−03 | 0.028418 | 0.474427 | 37,271,122 | A | G | 998 |
| rs7758422 | 9.06E−03 | 0.048429 | 0.321273 | 36,452,348 | C | T | 999 |
| rs2842627 | 9.08E−03 | −0.028431 | 0.52556 | 37,272,892 | A | C | 1000 |
| rs9394422 | 9.09E−03 | 0.028405 | 0.47436 | 37,271,266 | A | G | 1001 |
| rs9368987 | 9.09E−03 | −0.028409 | 0.52561 | 37,272,132 | G | T | 1002 |
| rs2788070 | 9.16E−03 | 0.028393 | 0.474357 | 37,272,829 | G | T | 1003 |
| rs6934844 | 9.20E−03 | 0.028494 | 0.477982 | 36,961,287 | C | T | 1004 |
| chr6: 36865377 | 9.23E−03 | −0.110627 | 0.023431 | 36,865,377 | A | G | 1005 |
| rs2842629 | 9.24E−03 | −0.028374 | 0.525681 | 37,273,634 | A | G | 1006 |
| rs10807180 | 9.25E−03 | −0.028374 | 0.525269 | 37,272,675 | C | T | 1007 |
| rs9349017 | 9.28E−03 | −0.028727 | 0.508663 | 36,919,578 | A | G | 1008 |
| rs2265486 | 9.31E−03 | 0.028357 | 0.474288 | 37,274,308 | C | T | 1009 |
| rs2482014 | 9.33E−03 | 0.028352 | 0.474279 | 37,274,517 | G | T | 1010 |
| rs2485942 | 9.36E−03 | −0.028345 | 0.525739 | 37,274,520 | G | T | 1011 |
| rs2776794 | 9.41E−03 | −0.028334 | 0.525753 | 37,275,308 | A | G | 1012 |
| rs1766696 | 9.42E−03 | −0.028342 | 0.525692 | 37,275,430 | A | G | 1013 |
| rs9368990 | 9.43E−03 | −0.029205 | 0.506307 | 37,272,485 | C | T | 1014 |
| rs2622887 | 9.47E−03 | −0.028289 | 0.525843 | 37,273,426 | C | T | 1015 |
| rs10947687 | 9.50E−03 | −0.04193 | 0.132995 | 37,715,365 | G | T | 1016 |
| chr6: 36966993 | 9.58E−03 | 0.213443 | 0.991851 | 36,966,993 | C | T | 1017 |
| rs6457879 | 9.61E−03 | −0.042455 | 0.133758 | 36,188,031 | A | G | 1018 |
| rs2842628 | 9.71E−03 | 0.028208 | 0.474001 | 37,273,352 | A | C | 1019 |
| rs2622888 | 9.74E−03 | −0.028195 | 0.52603 | 37,273,485 | A | T | 1020 |
| rs190201 | 9.79E−03 | −0.029667 | 0.388822 | 36,806,263 | C | G | 1021 |
| rs236462 | 9.83E−03 | −0.029689 | 0.387241 | 36,806,272 | A | G | 1022 |
| rs9470501 | 9.84E−03 | −0.057052 | 0.09482 | 37,221,862 | C | T | 1023 |
| rs2788071 | 9.85E−03 | 0.028229 | 0.472625 | 37,273,806 | C | T | 1024 |
| rs7747099 | 9.90E−03 | −0.035998 | 0.808038 | 36,478,232 | C | T | 1025 |
| rs58214515 | 9.93E−03 | −0.028242 | 0.526198 | 36,935,394 | C | T | 1026 |
| rs12190328 | 9.96E−03 | 0.111444 | 0.971504 | 36,438,491 | C | T | 1027 |
| rs2482013 | 9.96E−03 | −0.028119 | 0.5261 | 37,272,784 | C | T | 1028 |
| chr6: 37105058 | 9.99E−03 | −0.126391 | 0.01401 | 37,105,058 | A | G | 1029 |

Shown is marker identity, p-value of the association, magnitude of effect (in units of fractions of standard deviations), frequency of effect allele, position in NCBI Build 36, identity of effect allele, identity of other allele, SEQ ID for the marker. It should be noted that a positive value for an effect represents a predicted increase in the interval by the effect allele, while a negative value for the effect represents a decrease (i.e. the allele correlated with the negative effect is predictive of a decreased value of the measure).

TABLE 24
Association results for QRS interval in a 2 Mb
region flanking rs1733724 on chromosome 10.

| | | | | | effect | other | SEQ ID |
|---|---|---|---|---|---|---|---|
| marker | P-value | effect | freq | position | allele | allele | NO: |
| rs1194743 | 1.64E−06 | −0.06424 | 0.788225 | 53,882,603 | C | T | 254 |
| rs1733724 | 1.65E−06 | 0.064142 | 0.211653 | 53,893,983 | A | G | 255 |
| rs1660768 | 1.80E−06 | −0.086762 | 0.747763 | 53,927,411 | C | T | 1030 |
| rs1822689 | 1.44E−04 | 0.044958 | 0.334457 | 52,902,908 | A | G | 1031 |
| rs293300 | 2.36E−04 | 0.043373 | 0.344204 | 52,901,452 | C | T | 1032 |
| rs1444400 | 2.39E−04 | −0.043345 | 0.65583 | 52,904,161 | A | G | 1033 |
| rs1444401 | 2.40E−04 | −0.043337 | 0.655839 | 52,904,934 | C | T | 1034 |
| rs10762173 | 2.44E−04 | 0.043301 | 0.344145 | 52,906,742 | A | G | 1035 |
| rs6480276 | 2.52E−04 | −0.043208 | 0.656361 | 52,907,458 | A | G | 1036 |
| rs293303 | 2.60E−04 | −0.043132 | 0.65657 | 52,907,727 | G | T | 1037 |
| rs72804145 | 6.73E−04 | −0.044201 | 0.627238 | 54,643,559 | C | T | 1038 |
| rs6480277 | 8.33E−04 | −0.03797 | 0.403846 | 52,907,653 | A | G | 1039 |
| rs7086422 | 9.58E−04 | −0.041523 | 0.280999 | 52,904,183 | A | T | 1040 |
| rs11819049 | 1.47E−03 | 0.035725 | 0.557925 | 52,894,756 | G | T | 1041 |
| rs10997697 | 1.47E−03 | −0.035165 | 0.454045 | 52,903,131 | G | T | 1042 |
| rs10997653 | 1.47E−03 | −0.035073 | 0.454106 | 52,897,035 | C | T | 1043 |
| rs10823055 | 1.48E−03 | −0.035145 | 0.45406 | 52,902,042 | G | T | 1044 |
| rs10997698 | 1.48E−03 | 0.035172 | 0.546226 | 52,903,212 | A | G | 1045 |
| chr10: 53895018 | 1.55E−03 | −0.811344 | 0.005792 | 53,895,018 | A | G | 1046 |
| rs10823045 | 1.88E−03 | −0.034606 | 0.462034 | 52,894,951 | C | T | 1047 |
| chr10: 53318983 | 1.95E−03 | 0.063728 | 0.806981 | 53,318,983 | A | C | 1048 |
| rs10823046 | 2.02E−03 | −0.034273 | 0.44461 | 52,895,127 | A | G | 1049 |
| rs10997707 | 2.03E−03 | −0.034325 | 0.444256 | 52,904,635 | C | T | 1050 |
| rs2204758 | 2.05E−03 | −0.04152 | 0.474983 | 54,005,381 | A | G | 1051 |
| rs12763554 | 2.40E−03 | 0.040307 | 0.229695 | 54,626,487 | A | G | 1052 |
| rs4935405 | 2.42E−03 | 0.040323 | 0.229334 | 54,633,858 | A | T | 1053 |
| rs11003448 | 2.45E−03 | 0.040438 | 0.228231 | 54,634,574 | A | G | 1054 |
| rs1947757 | 2.47E−03 | −0.040444 | 0.772122 | 54,635,488 | G | T | 1055 |
| rs4935064 | 2.49E−03 | 0.040445 | 0.227671 | 54,635,847 | A | T | 1056 |
| rs4935063 | 2.49E−03 | 0.04043 | 0.227699 | 54,635,708 | C | T | 1057 |
| rs11003452 | 2.62E−03 | −0.040423 | 0.773734 | 54,638,264 | G | T | 1058 |
| rs1444399 | 2.63E−03 | −0.033236 | 0.44389 | 52,896,208 | C | T | 1059 |
| rs11003457 | 2.70E−03 | −0.040428 | 0.774327 | 54,639,837 | C | T | 1060 |
| rs11003459 | 2.78E−03 | 0.040479 | 0.224216 | 54,642,766 | A | C | 1061 |
| chr10: 54758364 | 2.80E−03 | 0.251508 | 0.988748 | 54,758,364 | G | T | 1062 |
| chr10: 53289538 | 2.96E−03 | −0.683953 | 0.004533 | 53,289,538 | A | C | 1063 |
| rs293299 | 3.32E−03 | 0.038394 | 0.595613 | 52,894,489 | C | T | 1064 |
| chr10: 53800759 | 3.40E−03 | −0.090529 | 0.965244 | 53,800,759 | A | G | 1065 |
| chr10: 53956020 | 3.42E−03 | 0.068705 | 0.122152 | 53,956,020 | A | C | 1066 |
| chr10: 53803027 | 3.43E−03 | −0.090426 | 0.965262 | 53,803,027 | G | T | 1067 |
| rs10997677 | 3.56E−03 | 0.032423 | 0.565971 | 52,900,194 | C | T | 1068 |
| rs10997674 | 3.56E−03 | −0.032422 | 0.434025 | 52,900,083 | A | G | 1069 |
| rs10997680 | 3.56E−03 | 0.032427 | 0.56598 | 52,900,449 | A | G | 1070 |
| rs10997689 | 3.56E−03 | 0.032445 | 0.566023 | 52,901,690 | C | T | 1071 |
| rs10997673 | 3.56E−03 | −0.032418 | 0.434016 | 52,900,082 | G | T | 1072 |
| rs10997704 | 3.56E−03 | 0.032473 | 0.566095 | 52,904,530 | C | G | 1073 |
| rs10823063 | 3.66E−03 | 0.039223 | 0.760675 | 52,905,308 | A | C | 1074 |
| rs7099012 | 3.68E−03 | 0.032483 | 0.567971 | 52,904,054 | C | T | 1075 |
| rs35487357 | 3.69E−03 | −0.090088 | 0.965369 | 53,795,318 | G | T | 1076 |
| rs7475776 | 3.83E−03 | −0.037792 | 0.75859 | 54,621,021 | C | T | 1077 |
| rs1904055 | 3.90E−03 | −0.037494 | 0.758355 | 54,633,501 | A | C | 1078 |
| rs2384163 | 3.96E−03 | −0.039157 | 0.770859 | 54,638,622 | A | G | 1079 |
| rs2384164 | 3.97E−03 | 0.039149 | 0.229147 | 54,638,607 | C | T | 1080 |
| rs7893316 | 4.06E−03 | −0.038081 | 0.763844 | 54,640,124 | A | G | 1081 |
| rs4427482 | 4.09E−03 | −0.039124 | 0.780109 | 54,653,691 | C | T | 1082 |
| rs10824948 | 4.09E−03 | −0.038227 | 0.764469 | 54,641,955 | A | G | 1083 |
| rs10824949 | 4.11E−03 | 0.038201 | 0.234842 | 54,642,249 | C | T | 1084 |
| chr10: 54855843 | 4.15E−03 | −0.264039 | 0.006158 | 54,855,843 | A | C | 1085 |
| chr10: 53792299 | 4.29E−03 | 0.089136 | 0.034414 | 53,792,299 | C | T | 1086 |
| chr10: 53486939 | 4.52E−03 | 0.212315 | 0.993198 | 53,486,939 | A | G | 1087 |
| rs11003469 | 4.53E−03 | −0.038259 | 0.760925 | 54,654,665 | C | G | 1088 |
| chr10: 53505292 | 4.88E−03 | −0.208992 | 0.007455 | 53,505,292 | A | G | 1089 |
| chr10: 54643702 | 4.94E−03 | 0.057459 | 0.12899 | 54,643,702 | A | C | 1090 |
| chr10: 54104367 | 4.99E−03 | 0.065668 | 0.849988 | 54,104,367 | C | T | 1091 |
| rs12768931 | 5.14E−03 | 0.036391 | 0.243814 | 54,708,651 | A | G | 1092 |
| rs11003470 | 5.34E−03 | 0.037437 | 0.226097 | 54,656,453 | C | G | 1093 |
| rs10997657 | 5.57E−03 | −0.036549 | 0.246223 | 52,898,289 | A | T | 1094 |
| rs4935068 | 5.82E−03 | 0.036971 | 0.224359 | 54,659,289 | C | T | 1095 |
| chr10: 54837827 | 5.85E−03 | 0.31234 | 0.991998 | 54,837,827 | A | G | 1096 |
| chr10: 54753670 | 5.89E−03 | −0.047288 | 0.845708 | 54,753,670 | C | T | 1097 |
| chr10: 54844375 | 5.89E−03 | −0.115062 | 0.021768 | 54,844,375 | A | C | 1098 |
| rs1733725 | 5.90E−03 | −0.035229 | 0.7186 | 53,963,609 | A | G | 1099 |
| chr10: 54847515 | 5.98E−03 | −0.114928 | 0.021613 | 54,847,515 | A | G | 1100 |
| rs10997699 | 6.01E−03 | −0.036377 | 0.246484 | 52,903,244 | C | T | 1101 |
| chr10: 52934667 | 6.17E−03 | −0.1564 | 0.982538 | 52,934,667 | A | T | 1102 |
| rs72789121 | 6.18E−03 | −0.086357 | 0.966061 | 53,788,026 | A | G | 1103 |
| chr10: 52938997 | 6.18E−03 | 0.469787 | 0.0058 | 52,938,997 | G | T | 1104 |
| rs10824952 | 6.24E−03 | 0.036597 | 0.224111 | 54,661,875 | C | T | 1105 |
| chr10: 53998005 | 6.38E−03 | −0.131232 | 0.978244 | 53,998,005 | A | G | 1106 |
| rs16937295 | 6.51E−03 | 0.036372 | 0.224785 | 54,663,124 | A | G | 1107 |
| rs1903948 | 6.58E−03 | −0.036308 | 0.774508 | 54,663,943 | C | G | 1108 |
| rs12359399 | 6.62E−03 | −0.036214 | 0.239139 | 52,905,074 | A | C | 1109 |
| rs72797304 | 6.73E−03 | −0.044705 | 0.84877 | 53,020,574 | C | T | 1110 |
| rs11003500 | 6.96E−03 | 0.034821 | 0.241728 | 54,703,213 | G | T | 1111 |
| rs10824972 | 6.99E−03 | −0.034791 | 0.757999 | 54,702,500 | C | T | 1112 |
| rs12571947 | 7.06E−03 | −0.03477 | 0.75811 | 54,704,784 | A | G | 1113 |
| rs12572462 | 7.10E−03 | 0.034744 | 0.242046 | 54,704,993 | A | T | 1114 |
| rs12572208 | 7.10E−03 | −0.034743 | 0.757947 | 54,704,925 | A | G | 1115 |
| rs12573223 | 7.17E−03 | 0.034693 | 0.242348 | 54,704,962 | A | G | 1116 |
| rs5017564 | 7.26E−03 | −0.034738 | 0.758547 | 54,709,907 | A | C | 1117 |
| rs61844342 | 7.27E−03 | −0.053561 | 0.117716 | 54,209,134 | A | T | 1118 |
| rs10824954 | 7.36E−03 | −0.034471 | 0.757108 | 54,668,332 | C | T | 1119 |
| rs7085418 | 7.37E−03 | −0.034689 | 0.758396 | 54,709,239 | A | G | 1120 |
| rs10824955 | 7.37E−03 | −0.034462 | 0.757067 | 54,668,441 | C | T | 1121 |
| rs10824996 | 7.37E−03 | 0.03541 | 0.262132 | 54,735,339 | C | G | 1122 |
| rs10824979 | 7.39E−03 | −0.03467 | 0.758265 | 54,711,371 | C | T | 1123 |
| rs11003490 | 7.42E−03 | −0.034382 | 0.755851 | 54,676,334 | A | C | 1124 |
| rs10824978 | 7.43E−03 | −0.034678 | 0.75856 | 54,711,364 | C | T | 1125 |
| chr10: 54809472 | 7.43E−03 | 0.04452 | 0.720526 | 54,809,472 | A | G | 1126 |
| rs10508992 | 7.44E−03 | −0.03435 | 0.756611 | 54,669,439 | C | T | 1127 |
| rs1871071 | 7.45E−03 | −0.034339 | 0.756569 | 54,669,520 | A | G | 1128 |
| rs10824977 | 7.48E−03 | −0.034835 | 0.761311 | 54,711,260 | A | G | 1129 |
| rs2384174 | 7.52E−03 | 0.034658 | 0.23997 | 54,709,408 | A | G | 1130 |
| rs12770584 | 7.54E−03 | −0.034646 | 0.75983 | 54,708,792 | C | T | 1131 |
| rs10824957 | 7.58E−03 | −0.034286 | 0.756479 | 54,673,637 | G | T | 1132 |
| rs61862234 | 7.58E−03 | 0.034611 | 0.239965 | 54,708,016 | A | G | 1133 |
| rs12220148 | 7.59E−03 | −0.034282 | 0.756415 | 54,676,593 | A | G | 1134 |
| rs11003481 | 7.60E−03 | 0.034263 | 0.243341 | 54,670,038 | A | G | 1135 |
| rs1903963 | 7.60E−03 | 0.034261 | 0.243348 | 54,670,581 | G | T | 1136 |
| rs12219998 | 7.62E−03 | −0.036049 | 0.768808 | 54,746,198 | A | G | 1137 |
| rs11003496 | 7.63E−03 | 0.034148 | 0.245616 | 54,694,659 | T | C | 1138 |
| rs2891448 | 7.64E−03 | −0.034241 | 0.756669 | 54,673,344 | C | G | 1139 |
| rs10824958 | 7.67E−03 | −0.034225 | 0.756709 | 54,674,351 | A | G | 1140 |
| rs11003485 | 7.68E−03 | −0.034225 | 0.756682 | 54,675,558 | C | T | 1141 |
| chr10: 53818831 | 7.69E−03 | −0.049709 | 0.78008 | 53,818,831 | G | T | 1142 |
| rs10824982 | 7.70E−03 | 0.034581 | 0.23982 | 54,713,288 | A | G | 1143 |
| rs10762958 | 7.72E−03 | 0.034551 | 0.239775 | 54,711,002 | C | T | 1144 |
| rs10997722 | 7.72E−03 | −0.035461 | 0.236524 | 52,906,616 | G | T | 1145 |
| rs10824959 | 7.72E−03 | 0.034199 | 0.243271 | 54,678,249 | A | G | 1146 |
| rs11003491 | 7.73E−03 | 0.034192 | 0.243253 | 54,678,709 | A | T | 1147 |
| rs10762959 | 7.74E−03 | 0.034536 | 0.239515 | 54,711,055 | A | T | 1148 |
| rs4592336 | 7.75E−03 | −0.034522 | 0.760739 | 54,710,375 | C | T | 1149 |
| rs9633602 | 7.75E−03 | −0.034082 | 0.754517 | 54,689,232 | A | G | 1150 |
| rs9633603 | 7.75E−03 | 0.034082 | 0.245483 | 54,689,244 | A | G | 1151 |
| rs11003506 | 7.75E−03 | 0.034512 | 0.239559 | 54,709,073 | C | T | 1152 |
| rs10824976 | 7.76E−03 | 0.034531 | 0.23959 | 54,711,201 | C | G | 1153 |
| rs2384176 | 7.78E−03 | −0.03454 | 0.760389 | 54,712,378 | C | T | 1154 |
| rs10824980 | 7.79E−03 | 0.034505 | 0.239252 | 54,712,074 | G | T | 1155 |
| rs10824981 | 7.81E−03 | 0.034497 | 0.239248 | 54,712,256 | A | G | 1156 |
| rs10508993 | 7.84E−03 | 0.034478 | 0.239347 | 54,712,286 | A | G | 1157 |
| rs11003528 | 7.86E−03 | −0.035143 | 0.765431 | 54,729,270 | G | T | 1158 |
| rs2384178 | 7.88E−03 | 0.03447 | 0.239886 | 54,712,546 | A | T | 1159 |
| rs4935409 | 7.89E−03 | −0.03411 | 0.756821 | 54,682,301 | A | G | 1160 |
| rs7071141 | 7.93E−03 | −0.033979 | 0.754597 | 54,692,215 | C | T | 1161 |
| rs61859787 | 7.98E−03 | 0.036453 | 0.675132 | 54,772,196 | C | T | 1162 |
| rs11003492 | 7.99E−03 | 0.034069 | 0.243318 | 54,685,508 | A | G | 1163 |
| rs10823056 | 8.01E−03 | −0.035397 | 0.239679 | 52,902,114 | A | G | 1164 |
| rs12242179 | 8.12E−03 | 0.034236 | 0.241032 | 54,707,877 | A | G | 1165 |
| rs12763687 | 8.14E−03 | −0.034244 | 0.759132 | 54,708,883 | C | T | 1166 |
| rs10824963 | 8.15E−03 | −0.033858 | 0.754671 | 54,697,979 | A | G | 1167 |
| rs10824964 | 8.15E−03 | −0.033858 | 0.754671 | 54,698,123 | A | G | 1168 |
| chr10: 54698130 | 8.15E−03 | 0.033858 | 0.245329 | 54,698,130 | A | C | 1169 |
| rs4611109 | 8.15E−03 | 0.033857 | 0.245338 | 54,699,757 | C | T | 1170 |
| rs10824966 | 8.15E−03 | −0.033856 | 0.75466 | 54,700,017 | C | T | 1171 |
| rs10824967 | 8.15E−03 | −0.033856 | 0.754659 | 54,700,119 | A | C | 1172 |
| rs7893466 | 8.16E−03 | −0.033855 | 0.754654 | 54,700,401 | A | G | 1173 |
| rs10824968 | 8.16E−03 | −0.033855 | 0.754653 | 54,700,462 | G | T | 1174 |
| rs10824969 | 8.16E−03 | 0.033853 | 0.24536 | 54,701,083 | C | T | 1175 |
| rs10824970 | 8.16E−03 | −0.033853 | 0.754639 | 54,701,151 | A | G | 1176 |
| rs10824971 | 8.16E−03 | −0.033852 | 0.754636 | 54,701,475 | A | C | 1177 |
| rs7083984 | 8.19E−03 | 0.04751 | 0.303352 | 53,957,509 | A | G | 1178 |
| rs11003533 | 8.30E−03 | −0.03577 | 0.764187 | 54,733,956 | C | T | 1179 |
| rs57122015 | 8.36E−03 | −0.03589 | 0.770868 | 54,716,162 | A | G | 1180 |
| rs11003542 | 8.42E−03 | −0.035727 | 0.76991 | 54,735,955 | C | T | 1181 |
| rs35530863 | 8.42E−03 | 0.222023 | 0.990196 | 54,803,235 | C | G | 1182 |
| rs11003534 | 8.72E−03 | −0.035096 | 0.758558 | 54,733,977 | C | T | 1183 |
| chr10: 54805534 | 8.76E−03 | 0.109695 | 0.978254 | 54,805,534 | A | G | 1184 |
| chr10: 53533502 | 8.95E−03 | 0.192529 | 0.991187 | 53,533,502 | A | G | 1185 |
| rs11003478 | 8.96E−03 | 0.033797 | 0.343418 | 54,667,926 | A | G | 1186 |
| rs12768758 | 9.39E−03 | 0.035004 | 0.24711 | 54,708,578 | C | G | 1187 |
| rs12416467 | 9.41E−03 | −0.033385 | 0.271106 | 54,606,243 | A | G | 1188 |
| chr10: 54392639 | 9.60E−03 | 0.196393 | 0.983403 | 54,392,639 | A | T | 1189 |
| rs57989578 | 9.69E−03 | 0.033334 | 0.729273 | 54,606,150 | A | T | 1190 |
| chr10: 54380757 | 9.80E−03 | −0.165294 | 0.016277 | 54,380,757 | A | G | 1191 |
| rs12781547 | 9.90E−03 | 0.033942 | 0.236316 | 54,743,740 | A | G | 1192 |
| chr10: 53952604 | 9.91E−03 | 0.177414 | 0.980264 | 53,952,604 | G | T | 1193 |
| rs12775366 | 9.93E−03 | 0.034566 | 0.228292 | 54,737,123 | A | G | 1194 |

Shown is marker identity, p-value of the association, magnitude of effect (in units of fractions of standard deviations), frequency of effect allele, position in NCBI Build 36, identity of effect allele, identity of other allele, SEQ ID for the marker. It should be noted that a positive value for an effect represents an increase conferred by the effect allele, while a negative value for the effect represents a decrease (i.e. the allele correlated with the negative effect is predictive of a decreased value of the measure).

1. A method of determining a susceptibility to a vascular condition selected from the group consisting of: an abnormal electrocardiogram measure, Atrial Fibrillation, Atrial Flutter, and Stroke, in a human individual, the method comprising:
obtaining sequence data about a human individual identifying at least one allele of at least one polymorphic marker, wherein different alleles of the at least one polymorphic marker are associated with different susceptibilities to the condition in humans, and
determining susceptibility to the condition from the sequence data,
wherein the at least one polymorphic marker is selected from the group consisting of rs3825214, rs6795970, rs3807989, rs7660702, rs132311, rs1733724 and rs365990, and markers in linkage disequilibrium therewith, wherein the linkage disequilibrium is characterized by a value for r² of at least 0.2.

2. The method of claim 1, wherein the abnormal electrocardiogram measure is selected from the group consisting of: an increased QRS interval, an increased PR interval, an increased QT interval, sick sinus syndrome and/or an increased heart rate.

3. The method of claim 1, wherein the sequence data is nucleic acid sequence data obtained from a biological sample containing nucleic acid from the human individual.

4. The method of claim 3, comprising analyzing nucleic acid sequence data about at least two polymorphic markers.

5. The method of claim 3, wherein the nucleic acid sequence data is obtained using a method that comprises at least one procedure selected from:
(i) amplification of nucleic acid from the biological sample;
(ii) hybridization assay using a nucleic acid probe and nucleic acid from the biological sample; and
(iii) hybridization assay using a nucleic acid probe and nucleic acid obtained by amplification of the biological sample.

6. (canceled)

7. (canceled)

8. (canceled)

9. The method of claim 1, wherein the sequence data is amino acid sequence data.

10. The method of claim 1, wherein obtaining sequence of the at least one polymorphic marker comprises determining the presence or absence of at least one at-risk allele of the at least one polymorphic marker for the condition.

11. The method of claim 1, further comprising a step of preparing a report containing results from the determination, wherein said report is written in a computer readable medium, printed on paper, or displayed on a visual display.

12. The method of claim 11, wherein the amino acid substitution is a Valine to Alanine substitution in position 1073 of a human SCN10A protein.

13. The method of claim 11, wherein the amino acid substitution is an Alanine to Valine substitution in position 1101 of a human MYH6 protein.

14. (canceled)

15. (canceled)

16. A method of assessing a subject's risk for a vascular condition selected from the group consisting of: an abnormal electrocardiogram measure, Atrial Fibrillation, Atrial Flutter, and Stroke, the method comprising:
a) obtaining nucleic acid sequence information about the individual identifying at least one allele of at least one polymorphic marker in the genome of the individual;
b) representing the nucleic acid sequence information as digital genetic profile data;
c) transforming the digital genetic profile data to generate a risk assessment report of the condition for the subject; and
d) displaying the risk assessment report on an output device;
wherein the at least one polymorphic marker comprises at least one marker selected from the group consisting of rs3825214, rs6795970, rs3807989, rs7660702, rs132311, rs1733724 and rs365990, and markers in linkage disequilibrium therewith, wherein the linkage disequilibrium is characterized by a value for r² of at least 0.2.

17. A method for determining a susceptibility to a vascular condition selected from the group consisting of: an abnormal electrocardiogram measure, Atrial Fibrillation, Atrial Flutter, and Stroke, in a human subject, comprising determining the presence or absence of at least one allele of at least one polymorphic marker in a nucleic acid sample obtained from the individual, wherein the at least one polymorphic marker is selected from the group consisting of rs3825214, rs6795970, rs3807989, rs7660702, rs132311, rs1733724 and rs365990, and markers in linkage disequilibrium therewith, wherein the linkage disequilibrium is characterized by a value for r² of at least 0.2, and wherein determination of the presence of the at least one allele is indicative of susceptibility to the condition.

18. The method of claim 16, further comprising assessing the frequency of at least one haplotype comprising at least two polymorphic markers in the subject.

19. A method of assessing a susceptibility to a vascular condition selected from the group consisting of: an abnormal electrocardiogram measure, Atrial Fibrillation, Atrial Flutter, and Stroke, in a human subject, comprising
i. obtaining sequence information about the subject for at least one polymorphic marker selected from the group consisting of rs3825214, rs6795970, rs3807989, rs7660702, rs132311, rs1733724 and rs365990, and markers in linkage disequilibrium therewith, wherein the linkage disequilibrium is characterized by a value for r² of at least 0.2, wherein different alleles of the at least one polymorphic marker are associated with different susceptibilities to the condition in humans;
ii. identifying the presence or absence of at least one allele in the at least one polymorphic marker that correlates with increased occurrence of the condition in humans;
wherein determination of the presence of the at least one allele identifies the subject as having elevated susceptibility to the condition, and
wherein determination of the absence of the at least one allele identifies the subject as not having the elevated susceptibility.

20. The method of claim 1, wherein:
markers in linkage disequilibrium with rs3825214 are selected from the group consisting of rs6489952, rs1895593, rs7966567, rs8181608, rs10744818, rs8181683, rs8181627, rs10744819, rs6489953, rs10744820, rs1895587, rs9669457, rs6489955, rs7309910, rs7308120, rs2384409, rs2891503, rs7977083, rs1895597, rs7316919, rs6489956, rs883079, rs2113433, rs3825214, rs12367410, rs10507248, rs7955405, rs10744823, rs7312625, rs4767237, rs7135659, rs1895585, rs1946295, rs1946293, rs3825215, rs1895582, rs7964303, and rs17731569;
markers in linkage disequilibrium with rs6795970 are selected from the group consisting of rs6599240, rs11129800, rs11129801, rs11710006, rs11924846, rs9990137, rs6805187, rs7617547, rs6771157, rs4076737, rs12632942, rs7430477, rs6795970, rs6801957, rs7433306, rs6780103, rs6790396, rs6800541, rs7615140, rs6599250, rs6599251, rs7430451, rs6599254, rs6599255, rs12630795, rs6798015, rs6763876, rs6599256, rs7641844, rs7432804, rs7430439, rs7651106, rs6599257, rs7610489, rs7650384, rs4414778, and rs10212338;
markers in linkage disequilibrium with rs3807989 are selected from the group consisting of rs2157799, rs721994, rs1728723, rs2049902, rs11772856, rs1858810, rs7781492, rs10464649, rs12706089, rs7782281, rs4727831, rs768108, rs717957, rs1883049, rs6959099, rs6975771, rs6976316, rs6954077, rs728690, rs10228178, rs2402081, rs2270188, rs10271007, rs4730743, rs4727833, rs2109513, rs6466579, rs3919515, rs975028, rs2215448, rs2742125, rs3779512, rs9649394, rs1474510, rs3807986, rs6466584, rs6466585, rs1476833, rs976739, rs3807989, rs3801995, rs3815412, rs11773845, rs9886215, rs9886219, rs2109516, rs3757732, rs3757733, rs7804372, rs729949, rs3807990, rs3807992, rs3807994, rs6466587, rs6466588, rs1049314, rs8713, rs6867, rs1049337, rs6961215, rs6961388, rs10280730, rs10232369, rs6959106, rs7802124, rs7802438, rs1860588, rs2052106, rs11979486, rs10273326, rs6466589, rs7795356, rs2109517, rs2056865, rs2191503, rs4727835, rs7800573, rs6955302, and rs6978354;
markers in linkage disequilibrium with rs7660702 are selected from the group consisting of rs7698203, rs6849659, rs2101134, rs10017047, rs12648692, rs13134382, rs17010599, rs7655100, rs7677064, rs10033273, rs7439720, rs900204, rs11731040, rs931195, rs17010632, rs1871864, rs1871865, rs1482085, rs11735639, rs4413396, rs13146939, rs13152150, rs13128115, rs12509904, rs12650494, rs10012090, rs7691602, rs7692808, rs7658797, rs17010697, rs343860, rs13108523, rs1482094, rs7676486, rs7660702, rs2062098, rs1482091, rs6813860, rs994285, rs343853, rs343849, rs3889735, rs2601855, rs2601857, rs7682971, rs10516755, rs1020584, rs13106553, rs12510813, rs12507272, rs13137008, rs13112493, rs4693735, rs12507198, rs13111662, rs11732231, rs11736641, rs11097071, rs7674888, rs1966862, rs12054628, rs17010839, rs11945319, rs6831420, rs7680588, rs17010851, rs17010857, rs4693736, rs13105921, rs17010887, rs17010892, rs17395020, rs17399123, rs10516756, rs1452681, rs9790823, rs7683733, rs7662174, rs7684607, rs13118915, rs17010925, rs12503243, rs7675429, rs7689056, and rs7693640;
markers in linkage disequilibrium with rs132311 are selected from the group consisting of rs6457931, rs12207916, rs1321313, rs4713994, rs1321311, rs1321310, rs4331968, rs9470361, rs6930671, rs11969445, rs9470366, rs6936993, rs9470367, rs7756236, rs9462207, rs9368950, rs9462208, rs9462209, rs9462210, rs10807170, rs4713996, rs9394368, rs4713999, rs4711457, rs6930083, rs4714001, rs1321309, rs733590, rs2395655, rs3176352, rs12207548, rs12191972, rs7767246, rs6937605, and rs7762245;
markers in linkage disequilibrium with rs1733724 are selected from the group consisting of rs1149782, rs1149781, rs1194673, rs1149776, rs1149775, rs1149772, rs1149769, rs1194671, rs1194670, rs1194669, rs1194668, rs6480837, rs1209265, rs1194664, rs1194663, rs1660760, rs12355839, rs1194743, and rs1733724; and
markers in linkage disequilibrium with rs365990 are selected from the group consisting of rs3811178, rs8022522, rs365990, rs445754, rs10149522, rs452036, rs412768, rs439735, rs388914, rs440466, rs2277474, rs7143356, rs12147570, rs2284651, rs7149517, rs2331979, rs3729833, rs765021, rs7140721, rs3729829, rs3729828, rs3729825, rs7159367, rs12894524, rs2277475, rs12147533, rs743567, rs7157716, and rs2754163.

21. The method of claim 1, wherein the presence of at least one allele selected from the group consisting of the G allele of rs3825214, the A allele of rs6795970, the A allele of rs3807989, the T allele of rs7660702, the T allele of rs132311, the T allele of rs1733724 and the G allele of rs365990 is indicative of an increased susceptibility to a condition selected from the group of: an increased QRS interval, an increased PR interval, an increased QT interval, sick sinus syndrome, and an increased heart rate.

22. The method of claim 1, wherein the presence of at least one allele selected from the group consisting of the G allele of rs3825214, the A allele of rs6795970, the A allele of rs3807989, and the T allele of rs7660702 is indicative of susceptibility to an increased PR interval in the subject.

23. The method of claim 1, wherein the presence of at least one allele selected from the group consisting of the T allele of rs132311, the T allele of rs1733724 and the G allele of rs365990 is indicative of susceptibility to an increased QRS interval in the subject.

24. The method of claim 1, wherein the presence of the G allele of rs365990 is indicative of susceptibility to an increased heart rate in the subject.

25. The method of claim 1, wherein the presence of the G allele of rs382514 is indicative of susceptibility to an increased QT interval in the subject.

26. The method of claim 1, wherein the presence of the G allele of rs3825214 is indicative of susceptibility to advanced atrioventricular block (AVB) in the subject.

27. The method of claim 1, wherein the presence of the G allele of rs3825214 is indicative of susceptibility to pacemaker placement in the subject.

28. The method of claim 1, wherein the presence of the G allele of rs3825214 is indicative of susceptibility to a condition selected from the group consisting of: increased PR interval, increased QRS interval, increased QT interval, atrioventricular block, and pacemaker placement, in the subject.

29. The method of claim 1, wherein the presence of at least one allele selected from the group consisting of: the A allele of rs3825214 and the G allele of rs3807989, is indicative of increased susceptibility to Atrial Fibrillation or Atrial Flutter in the subject.

30. The method of claim 1, wherein the at least one allele is associated with a decreased susceptibility of the condition in humans.

31. A method of determining a susceptibility to a vascular condition selected from the group consisting of: an abnormal electrocardiogram measure, Atrial Fibrillation, Atrial Flutter, and Stroke, the method comprising:
obtaining sequence data about a human individual identifying at least one allele of at least one polymorphic marker, wherein different alleles of the at least one polymorphic marker are associated with different susceptibilities to the condition in humans, and
determining a susceptibility to the condition from the sequence data,
wherein the at least one polymorphic marker is a marker associated with a gene selected from the group consisting of: the human TBXS gene, the human SCN10A gene, the human CAV1 gene, the human ARHGAP24 gene, the human CDKN1A gene and the human MYH6 gene.

32. The method of claim 31, wherein the at least one polymorphic marker is selected from the group consisting of rs3825214, rs6795970, rs3807989, rs7660702, rs132311, rs1733724 and rs365990, and markers in linkage disequilibrium therewith, wherein the linkage disequilibrium is characterized by a value for r² of at least 0.2.

33. The method of claim 32, wherein:
the at least one marker associated with the human TBX5 gene is selected from the group consisting of rs3825214, and markers in linkage disequilibrium therewith, wherein the linkage disequilibrium is characterized by a value for r² of at least 0.2;
the at least one marker associated with the human SCN10A gene is selected from the group consisting of rs6795970, and markers in linkage disequilibrium therewith, wherein the linkage disequilibrium is characterized by a value for r² of at least 0.2;
the at least one marker associated with the human CAV1 gene is selected from the group consisting of rs3807989, and markers in linkage disequilibrium therewith, wherein the linkage disequilibrium is characterized by a value for r² of at least 0.2;
the at least one marker associated with the human ARHGAP24 gene is selected from the group consisting of rs7660702, and markers in linkage disequilibrium therewith, wherein the linkage disequilibrium is characterized by a value for r² of at least 0.2;
the at least one marker associated with the human CDKN1A gene is selected from the group consisting of rs132311, and markers in linkage disequilibrium therewith, wherein the linkage disequilibrium is characterized by a value for r² of at least 0.2; and
the at least one marker associated with the human MYH6 gene is selected from the group consisting of rs365990, and markers in linkage disequilibrium therewith, wherein the linkage disequilibrium is characterized by a value for r² of at least 0.2.

34. The method of claim 1, further comprising reporting the susceptibility to at least one entity selected from the group consisting of the individual, a guardian of the individual, a genetic service provider, a physician, a medical organization, and a medical insurer.

35. A method of identification of a marker for use in assessing susceptibility to a vascular condition selected from the group consisting of: an abnormal electrocardiogram measure, Atrial Fibrillation, Atrial Flutter, and Stroke, in human individuals, the method comprising
a. identifying at least one polymorphic marker in linkage disequilibrium with at least one marker selected from the group consisting of rs3825214, rs6795970, rs3807989, rs7660702, rs132311, rs1733724 and rs365990, wherein the linkage disequilibrium is characterized by a value for r² of at least 0.2;
b. obtaining sequence information about the at least one polymorphic marker in a group of individuals diagnosed with the condition; and
c. obtaining sequence information about the at least one polymorphic marker in a group of control individuals;
wherein determination of a significant difference in frequency of at least one allele in the at least one polymorphism in individuals diagnosed with the condition as compared with the frequency of the at least one allele in the control group is indicative of the at least one polymorphism being useful for assessing susceptibility to the condition.

36. The method of claim 35, wherein an increase in frequency of the at least one allele in the at least one polymorphism in individuals diagnosed with the condition, as compared with the frequency of the at least one allele in the control group, is indicative of the at least one polymorphism being useful for assessing increased susceptibility to the condition.

37. The method of claim 35, wherein a decrease in frequency of the at least one allele in the at least one polymorphism in individuals diagnosed with the condition, as compared with the frequency of the at least one allele in the control group, is indicative of the at least one polymorphism being useful for assessing decreased susceptibility to, or protection against, the condition.

38-43. (canceled)

44. The method of claim 1, further comprising determining at least one biomarker in a sample from the individual.

45. The method of claim 44, wherein the biomarker is a protein biomarker selected from the group consisting of fibrin D-dimer, prothrombin activation fragment 1.2 (F1.2), thrombin-antithrombin III complexes (TAT), fibrinopeptide A (FPA), lipoprotein-associated phospholipase A2 (1p-PLA2), beta-thromboglobulin, platelet factor 4, P-selectin, von Willebrand Factor, pro-natriuretic peptide (BNP), matrix metalloproteinase-9 (MMP-9), PARK7, nucleoside diphosphate kinase (NDKA), tau, neuron-specific enolase, B-type neurotrophic growth factor, astroglial protein S-100b, glial fibrillary acidic protein, C-reactive protein, serum amyloid A, matrix metalloproteinase-9, vascular and intracellular cell adhesion molecules, tumor necrosis factor alpha, and interleukins, including interleukin-1, -6, and -8.

46. A kit for assessing susceptibility to a vascular condition selected from the group consisting of: an abnormal electrocardiogram measure, Atrial Fibrillation, Atrial Flutter, and Stroke, the kit comprising:
reagents for selectively detecting at least one allele of at least one polymorphic marker in the genome of the individual, wherein the polymorphic marker is selected from the group consisting of rs3825214, rs6795970, rs3807989, rs7660702, rs132311, rs1733724 and rs365990, and markers in linkage disequilibrium therewith, wherein the linkage disequilibrium is characterized by a value for r² of at least 0.2, and
a collection of data comprising correlation data between the at least one polymorphism and susceptibility to the condition.

47. The kit of claim 46, wherein the collection of data is on a computer-readable medium.

48. The kit of claim 46, wherein the kit comprises reagents for detecting no more than 100 alleles, or no more than 20 alleles, in the genome of the individual.

49-52. (canceled)

53. A computer-readable medium having computer executable instructions for determining susceptibility to a vascular condition selected from the group consisting of: an abnormal electrocardiogram measure, Atrial Fibrillation, Atrial Flutter, and Stroke, the computer readable medium comprising:
data indicative of at least one polymorphic marker;
a routine stored on the computer readable medium and adapted to be executed by a processor to determine risk of developing the condition for the at least one polymorphic marker;
wherein the at least one polymorphic marker is selected from the group consisting of rs3825214, rs6795970, rs3807989, rs7660702, rs132311, rs1733724 and rs365990, and markers in linkage disequilibrium therewith, wherein the linkage disequilibrium is characterized by a value for r² of at least 0.2.

54. The computer-readable medium of claim 53, wherein the medium contains data indicative of at least two polymorphic markers.

55. (canceled)

56. An apparatus for determining a genetic indicator for a vascular condition selected from the group consisting of: an abnormal electrocardiogram measure, Atrial Fibrillation, Atrial Flutter, and Stroke, in a human individual, comprising:
a processor;
a computer readable memory having computer executable instructions adapted to be executed on the processor to analyze marker and/or haplotype information for at least one human individual with respect to at least one polymorphic marker selected from the group consisting of rs3825214, rs6795970, rs3807989, rs7660702, rs132311, rs1733724 and rs365990, and markers in linkage disequilibrium therewith, wherein the linkage disequilibrium is characterized by a value for r² of at least 0.2, and generate an output based on the marker or haplotype information, wherein the output comprises a measure of susceptibility of the at least one marker or haplotype as a genetic indicator of the condition for the human individual.

57. The apparatus according to claim 56, wherein the computer readable memory further comprises data indicative of the risk of developing the condition associated with at least one allele of at least one polymorphic marker or at least one haplotype, and wherein a risk measure for the human individual is based on a comparison of the at least one marker and/or haplotype status for the human individual to the risk of the condition associated with the at least one allele of the at least one polymorphic marker or the at least one haplotype.

58. The apparatus according to claim 56, wherein the computer readable memory further comprises data indicative of the frequency of at least one allele of at least one
polymorphic marker or at least one haplotype in a plurality of individuals diagnosed with the condition, and data indicative of the frequency of at the least one allele of at least one polymorphic marker or at least one haplotype in a plurality of reference individuals, and wherein risk of developing the condition is based on a comparison of the frequency of the at least one allele or haplotype in individuals diagnosed with the condition and reference individuals.

59. The apparatus according to claim 56, wherein the risk measure is characterized by an Odds Ratio (OR) or a Relative Risk (RR).

60-64. (canceled)
